(19) United States
(12) Patent Application Publication
(10) Pub. No.: US 2013/0165464 A1
(43) Pub. Date: Jun. 27, 2013

(54) HETEROARYLS AND USES THEREOF

(71) Applicant: MILLENNIUM PHARMACEUTICALS, INC., Cambridge, MA (US)

(72) Inventors: Ryan W. Chau, Somerville, MA (US); Courtney A. Cullis, Bedford, MA (US); Matthew O. Duffey, Cambridge, MA (US); Krista E. Gipson, Medford, MA (US); Yongbo Hu, Winchester, MA (US); Gang Li, Westborough, MA (US); Michael D. Sintchak, Winchester, MA (US); Tricia J. Vos, Winchester, MA (US)

(73) Assignee: MILLENNIUM PHARMACEUTICALS, INC., Cambridge, MA (US)

(21) Appl. No.: 13/722,134

(22) Filed: Dec. 20, 2012

Related U.S. Application Data
(60) Provisional application No. 61/579,711, filed on Dec. 23, 2011.
(60) Provisional application No. 61/672,030, filed on Jul. 16, 2012.
(60) Provisional application No. 61/716,172, filed on Oct. 19, 2012.

(51) Int. Cl. C07D 401/14; C07D 401/04; C07D 471/04; A61K 31/444; C07D 403/04; A61K 31/506; A61K 45/06; A61K 31/4439; A61K 31/437

(52) U.S. Cl. CPC C07D 401/14; A61K 31/4439; C07D 401/04; C07D 471/04; A61K 31/437; C07D 403/04; A61K 31/506; A61K 45/06; A61K 31/444
USPC 514/275; 546/272.4; 514/341; 546/279.1; 514/343; 546/113; 514/300; 546/275.4; 544/324; 546/272.7; 546/276.4; 546/256; 514/333

(57) ABSTRACT

This invention provides compounds of formula IB:

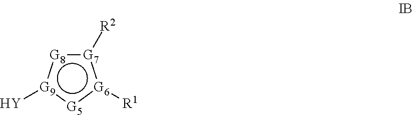

and also provides compounds of formulas ID, IIB, VB, and IIC:

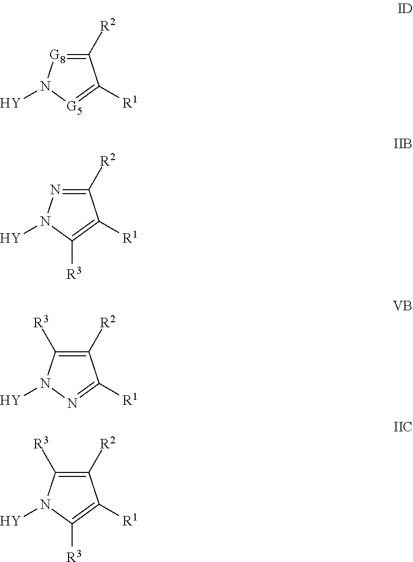

wherein HY, R¹, R², G₅, G₆, G₇, G₈, and G₉ are as described in the specification. The compounds are inhibitors of VPS34 and/or PI3K and are thus useful for treating proliferative, inflammatory, or cardiovascular disorders.

This application claims priority from U.S. Provisional Patent Application Ser. No. 61/579,711, filed on Dec. 23, 2011, U.S. Provisional Patent Application Ser. No. 61/672,030, filed on Jul. 16, 2012, and U.S. Provisional Patent Application Ser. No. 61/716,172, filed on Oct. 19, 2012.

### BACKGROUND OF THE INVENTION

Phosphatidylinositol 3-kinase (PI3K) is a family of lipid kinases that phosphorylate phosphatidylinositol at the 3′ position of the inositol ring. PI3K is comprised of several classes of genes, including Class IA, IB, II and III and some of these classes contain several isoforms (reviewed in Engelman et al., Nature Review Genetics 7:606-619 (2006)). Adding to the complexity of this family is the fact that PI3Ks function as heterodimers, comprising a catalytic domain and a regulatory domain. The PI3K family is structurally related to a larger group of lipid and serine/threonine protein kinases known as the phosphatidylinositol 3-kinase like kinases (PIKKs), which also includes DNA-PK, ATM, ATR, mTOR, TRRAP and SMG1.

PI3K is activated downstream of various mitogenic signals mediated through receptor tyrosine kinases, and subsequently stimulates a variety of biological outcomes; including increased cell survival, cell cycle progression, cell growth, cell metabolism, cell migration and angiogenesis (reviewed in Cantley, Science 296:1655-57 (2002); Hennessy et al., Nature Reviews Drug Discovery 4:988-1004 (2005); Engelman et al., Nature Review Genetics 7:606-619 (2006)). Thus, PI3K hyper-activation is associated with a number of hyper-proliferative, inflammatory, or cardiovascular disorders; including cancer, inflammation, and cardiovascular disease.

There are a number of genetic aberrations that lead to constitutive PI3K signaling; including activating mutations in PI3K itself (Hennessy et al., Nature Reviews Drug Discovery 4:988-1004 (2005); reviewed in Bader et al., Nature Reviews Cancer 5:921-9 (2005)); RAS (reviewed in Downward Nature Reviews Cancer 3:11-22 (2003)) and upstream receptor tyrosine kinases (reviewed in Zwick et al., Trends in Molecular Medicine 8:17-23 (2002)) as well as inactivating mutations in the tumor suppressor PTEN (reviewed in Cully et al., Nature Reviews Cancer 6:184-92 (2006)). Mutations in each of these gene classes have proven to be oncogenic and are commonly found in a variety of cancers.

The molecules defined within this invention inhibit the activity of PI3K, and therefore may be useful for the treatment of proliferative, inflammatory, or cardiovascular disorders. Cases where PI3K pathway mutations have been linked to proliferative disorders where the molecules defined within this invention may have a therapeutic benefit include benign and malignant tumors and cancers from diverse lineage, including but not limited to those derived from colon (Samuels et al., Science 304:554 (2004); reviewed in Karakas et al., British Journal of Cancer 94: 455-59 (2006)), liver (reviewed in Karakas et al., British Journal of Cancer 94: 455-59 (2006)), intestine (reviewed in Hennessy et al., Nature Reviews Drug Discovery 4:988-1004 (2005)), stomach (Samuels et al., Science 304:554 (2004); reviewed in Karakas et al., British Journal of Cancer 94: 455-59 (2006)), esophagus (Phillips et al., International Journal of Cancer 118:2644-6 (2006)); pancreas (reviewed in Downward Nature Reviews Cancer 3:11-22 (2003)); skin (reviewed in Hennessy et al., Nature Reviews Drug Discovery 4:988-1004 (2005)), prostate (reviewed in Hennessy et al., Nature Reviews Drug Discovery 4:988-1004 (2005)), lung (Samuels et al., Science 304:554 (2004); reviewed in Karakas et al., British Journal of Cancer 94: 455-59 (2006)), breast (Samuels et al., Science 304:554 (2004); Isakoff et al., Can Res 65:10992-1000 (2005); reviewed in Karakas et al., British Journal of Cancer 94: 455-59 (2006)), endometrium (Oda et al., Can Res 65:10669-73 (2005); reviewed in Hennessy et al., Nature Reviews Drug Discovery 4:988-1004 (2005)), cervix (reviewed in Hennessy et al., Nature Reviews Drug Discovery 4:988-1004 (2005)); ovary (Shayesteh et al., Nature Genetics 21:99-102 (1999); reviewed in Karakas et al., British Journal of Cancer 94: 455-59 (2006)), testes (Moul et al., Genes Chromosomes Cancer 5:109-18 (1992); Di Vizio et al., Oncogene 24:1882-94 (2005)), hematological cells (reviewed in Karakas et al., British Journal of Cancer 94: 455-59 (2006); Hennessy et al., Nature Reviews Drug Discovery 4:988-1004 (2005)), pancreas (reviewed in Downward Nature Reviews Cancer 3:11-22 (2003)), thyroid (reviewed in Downward Nature Reviews Cancer 3:11-22 (2003); reviewed in Hennessy et al., Nature Reviews Drug Discovery 4:988-1004 (2005)); brain (Samuels et al., Science 304:554 (2004); reviewed in Karakas et al., British Journal of Cancer 94: 455-59 (2006)), bladder (Lopez-Knowles et al., Cancer Research 66:7401-7404 (2006); Hennessy et al., Nature Reviews Drug Discovery 4:988-1004 (2005)); kidney (reviewed in Downward Nature Reviews Cancer 3:11-22 (2003)) and Head and Neck (reviewed in Engelman et al., Nature Reviews Genetics 7:606-619 (2006)).

Other classes of disorders with aberrant PI3K pathway signaling where the molecules defined within this invention may have a therapeutic benefit include inflammatory and cardiovascular diseases, including but not limited to allergies/anaphylaxis (reviewed in Rommel et al., Nature Reviews Immunology 7:191-201 (2007)), acute and chronic inflammation (reviewed in Ruckle et al., Nature Reviews Drug Discovery 5:903-12 (2006); reviewed in Rommel et al., Nature Reviews Immunology 7:191-201 (2007)), rheumatoid arthritis (reviewed in Rommel et al., Nature Reviews Immunology 7:191-201 (2007)); autoimmunity disorders (reviewed in Ruckle et al., Nature Reviews Drug Discovery 5:903-12 (2006)), thrombosis (Jackson et al., Nature Medicine 11:507-14 (2005); reviewed in Ruckle et al., Nature Reviews Drug Discovery 5:903-12 (2006)), hypertension (reviewed in Ruckle et al., Nature Reviews Drug Discovery 5:903-12 (2006)), cardiac hypertrophy (reviewed in Proud et al., Cardiovascular Research 63:403-13 (2004)), and heart failure (reviewed in Mocanu et al., British Journal of Pharmacology 150:833-8 (2007)).

Vacuolar Protein Sorting 34 (VPS34) is the sole Class III PI3K family member. VPS34 functions in the formation and trafficking of multiple intracellular vesicles, including vacuoles, endosomes, multivessicular bodies, lysosomes and autophagosomes (reviewed in Backer Biochem J 2008; Yan and Backer Biochem J 2007). VPS34 carries out these activities by phosphorylating PtdIns forming PtdIns3P, resulting in the recruitment and localization of a variety of FYVE and PX domain containing effector proteins that facilitate vesicular formation, elongation and movement. At a cellular level, inhibition of VPS34 results in defects in protein sorting and autophagy. Broadly defined, autophagy is a regulated process whereby cells catabolize subcellular components targeted for degradation by enclosing them in double-membrane vesicles which then fuse with lysosomes. Autophagy has been best characterized as occurring during times of nutrient deprivation, but also plays a role in normal cellular and tissue homeostasis and functions, including the development of multiple tissue types, the immune response, clearance of neuronal aggregates and tumor suppression. In addition to functioning in vesicle formation and movement, VPS34 may also participate in several signal transduction pathways (reviewed in Backer Biochem J 2008). Given that VPS34 plays an important role in many critical cellular processes including autophagy, inhibitors of VPS34 may have therapeutic application in a number of diseases, including but not limited to cancer, muscular disorders, neurodegeneration, inflammatory disease, infectious disease and other age related illnesses (reviewed in Shintani and Klionshy Science 2004; Kondo et al Nat Rev Cancer 2005; Delgato et al Immunol Rev 2009).

Clearly, it would be beneficial to provide novel VPS34 and/or PI3K inhibitors that possess good therapeutic properties, especially for the treatment of proliferative, inflammatory, or cardiovascular disorders.

1. General Description of Compounds of the Invention:

This invention provides compounds that are inhibitors of VPS34 and/or PI3K, and accordingly are useful for the treatment of proliferative, inflammatory, or cardiovascular disorders. The compounds of this invention are represented by formula IB:

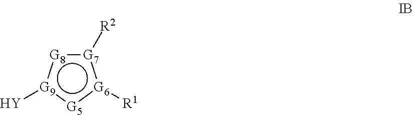

or a pharmaceutically acceptable salt thereof, wherein:

-G₅-G₆-G₇-G₈-G₉ is —CR³═C—C═N—N, —N═C—C═CR³—N, or —CR³═C—C═CR³—N;

each occurrence of R³ is independently hydrogen, —CN, halogen, —Z—R⁵, or an optionally substituted group selected from C₁₋₆ aliphatic and 3-10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
R^{3a} is hydrogen or an optionally substituted C₁₋₄ aliphatic, and
R⁵ is an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

R¹ is —CN, —C(O)N(R⁴)₂, —C(O)OR⁴, —C(NH)N(R⁴)₂, —NHCOR⁴, —NHSO₂R⁴, —NHCON(R⁴)₂, —NHCOOR⁴, —NHSO₂N(R⁴)₂, —CH₂OH, —CH₂N(R⁴)₂, —CH₂NHC(O)CH₃, —SO₂NR⁴₂, —CONHC(═NH)N(R⁴)₂, —NHSO₂OR⁴, or CY, wherein CY is an optionally substituted group selected from a 3-7-membered cycloaliphatic; a 4-10-membered heterocyclyl having 1-5 heteroatoms independently selected from nitrogen, oxygen, or sulfur; a 5-6-membered aryl, or 5-10-membered heteroaryl having 1-5 heteroatoms independently selected from nitrogen, oxygen, or sulfur; wherein:
R⁴ is hydrogen, —OH, or an optionally substituted group selected from C₁₋₆ aliphatic, 3-10-membered cycloaliphatic, 6-10-membered aryl, or 5-10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, or an optionally substituted group selected from C₁₋₆ aliphatic, —NH₂, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur; or
two occurrences of R⁴, taken together with a nitrogen atom to which they are bound, form an optionally substituted 4-7-membered heterocyclyl ring having 0-1 additional heteroatoms independently selected from nitrogen, oxygen, or sulfur;

R² is an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}, -T₂-R^{12a}, or —V₂-T₂-R^{12d}, and:

each occurrence of R^{12a} is independently halogen, —CN, —NO₂, —R^{12c}, —N(R^{12b})₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})C(O)R^{12b}, —N(R^{12e})C(O)N(R^{12b})₂, or —N(R^{12e})SO₂N(R^{12b})₂, or two occurrences of R^{12b}, taken together with a nitrogen atom to which they are bound, form an optionally substituted 4-7-membered heterocyclyl ring having 0-1 additional heteroatoms selected from nitrogen, oxygen, or sulfur;
each occurrence of R^{12b} is independently hydrogen or an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12c} is independently an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12d} is independently hydrogen or an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, —N(R^{12e})C(O)—, —N(R^{12e})SO₂—, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3-7 membered cycloaliphatic or heterocyclyl ring, wherein R¹³ is hydrogen or an optionally substituted C₁₋₄aliphatic group; and

HY is an optionally substituted group selected from:

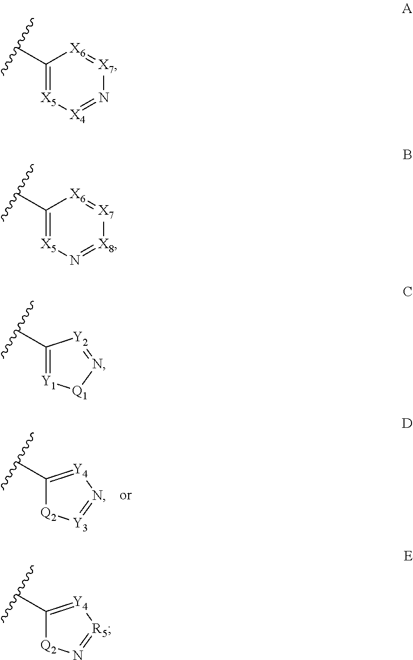

wherein each occurrence of X₄, X₅, X₆, X₇, and X₈ is independently —CR¹⁰ or N, provided no more than one occurrence of X₄, X₅, X₆, X₇, and X₈ is N, and at least two occurrences of CR¹⁰ are CH;

each occurrence of Q₁ and Q₂ is independently S, O or —NR⁹;

each occurrence of Y₁, Y₂, Y₃, Y₄, Y₅, Y₆, Y₇, and Y₈ is —CR¹⁰;
or wherein two adjacent occurrences of X₄ and X₅, X₆ and X₇, X₇ and X₈, Y₁ and Q₁, Y₃ and Q₂, or Y₄ and Y₅, taken together with the atom to which they are bound, form an optionally substituted fused group selected from 5-6-membered aryl, or 5-6-membered heteroaryl having 1-5 heteroatoms independently selected from nitrogen, oxygen, or sulfur;
   wherein R¹⁰ is —R^{10b}, —V₁—R^{10c}, -T₁-R^{10b}, or —V₁-T₁-R^{10b} wherein:
V₁ is —NR¹¹—, —NR¹¹—C(O)—, NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, NR¹¹C(NR¹¹)NR¹¹—, —NR¹¹S(O)₂—, —NR¹¹S(O)₂NR¹¹—, —C(O)—, —C(O)NR¹¹C(O)NR¹¹O—, —SO₂—, or —SO₂NR¹¹—;
each occurrence of R^{10a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O, N(R^{10a})C(O)N(R^{10a})N(R^{10a})S(O)₂N(R^{10a})—, —OC(O)—, or —C(O)N(R¹¹)—O— or wherein T₁ forms part of an optionally substituted 3-7 membered cycloaliphatic or heterocyclyl ring;
each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{10c} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, or R^{10a} and R^{10b}, taken together with a nitrogen atom to which they are bound, form an optionally substituted 4-7-membered heterocyclyl ring having 0-1 additional heteroatoms independently selected from nitrogen, oxygen, or sulfur;

each occurrence of R¹¹ is independently hydrogen, —C(O)R^{11a}, —CO₂R^{11a}, —C(O)N(R^{11a})₂, —C(O)N(R^{11a})—OR^{11a}, —SO₂R^{11a}, —SO₂N(R^{11a})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
wherein each occurrence of R^{11a} is independently hydrogen or an optionally substituted group selected from C₁₋₆aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;
   wherein each occurrence of R^{9a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;
wherein each occurrence of R^{9b} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3-6-membered heterocyclyl having 1-5 heteroatoms independently selected from nitrogen, oxygen, or sulfur, or 5-10-membered heteroaryl having 1-5 heteroatoms independently selected from nitrogen, oxygen, or sulfur; and
provided that R¹ is not an unsubstituted phenyl or a phenyl substituted only with one or two groups selected from methyl, tert-butyl, —CF₃ or halogen; and
R¹, R², and Hy are not all simultaneously pyridyl; and
provided that:
a) when Hy is selected from

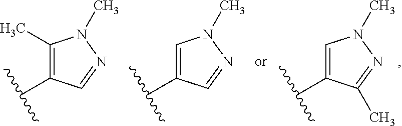

then
neither R¹ nor R² is the same as Hy;
b) when Hy is pyridazinyl and R² is phenyl, R¹ is not —CO₂Et;
c) Hy is not quinoxalinyl substituted with a sulfur containing group, or an optionally substituted

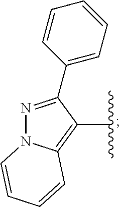 [SMILES: *c1c(-c2ccccc2)nn2ccccc12]

d) when R¹ is —CO₂H, then R² is not an optionally substituted ring selected from thienyl, furanyl, or cyclohexyl;
e) when R¹ is CN, then R² is not an unsubstituted cyclopropyl, or an optionally substituted ring selected from -phenyl-NH—CH₂-phenyl, -phenyl-NH—CH₂-pyridinyl, -phenyl-NH—C(O)-phenyl, or -phenyl-NH—C(O)-pyridyl;
f) R¹ is not an optionally substituted ring selected from

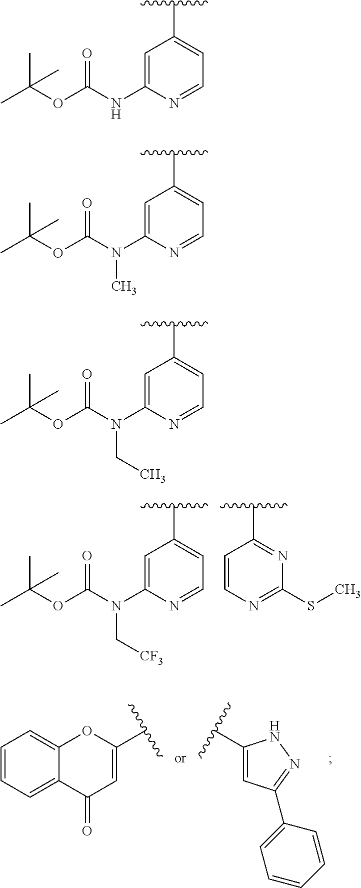

g) R¹ is not phenyl substituted with —C(O)N(H)C(H)(benzyl-OH)C(O)NH₂;
h) R¹ is not —NHC(O)CH₂N(isopropyl)C(O)—;
i) R¹ is not optionally substituted —CH₂NH-pyridyl;
j) neither R¹ nor R² is an optionally substituted ring selected from dibenzofuran, or

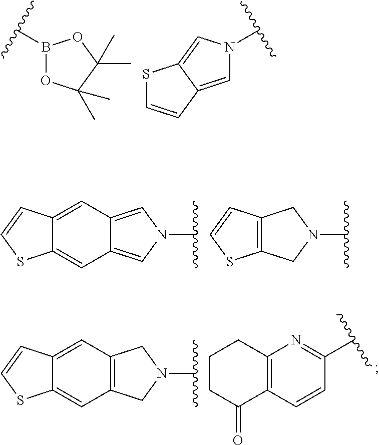

k) when either R¹ or R² is cyclopropyl, then the other of R¹ or R² is not phenyl substituted with —CF₃ or —OCF₃;
l) when R² is cyclopropyl, R³ is not chloro;
m) when R² is an optionally substituted phenyl, R¹ and R³ are not both —CO₂CH₃ or —CH₂OH;
n) when R² is dichlorophenyl, then R¹ is not an optionally substituted cyclobutyl or —CH₂—NH—CH₂—;
o) R² is not an optionally substituted

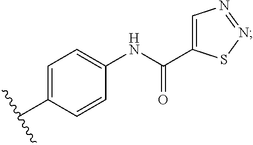 [SMILES: *c1ccc(NC(=O)c2cnns2)cc1]

p) R³ is not an optionally substituted

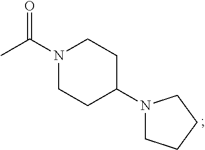

q) when -G₅-G₆-G₇-G₈-G₉ is —CR³═C—C═CR³—N, then R¹ is not —CN;
r) when -G₅-G₆-G₇-G₈-G₉ is —CR³═C—C═CR³—N, and Hy is quinolinyl, R² is not cyclopropyl;
s) the compound is other than:

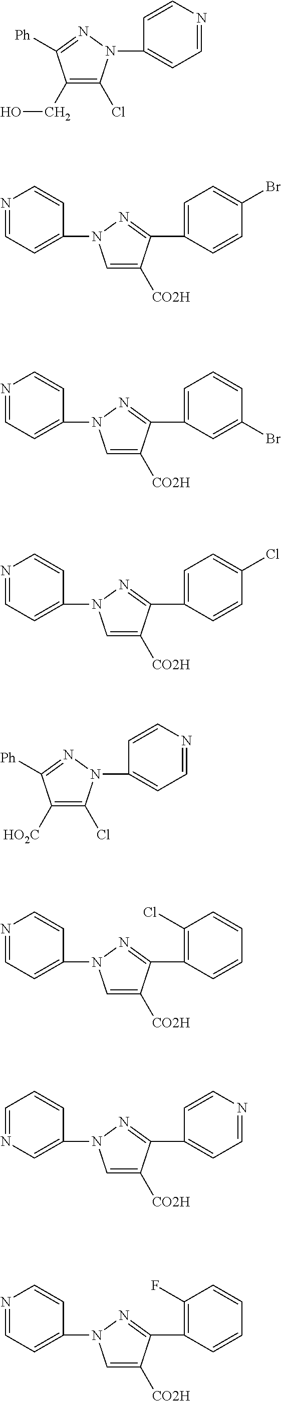

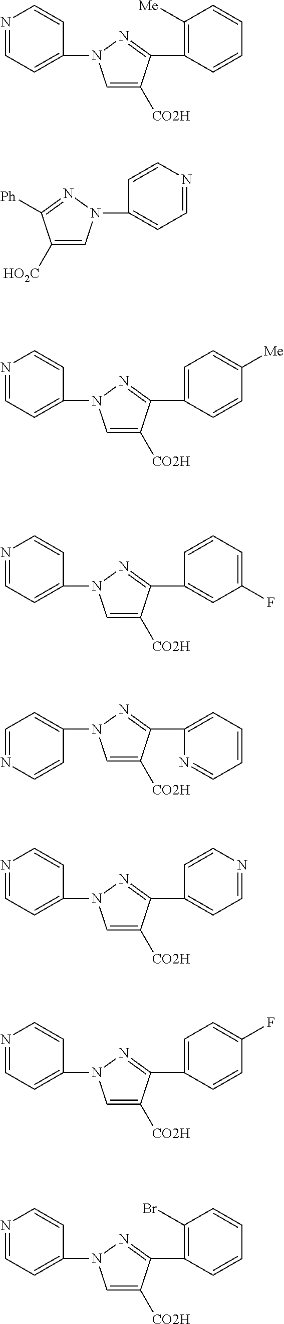

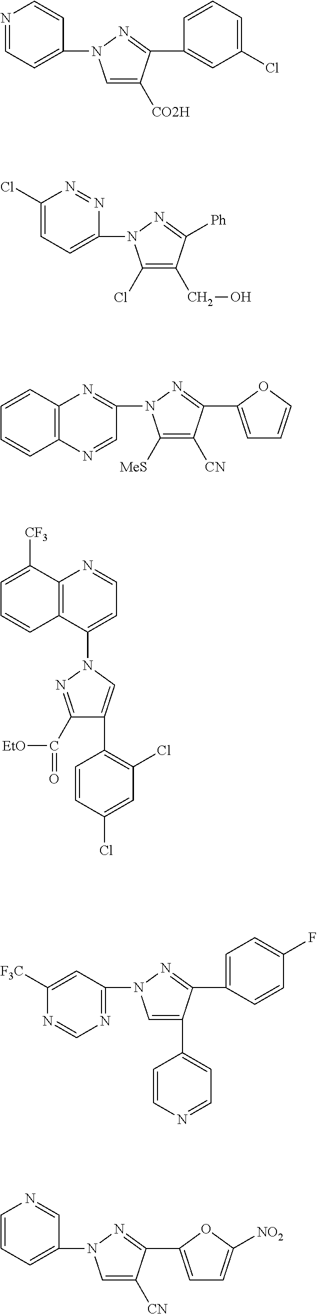 [SMILES: O=C(O)c1cn(-c2ccncc2)nc1-c1cccc(Cl)c1 ; OCc1c(-c2ccccc2)nn(-c2ccc(Cl)nn2)c1Cl ; CSc1c(C#N)c(-c2ccco2)nn1-c1cnc2ccccc2n1 ; CCOC(=O)c1nn(-c2ccnc3c(C(F)(F)F)cccc23)cc1-c1ccc(Cl)cc1Cl ; Fc1ccc(-c2nn(-c3cc(C(F)(F)F)ncn3)cc2-c2ccncc2)cc1 ; N#Cc1cn(-c2cccnc2)nc1-c1ccc([N+](=O)[O-])o1]

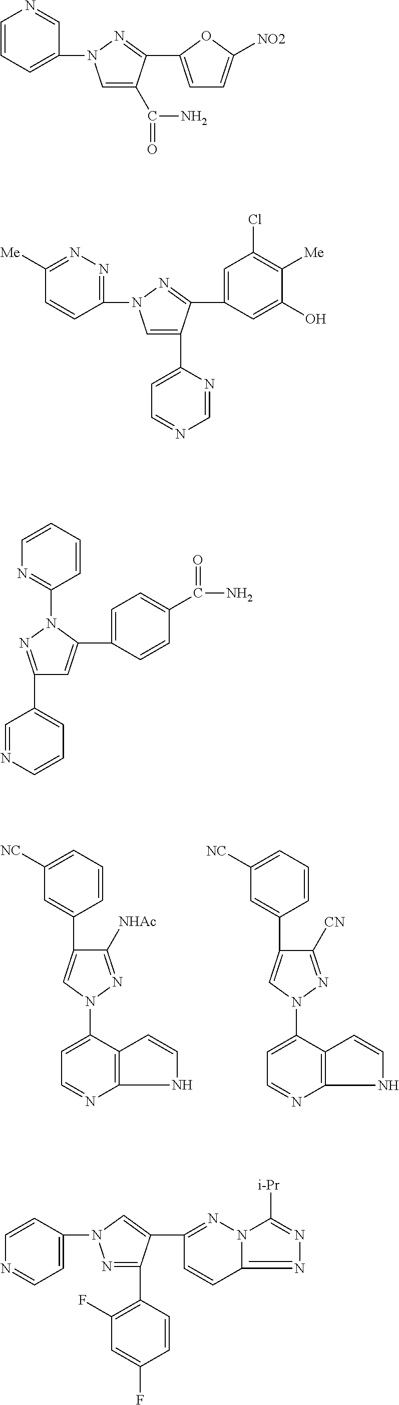 [SMILES: NC(=O)c1cn(-c2cccnc2)nc1-c1ccc([N+](=O)[O-])o1 ; Cc1ccc(-n2cc(-c3ccncn3)c(-c3cc(O)c(C)c(Cl)c3)n2)nn1 ; NC(=O)c1ccc(-c2cc(-c3cccnc3)nn2-c2ccccn2)cc1 ; CC(=O)Nc1nn(-c2ccnc3[nH]ccc23)cc1-c1cccc(C#N)c1 ; N#Cc1cccc(-c2cn(-c3ccnc4[nH]ccc34)nc2C#N)c1 ; CC(C)c1nnc2ccc(-c3cn(-c4ccncc4)nc3-c3ccc(F)cc3F)nn12]

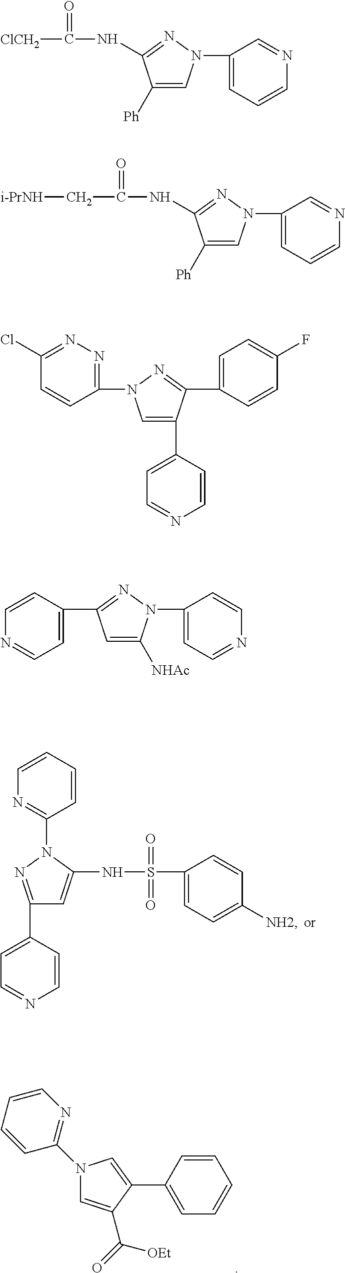

In another aspect, the compounds of this invention are represented by formula IB:

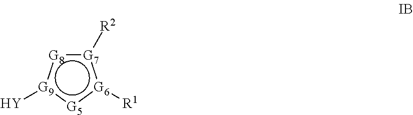

or a pharmaceutically acceptable salt thereof, wherein:

-G₅-G₆-G₇-G₈-G₉ is —CR³═C—C═N—N, —N═C—C═CR³—N, or —CR³═C—C═CR³—N;

each occurrence of R³ is independently hydrogen, —CN, halogen, —Z—R⁵, or an optionally substituted group selected from C₁₋₆ aliphatic and 3-10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
R^{3a} is hydrogen or an optionally substituted C₁₋₄ aliphatic, and
R⁵ is an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

R¹ is —C(O)N(R⁴)₂, —C(O)OR⁴, —C(NH)N(R⁴)₂, —NHCOR⁴, —NHSO₂R⁴, —NHCON(R⁴)₂, —NHCOOR⁴, —NHSO₂N(R⁴)₂, —CH₂OH, —CH₂N(R⁴)₂, —CH₂NHC(O)CH₃, —SO₂NR⁴₂, —CONHC(═NH)N(R⁴)₂, —NHSO₂OR⁴, or CY, wherein CY is an optionally substituted group selected from a 3-7-membered cycloaliphatic; a 4-10-membered heterocyclyl having 1-5 heteroatoms independently selected from nitrogen, oxygen, or sulfur; a 5-6-membered aryl, or 5-10-membered heteroaryl having 1-5 heteroatoms independently selected from nitrogen, oxygen, or sulfur; wherein:
R⁴ is hydrogen, —OH, or an optionally substituted group selected from C₁₋₆ aliphatic, 3-10-membered cycloaliphatic, 6-10-membered aryl, or 5-10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, or an optionally substituted group selected from C₁₋₆ aliphatic, —NH₂, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur; or
two occurrences of R⁴, taken together with a nitrogen atom to which they are bound, form an optionally substituted 4-7-membered heterocyclyl ring having 0-1 additional heteroatoms independently selected from nitrogen, oxygen, or sulfur;

R² is an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}-T₂-R^{12a} or —V₂-T₂-R^{12d}, and:
each occurrence of R^{12a} is independently halogen, —CN, —NO₂, —R^{12c}, —N(R^{12b})₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})SO₂R^{12c}, —N(R^{12e})C(O)OR^{12b}, —N(R^{12e})C(O)N(R^{12b})₂, or —N(R^{12e})SO₂N(R^{12b})₂, or two occurrences of R^{12b}, taken together with a nitrogen atom to which they are bound, form an optionally substituted 4-7-membered heterocyclyl ring having 0-1 additional heteroatoms selected from nitrogen, oxygen, or sulfur;
each occurrence of R^{12b} is independently hydrogen or an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12c} is independently an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12d} is independently hydrogen or an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, N(R^{12e})C(O)—, N(R^{12e})SO₂—, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3-7 membered cycloaliphatic or heterocyclyl ring, wherein R¹³ is hydrogen or an optionally substituted C₁₋₄aliphatic group; and

HY is an optionally substituted group selected from:

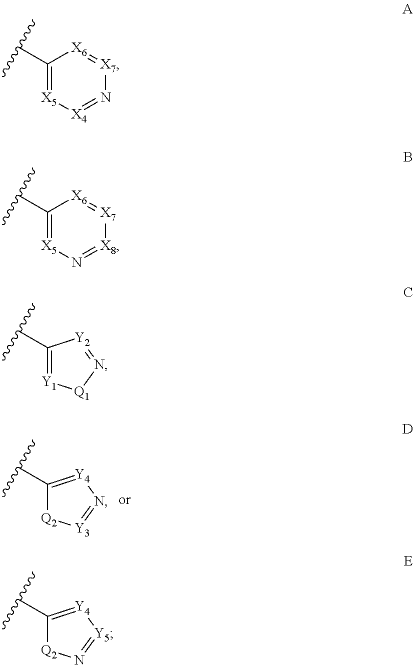

wherein each occurrence of X₄, X₅, X₆, X₇, and X₈ is independently —CR¹⁰ or N, provided no more than one occurrence of X₄, X₅, X₆, X₇, and X₈ is N, and at least two occurrences of CR¹⁰ are CH;

each occurrence of Q₁ and Q₂ is independently S, O or —NR⁹;

each occurrence of Y₁, Y₂, Y₃, Y₄, Y₅, Y₆, Y₇, and Y₈ is —CR¹⁰;
or wherein two adjacent occurrences of X₄ and X₅, X₆ and X₇, X₇ and X₈, Y₁ and Q₁, Y₃ and Q₂, or Y₄ and Y₅, taken together with the atom to which they are bound, form an optionally substituted fused group selected from 5-6-membered aryl, or 5-6-membered heteroaryl having 1-5 heteroatoms independently selected from nitrogen, oxygen, or sulfur;
   wherein R¹⁰ is R^{10b}, —V₁—R^{10c}, -T₁-R^{10b}, or —V₁-T₁-R^{10b} wherein:
V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
each occurrence of R^{10a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, —N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3-7 membered cycloaliphatic or heterocyclyl ring;
each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{10c} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, or R^{10a} and R^{10b}, taken together with a nitrogen atom to which they are bound, form an optionally substituted 4-7-membered heterocyclyl ring having 0-1 additional heteroatoms independently selected from nitrogen, oxygen, or sulfur;

each occurrence of R¹¹ is independently hydrogen, —C(O)R^{11a}, —CO₂R^{11a}, —C(O)N(R^{11a})₂, —C(O)N(R^{11a})—OR^{11a}, —SO₂R^{11a}, —SO₂N(R^{11a})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
wherein each occurrence of R^{11a} is independently hydrogen or an optionally substituted group selected from C₁₋₆aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;
   wherein each occurrence of R^{9a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;

wherein each occurrence of R^{9b} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3-6-membered heterocyclyl having 1-5 heteroatoms independently selected from nitrogen, oxygen, or sulfur, or 5-10-membered heteroaryl having 1-5 heteroatoms independently selected from nitrogen, oxygen, or sulfur; and
provided that R¹ is not an unsubstituted phenyl or a phenyl substituted only with one or two groups selected from methyl, tert-butyl, —CF₃ or halogen; and
R¹, R², and Hy are not all simultaneously pyridyl; and
provided that:
a) when Hy is selected from

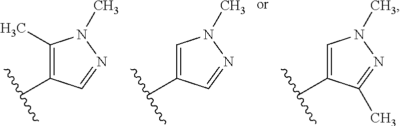

then
neither R¹ nor R² is the same as Hy;
b) when Hy is pyridazinyl and R² is phenyl, R¹ is not —CO₂Et;
c) Hy is not quinoxalinyl substituted with a sulfur containing group, or an optionally substituted

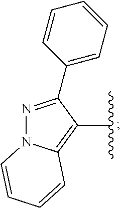 [SMILES: *c1c(-c2ccccc2)nn2ccccc12 ; ";"]

d) when R¹ is —CO₂H, then R² is not an optionally substituted ring selected from thienyl, furanyl, or cyclohexyl;
e) R¹ is not an optionally substituted ring selected from

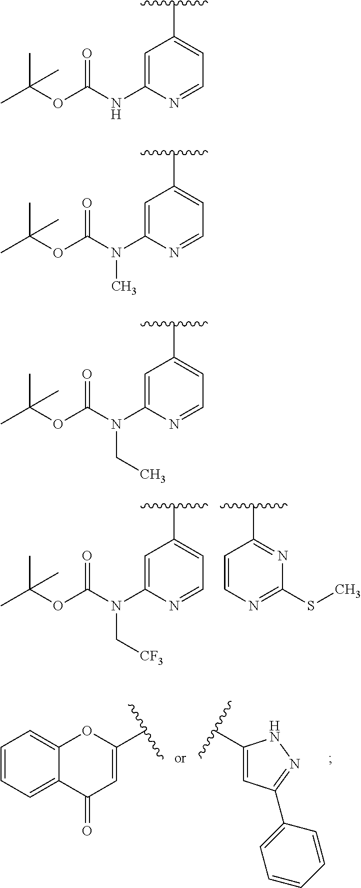

f) R¹ is not phenyl substituted with —C(O)N(H)C(H)(benzyl-OH)C(O)NH₂;
g) R¹ is not —NHC(O)CH₂N(isopropyl)C(O)—;
h) R¹ is not optionally substituted —CH₂NH-pyridyl;
i) neither R¹ nor R² is an optionally substituted ring selected from dibenzofuran, or

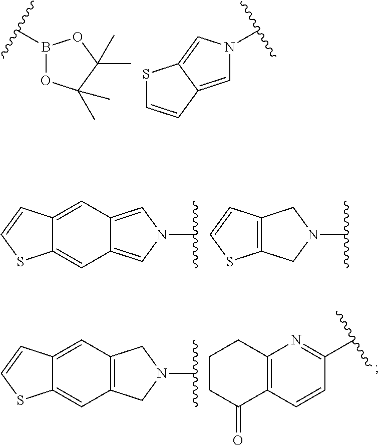

j) when either R¹ or R² is cyclopropyl, then the other of R¹ or R² is not phenyl substituted with —CF₃ or —OCF₃;
k) when R² is cyclopropyl, R³ is not chloro;
l) when R² is an optionally substituted phenyl, R¹ and R³ are not both —CO₂CH₃ or —CH₂OH;
m) when R² is dichlorophenyl, then R¹ is not an optionally substituted cyclobutyl or —CH₂—NH—CH₂—;
n) R² is not an optionally substituted

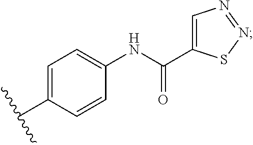 [SMILES: *c1ccc(NC(=O)c2cnns2)cc1]

o) R³ is not an optionally substituted

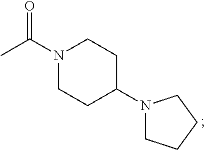

p) when -G₅-G₆-G₇-G₈-G₉ is —CR³═C—C═CR³—N, and Hy is quinolinyl, R² is not cyclopropyl;
q) the compound is other than:

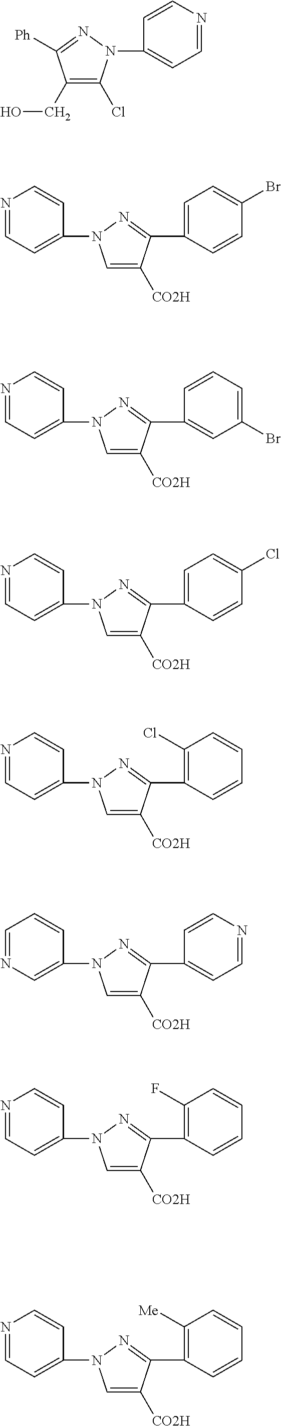

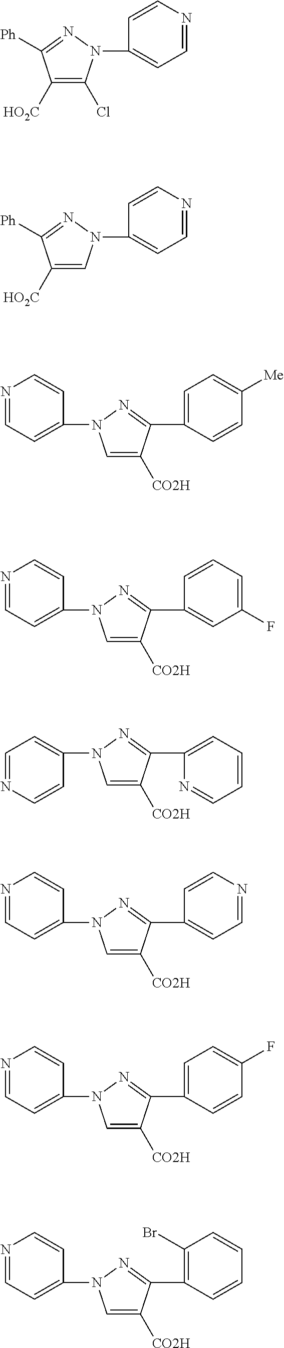

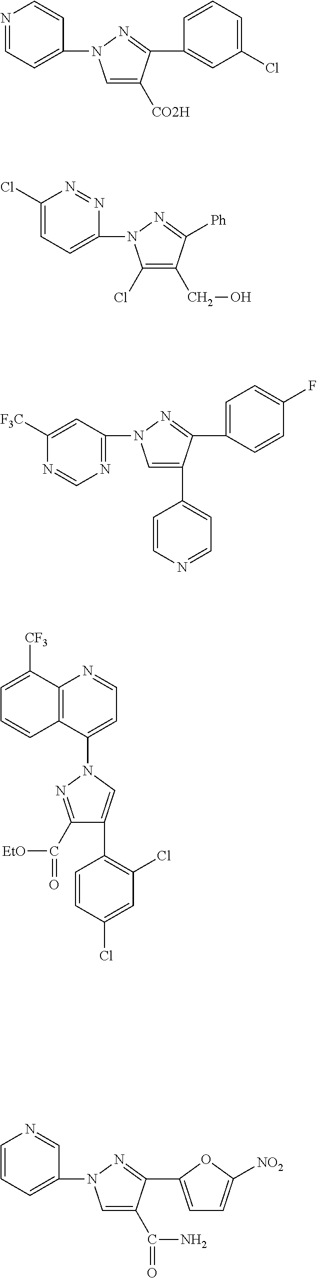 [SMILES: O=C(O)c1cn(-c2ccncc2)nc1-c1cccc(Cl)c1 ; OCc1c(-c2ccccc2)nn(-c2ccc(Cl)nn2)c1Cl ; Fc1ccc(-c2nn(-c3cc(C(F)(F)F)ncn3)cc2-c2ccncc2)cc1 ; CCOC(=O)c1nn(-c2ccnc3c(C(F)(F)F)cccc23)cc1-c1ccc(Cl)cc1Cl ; NC(=O)c1cn(-c2cccnc2)nc1-c1ccc([N+](=O)[O-])o1]

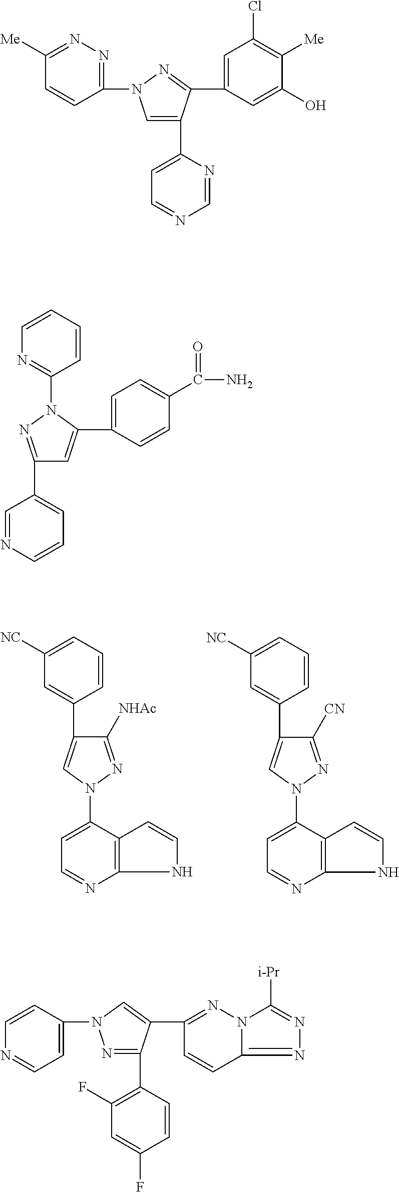 [SMILES: Cc1ccc(-n2cc(-c3ccncn3)c(-c3cc(O)c(C)c(Cl)c3)n2)nn1 ; NC(=O)c1ccc(-c2cc(-c3cccnc3)nn2-c2ccccn2)cc1 ; CC(=O)Nc1nn(-c2ccnc3[nH]ccc23)cc1-c1cccc(C#N)c1 ; N#Cc1cccc(-c2cn(-c3ccnc4[nH]ccc34)nc2C#N)c1 ; CC(C)c1nnc2ccc(-c3cn(-c4ccncc4)nc3-c3ccc(F)cc3F)nn12]

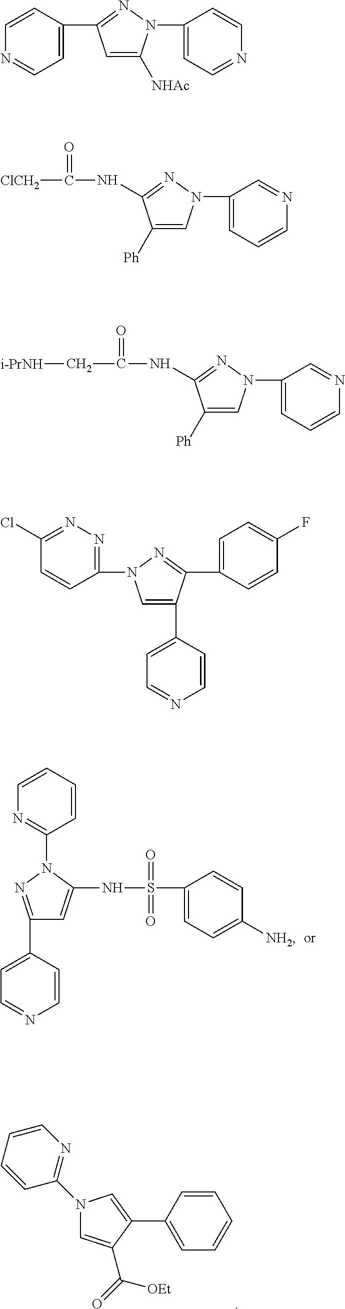

In another aspect, the compounds of this invention are represented by formula IB:

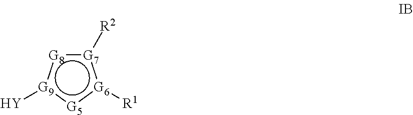

or a pharmaceutically acceptable salt thereof, wherein:

-G₅-G₆-G₇-G₈-G₉ is —CR³═C—C═N—N, —N═C—C═CR³—N, or —CR³═C—C═CR³—N;

each occurrence of R³ is independently hydrogen, —CN, halogen, —Z—R⁵, or an optionally substituted group selected from C₁₋₆ aliphatic and 3-10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
R^{3a} is hydrogen or an optionally substituted C₁₋₄ aliphatic, and
R⁵ is an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

R¹ is —CN, —C(O)N(R⁴)₂, —C(O)OR⁴¹, —C(NH)N(R⁴)₂, —NHCOR⁴, —NHSO₂R⁴, —NHCON(R⁴)₂, —NHCOOR⁴, —NHSO₂N(R⁴)₂, —CH₂OH, —CH₂N(R⁴)₂, —CH₂NHC(O)CH₃, —SO₂NR⁴₂, —CONHC(═NH)N(R⁴)₂, —NHSO₂OR⁴, or CY, wherein CY is an optionally substituted group selected from a 3-7-membered cycloaliphatic; a 4-10-membered heterocyclyl having 1-5 heteroatoms independently selected from nitrogen, oxygen, or sulfur; a 5-6-membered aryl, or 5-10-membered heteroaryl having 1-5 heteroatoms independently selected from nitrogen, oxygen, or sulfur; wherein:
R⁴¹ is an optionally substituted group selected from C₁₋₆ aliphatic, 3-10-membered cycloaliphatic, 6-10-membered aryl, or 5-10-membered heteroaryl having 1-5 heteroatoms independently selected from nitrogen, oxygen, or sulfur;
R⁴ is hydrogen, —OH, or an optionally substituted group selected from C₁₋₆ aliphatic, 3-10-membered cycloaliphatic, 6-10-membered aryl, or 5-10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, or an optionally substituted group selected from C₁₋₆ aliphatic, —NH₂, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur; or
two occurrences of R⁴, taken together with a nitrogen atom to which they are bound, form an optionally substituted 4-7-membered heterocyclyl ring having 0-1 additional heteroatoms independently selected from nitrogen, oxygen, or sulfur;

R² is an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}, -T₂-R^{12a}, or —V₂-T₂-R^{12d}, and:
each occurrence of R^{12a} is independently halogen, —CN, —NO₂, —R^{12c}, —N(R^{12b})₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})SO₂R^{12c}, —N(R^{12e})C(O)OR^{12b}, N(R^{12e})C(O)N(R^{12b})₂, or —N(R^{12e})SO₂N(R^{12b})₂, or two occurrences of R^{12b}, taken together with a nitrogen atom to which they are bound, form an optionally substituted 4-7-membered heterocyclyl ring having 0-1 additional heteroatoms selected from nitrogen, oxygen, or sulfur;
each occurrence of R^{12b} is independently hydrogen or an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12c} is independently an optionally substituted group selected from C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12d} is independently hydrogen or an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, N(R^{12e})C(O)—, —N(R^{12e})SO₂—, —N(R^{12e})C(O)O—, —N(R^{12e})C(O)N(R^{12e})—, N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3-7 membered cycloaliphatic or heterocyclyl ring, wherein R¹³ is hydrogen or an optionally substituted C₁₋₄aliphatic group; and

HY is an optionally substituted group selected from:

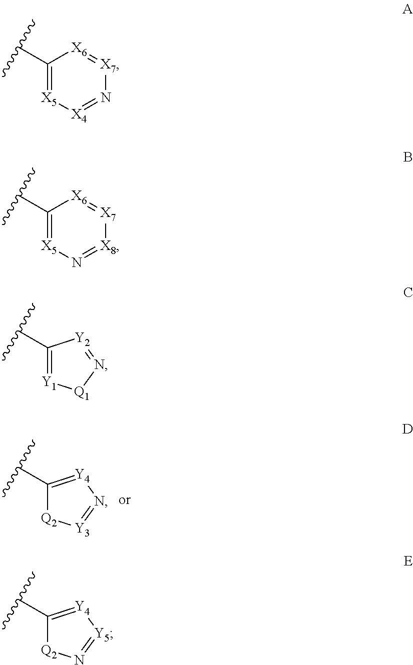

wherein each occurrence of X₄, X₅, X₆, X₇, and X₈ is independently —CR¹⁰ or N, provided no more than one occurrence of X₄, X₅, X₆, X₇, and X₈ is N, and at least two occurrences of CR¹⁰ are CH;

each occurrence of Q₁ and Q₂ is independently S, O or —NR⁹;

each occurrence of Y₁, Y₂, Y₃, Y₄, Y₅, Y₆, Y₇, and Y₈ is —CR¹⁰;
or wherein two adjacent occurrences of X₄ and X₅, X₆ and X₇, X₇ and X₈, Y₁ and Q₁, Y₃ and Q₂, or Y₄ and Y₅, taken together with the atom to which they are bound, form an optionally substituted fused group selected from 5-6-membered aryl, or 5-6-membered heteroaryl having 1-5 heteroatoms independently selected from nitrogen, oxygen, or sulfur;
   wherein R¹⁰ is —R^{10b}, —V₁—R^{10c}, -T₁-R^{10b}, or —V₁-T₁-R^{10b} wherein:
V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
each occurrence of R^{10a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂, —N(R^{11a})C(O)O—, —N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3-7 membered cycloaliphatic or heterocyclyl ring;
each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{10c} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, or
R^{10a} and R^{10b}, taken together with a nitrogen atom to which they are bound, form an optionally substituted 4-7-membered heterocyclyl ring having 0-1 additional heteroatoms independently selected from nitrogen, oxygen, or sulfur;

each occurrence of R¹¹ is independently hydrogen, —C(O)R^{11a}, —CO₂R^{11a}, —C(O)N(R^{11a})₂, C(O)N(R^{11a})—OR^{11a}, —SO₂R^{11a}, —SO₂N(R^{11a})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
wherein each occurrence of R^{11a} is independently hydrogen or an optionally substituted group selected from C₁₋₆aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;
wherein each occurrence of R^{9a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;

wherein each occurrence of R^{9b} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3-6-membered heterocyclyl having 1-5 heteroatoms independently selected from nitrogen, oxygen, or sulfur, or 5-10-membered heteroaryl having 1-5 heteroatoms independently selected from nitrogen, oxygen, or sulfur; and

provided that R¹ is not an unsubstituted phenyl or a phenyl substituted only with one or two groups selected from methyl, tert-butyl, —CF₃ or halogen; and

R¹, R², and Hy are not all simultaneously pyridyl; and

provided that:
a) when Hy is selected from

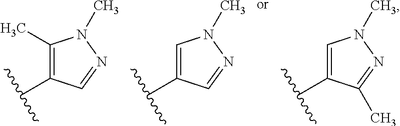

then neither R¹ nor R² is the same as Hy;
b) when Hy is pyridazinyl and R² is phenyl, R¹ is not —CO₂Et;
c) Hy is not quinoxalinyl substituted with a sulfur containing group, or an optionally substituted

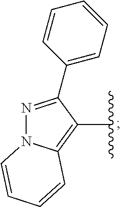 [SMILES: *c1c(-c2ccccc2)nn2ccccc12 ; ";"]

d) when R¹ is CN, then R² is not an unsubstituted cyclopropyl, or an optionally substituted ring selected from -phenyl-NH—CH₂-phenyl, -phenyl-NH—CH₂-pyridinyl, -phenyl-NH—C(O)-phenyl, or -phenyl-NH—C(O)-pyridyl;
e) R¹ is not an optionally substituted ring selected from

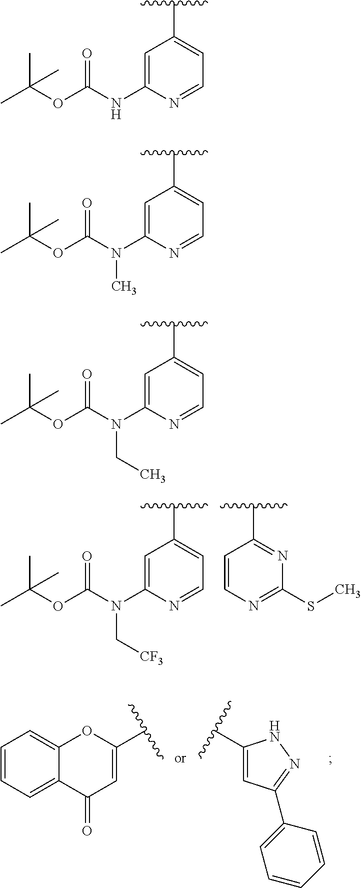

f) R¹ is not phenyl substituted with —C(O)N(H)C(H)(benzyl-OH)C(O)NH₂;
g) R¹ is not —NHC(O)CH₂N(isopropyl)C(O)—;
h) R¹ is not optionally substituted —CH₂NH-pyridyl;
i) neither R¹ nor R² is an optionally substituted ring selected from dibenzofuran, or

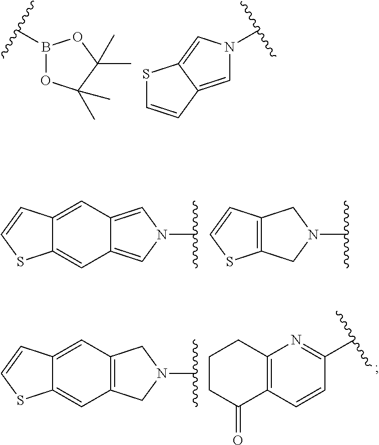

j) when either R¹ or R² is cyclopropyl, then the other of R¹ or R² is not phenyl substituted with —CF₃ or —OCF₃;
k) when R² is cyclopropyl, R³ is not chloro;
l) when R² is an optionally substituted phenyl, R¹ and R³ are not both —CO₂CH₃ or —CH₂OH;
m) when R² is dichlorophenyl, then R¹ is not an optionally substituted cyclobutyl or —CH₂—NH—CH₂—;
n) R² is not an optionally substituted

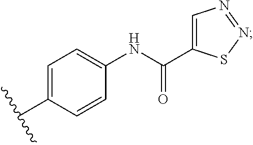 [SMILES: *c1ccc(NC(=O)c2cnns2)cc1]

o) R³ is not an optionally substituted

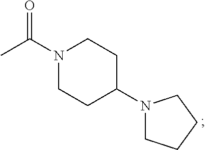

P) when -G₅-G₆-G₇-G₈-G₉ is —CR³═C—C═CR³—N, then R¹ is not —CN;
q) when -G₅-G₆-G₇-G₈-G₉ is —CR³═C—C═CR³—N, and Hy is quinolinyl, R² is not cyclopropyl;
r) the compound is other than:

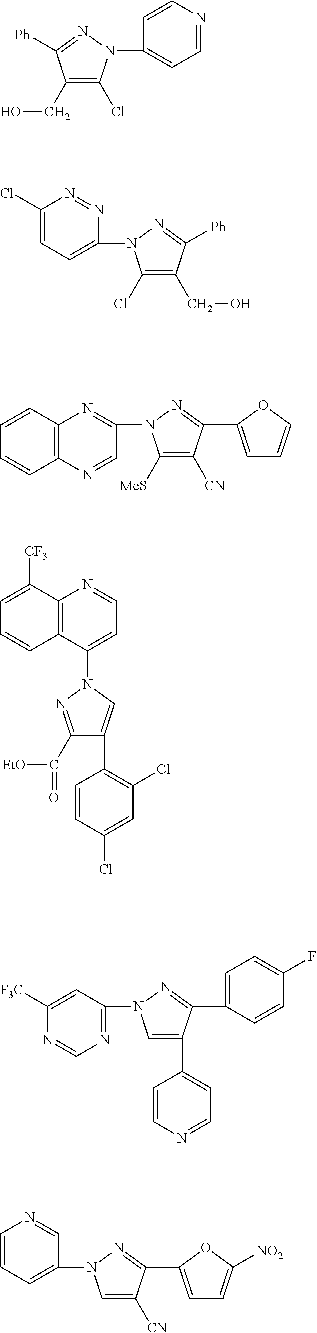 [SMILES: OCc1c(-c2ccccc2)nn(-c2ccncc2)c1Cl ; OCc1c(-c2ccccc2)nn(-c2ccc(Cl)nn2)c1Cl ; CSc1c(C#N)c(-c2ccco2)nn1-c1cnc2ccccc2n1 ; CCOC(=O)c1nn(-c2ccnc3c(C(F)(F)F)cccc23)cc1-c1ccc(Cl)cc1Cl ; Fc1ccc(-c2nn(-c3cc(C(F)(F)F)ncn3)cc2-c2ccncc2)cc1 ; N#Cc1cn(-c2cccnc2)nc1-c1ccc([N+](=O)[O-])o1]

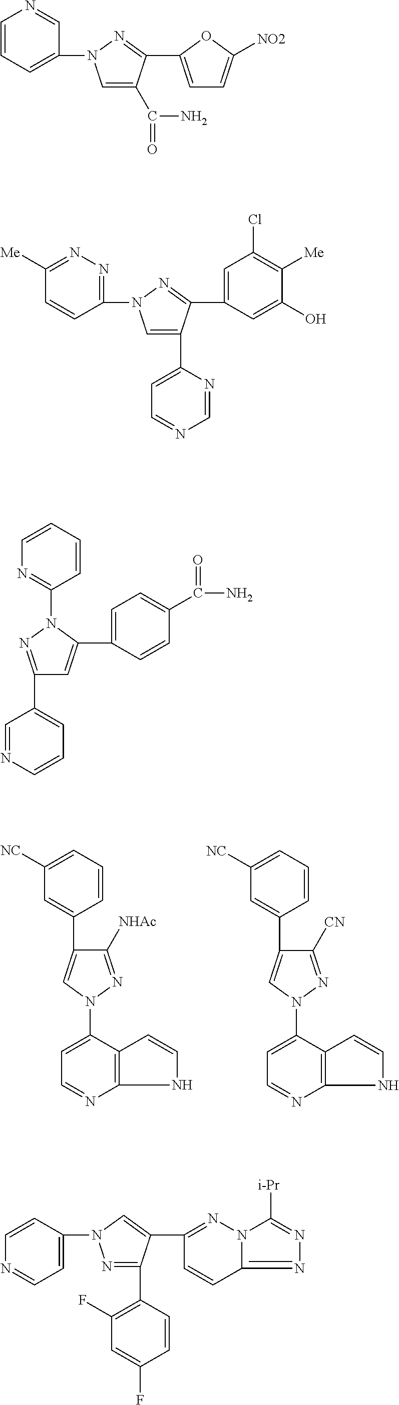 [SMILES: NC(=O)c1cn(-c2cccnc2)nc1-c1ccc([N+](=O)[O-])o1 ; Cc1ccc(-n2cc(-c3ccncn3)c(-c3cc(O)c(C)c(Cl)c3)n2)nn1 ; NC(=O)c1ccc(-c2cc(-c3cccnc3)nn2-c2ccccn2)cc1 ; CC(=O)Nc1nn(-c2ccnc3[nH]ccc23)cc1-c1cccc(C#N)c1 ; N#Cc1cccc(-c2cn(-c3ccnc4[nH]ccc34)nc2C#N)c1 ; CC(C)c1nnc2ccc(-c3cn(-c4ccncc4)nc3-c3ccc(F)cc3F)nn12]

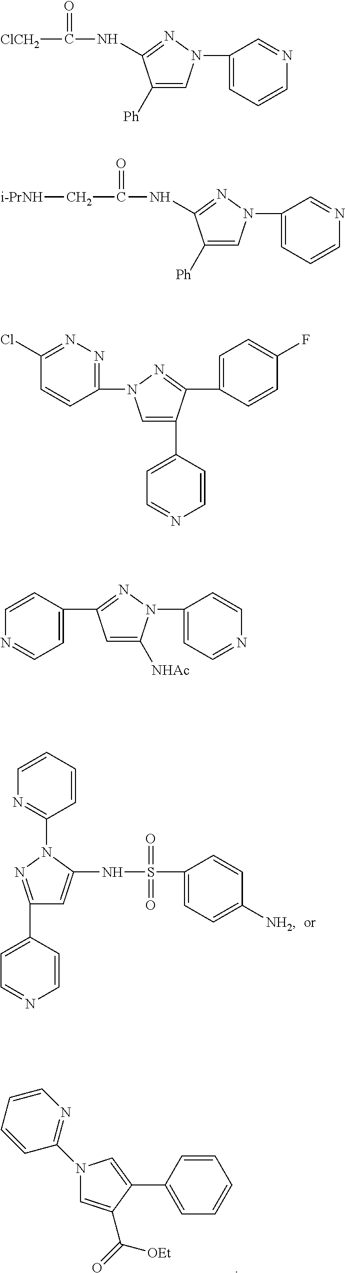

In another aspect, the compounds of this invention are represented by formula IB:

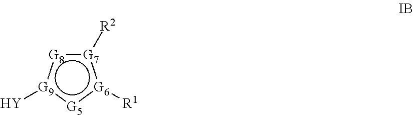

or a pharmaceutically acceptable salt thereof, wherein:

-G₅-G₆-G₇-G₈-G₉ is —CR³═C—C═N—N, —N═C—C═CR³—N, or —CR³═C—C═CR³—N;

each occurrence of R³ is independently hydrogen, —CN, halogen, —Z—R⁵, or an optionally substituted group selected from C₁₋₆ aliphatic and 3-10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
R^{3a} is hydrogen or an optionally substituted C₁₋₄ aliphatic, and
R⁵ is an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

R¹ is —CN, —C(O)N(R⁴)₂, —C(O)OR⁴, —C(NH)N(R⁴)₂, —NHCOR⁴, —NHSO₂R⁴, —NHCON(R⁴)₂, —NHCOOR⁴, —NHSO₂N(R⁴)₂, —CH₂OH, —CH₂N(R⁴)₂, —CH₂NHC(O)CH₃, —SO₂NR⁴₂, —CONHC(═NH)N(R⁴)₂, —NHSO₂OR⁴, or CY, wherein CY is an optionally substituted group selected from a 3-7-membered cycloaliphatic; a 4-10-membered heterocyclyl having 1-5 heteroatoms independently selected from nitrogen, oxygen, or sulfur; a 5-6-membered aryl, or 5-10-membered heteroaryl having 1-5 heteroatoms independently selected from nitrogen, oxygen, or sulfur; wherein:
R⁴ is hydrogen, —OH, or an optionally substituted group selected from C₁₋₆ aliphatic, 3-10-membered cycloaliphatic, 6-10-membered aryl, or 5-10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, or an optionally substituted group selected from C₁₋₆ aliphatic, —NH₂, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur; or
two occurrences of R⁴, taken together with a nitrogen atom to which they are bound, form an optionally substituted 4-7-membered heterocyclyl ring having 0-1 additional heteroatoms independently selected from nitrogen, oxygen, or sulfur;

R² is an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}, -T₂-R^{12a}, or —V₂-T₂-R^{12d}, and:
each occurrence of R^{12a} is independently halogen, —CN, —NO₂, —R^{12c}, —N(R^{12b})₂, —OR^{12b}), —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})SO₂R^{12c}, —N(R^{12e})C(O)OR^{12b}, —N(R^{12e})C(O)N(R^{12b})₂, or —N(R^{12e})SO₂N(R^{12b})₂, or two occurrences of R^{12b}, taken together with a nitrogen atom to which they are bound, form an optionally substituted 4-7-membered heterocyclyl ring having 0-1 additional heteroatoms selected from nitrogen, oxygen, or sulfur;
each occurrence of Rⁱⁿ is independently hydrogen or an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12c} is independently an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12d} is independently hydrogen or an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, —N(R^{12e})C(O)—, —N(R^{12e})SO₂—, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3-7 membered cycloaliphatic or heterocyclyl ring, wherein R¹³ is hydrogen or an optionally substituted C₁₋₄aliphatic group; and

HY is an optionally substituted group selected from:

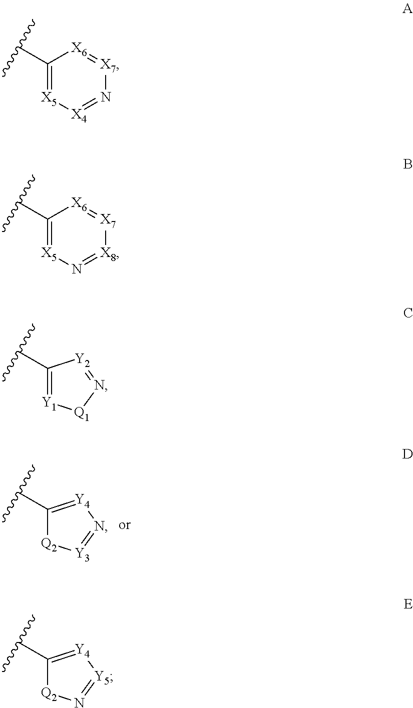

wherein each occurrence of X₄, X₅, X₆, X₇, and X₈ is independently —CR¹⁰ or N, provided no more than one occurrence of X₄, X₅, X₆, X₇, and X₈ is N, and at least two occurrences of CR¹⁰ are CH;

each occurrence of Q₁ and Q₂ is independently S, O or —NR⁹;

each occurrence of Y₁, Y₂, Y₃, Y₄, Y₅, Y₆, Y₇, and Y₈ is —CR¹⁰;
or wherein two adjacent occurrences of X₄ and X₅, X₆ and X₇, X₇ and X₈, Y₁ and Q₁, Y₃ and Q₂, or Y₄ and Y₅, taken together with the atom to which they are bound, form an optionally substituted fused group selected from 5-6-membered aryl, or 5-6-membered heteroaryl having 1-5 heteroatoms independently selected from nitrogen, oxygen, or sulfur;
   wherein R¹⁰ is —R^{10b}, —V₁—R^{10c}, -T₁-R^{10b}, or —V₁-T₁-R^{10b} wherein:
V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
each occurrence of R^{10a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, —N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3-7 membered cycloaliphatic or heterocyclyl ring;
each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{10c} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, or
R^{10a} and R^{10b}, taken together with a nitrogen atom to which they are bound, form an optionally substituted 4-7-membered heterocyclyl ring having 0-1 additional heteroatoms independently selected from nitrogen, oxygen, or sulfur;

each occurrence of R¹¹ is independently hydrogen, —C(O)R^{11a}, —CO₂R^{11a}, —C(O)N(R^{11a})₂, —C(O)N(R^{11a})—OR^{11a}, SO₂R^{11a}, —SO₂N(R^{11a})₂, or an option ally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
wherein each occurrence of R^{11a} is independently hydrogen or an optionally substituted group selected from C₁₋₆aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;
wherein each occurrence of R^{9a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;

wherein each occurrence of R^{9b} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3-6-membered heterocyclyl having 1-5 heteroatoms independently selected from nitrogen, oxygen, or sulfur, or 5-10-membered heteroaryl having 1-5 heteroatoms independently selected from nitrogen, oxygen, or sulfur; and
provided that R¹ is not an optionally substituted phenyl; and
R¹, R², and Hy are not all simultaneously pyridyl; and
provided that:
a) when Hy is selected from

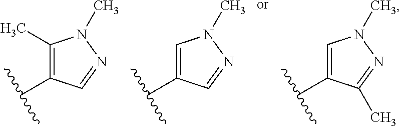

then neither R¹ nor R² is the same as Hy;
b) when Hy is pyridazinyl and R² is phenyl, R¹ is not —CO₂Et;
c) Hy is not quinoxalinyl substituted with a sulfur containing group, or an optionally substituted

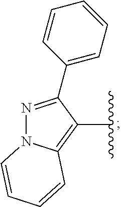 [SMILES: *c1c(-c2ccccc2)nn2ccccc12 ; ";"]

d) when R¹ is —CO₂H, then R² is not an optionally substituted ring selected from thienyl, furanyl, or cyclohexyl;
e) when R¹ is CN, then R² is not an unsubstituted cyclopropyl, or an optionally substituted ring selected from -phenyl-NH—CH₂-phenyl, -phenyl-NH—CH₂-pyridinyl, -phenyl-NH—C(O)-phenyl, or -phenyl-NH—C(O)-pyridyl;
f) R¹ is not an optionally substituted ring selected from

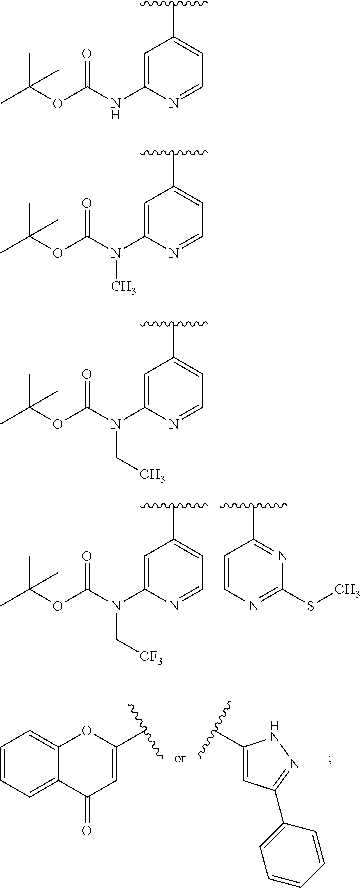

g) R¹ is not —NHC(O)CH₂N(isopropyl)C(O)—;
h) R¹ is not optionally substituted —CH₂NH-pyridyl;
i) neither R¹ nor R² is an optionally substituted ring selected from dibenzofuran, or

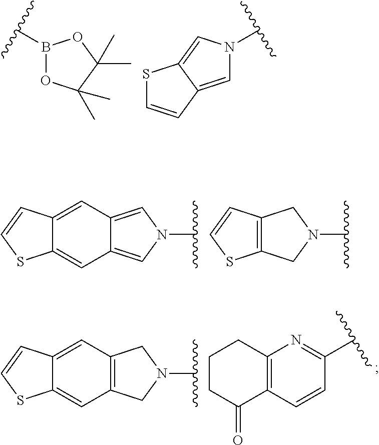

j) when R¹ is cyclopropyl, then R² is not phenyl substituted with —CF₃ or —OCF₃;
k) when R² is cyclopropyl, R³ is not chloro;
l) when R² is an optionally substituted phenyl, R¹ and R³ are not both —CO₂CH₃ or —CH₂OH;
m) when R² is dichlorophenyl, then R¹ is not an optionally substituted cyclobutyl or —CH₂—NH—CH₂—;
n) R² is not an optionally substituted

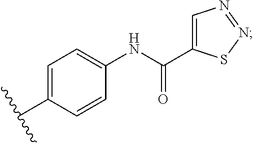 [SMILES: *c1ccc(NC(=O)c2cnns2)cc1]

o) R³ is not an optionally substituted

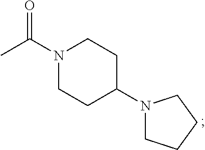

P) when -G₅-G₆-G₇-G₈-G₉ is —CR³═C—C═CR³—N, then R¹ is not —CN;
q) when -G₅-G₆-G₇-G₈-G₉ is —CR³═C—C═CR³—N, and Hy is quinolinyl, R² is not cyclopropyl;
r) the compound is other than:

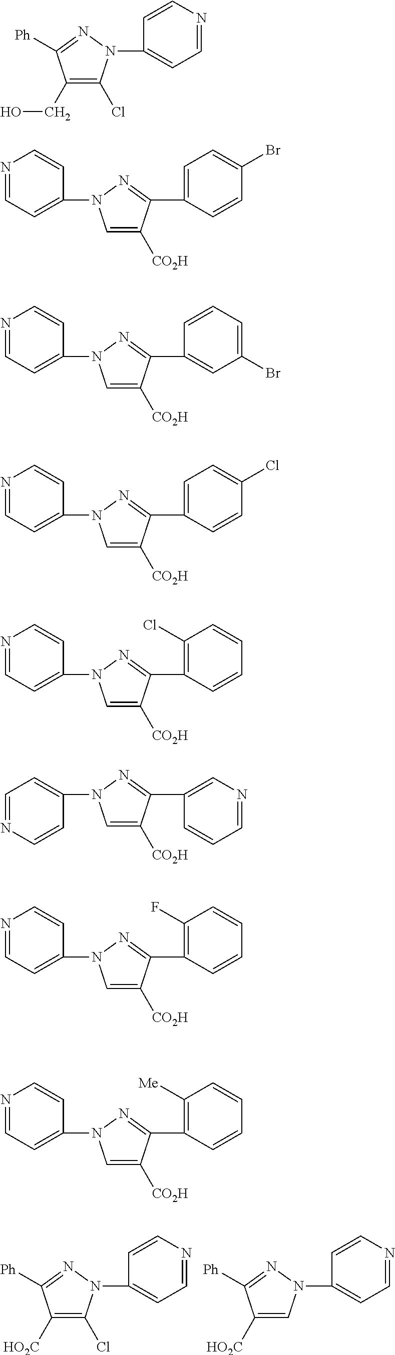

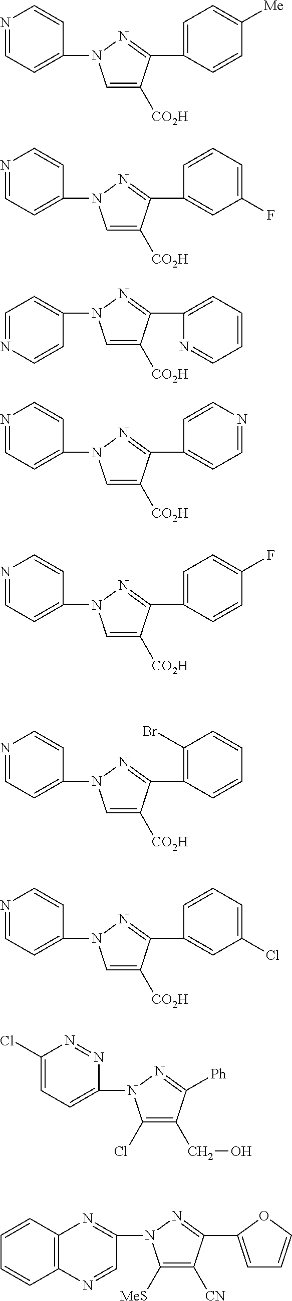

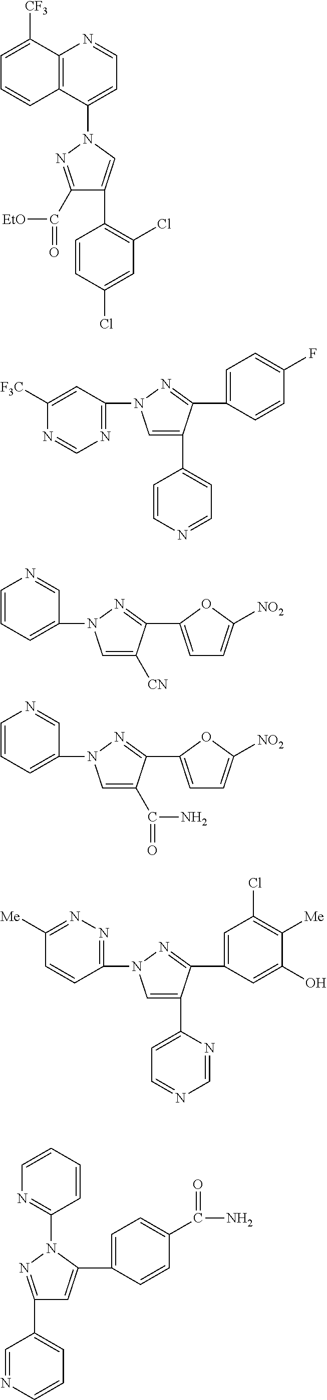 [SMILES: CCOC(=O)c1nn(-c2ccnc3c(C(F)(F)F)cccc23)cc1-c1ccc(Cl)cc1Cl ; Fc1ccc(-c2nn(-c3cc(C(F)(F)F)ncn3)cc2-c2ccncc2)cc1 ; N#Cc1cn(-c2cccnc2)nc1-c1ccc([N+](=O)[O-])o1 ; NC(=O)c1cn(-c2cccnc2)nc1-c1ccc([N+](=O)[O-])o1 ; Cc1ccc(-n2cc(-c3ccncn3)c(-c3cc(O)c(C)c(Cl)c3)n2)nn1 ; NC(=O)c1ccc(-c2cc(-c3cccnc3)nn2-c2ccccn2)cc1]

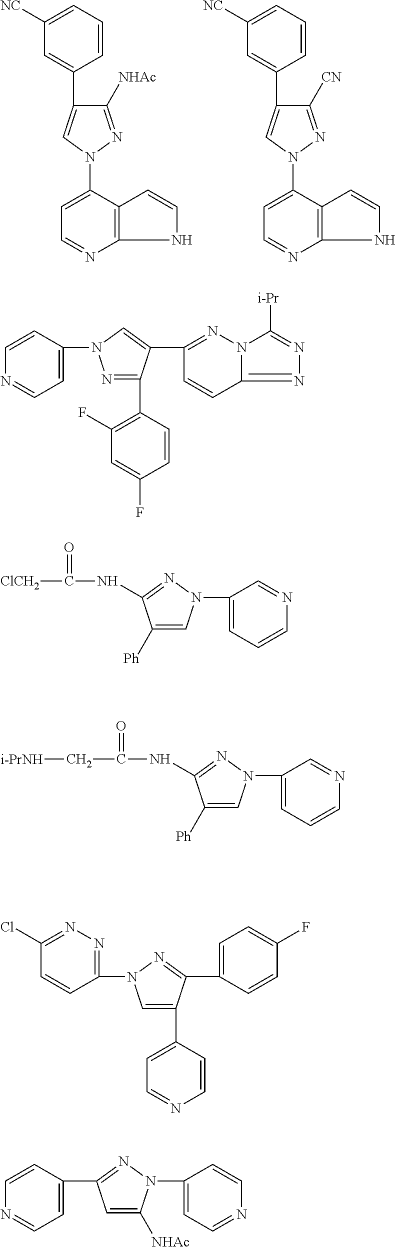

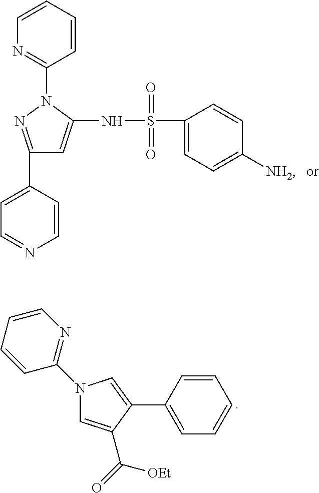 [SMILES: Nc1ccc(S(=O)(=O)Nc2cc(-c3ccncc3)nn2-c2ccccn2)cc1 ; CCOC(=O)c1cn(-c2ccccn2)cc1-c1ccccc1]

In another aspect, the compounds of this invention are represented by formula IB:

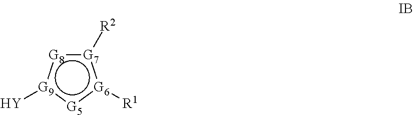

or a pharmaceutically acceptable salt thereof, wherein:

-G₅-G₆-G₇-G₈-G₉ is —CR³═C—C═N—N, —N═C—C═CR³—N, or —CR³═C—C═CR³—N;

each occurrence of R³ is independently hydrogen, —CN, halogen, —Z—R⁵, or an optionally substituted group selected from C₁₋₆ aliphatic and 3-10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
R^{3a} is hydrogen or an optionally substituted C₁₋₄ aliphatic, and
R⁵ is an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

R¹ is —CN, —C(O)N(R⁴)₂, —C(O)OR⁴, —C(NH)N(R⁴)₂, —NHCOR⁴, —NHCOOR⁴, —NHSO₂N(R⁴)₂, —CH₂OH, —CH₂N(R⁴)₂, —CH₂NHC(O)CH₃, —SO₂NR⁴₂, —CONHC(═NH)N(R⁴)₂, —NHSO₂OR⁴, or CY, wherein CY is an optionally substituted group selected from a 3-7-membered cycloaliphatic; a 4-10-membered heterocyclyl having 1-5 heteroatoms independently selected from nitrogen, oxygen, or sulfur; a 5-6-membered aryl, or 5-10-membered heteroaryl having 1-5 heteroatoms independently selected from nitrogen, oxygen, or sulfur; wherein:
R⁴ is hydrogen, —OH, or an optionally substituted group selected from C₁₋₆ aliphatic, 3-10-membered cycloaliphatic, 6-10-membered aryl, or 5-10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, or an optionally substituted group selected from C₁₋₆ aliphatic, —NH₂, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur; or
two occurrences of R⁴, taken together with a nitrogen atom to which they are bound, form an optionally substituted 4-7-membered heterocyclyl ring having 0-1 additional heteroatoms independently selected from nitrogen, oxygen, or sulfur;

R² is an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, wherein R² is optionally substituted with 1-4 occurrences of R^{2a}, wherein each occurrence of R² is independently —R^{12a}, -T₂-R^{12d}, -T₂-R^{12a}, or —V₂-T₂-R^{12d}, and:
each occurrence of R^{12a} is independently halogen, —CN, —NO₂, —R^{12c}, —N(R^{12b})₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R¹²)₂, —N(R^{12e})C(O)R^{12b}, —N(R^{12e})SO₂R^{12c}, —N(R^{12e})C(O)OR^{12b}, —N(R^{12e})C(O)N(R^{12b})₂, or —N(R^{12e})SO₂N(R^{12b})², or two occurrences of R^{12b}, taken together with a nitrogen atom to which they are bound, form an optionally substituted 4-7-membered heterocyclyl ring having 0-1 additional heteroatoms selected from nitrogen, oxygen, or sulfur;
each occurrence of R^{12b} is independently hydrogen or an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12c} is independently an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12d} is independently hydrogen or an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, N(R^{12e})C(O)—, —N(R^{12e})SO₂—, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3-7 membered cycloaliphatic or heterocyclyl ring, wherein R¹³ is hydrogen or an optionally substituted C₁₋₄aliphatic group; and

HY is an optionally substituted group selected from:

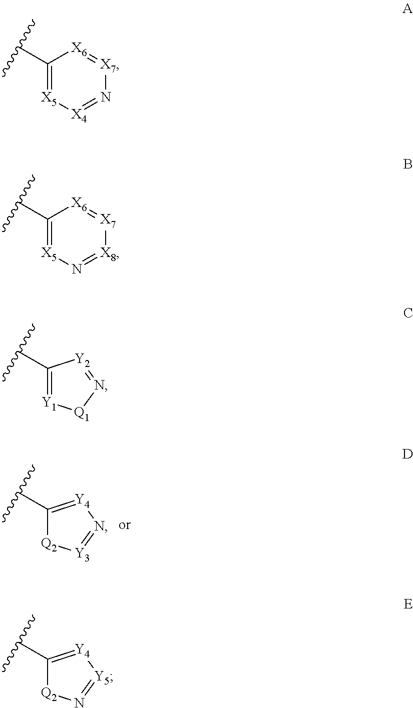

wherein each occurrence of X₄, X₅, X₆, X₇, and X₈ is independently —CR¹⁰ or N, provided no more than one occurrence of X₄, X₅, X₆, X₇, and X₈ is N, and at least two occurrences of CR¹⁰ are CH;

each occurrence of Q₁ and Q₂ is independently S, O or —NR⁹;

each occurrence of Y₁, Y₂, Y₃, Y₄, Y₅, Y₆, Y₇, and Y₈ is —CR¹⁰;
or wherein two adjacent occurrences of X₄ and X₅, X₆ and X₇, X₇ and X₈, Y₁ and Q₁, Y₃ and Q₂, or Y₄ and Y₅, taken together with the atom to which they are bound, form an optionally substituted fused group selected from 5-6-membered aryl, or 5-6-membered heteroaryl having 1-5 heteroatoms independently selected from nitrogen, oxygen, or sulfur;
   wherein R¹⁰ is R^{10b}, —V₁—R^{10c}, -T₁-R^{10b}, or —V₁-T₁-R^{10b} wherein:
V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
each occurrence of R^{10a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, —N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3-7 membered cycloaliphatic or heterocyclyl ring;
each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{10b} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, or
R^{10a} and R^{10b}, taken together with a nitrogen atom to which they are bound, form an optionally substituted 4-7-membered heterocyclyl ring having 0-1 additional heteroatoms independently selected from nitrogen, oxygen, or sulfur;

each occurrence of R¹¹ is independently hydrogen, —C(O)R^{11a}, —CO₂R^{11a}, —C(O)N(R^{11a})₂, C(O)N(R^{11a})—OR^{11a}, —SO₂R^{11a}, —SO₂N(R^{11a})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
wherein each occurrence of R^{11a} is independently hydrogen or an optionally substituted group selected from C₁₋₆aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;
wherein each occurrence of R^{9a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;

wherein each occurrence of R^{9b} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3-6-membered heterocyclyl having 1-5 heteroatoms independently selected from nitrogen, oxygen, or sulfur, or 5-10-membered heteroaryl having 1-5 heteroatoms independently selected from nitrogen, oxygen, or sulfur; and

provided that R¹ is not an unsubstituted phenyl or a phenyl substituted only with one or two groups selected from methyl, tert-butyl, —CF₃ or halogen; and

R¹, R², and Hy are not all simultaneously pyridyl; and

provided that:
a) when Hy is selected from

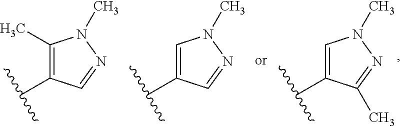

then neither R¹ nor R² is the same as Hy;
b) when Hy is pyridazinyl and R² is phenyl, R¹ is not —CO₂Et;
c) Hy is not quinoxalinyl substituted with a sulfur containing group, or an optionally substituted

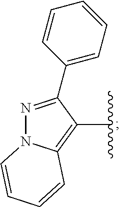 [SMILES: *c1c(-c2ccccc2)nn2ccccc12]

d) when R¹ is —CO₂H, then R² is not an optionally substituted ring selected from thienyl, furanyl, or cyclohexyl;
e) when R¹ is CN, then R² is not an unsubstituted cyclopropyl, or an optionally substituted ring selected from -phenyl-NH—CH₂-phenyl, -phenyl-NH—CH₂-pyridinyl, -phenyl-NH—C(O)-phenyl, or -phenyl-NH—C(O)-pyridyl;
f) R¹ is not an optionally substituted ring selected from

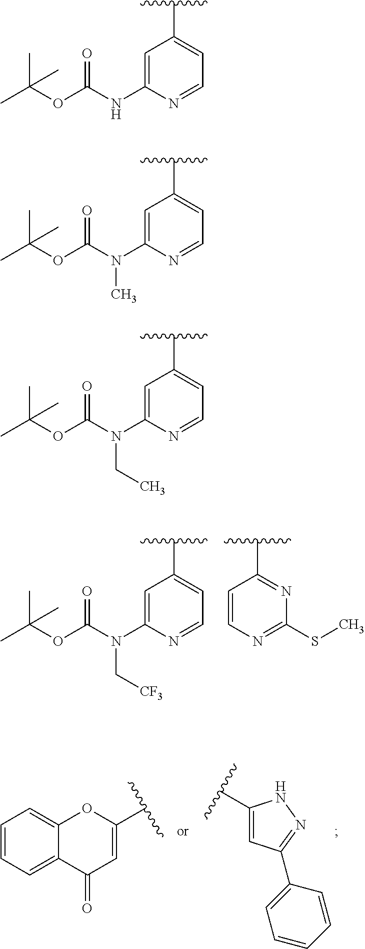

g) R¹ is not phenyl substituted with —C(O)N(H)C(H)(benzyl-OH)C(O)NH₂;
h) R¹ is not —NHC(O)CH₂N(isopropyl)C(O)—;
i) R¹ is not optionally substituted —CH₂NH-pyridyl;
j) neither R¹ nor R² is an optionally substituted ring selected from dibenzofuran, or

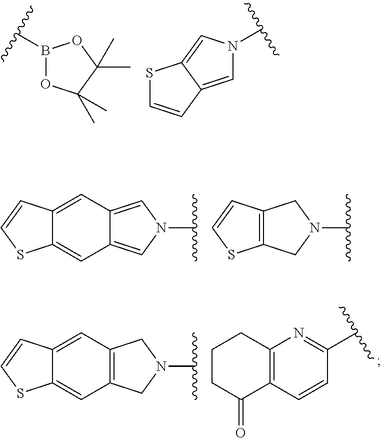

k) when either R¹ or R² is cyclopropyl, then the other of R¹ or R² is not phenyl substituted with —CF₃ or —OCF₃;
l) when R² is cyclopropyl, R³ is not chloro;
m) when R² is an optionally substituted phenyl, R¹ and R³ are not both —CO₂CH₃ or —CH₂OH;
n) when R² is dichlorophenyl, then R¹ is not an optionally substituted cyclobutyl or —CH₂—NH—CH₂—;
o) R² is not an optionally substituted

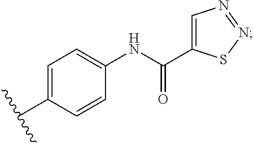 [SMILES: *c1ccc(NC(=O)c2cnns2)cc1]

p) R³ is not an optionally substituted

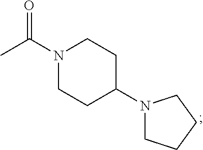

q) when -G₅-G₆-G₇-G₈-G₉ is —CR³═C—C═CR³—N, then R¹ is not —CN;
r) when -G₅-G₆-G₇-G₈-G₉ is —CR³═C—C═CR³—N, and Hy is quinolinyl, R² is not cyclopropyl;
s) the compound is other than:

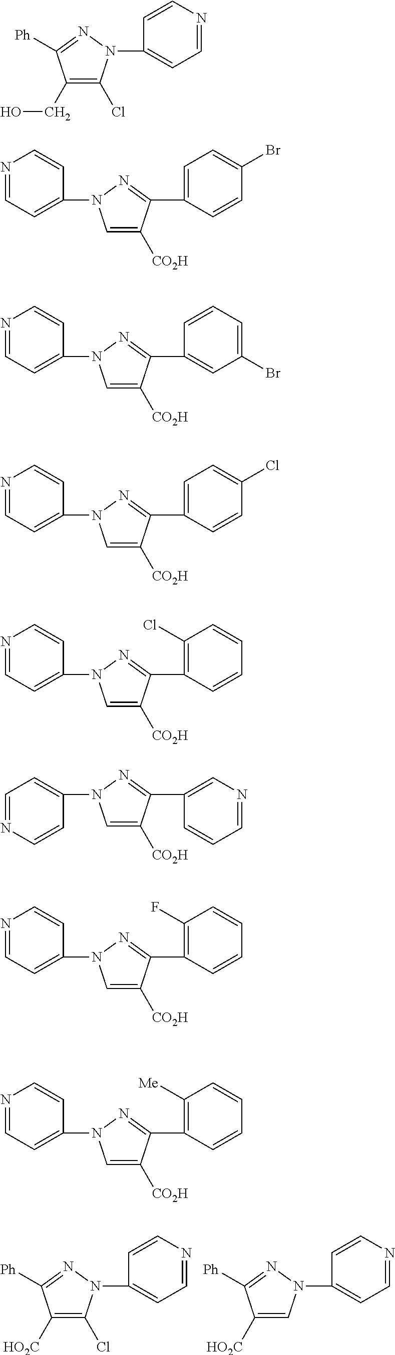

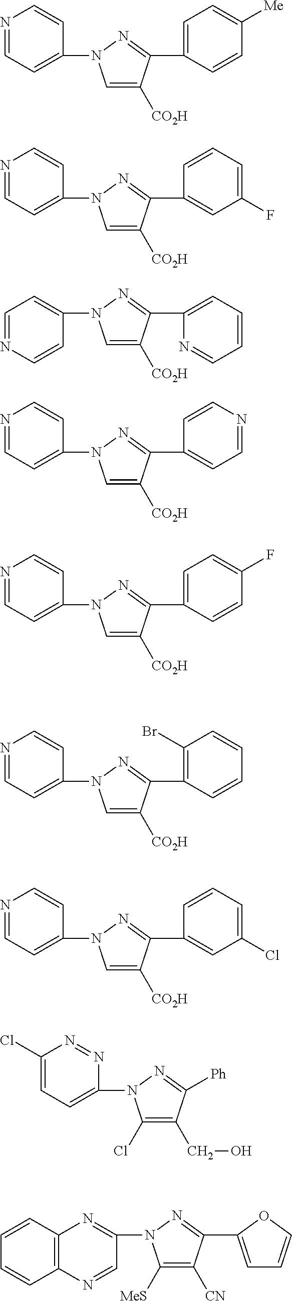

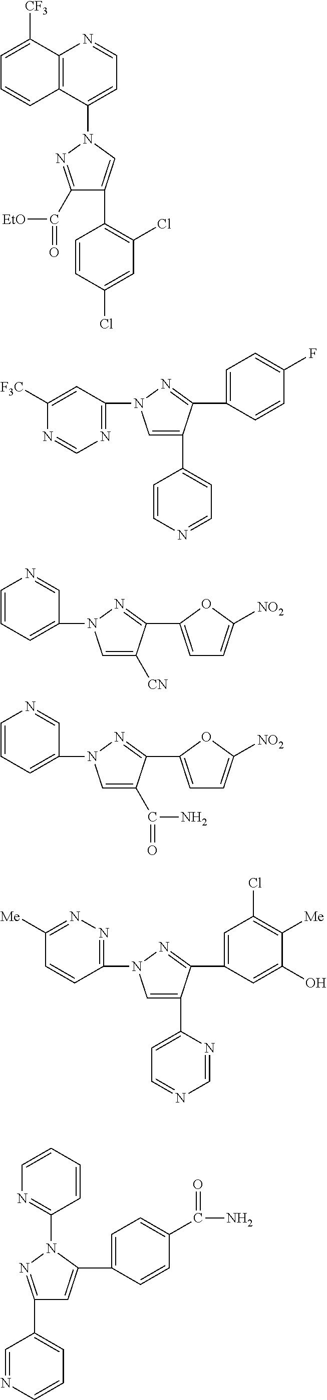 [SMILES: CCOC(=O)c1nn(-c2ccnc3c(C(F)(F)F)cccc23)cc1-c1ccc(Cl)cc1Cl ; Fc1ccc(-c2nn(-c3cc(C(F)(F)F)ncn3)cc2-c2ccncc2)cc1 ; N#Cc1cn(-c2cccnc2)nc1-c1ccc([N+](=O)[O-])o1 ; NC(=O)c1cn(-c2cccnc2)nc1-c1ccc([N+](=O)[O-])o1 ; Cc1ccc(-n2cc(-c3ccncn3)c(-c3cc(O)c(C)c(Cl)c3)n2)nn1 ; NC(=O)c1ccc(-c2cc(-c3cccnc3)nn2-c2ccccn2)cc1]

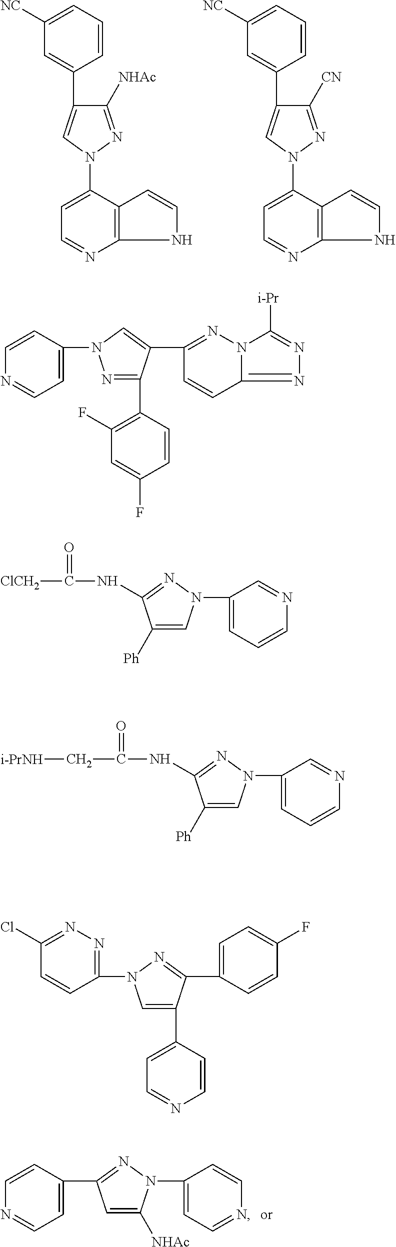

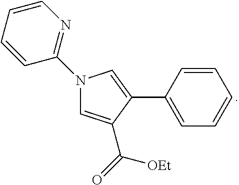 [SMILES: CCOC(=O)c1cn(-c2ccccn2)cc1-c1ccccc1]

In certain other embodiments, compounds of formula ID are provided:

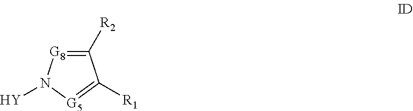

or a pharmaceutically acceptable salt thereof, wherein:

both of G₅ and G₈ are CR³, or one of G₅ and G₈ is N and the other is CR³;

when one of G₅ or G₈ is N, R³ is hydrogen, —CN, halogen, —Z—R⁵, or an optionally substituted group selected from C₁₋₆ aliphatic and 3-10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
R^{3a} is hydrogen or an optionally substituted C₁₋₄ aliphatic, and
R⁵ is an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

when G₅ and G₈ are both CR³, each occurrence of R³ is independently hydrogen, CN, or an optionally substituted C₁₋₃ aliphatic;

R¹ is —CN, —C(O)N(R⁴)₂, —C(O)OR⁴, —C(NH)N(R⁴)₂, —NHCOR⁴, —NHSO₂R⁴, —NHCON(R⁴)₂, —NHCOOR⁴, —NHSO₂N(R⁴)₂, —CH₂OH, —CH₂N(R⁴)₂, —CH₂NHC(O)CH₃, —SO₂NR⁴₂, —CONHC(═NH)N(R⁴)₂, —NHSO₂OR⁴, or CY, wherein CY is an optionally substituted group selected from a 3-7-membered cycloaliphatic; a 4-10-membered heterocyclyl having 1-5 heteroatoms independently selected from nitrogen, oxygen, or sulfur; a 5-6-membered aryl, or 5-10-membered heteroaryl having 1-5 heteroatoms independently selected from nitrogen, oxygen, or sulfur; wherein:
R⁴ is hydrogen, —OH, or an optionally substituted group selected from C₁₋₆ aliphatic, 3-10-membered cycloaliphatic, 6-10-membered aryl, or 5-10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, or an optionally substituted group selected from C₁₋₆ aliphatic, —NH₂, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur; or
two occurrences of R⁴, taken together with a nitrogen atom to which they are bound, form an optionally substituted 4-7-membered heterocyclyl ring having 0-1 additional heteroatoms independently selected from nitrogen, oxygen, or sulfur;

R² is an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}, -T₂-R^{12a}, or —V₂-T₂-R^{12d}, and:
each occurrence of R^{12a} is independently halogen, —CN, —NO₂, —R^{12c}, —N(R¹²)₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R¹²)₂, N(R^{12e})C(O)R^{12b}, —N(R^{12e})SO₂R^{12c}, —N(R^{12e})C(O)OR^{12b}, —N(R^{12e})C(O)N(R^{12b})₂, —N(R^{12e})SO₂N(R^{12b})₂, or two occurrences of R^{12b}, taken together with a nitrogen atom to which they are bound, form an optionally substituted 4-7-membered heterocyclyl ring having 0-1 additional heteroatoms selected from nitrogen, oxygen, or sulfur;
each occurrence of R^{12b} is independently hydrogen or an optionally substituted group selected from C₁-C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12c} is independently an optionally substituted group selected from C₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12d} is independently hydrogen or an optionally substituted group selected from 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —N(R^{12e})C(O)—, —N(R^{12e})SO₂—, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3-7 membered cycloaliphatic or heterocyclyl ring, wherein R¹³ is hydrogen or an optionally substituted C₁₋₄aliphatic group; and

HY is an optionally substituted group selected from:

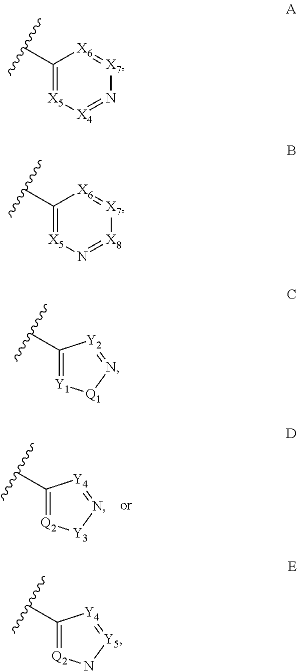

wherein each occurrence of X₄, X₅, X₆, X₇, and X₈ is independently —CR¹⁰ or N, provided no more than one occurrence of X₄, X₅, X₆, X₇, and X₈ is N, and at least two occurrences of CR¹⁰ are CH;

each occurrence of Q₁ and Q₂ is independently S, O or —NR⁹;

each occurrence of Y₁, Y₂, Y₃, Y₄, Y₅, Y₆, Y₇, and Y₈ is —CR¹⁰;
or wherein two adjacent occurrences of X₄ and X₅, X₆ and X₇, X₇ and X₈, Y₁ and Q₁, Y₃ and Q₂, or Y₄ and Y₅, taken together with the atom to which they are bound, form an optionally substituted fused group selected from 5-6-membered aryl, or 5-6-membered heteroaryl having 1-5 heteroatoms independently selected from nitrogen, oxygen, or sulfur;
   wherein R¹⁰ is —R^{10b}, —V₁—R^{10c}, -T₁-R^{10b}, or —V₁-T₁-R^{10b} wherein:
V₁ is NR¹¹—, —NR¹¹C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —C(O)NR¹¹—, —C(O)NR¹¹O—, —SO₂—, or —SO₂NR¹¹—;
each occurrence of R^{10a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, N(R^{10a})C(O)N(R^{10a})N(R^{10a})S(O)₂N(R^{10a})—, —OC(O)—, or —C(O)N(R¹¹)—O— or wherein T₁ forms part of an optionally substituted 3-7 membered cycloaliphatic or heterocyclyl ring;
each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R^{10c} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur, or
R^{10a} and R^{10b}, taken together with a nitrogen atom to which they are bound, form an optionally substituted 4-7-membered heterocyclyl ring having 0-1 additional heteroatoms independently selected from nitrogen, oxygen, or sulfur;
each occurrence of R¹¹ is independently hydrogen, —C(O)R^{11a}, —CO₂R^{11a}, —C(O)N(R^{11a})₂, —C(O)N(R^{11a})—OR^{11a}, —SO₂R^{11a}, —SO₂N(R^{11a})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
wherein each occurrence of R^{11a} is independently hydrogen or an optionally substituted group selected from C₁₋₆aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;

each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;
wherein each occurrence of R^{9a} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur;

wherein each occurrence of R^{9b} is independently hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3-6-membered heterocyclyl having 1-5 heteroatoms independently selected from nitrogen, oxygen, or sulfur, or 5-10-membered heteroaryl having 1-5 heteroatoms independently selected from nitrogen, oxygen, or sulfur.

In certain other embodiments, compounds of formula ID are provided:

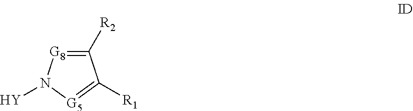

or a pharmaceutically acceptable salt thereof are provided, wherein:

both of G₅ and G₈ are CR³, or one of G₅ and G₈ is N and the other is CR³; when one of G₅ or G₈ is N, R³ is hydrogen, —CN, halogen, —Z—R⁵, or an optionally substituted group selected from C₁₋₆ aliphatic and 3- to 10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
   R^{3a} is hydrogen or an optionally substituted C₁₋₄ aliphatic, and

R⁵ is hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;

when G₅ and G₈ are both CR³, each occurrence of R³ is independently hydrogen, CN, or an optionally substituted C₁₋₃ aliphatic;

R¹ is —CN, —C(O)N(R⁴)₂, —C(O)OR⁴, —C(NR⁴)N(R⁴)₂, —NHCOR⁴, —NHSO₂R⁴, —NHCON(R⁴)₂, —NHCOOR⁴, —NHSO₂N(R⁴)₂, —CH₂OR⁴, —CH₂N(R⁴)₂, —CH₂NHC(O)R⁴, —SO₂NR⁴₂, —CONHC(═NH)N(R⁴)₂, —NHSO₂OR⁴, or CY, wherein CY is an optionally substituted group selected from a 3- to −7-membered cycloaliphatic; a 4- to 10-membered heterocyclyl having 1-5 heteroatoms independently selected from nitrogen, oxygen, or sulfur; a 6-10 membered aryl, or 5- to 10-membered heteroaryl having 1-5 heteroatoms independently selected from nitrogen, oxygen, or sulfur; wherein:
each R⁴ is independently selected from hydrogen, —OH, or an optionally substituted group selected from C₁₋₆ aliphatic, 3- to 10-membered cycloaliphatic, 6- to 10-membered aryl, or 5- to 10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, —NH₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur; or
two occurrences of R⁴, taken together with a nitrogen atom to which they are bound, form an optionally substituted 4- to −7-membered heterocyclyl ring having 0-1 additional heteroatoms independently selected from nitrogen, oxygen, or sulfur;
   R² is hydrogen, halo or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}, -T₂-R^{12a}, or —V₂-T₂-R^{12d}, and:
each occurrence of R^{12a} is independently halogen, —CN, —NO₂, —R^{12c}, —N(R^{12b})₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)^{12b}, —N(R^{12e})SO₂R^{12c}, —N(R^{12e})C(O)OR^{12b}, —N(R^{12e})C(O)N(R^{12b})₂, or —N(R^{12e})SO₂N(R^{12b})₂, or an optionally substituted C₁-C₆ aliphatic or C₁-C₆haloaliphatic;
each occurrence of R^{12b} is independently hydrogen or an optionally substituted group selected from C₁-C₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or two occurrences of R^{12b}, taken together with a nitrogen atom to which they are bound, form an optionally substituted 4- to −7-membered heterocyclyl ring having 0-1 additional heteroatoms selected from nitrogen, oxygen, or sulfur;
each occurrence of R^{12e} is independently hydrogen or an optionally substituted group selected from C₁-C₆ aliphatic, C₁-C₆ haloaliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12d} is independently hydrogen or an optionally substituted group selected from 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, —N(R^{12e})C(O)—, —N(R^{12e})SO₂—, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3- to −7 membered cycloaliphatic or heterocyclyl ring, wherein R¹³ is hydrogen or an optionally substituted C₁₋₄ aliphatic group; and

HY is a group selected from:

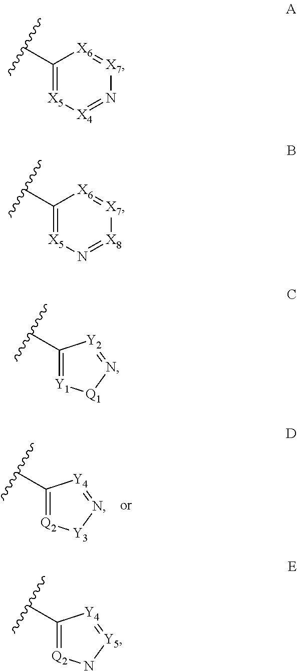

wherein
each occurrence of X₄, X₅, X₆, X₇, and X₈ is independently —CR¹⁰, —CR^{10′}, or N, provided no more than two occurrences of X₄, X₅, X₆, X₇, and X₈ is N;
each occurrence of Y₁, Y₂, Y₃, Y₄, and Y₅ is —CR¹⁰;
each occurrence of Q₁ and Q₂ is independently S, O or —NR⁹;
two adjacent occurrences of X₄ and X₅, X₆ and X₇, X₇ and X₈, Y₁ and —NR⁹, Y₃ and —NR⁹, or Y₄ and Y₅, may be taken together with the atom to which they are bound, to form an unsubstituted fused group having 8 to 10 ring atoms selected from an aryl group, or a heteroaryl group having 1-5 heteroatoms independently selected from nitrogen, oxygen, or sulfur;
each occurrence of R¹⁰ or R^{10′} is independently —R^{10b}, —V₁—R^{10c} or —V₁-T₁-R^{10b}, or —V₁-T₁-R^{10b}, wherein:
   V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
   each occurrence of R^{10a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, —N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3- to −7 membered cycloaliphatic or heterocyclyl ring;
   each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   each occurrence of R^{10c} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or
R^{10a} and R^{10b}, taken together with a nitrogen atom to which they are bound, form an optionally substituted 4- to −7-membered heterocyclyl ring having 0-1 additional heteroatoms independently selected from nitrogen, oxygen, or sulfur;
   each occurrence of R¹¹ is independently hydrogen, —C(O)R^{11a}, —CO₂R^{11a}, —C(O)N(R^{11a})₂, —C(O)N(R^{11a})—OR^{11a}, —SO₂R^{11a}, —SO₂N(R^{11a})₂, or an option ally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
wherein each occurrence of R^{11a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
wherein each occurrence of R^{9a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
   wherein each occurrence of R^{9b} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3- to 6-membered heterocyclyl having 1-5 heteroatoms independently selected from nitrogen, oxygen, or sulfur, or 5- to 10-membered heteroaryl having 1-5 heteroatoms independently selected from nitrogen, oxygen, or sulfur; and

provided that when HY is a non-fused group then HY is substituted with at least one occurrence of R¹⁰ or R^{10′}, wherein R¹⁰ or R^{10′} is:
—N(R¹¹)C(O)R^{10a}, —C(O)N(R¹¹)₂, or —NR¹¹C(O)OR^{10a}; or
—V₁-T₁-R^{10b}, wherein V₁ is −NR¹¹—, T₁ is a C₁-C₃ alkylene chain, and R^{10b} is an optionally substituted 6- to 10-membered aryl ring or a 5- to 10-membered heteroaryl ring having 1-5 heteroatoms independently selected from nitrogen, oxygen, or sulfur, or V₁ is —NR¹¹C(O)NR¹¹—, T₁ is a C₁-C₃ alkylene chain, and R^{10b} is —OR^{10a}; or
—V₁—R^{10c}, wherein V₁ is —NR¹¹—, and R^{10c} is a 5- to 10-membered heteroaryl ring having 1-5 heteroatoms independently selected from nitrogen, oxygen, or sulfur; and
   provided that:

a) for compounds where -G₅-G₆-G₇-G₈-G₉ is —CR³═C—C═N—N or —CR³═C—C═CR³N:
(i) when G_{g} is N, R² is methyl, and HY is

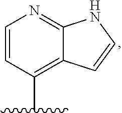 [SMILES: *c1ccnc2[nH]ccc12]

then R¹ is not an optionally substituted phenyl;
(ii) when G₈ is CH, then HY is not

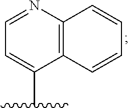 [SMILES: *c1ccnc2ccccc12]

(iii) when G₈ is CH, R² is hydrogen, and HY is 3-pyridyl, then HY is not substituted with

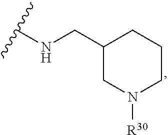

wherein R³⁰ is hydrogen, or —CO₂-tert-butyl;
(iv) provided that for compounds where G₈ is N and R² is hydrogen:
   aa) when HY is 4-pyridyl, then R¹ is not —CO₂H;
   bb) HY is not substituted with:

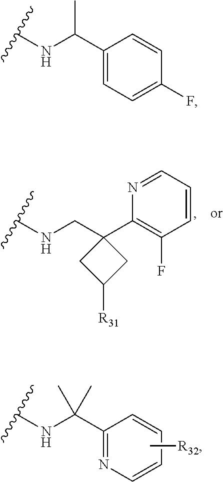

wherein R³¹ is hydrogen or fluoro;
R³² is fluoro, chloro, or —OCHF₂;
   cc) when G₅ is C—R³, and R³ is —CH₃ or —NH₂, then R¹ is not —CO₂Et; and

b) for compounds where -G₅-G₆-G₇-G₈-G₉ is —N═C—C═CR³—N:
(i) when R³ is

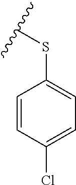 [SMILES: *Sc1ccc(Cl)cc1]

and R² is H, then R¹ is not

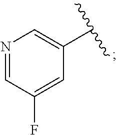 [SMILES: *c1cncc(F)c1]

(ii) when R² is methyl or hydrogen and R³ is hydrogen, then HY is not

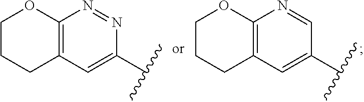 [SMILES: *c1cc2c(nn1)OCCC2 ; *c1cnc2c(c1)CCCO2]

(iii) when R² and R³ are both hydrogen then HY is not

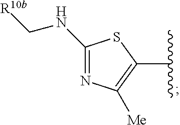

(iv) when R² is hydrogen and R³ is —CF₃, then R¹ is not optionally substituted 3-pyridinyl, 1,6-dihydro-6-oxo-3-pyridinyl,tetrahydro-2H-pyran-4-yl or thiazolyl;
(v) when R² is hydrogen and R³ is —CF₃ or —NH₂, then HY is not

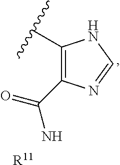

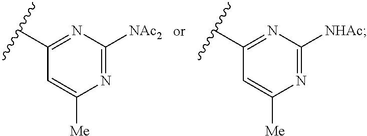

(vi) when R² and R³ are both hydrogen and HY is

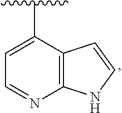 [SMILES: *c1ccnc2[nH]ccc12]

then R¹ is not an optionally substituted phenyl ring;
(vii) when R¹ is unsubstituted thiazolyl, then HY is not substituted with —CH₂CH₂OH or —CH₂CH₂OSiMe₂t-Bu;
(viii) when R³ is —SCH₃, and R² is hydrogen, then R¹ is not substituted phenyl;
(ix) when R¹ is —CO₂R⁴, R² is hydrogen, and HY is

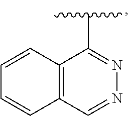 [SMILES: *c1nncc2ccccc12]

then R³ is not —CR¹⁰¹═CHR¹⁰² where R¹⁰¹ is hydrogen, methyl, or phenyl and R¹⁰² is an optionally substituted ring; and

c) provided that the compound is other than:

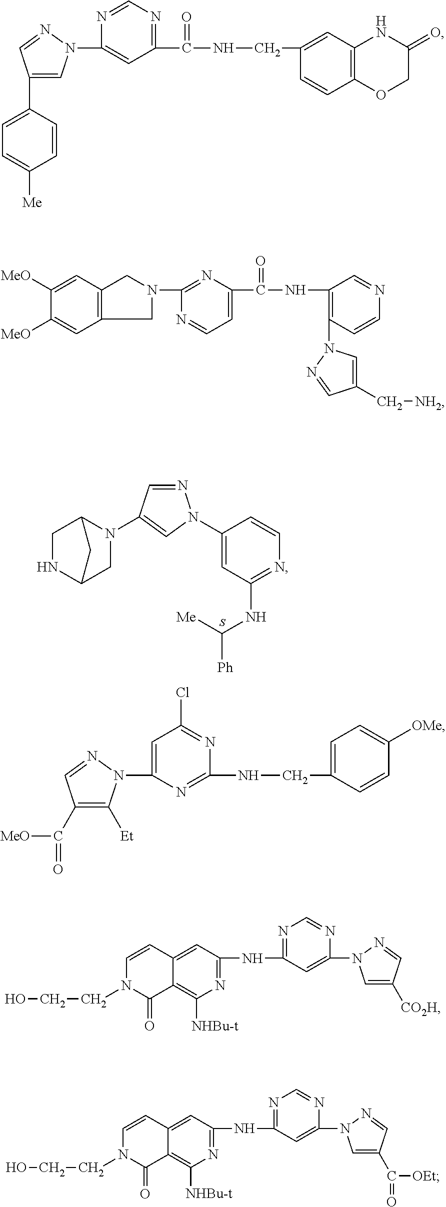

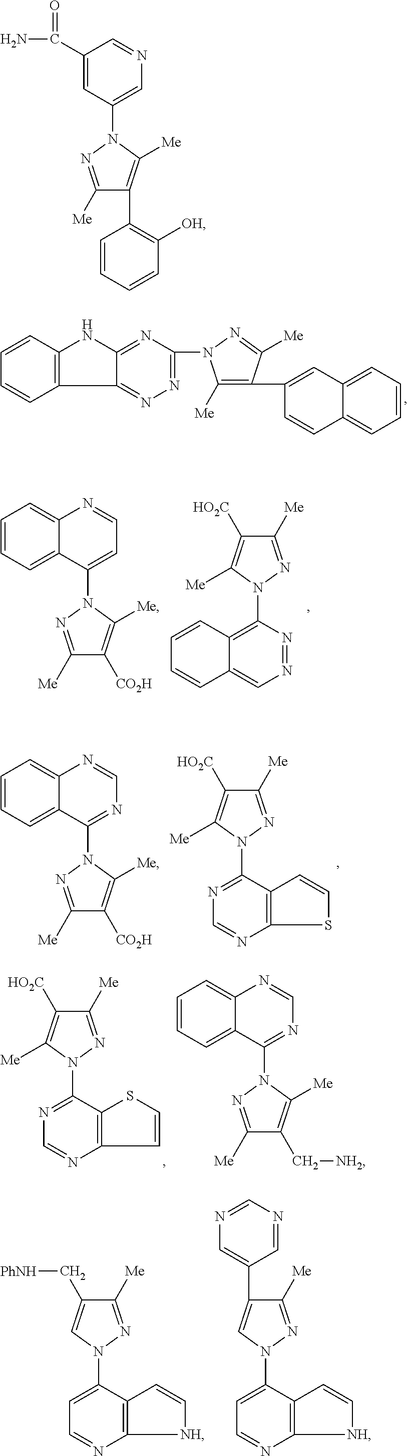

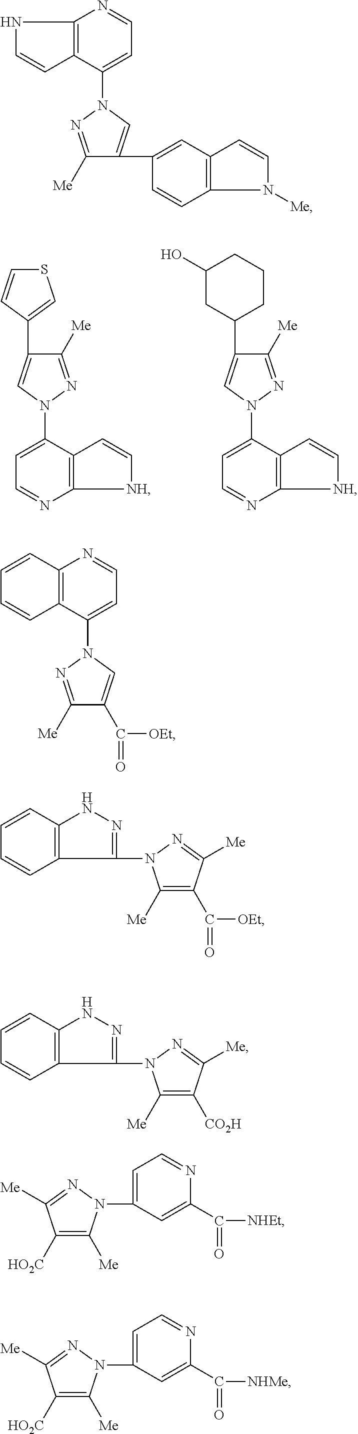

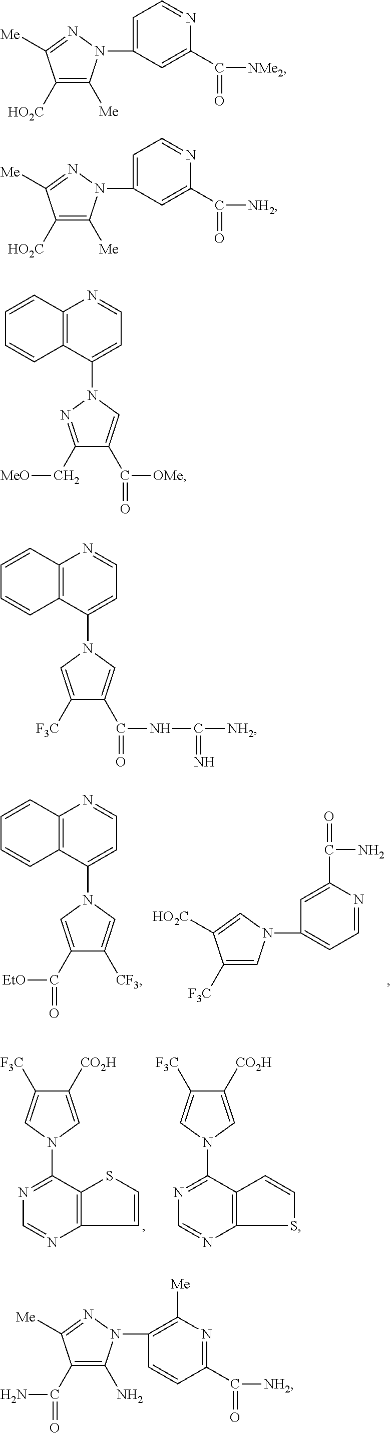

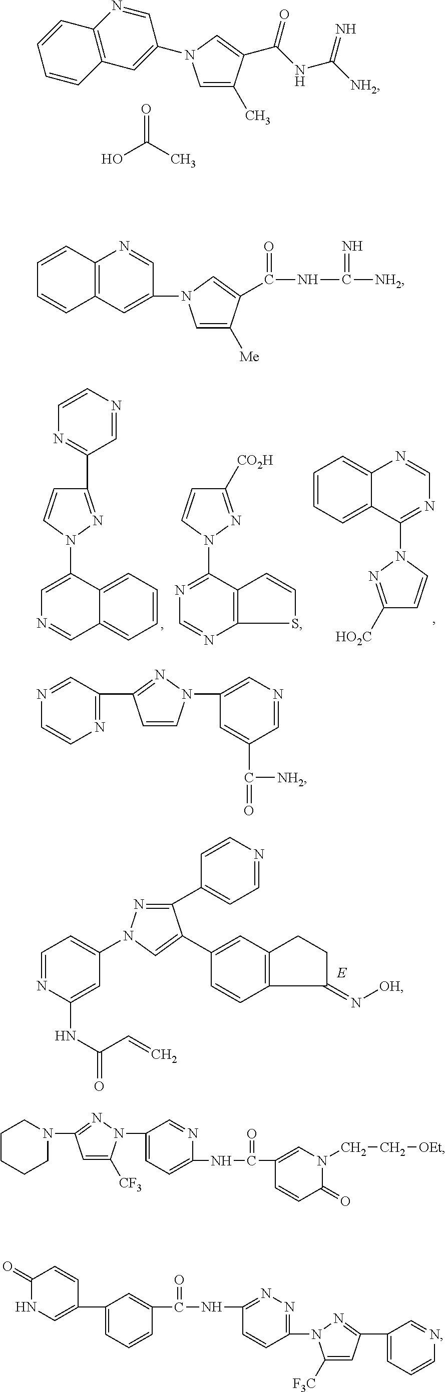

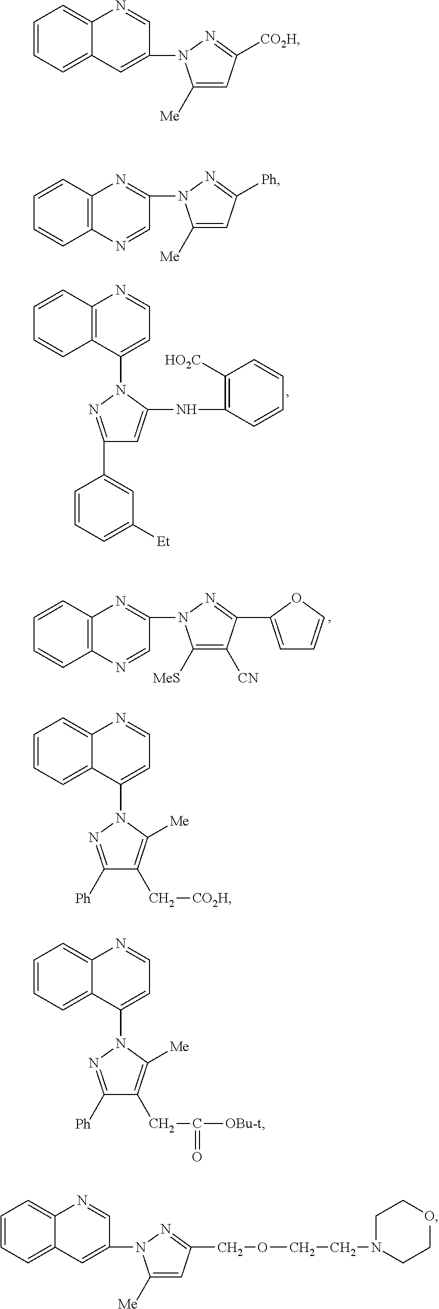

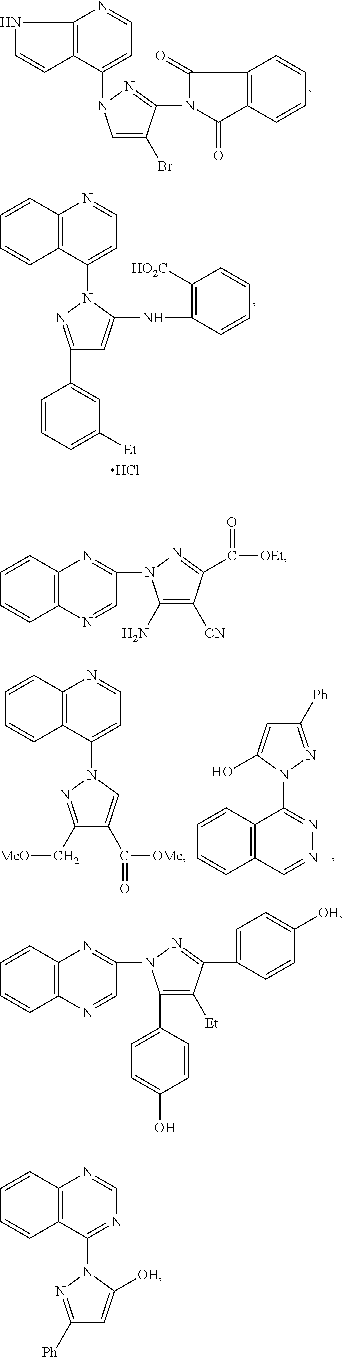

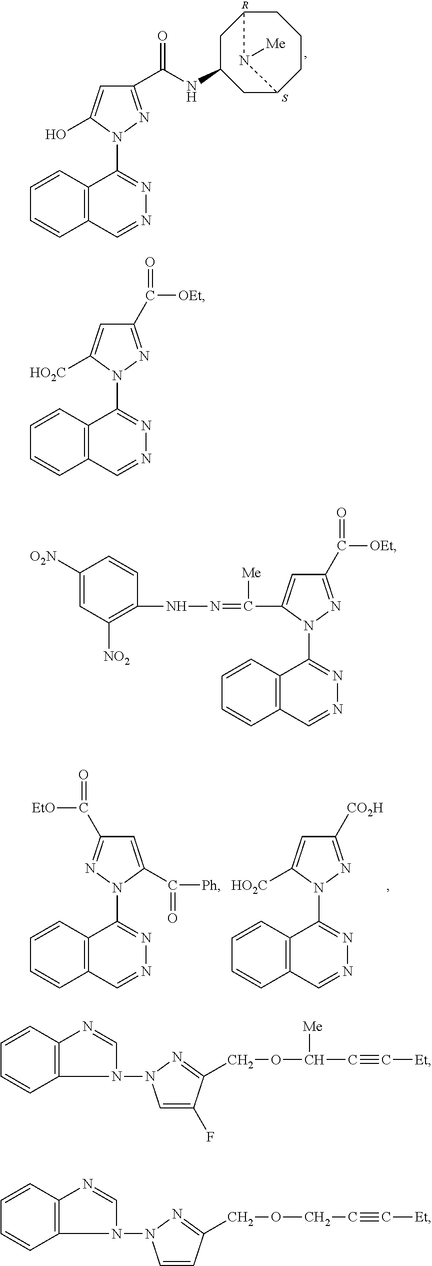

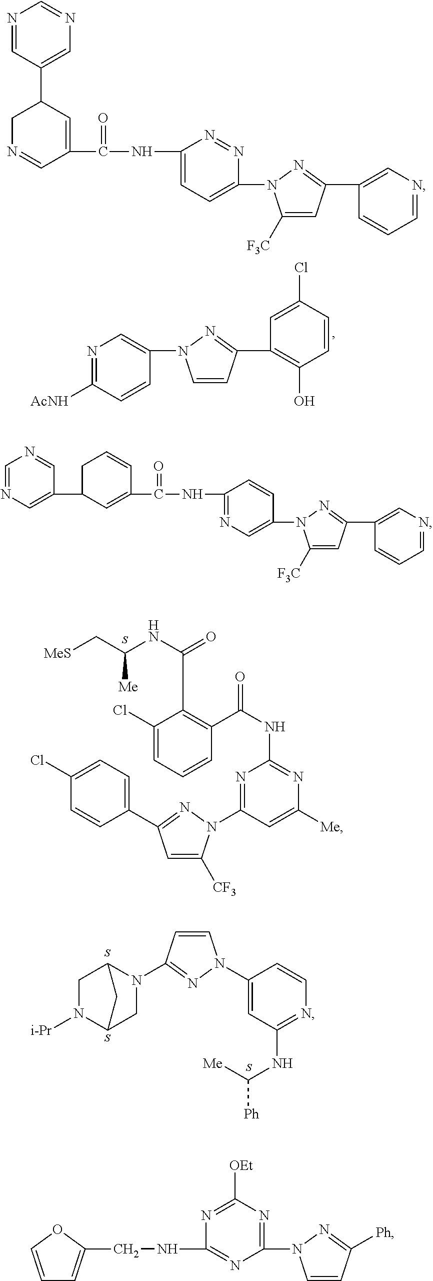

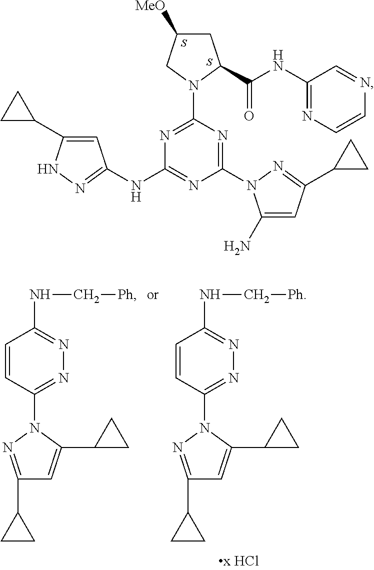

In certain other embodiments, compounds of formula ID are provided:

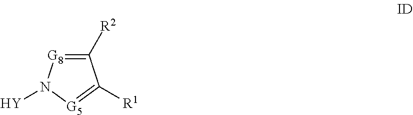

or a pharmaceutically acceptable salt thereof, wherein:

both of G₅ and G₈ are CR³, or one of G₅ and G₈ is N and the other is CR³;

when one of G₅ or G₈ is N, R³ is hydrogen, —CN, halogen, —Z—R⁵, or an optionally substituted group selected from C₁₋₆ aliphatic and 3- to 10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
   R^{3a} is hydrogen or an optionally substituted C₁₋₄ aliphatic, and
R⁵ is hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;

when G₅ and G₈ are both CR³, each occurrence of R³ is independently hydrogen, CN, or an optionally substituted C₁₋₃ aliphatic;

R¹ is —CN, —C(O)N(R⁴)₂, —C(O)OR⁴, —C(NR⁴)N(R⁴)₂, —NHCOR⁴, —NHSO₂R⁴, —NHCON(R⁴)₂, —NHCOOR⁴, —NHSO₂N(R⁴)₂, —CH₂OR⁴, —CH₂N(R⁴)₂, —CH₂NHC(O)R⁴, —SO₂NR⁴₂, —CONHC(═NH)N(R⁴)₂, —NHSO₂OR⁴, or CY, wherein CY is an optionally substituted group selected from a 3- to −7-membered cycloaliphatic; a 4- to 10-membered heterocyclyl having 1-5 heteroatoms independently selected from nitrogen, oxygen, or sulfur; a 6- to 10-membered aryl, or 5- to 10-membered heteroaryl having 1-5 heteroatoms independently selected from nitrogen, oxygen, or sulfur; wherein:
each R⁴ is independently selected from hydrogen, —OH, or an optionally substituted group selected from C₁₋₆ aliphatic, 3- to 10-membered cycloaliphatic, 6- to 10-membered aryl, or 5- to 10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, —NH₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur; or
two occurrences of R⁴, taken together with a nitrogen atom to which they are bound, form an optionally substituted 4- to −7-membered heterocyclyl ring having 0-1 additional heteroatoms independently selected from nitrogen, oxygen, or sulfur;
R² is halo or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}, -T₂-R^{12a} or —V₂-T₂-R^{12d}, and:
each occurrence of R^{12a} is independently halogen, —CN, —NO₂, —R^{12c}, —N(R^{12b})₂, —OR^{12b}), —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})SO₂R^{12c}, —N(R^{12e})C(O)OR^{12b}, —N(R^{12e})C(O)N(R^{12b})₂, or —N(R^{12e})SO₂N(R^{12b})₂, or an optionally substituted C₁-C₆ aliphatic or C₁-C₆haloaliphatic;
each occurrence of R^{12b} is independently hydrogen or an optionally substituted group selected from C₁-C₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or two occurrences of R^{12b}, taken together with a nitrogen atom to which they are bound, form an optionally substituted 4- to −7-membered heterocyclyl ring having 0-1 additional heteroatoms selected from nitrogen, oxygen, or sulfur;
each occurrence of R^{12c} is independently hydrogen or an optionally substituted group selected from C₁-C₆ aliphatic, C₁-C₆ haloaliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12d} is independently hydrogen or an optionally substituted group selected from 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, —N(R^{12e})C(O)—, —N(R^{12e})SO₂—, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3- to −7 membered cycloaliphatic or heterocyclyl ring, wherein R¹³ is hydrogen or an optionally substituted C₁₋₄ aliphatic group; and

HY is a group selected from:

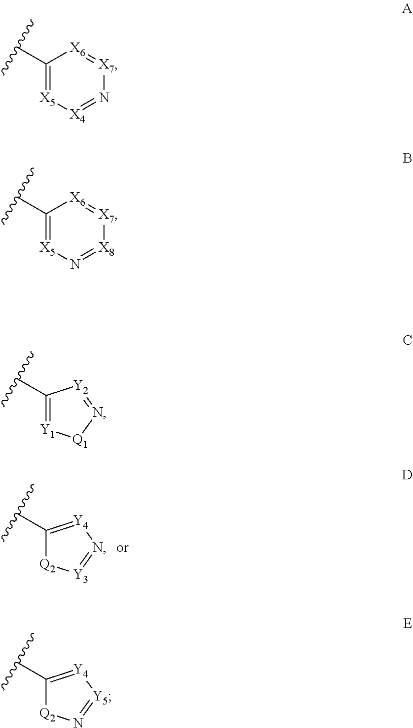

wherein
each occurrence of X₄, X₅, X₆, X₇, and X₈ is independently —CR¹⁰, —CR^{10′}, or N, provided no more than two occurrences of X₄, X₅, X₆, X₇, and X₈ is N;
each occurrence of Y₁, Y₂, Y₃, Y₄, and Y₅ is —CR¹⁰;
each occurrence of Q₁ and Q₂ is independently S, O or —NR⁹;
two adjacent occurrences of X₄ and X₅, X₆ and X₇, X₇ and X₈, Y₁ and —NR⁹, Y₃ and —NR⁹, or Y₄ and Y₅, may be taken together with the atoms to which they are bound, to form an unsubstituted fused heteroaryl or heterocyclyl group having 8 to 10 ring atoms and having 1-5 heteroatoms independently selected from nitrogen, oxygen, or sulfur;
each occurrence of R¹⁰ or R^{10′} is independently —R^{10b}, −V₁—R^{10c}, -T₁-R^{10b}, or −V₁-T₁-R^{10b}, wherein:
   V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
   each occurrence of R^{10a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3- to −7 membered cycloaliphatic or heterocyclyl ring;
   each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   each occurrence of R^{10c} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or
R^{10a} and R^{10b}, taken together with a nitrogen atom to which they are bound, form an optionally substituted 4- to −7-membered heterocyclyl ring having 0-1 additional heteroatoms independently selected from nitrogen, oxygen, or sulfur;
each occurrence of R¹¹ is independently hydrogen, —C(O)R^{11a}, —CO₂R^{11a}, —C(O)N(R^{11a})₂, —C(O)N(R^{11a})—OR^{11a}, —SO₂R^{11a}, —SO₂N(R^{11a})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   wherein each occurrence of R^{11a} is independently hydrogen or an optionally substituted group selected from C₁₋₆aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
   wherein each occurrence of R^{9a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
   wherein each occurrence of R^{9b} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3- to 6-membered heterocyclyl having 1-5 heteroatoms independently selected from nitrogen, oxygen, or sulfur, or 5- to 10-membered heteroaryl having 1-5 heteroatoms independently selected from nitrogen, oxygen, or sulfur; and
provided that when HY is a non-fused group then HY is substituted with at least one occurrence of R¹⁰ or R^{10′}, wherein R¹⁰ or R^{10′} is:
   —N(R¹¹)C(O)R^{10a}, —C(O)N(R¹¹)₂, or —NR¹¹C(O)OR^{10a}, or
   —V₁-T_{1-R}^{10b}, (wherein V₁ is —NR¹¹—, T₁ is a C₁-C₃ alkylene chain, and R^{10b} is an optionally substituted 6- to 10-membered aryl ring or a 5- to 10-membered heteroaryl ring having 1-5 heteroatoms independently selected from nitrogen, oxygen, or sulfur, or V₁ is —NR¹¹C(O)NR¹¹—, T₁ is a C₁-C₃ alkylene chain, and R^{10b} is —OR^{10a}; or
   —V₁—R^{10c}, wherein V₁ is —NR¹¹—, and R^{10c} is a 5- to 10-membered heteroaryl ring having 1-5 heteroatoms independently selected from nitrogen, oxygen, or sulfur; and
      provided that:

a) for compounds where -G₅-G₆-G₇-G₈-G₉ is —CR³═C—C═N—N or —CR³═C—C═CR³N:
(i) when G_{g} is N, R² is methyl, and HY is

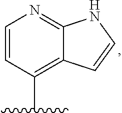 [SMILES: *c1ccnc2[nH]ccc12]

then R¹ is not an optionally substituted phenyl;
(ii) when G₈ is CH, then HY is not

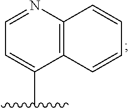 [SMILES: *c1ccnc2ccccc12]

and

b) for compounds where -G₅-G₆-G₇-G₈-G₉ is —N═C—C═CR³—N:
(i) when R² is methyl and R³ is hydrogen, then HY is not

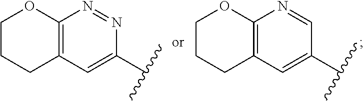 [SMILES: *c1cc2c(nn1)OCCC2 ; *c1cnc2c(c1)CCCO2]

(ii) when R¹ is unsubstituted thiazolyl, then HY is not substituted with —CH₂CH₂OH or —CH₂CH₂OSiMe₂t-Bu; and

c) provided that the compound is other than:

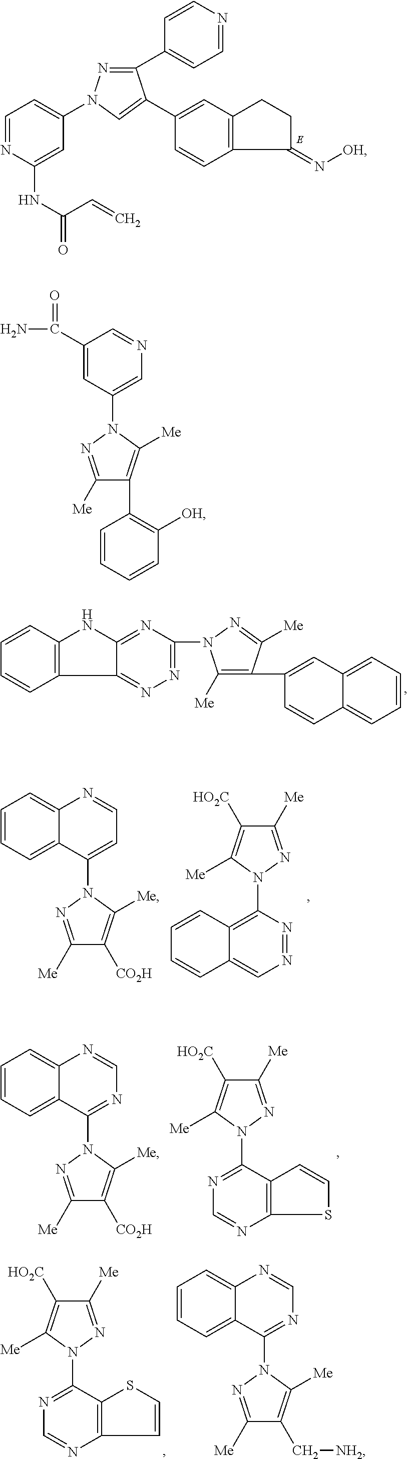

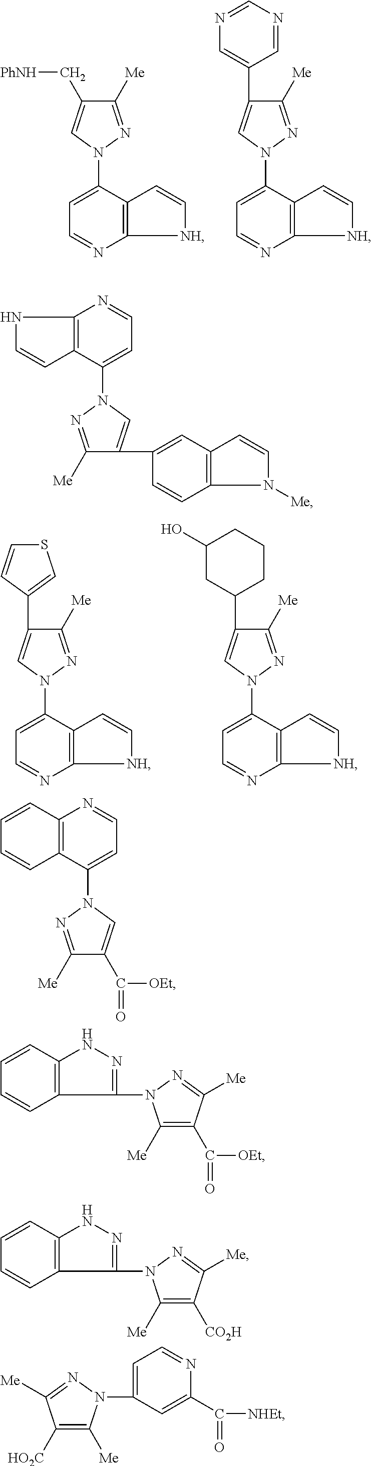

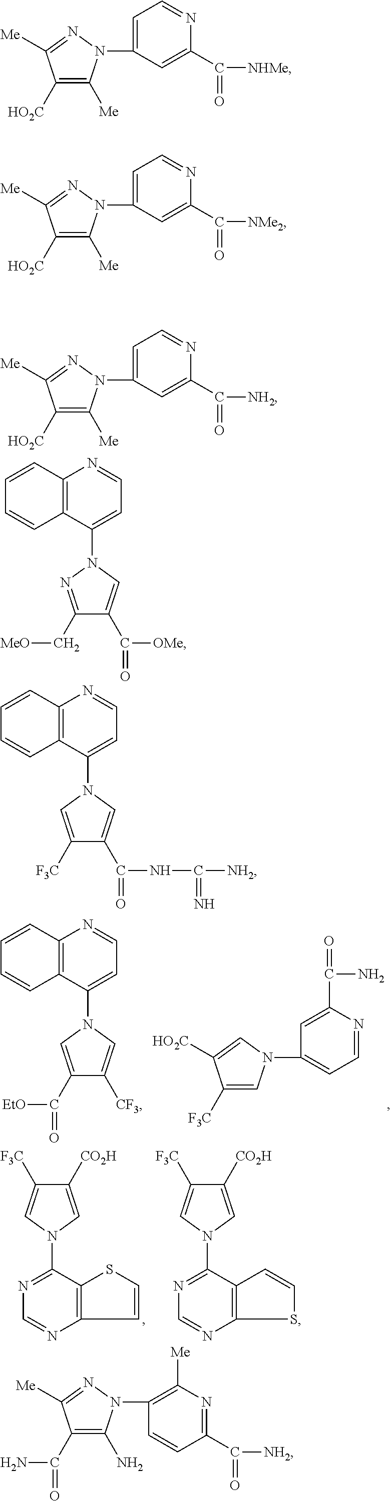

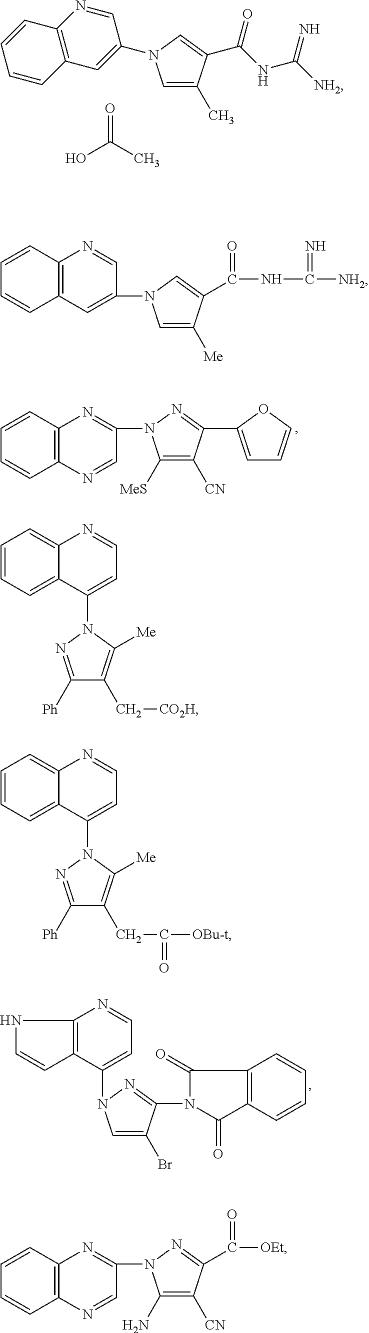

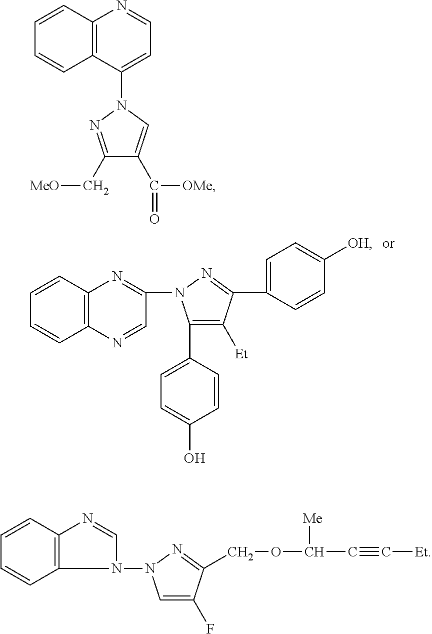 [SMILES: COCc1nn(-c2ccnc3ccccc23)cc1C(=O)OC ; CCc1c(-c2ccc(O)cc2)nn(-c2cnc3ccccc3n2)c1-c1ccc(O)cc1 ; CCC#CC(C)OCc1nn(-n2cnc3ccccc32)cc1F]

In certain other embodiments, compounds of formula ID are provided:

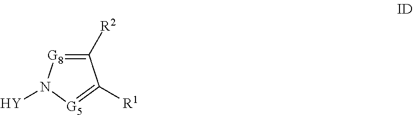

or a pharmaceutically acceptable salt thereof are provided, wherein:

both of G₅ and G₈ are CR³, or one of G₅ and G₈ is N and the other is CR³; when one of G₅ or G₈ is N, R³ is hydrogen, —CN, halogen, —Z—R⁵, or an optionally substituted group selected from C₁₋₆ aliphatic and 3- to 10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
   R^{3a} is hydrogen or an optionally substituted C₁₋₄ aliphatic, and
R⁵ is hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;

when G₅ and G₈ are both CR³, each occurrence of R³ is independently hydrogen, CN, or an optionally substituted C₁₋₃ aliphatic;

R¹ is —CN, —C(O)N(R⁴)₂, —C(O)OR⁴, —C(NR⁴)N(R⁴)₂, —NHCOR⁴, —NHSO₂R⁴, —NHCON(R⁴)₂, —NHCOOR⁴, —NHSO₂N(R⁴)₂, —CH₂OR⁴, —CH₂N(R⁴)₂, —CH₂NHC(O)R⁴, —SO₂NR⁴₂, —C(O)NHC(═NH)NR⁴₂, —NHSO₂OR⁴, or CY, wherein CY is an optionally substituted group selected from a 3- to −7-membered cycloaliphatic; a 4- to 10-membered heterocyclyl having 1-5 heteroatoms independently selected from nitrogen, oxygen, or sulfur; a 6-10 membered aryl, or 5- to 10-membered heteroaryl having 1-5 heteroatoms independently selected from nitrogen, oxygen, or sulfur; wherein:
each R⁴ is independently selected from hydrogen, —OH, or an optionally substituted group selected from C₁₋₆ aliphatic, 3- to 10-membered cycloaliphatic, 6- to 10-membered aryl, or 5- to 10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, —NH₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur; or
two occurrences of R⁴, taken together with a nitrogen atom to which they are bound, form an optionally substituted 4- to −7-membered heterocyclyl ring having 0-1 additional heteroatoms independently selected from nitrogen, oxygen, or sulfur;
R² is an optionally substituted group selected from a 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12a}, -T₂-R^{12a}, or —V₂-T₂-R^{12d}, and:
each occurrence of R^{12a} is independently halogen, —CN, —NO₂, —R^{12c}, —N(R¹²)₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})SO₂R^{12c}, —N(R^{12e})C(O)OR^{12b}, —N(R^{12e})C(O)N(R^{12b})₂, or —N(R^{12e})SO₂N(R^{12b})₂, or an optionally substituted C₁-C₆ aliphatic or C₁-C₆haloaliphatic;
each occurrence of R^{12b} is independently hydrogen or an optionally substituted group selected from C₁-C₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or two occurrences of R^{12b}, taken together with a nitrogen atom to which they are bound, form an optionally substituted 4- to −7-membered heterocyclyl ring having 0-1 additional heteroatoms selected from nitrogen, oxygen, or sulfur;
each occurrence of R^{12c} is independently hydrogen or an optionally substituted group selected from C₁-C₆ aliphatic, C₁-C₆ haloaliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12d} is independently hydrogen or an optionally substituted group selected from 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12c} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})OC(O)N(R^{12e})—, N(R^{12e})C(O)—, —N(R^{12e})SO₂N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3- to −7 membered cycloaliphatic or heterocyclyl ring, wherein R¹³ is hydrogen or an optionally substituted C₁₋₄ aliphatic group; and

HY is a group selected from:

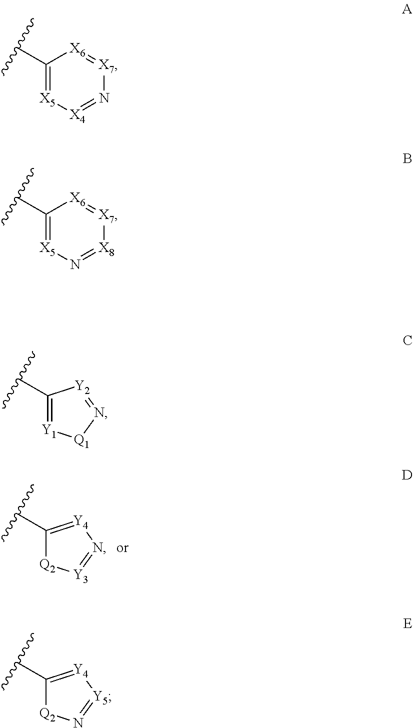

wherein
each occurrence of X₄, X₅, X₆, X₇, and X₈ is independently —CR¹⁰, —CR^{10′} or N, provided no more than two occurrences of X₄, X₅, X₆, X₇, and X₈ is N;
each occurrence of Y₁, Y₂, Y₃, Y₄, and Y₅ is —CR¹⁰;
each occurrence of Q₁ and Q₂ is independently S, O or —NR⁹;
two adjacent occurrences of X₄ and X₅, X₆ and X₇, X₇ and X₈, Y₁ and —NR⁹, Y₃ and —NR⁹, or Y₄ and Y₅, may be taken together with the atoms to which they are bound, to form an unsubstituted fused heteroaryl or heterocyclyl group having 8 to 10 ring atoms and having 1-5 heteroatoms independently selected from nitrogen, oxygen, or sulfur;
each occurrence of R¹⁰ or R ^{′} is independently —R^{10b}, —V₁—R^{10c}, -T₁-R^{10b}, or —V₁-T₁-R^{10b}, wherein:
   V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
   each occurrence of R^{10a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, —N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3- to −7 membered cycloaliphatic or heterocyclyl ring;
   each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   each occurrence of R^{10c} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or
R^{10a} and R^{10b}, taken together with a nitrogen atom to which they are bound, form an optionally substituted 4- to −7-membered heterocyclyl ring having 0-1 additional heteroatoms independently selected from nitrogen, oxygen, or sulfur;
each occurrence of R¹¹ is independently hydrogen, —C(O)R^{11a}, —CO₂R^{11a}, —C(O)N(R^{11a})₂, C(O)N(R^{11a})—OR^{11a}, —SO₂R^{11a}, —SO₂(R^{11a})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   wherein each occurrence of R^{11a} is independently hydrogen or an optionally substituted group selected from C₁₋₆aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
   wherein each occurrence of R^{9a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
   wherein each occurrence of R^{9b} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3- to 6-membered heterocyclyl having 1-5 heteroatoms independently selected from nitrogen, oxygen, or sulfur, or 5- to 10-membered heteroaryl having 1-5 heteroatoms independently selected from nitrogen, oxygen, or sulfur; and
provided that when HY is a non-fused group then HY is substituted with at least one occurrence of R¹⁰ or R^{10′}, wherein R¹⁰ or R^{10′} is:
   —N(R¹¹)C(O)R^{10a}, —C(O)N(R¹¹)₂, or —NR¹¹C(O)OR^{10a}; or
   —V₁-T₁-R^{10b}, wherein V₁ is —NR¹¹—, T₁ is a C₁-C₃ alkylene chain, and R^{10b} is an optionally substituted 6- to 10-membered aryl ring or a 5- to 10-membered heteroaryl ring having 1-5 heteroatoms independently selected from nitrogen, oxygen, or sulfur, or V₁ is —NR¹¹C(O)NR¹¹—, T₁ is a C₁-C₃ alkylene chain, and R^{10b} is —OR^{10a}; or
   —V₁—R^{10c}, wherein V₁ is —NR¹¹—, and R^{10c} is a 5- to 10-membered heteroaryl ring having 1-5 heteroatoms independently selected from nitrogen, oxygen, or sulfur; and
      provided that:

a) for compounds where -G₅-G₆-G₇-G₈-G₉ is —CR³═C—C═N—N or —CR³═C—C═CR³N:
(i) when G₈ is CH, then HY is not

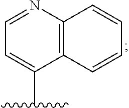 [SMILES: *c1ccnc2ccccc12]

and

b) for compounds where -G₅-G₆-G₇-G₈-G₉ is —N═C—C═CR³—N:
(i) when R¹ is unsubstituted thiazolyl, then HY is not substituted with —CH₂CH₂OH or —CH₂CH₂OSiMe₂t-Bu;

provided that the compound is other than:

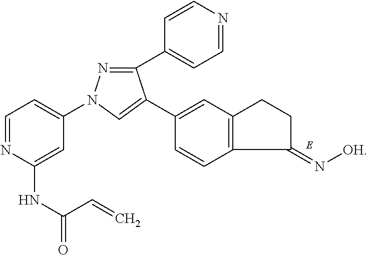 [SMILES: C=CC(=O)Nc1cc(-n2cc(-c3ccc4c(c3)CC/C4=N\O)c(-c3ccncc3)n2)ccn1]

In certain other embodiments, compounds of formula ID are provided:

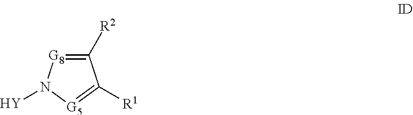

or a pharmaceutically acceptable salt thereof are provided, wherein:

G₅ is CR³;

G₈ is N or CR³;

when G₈ is N, R³ is hydrogen, —CN, halogen, —Z—R⁵, or an optionally substituted group selected from C₁₋₆aliphatic and 3- to 10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
   R^{3a} is hydrogen or an optionally substituted C₁₋₄ aliphatic, and
R⁵ is hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;

when G₈ is CR³, each occurrence of R³ is independently hydrogen, CN, or an optionally substituted C₁₋₃ aliphatic;

R¹ is —CN, —C(O)N(R⁴)₂, —C(O)OR⁴, —C(NR⁴)N(R⁴)₂, —NHCOR⁴, —NHSO₂R⁴, —NHCON(R⁴)₂, —NHCOOR⁴, —NHSO₂N(R⁴)₂, —CH₂OR⁴, —CH₂N(R⁴)₂, —CH₂NHC(O)R⁴, —SO₂NR⁴₂, —CONHC(═NH)N(R⁴)₂, —NHSO₂OR⁴, or CY, wherein CY is an optionally substituted group selected from a 3- to −7-membered cycloaliphatic; a 4- to 10-membered heterocyclyl having 1-5 heteroatoms independently selected from nitrogen, oxygen, or sulfur; a 6-10 membered aryl, or 5- to 10-membered heteroaryl having 1-5 heteroatoms independently selected from nitrogen, oxygen, or sulfur; wherein:
each R⁴ is independently selected from hydrogen, —OH, or an optionally substituted group selected from C₁₋₆ aliphatic, 3- to 10-membered cycloaliphatic, 6- to 10-membered aryl, or 5- to 10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, —NH₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur; or
two occurrences of R⁴, taken together with a nitrogen atom to which they are bound, form an optionally substituted 4- to −7-membered heterocyclyl ring having 0-1 additional heteroatoms independently selected from nitrogen, oxygen, or sulfur;
R² is hydrogen, halo or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, T₂-R^{12d}, -T₂-R^{12a}, or —V₂-T₂-R^{12d}, and:
each occurrence of R^{12a} is independently halogen, —CN, —NO₂, —R^{12c}, —N(R^{12b})₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})SO₂R^{12c}, —N(R^{12e})C(O)OR^{12b}, —N(R^{12e})C(O)N(R^{12b})₂, or —N(R^{12e})SO₂N(R^{12b})₂, or an optionally substituted C₁-C₆ aliphatic or C₁-C₆haloaliphatic;
each occurrence of R^{12b} is independently hydrogen or an optionally substituted group selected from C₁-C₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or two occurrences of R^{12b}, taken together with a nitrogen atom to which they are bound, form an optionally substituted 4- to −7-membered heterocyclyl ring having 0-1 additional heteroatoms selected from nitrogen, oxygen, or sulfur;
each occurrence of R^{12c} is independently hydrogen or an optionally substituted group selected from C₁-C₆ aliphatic, C₁-C₆ haloaliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12d} is independently hydrogen or an optionally substituted group selected from 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, —N(R^{12e})C(O)—, —N(R^{12e})SO₂—, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3- to −7 membered cycloaliphatic or heterocyclyl ring, wherein R¹³ is hydrogen or an optionally substituted C₁₋₄ aliphatic group; and

HY is a group selected from:

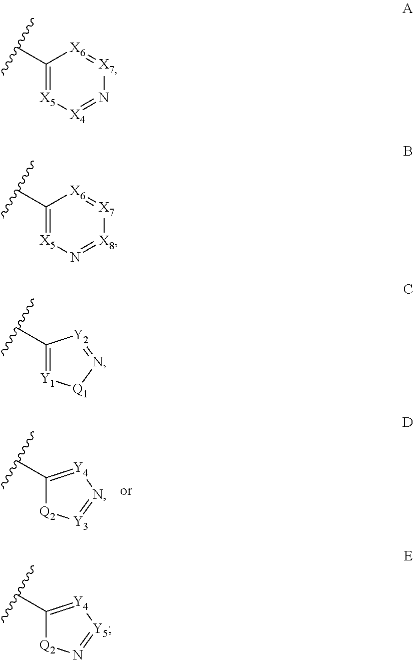

wherein
each occurrence of X₄, X₅, X₆, X₇, and X₈ is independently —CR¹⁰, —CR^{10′}, or N, provided no more than two occurrences of X₄, X₅, X₆, X₇, and X₈ is N;
each occurrence of Y₁, Y₂, Y₃, Y₄, and Y₅ is —CR¹⁰;
each occurrence of Q₁ and Q₂ is independently S, O or —NR⁹;
two adjacent occurrences of X₄ and X₅, X₆ and X₇, X₇ and X₈, Y₁ and —NR⁹, Y₃ and —NR⁹, or Y₄ and Y₅, may be taken together with the atom to which they are bound, to form an unsubstituted fused group having 8 to 10 ring atoms selected from an aryl group, or a heteroaryl group having 1-5 heteroatoms independently selected from nitrogen, oxygen, or sulfur;
each occurrence of R¹⁰ or R^{10′} is independently —R^{10b}, —V₁—R^{10c}, or —V₁-T₁-R^{10b}, or —V₁-T₁-R^{10b}, wherein:
   V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
   each occurrence of R^{10a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, —N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3- to −7 membered cycloaliphatic or heterocyclyl ring;
   each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   each occurrence of R^{10c} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or
R^{10a} and R^{10b}, taken together with a nitrogen atom to which they are bound, form an optionally substituted 4- to −7-membered heterocyclyl ring having 0-1 additional heteroatoms independently selected from nitrogen, oxygen, or sulfur; each occurrence of R¹¹ is independently hydrogen, —C(O)R^{11a}, —CO₂R^{11a}, —C(O)N(R^{11a})₂, C(O)N(R^{11a})—OR^{11a}, —SO₂R^{11a}, —SO₂N(R^{11a})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;

wherein each occurrence of R^{11a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
   wherein each occurrence of R^{9a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
   wherein each occurrence of R^{9b} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3- to 6-membered heterocyclyl having 1-5 heteroatoms independently selected from nitrogen, oxygen, or sulfur, or 5- to 10-membered heteroaryl having 1-5 heteroatoms independently selected from nitrogen, oxygen, or sulfur; and

provided that when HY is a non-fused group then HY is substituted with at least one occurrence of R¹⁰ or R^{10′}, wherein R¹⁰ or R^{10′} is:
—N(R¹¹)C(O)R^{10a}, —C(O)N(R¹¹)₂, or —NR¹¹C(O)OR^{10a}; or
—V₁-T₁-R^{10b}, wherein V₁ is —NR¹¹—, T₁ is a C₁-C₃ alkylene chain, and R^{10b} is an optionally substituted 6- to 10-membered aryl ring or a 5- to 10-membered heteroaryl ring having 1-5 heteroatoms independently selected from nitrogen, oxygen, or sulfur; or
—V₁—R^{10c}, wherein V₁ is —NR¹¹—, and R^{10c} is a 5- to 10-membered heteroaryl ring having 1-5 heteroatoms independently selected from nitrogen, oxygen, or sulfur; and
   provided that:

when G₈ is N, R² is methyl, and HY is

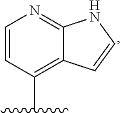 [SMILES: *c1ccnc2[nH]ccc12]

then R¹ is not an optionally substituted phenyl;

when G₈ is CH, then HY is not

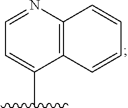 [SMILES: *c1ccnc2ccccc12]

when G₈ is CH, R² is hydrogen, and HY is 3-pyridyl, then HY is not substituted with

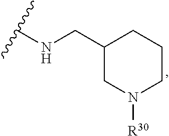

wherein R³⁰ is hydrogen, or —CO₂-tert-butyl;

provided that for compounds where G₈ is N and R² is hydrogen:
(x) when HY is 4-pyridyl, then R¹ is not —CO₂H;
(xi) HY is not substituted with:

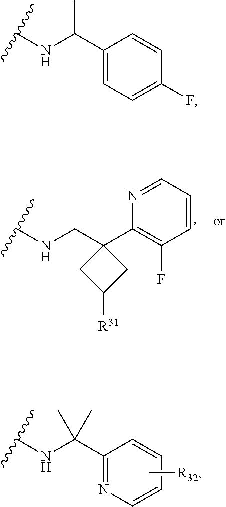

wherein R³¹ is hydrogen or fluoro;
R³² is fluoro, chloro, or —OCHF₂;
(xii) when G₅ is C—R³, and R³ is —CH₃ or —NH₂, then R¹ is not —CO₂Et; and
   provided that the compound is other than:

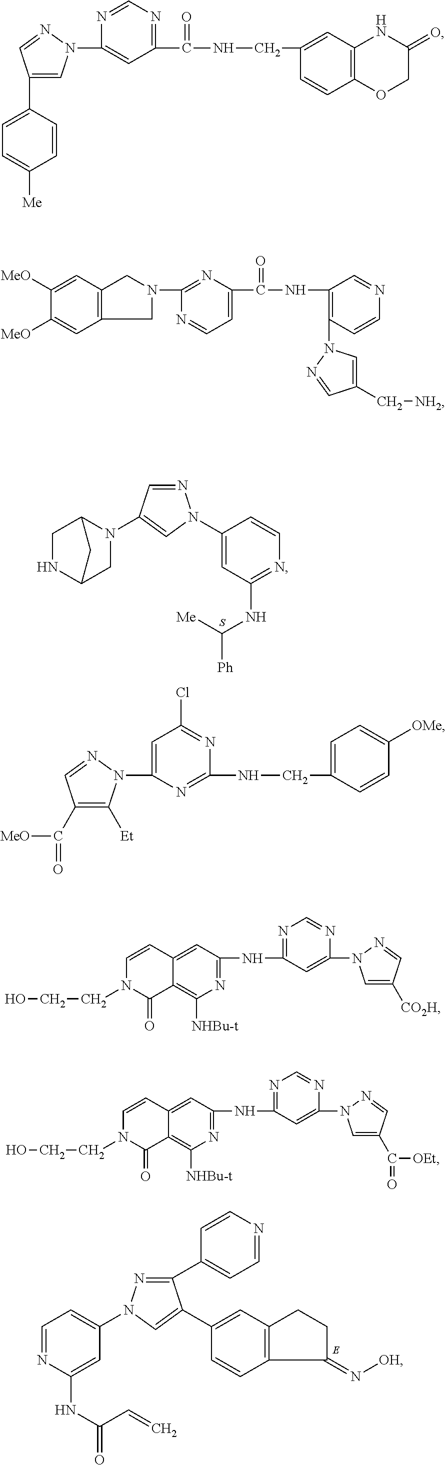

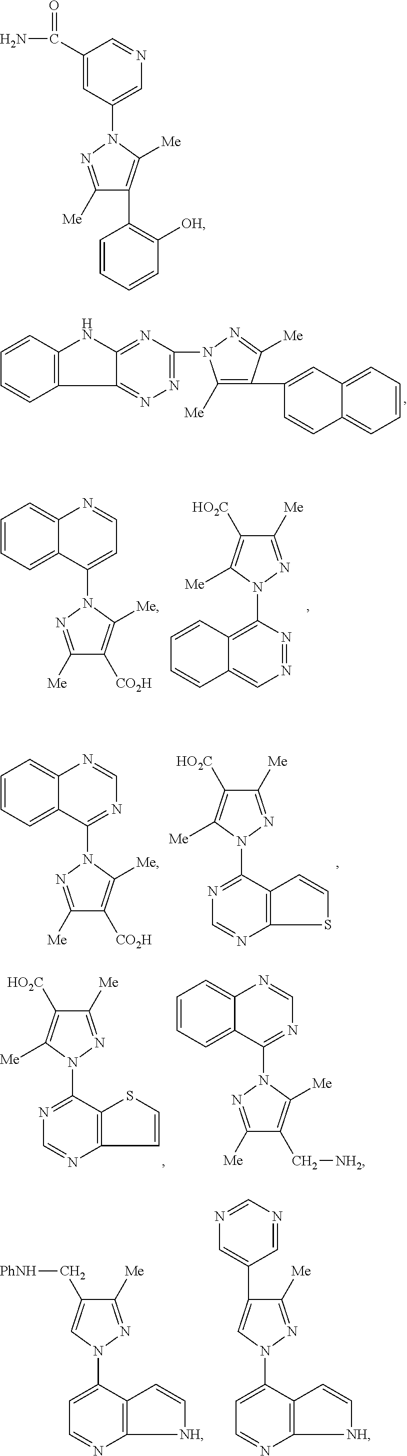

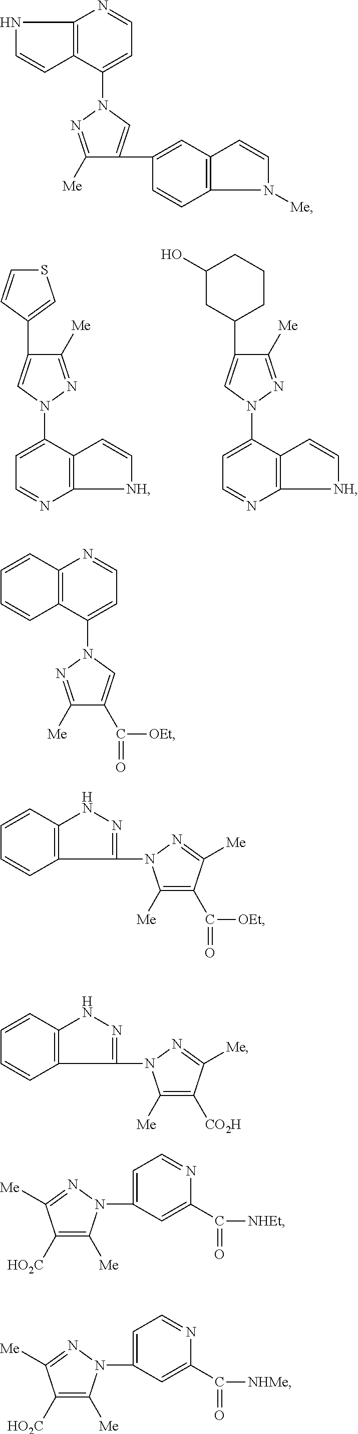

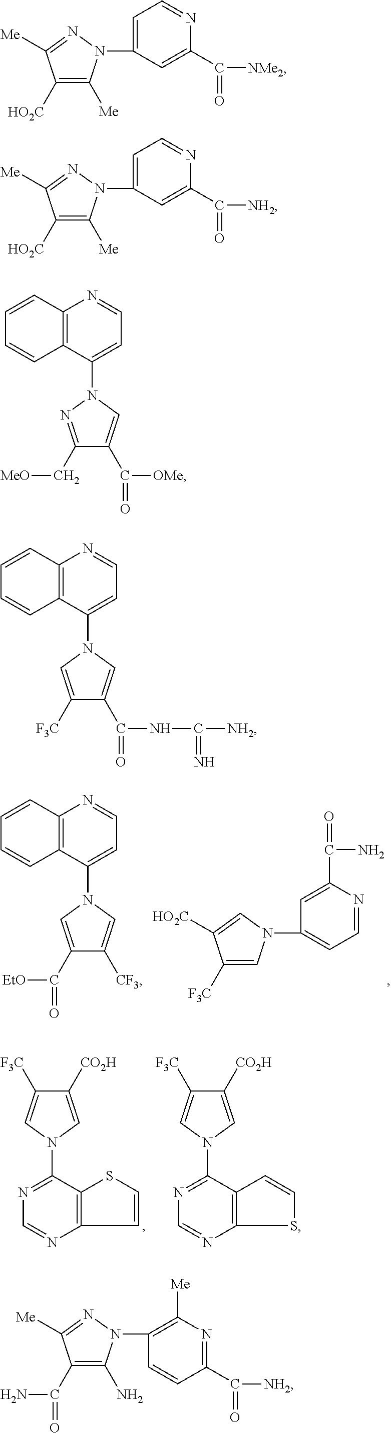

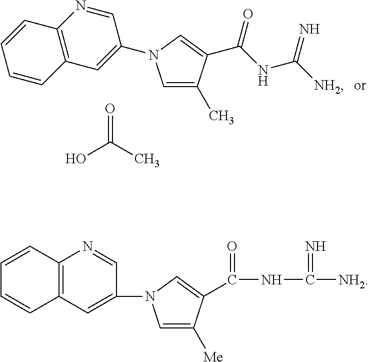

In certain other embodiments, compounds of formula ID are provided:

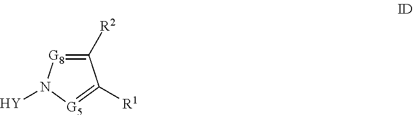

or a pharmaceutically acceptable salt thereof are provided, wherein:

G₅ is CR³;

G₈ is N or CR³;

when G₈ is N, R³ is hydrogen, —CN, halogen, —Z—R⁵, or an optionally substituted group selected from C₁₋₆aliphatic and 3- to 10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
   R^{3a} is hydrogen or an optionally substituted C₁₋₄ aliphatic, and
R⁵ is hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;

when G₈ is CR³, each occurrence of R³ is independently hydrogen, CN, or an optionally substituted C₁₋₃ aliphatic;

R¹ is —CN, —C(O)N(R⁴)₂, —C(O)OR⁴, —C(NR⁴)N(R⁴)₂, —NHCOR⁴, —NHSO₂R⁴, —NHCON(R⁴)₂, —NHCOOR⁴, —NHSO₂N(R⁴)₂, —CH₂OR⁴, —CH₂N(R⁴)₂, —CH₂NHC(O)R⁴, —SO₂NR⁴₂, —C(O)NHC(═NH)NR⁴₂, —NHSO₂OR⁴, or CY, wherein CY is an optionally substituted group selected from a 3- to −7-membered cycloaliphatic; a 4- to 10-membered heterocyclyl having 1-5 heteroatoms independently selected from nitrogen, oxygen, or sulfur; a 6-10 membered aryl, or 5- to 10-membered heteroaryl having 1-5 heteroatoms independently selected from nitrogen, oxygen, or sulfur; wherein:
each R⁴ is independently selected from hydrogen, —OH, or an optionally substituted group selected from C₁₋₆ aliphatic, 3- to 10-membered cycloaliphatic, 6- to 10-membered aryl, or 5- to 10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, —NH₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur; or
two occurrences of R⁴, taken together with a nitrogen atom to which they are bound, form an optionally substituted 4- to −7-membered heterocyclyl ring having 0-1 additional heteroatoms independently selected from nitrogen, oxygen, or sulfur;
R² is halo or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}, -T₂-R^{12a}, or —V₂-T₂-R^{12d}, and:
each occurrence of R^{12a} is independently halogen, —CN, —NO₂, —R^{12c}, —N(R^{12b})₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})SO₂R^{12c}, —N(R^{12e})C(O)OR^{12b}, —N(R^{12e})C(O)N(R^{12b})₂, —N(R^{12e})SO₂N(R^{12b})₂, or an optionally substituted C₁-C₆ aliphatic or C₁-C₆haloaliphatic;
each occurrence of R^{12b} is independently hydrogen or an optionally substituted group selected from C₁-C₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or two occurrences of R^{12b}, taken together with a nitrogen atom to which they are bound, form an optionally substituted 4- to −7-membered heterocyclyl ring having 0-1 additional heteroatoms selected from nitrogen, oxygen, or sulfur;
each occurrence of R^{12c} is independently hydrogen or an optionally substituted group selected from C₁-C₆ aliphatic, C₁-C₆ haloaliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12d} is independently hydrogen or an optionally substituted group selected from 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, —N(R^{12e})C(O)—, —N(R^{12e})SO₂, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3- to −7 membered cycloaliphatic or heterocyclyl ring, wherein R¹³ is hydrogen or an optionally substituted C₁₋₄ aliphatic group; and

HY is a group selected from:

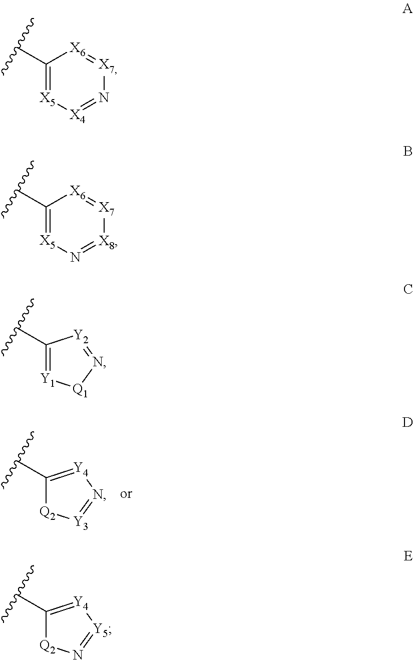

wherein
each occurrence of X₄, X₅, X₆, X₇, and X₈ is independently —CR¹⁰, —CR^{10′}, or N, provided no more than two occurrences of X₄, X₅, X₆, X₇, and X₈ is N;
each occurrence of Y₁, Y₂, Y₃, Y₄, and Y₅ is —CR¹⁰;
each occurrence of Q₁ and Q₂ is independently S, O or —NR⁹;
two adjacent occurrences of X₄ and X₅, X₆ and X₇, X₇ and X₈, Y₁ and —NR⁹, Y₃ and —NR⁹, or Y₄ and Y₅, may be taken together with the atoms to which they are bound, to form an unsubstituted fused heteroaryl or heterocyclyl group having 8 to 10 ring atoms and having 1-5 heteroatoms independently selected from nitrogen, oxygen, or sulfur;
each occurrence of R¹⁰ or R^{10′} is independently —R^{10b}, —V₁—R^{10c}, -T₁-R^{10b}, or —V₁-T₁-R^{10b}, wherein:
   V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
   each occurrence of R^{10a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, N(R^{10a})C(O)N(R^{10a})—, —OC(O)—, or —C(O)N(R¹¹)—O— or wherein T₁ forms part of an optionally substituted 3- to −7 membered cycloaliphatic or heterocyclyl ring;
   each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   each occurrence of R^{10c} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or
R^{10a} and R^{10b}, taken together with a nitrogen atom to which they are bound, form an optionally substituted 4- to −7-membered heterocyclyl ring having 0-1 additional heteroatoms independently selected from nitrogen, oxygen, or sulfur;
each occurrence of R¹¹ is independently hydrogen, —C(O)R^{11a}, —CO₂R^{11a}, —C(O)N(R^{11a})₂, C(O)N(R^{11a})—OR^{11a}, —SO₂R^{11a}, —SO₂N(R^{11a})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
wherein each occurrence of R^{11a} is independently hydrogen or an optionally substituted group selected from C₁₋₆aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;

each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
wherein each occurrence of R^{9a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
wherein each occurrence of R^{9b} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3- to 6-membered heterocyclyl having 1-5 heteroatoms independently selected from nitrogen, oxygen, or sulfur, or 5- to 10-membered heteroaryl having 1-5 heteroatoms independently selected from nitrogen, oxygen, or sulfur; and

provided that when HY is a non-fused group then HY is substituted with at least one occurrence of R¹⁰ or R^{10′}, wherein R¹⁰ or R^{10′} is:
—N(R¹¹)C(O)R^{10a}, —C(O)N(R¹¹) or —NR¹¹C(O)OR^{10a}; or
—V₁-T₁-R^{10b}, wherein V₁ is —NR¹¹—, T₁ is a C₁-C₃ alkylene chain, and R^{10b} is an optionally substituted 6- to 10-membered aryl ring or a 5- to 10-membered heteroaryl ring having 1-5 heteroatoms independently selected from nitrogen, oxygen, or sulfur; or
V₁—R^{10c}, wherein V₁ is —NR¹¹—, and R^{10c} is a 5- to 10-membered heteroaryl ring having 1-5 heteroatoms independently selected from nitrogen, oxygen, or sulfur; and
   provided that:

when G₈ is N, R² is methyl, and HY is

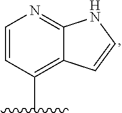 [SMILES: *c1ccnc2[nH]ccc12]

then R¹ is not an optionally substituted phenyl;

when G₈ is CH, then HY is not

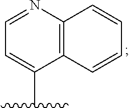 [SMILES: *c1ccnc2ccccc12]

and

provided that the compound is other than:

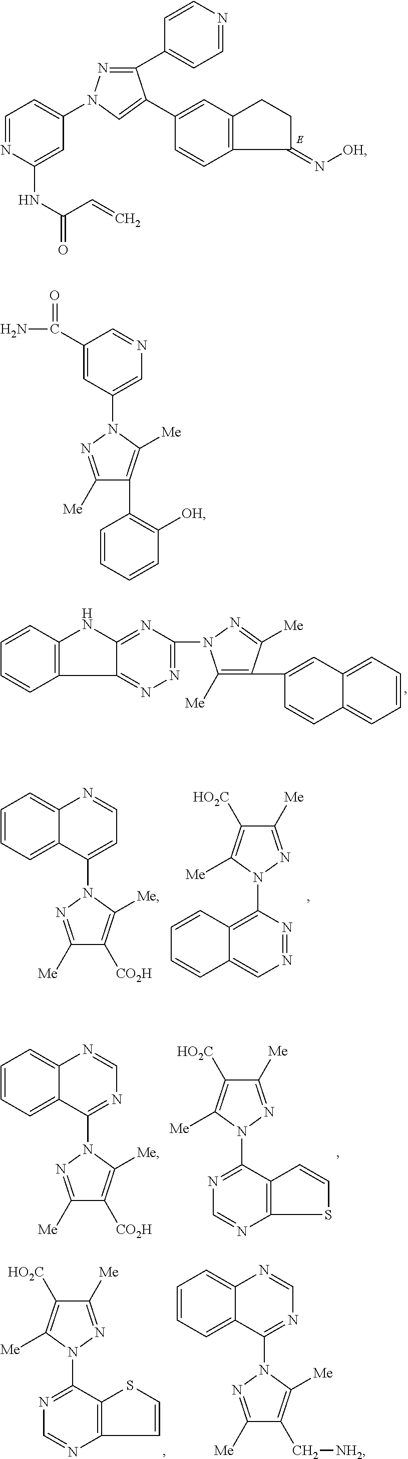

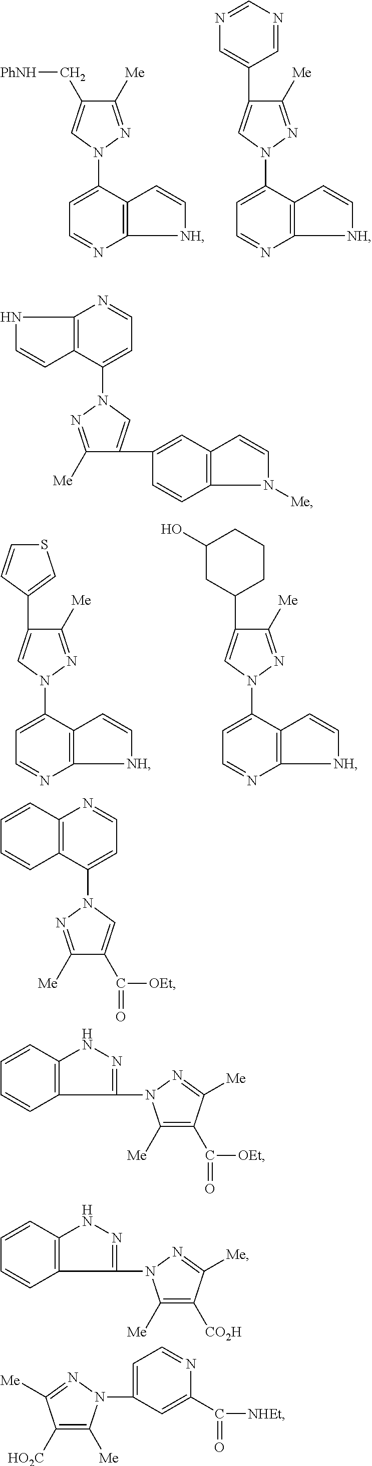

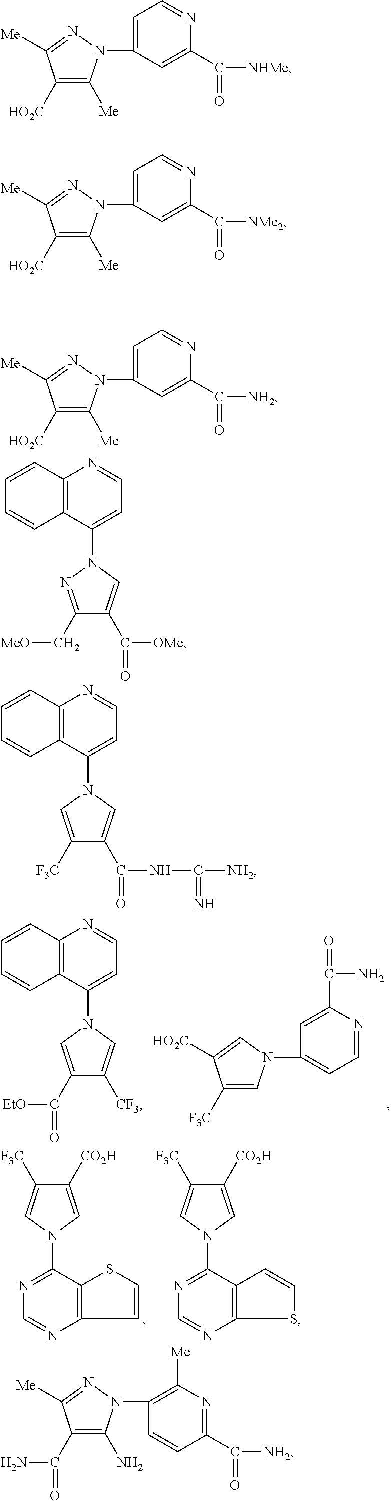

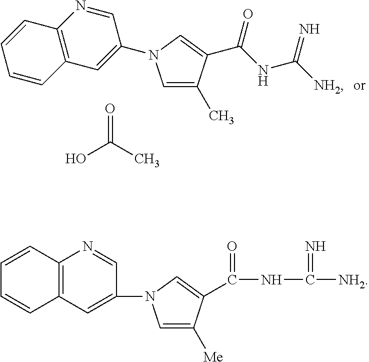

In certain other embodiments, compounds of formula ID are provided:

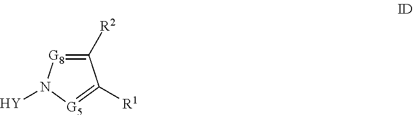

or a pharmaceutically acceptable salt thereof are provided, wherein:

G₅ is CR³;

G₈ is N or CR³;

when G₈ is N, R³ is hydrogen, —CN, halogen, —Z—R⁵, or an optionally substituted group selected from C₁₋₆ aliphatic and 3- to 10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
   R^{3a} is hydrogen or an optionally substituted C₁₋₄ aliphatic, and
R⁵ is hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;

when G₈ is CR³, each occurrence of R³ is independently hydrogen, CN, or an optionally substituted C₁₋₃ aliphatic;

R¹ is —CN, —C(O)N(R⁴)₂, —C(O)OR⁴, —C(NR⁴)N(R⁴)₂, —NHCOR⁴, —NHSO₂R⁴, —NHCON(R⁴)₂, —NHCOOR⁴, —NHSO₂N(R⁴)₂, —CH₂OR⁴, —CH₂N(R⁴)₂, —CH₂NHC(O)R⁴, —SO₂NR⁴₂, —C(O)NHC(═NH)NR⁴₂, —NHSO₂OR⁴, or CY, wherein CY is an optionally substituted group selected from a 3- to −7-membered cycloaliphatic; a 4- to 10-membered heterocyclyl having 1-5 heteroatoms independently selected from nitrogen, oxygen, or sulfur; a 6-10 membered aryl, or 5- to 10-membered heteroaryl having 1-5 heteroatoms independently selected from nitrogen, oxygen, or sulfur; wherein:
each R⁴ is independently selected from hydrogen, —OH, or an optionally substituted group selected from C₁₋₆ aliphatic, 3- to 10-membered cycloaliphatic, 6- to 10-membered aryl, or 5- to 10-membered heteroaryl having 1-5 heteroatoms independently selected from nitrogen, oxygen, or sulfur; or
R⁴ is —Z₂—R⁶ wherein:
   Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
   R^{4a} is hydrogen or an optionally substituted C₁₋₄ aliphatic, and
   R⁶ is hydrogen, —NH₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur; or
two occurrences of R⁴, taken together with a nitrogen atom to which they are bound, form an optionally substituted 4- to −7-membered heterocyclyl ring having 0-1 additional heteroatoms independently selected from nitrogen, oxygen, or sulfur;
R² is an optionally substituted group selected from a 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}, -T₂-R^{12a}, or —V₂-T₂-R^{12d}, and:
each occurrence of R^{12a} is independently halogen, —CN, —NO₂, —R^{12c}, —N(R^{12b})₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R¹²)₂, —N(R^{12e})C(O)R^{12b}, —N(R^{12e})SO₂R^{12c}, —N(R^{12e})C(O)OR^{12b}, —N(R^{12e})C(O)N(R^{12b})₂, or —N(R^{12e})SO₂N(R^{12b})₂, or an optionally substituted C₁-C₆ aliphatic or C₁-C₆ haloaliphatic;
each occurrence of R^{12b} is independently hydrogen or an optionally substituted group selected from C₁-C₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or two occurrences of R^{12b}, taken together with a nitrogen atom to which they are bound, form an optionally substituted 4- to −7-membered heterocyclyl ring having 0-1 additional heteroatoms selected from nitrogen, oxygen, or sulfur;
each occurrence of R^{12c} is independently hydrogen or an optionally substituted group selected from C₁-C₆ aliphatic, C₁-C₆ haloaliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12d} is independently hydrogen or an optionally substituted group selected from 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, —N(R^{12e})C(O)—, —N(R^{12e})SO₂—, —N(R^{12e})C(O)O—, —N(R^{12e})C(O)N(R^{12e})—, —N(R^{12e})SO₂N(R^{12e})—, —OC(O)—, or —C(O)N(R^{12e})—O—; and

T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3- to −7 membered cycloaliphatic or heterocyclyl ring, wherein R¹³ is hydrogen or an optionally substituted C₁₋₄ aliphatic group; and

HY is a group selected from:

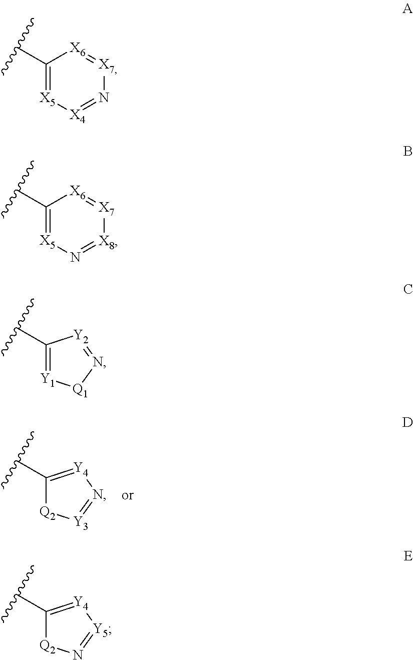

wherein
each occurrence of X₄, X₅, X₆, X₇, and X₈ is independently —CR¹⁰, —CR^{10′}, or N, provided no more than two occurrences of X₄, X₅, X₆, X₇, and X₈ is N;
each occurrence of Y₁, Y₂, Y₃, Y₄, and Y₅ is —CR¹⁰;
each occurrence of Q₁ and Q₂ is independently S, O or —NR⁹;
two adjacent occurrences of X₄ and X₅, X₆ and X₇, X₇ and X₈, Y₁ and —NR⁹, Y₃ and —NR⁹, or Y₄ and Y₅, may be taken together with the atoms to which they are bound, to form an unsubstituted fused heteroaryl or heterocyclyl group having 8 to 10 ring atoms and having 1-5 heteroatoms independently selected from nitrogen, oxygen, or sulfur;
each occurrence of R¹⁰ or R^{10′} is independently —R^{10b}, —V₁—R^{10c}, -T₁-R^{10b}, or —V₁-T₁-R^{10b}, wherein:
   V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, C(O)NR¹¹—, —C(O)NR¹¹O—, —SO₂—, or —SO₂NR¹¹—;
   each occurrence of R^{10a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, —N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3- to −7 membered cycloaliphatic or heterocyclyl ring;
   each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂ or —N(R¹¹)SO₂N(R¹¹)₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
   each occurrence of R^{10c} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or
R^{10a} and R^{10b}, taken together with a nitrogen atom to which they are bound, form an optionally substituted 4- to −7-membered heterocyclyl ring having 0-1 additional heteroatoms independently selected from nitrogen, oxygen, or sulfur;
each occurrence of R¹¹ is independently hydrogen, —C(O)R^{11a}, —CO₂R^{11a}, —C(O)N(R^{11a})₂, —C(O)N(R^{11a})—OR^{11a}, —SO₂R^{11a}, —SO₂N(R^{11a})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
wherein each occurrence of R^{11a} is independently hydrogen or an optionally substituted group selected from C₁₋₆aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
   wherein each occurrence of R^{9a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
   wherein each occurrence of R^{9b} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3- to 6-membered heterocyclyl having 1-5 heteroatoms independently selected from nitrogen, oxygen, or sulfur, or 5- to 10-membered heteroaryl having 1-5 heteroatoms independently selected from nitrogen, oxygen, or sulfur; and
provided that when HY is a non-fused group then HY is substituted with at least one occurrence of R¹⁰ or R^{10′}, wherein R¹⁰ or R^{10′} is:
—N(R¹¹)C(O)R^{10a}, —C(O)N(R¹¹)₂, or —NR¹¹C(O)OR^{10a}; or
—V₁-T₁-R^{10b}, wherein V₁ is —NR¹¹—, T₁ is a C₁-C₃ alkylene chain, and R^{10b} is an optionally substituted 6- to 10-membered aryl ring or a 5- to 10-membered heteroaryl ring having 1-5 heteroatoms independently selected from nitrogen, oxygen, or sulfur; or
—V₁—R^{10c}, wherein V₁ is —NR¹¹—, and R^{10c} is a 5- to 10-membered heteroaryl ring having 1-5 heteroatoms independently selected from nitrogen, oxygen, or sulfur; and
   provided that:

when G₈ is CH, then HY is not

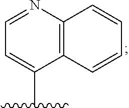 [SMILES: *c1ccnc2ccccc12]

and

provided that the compound is other than:

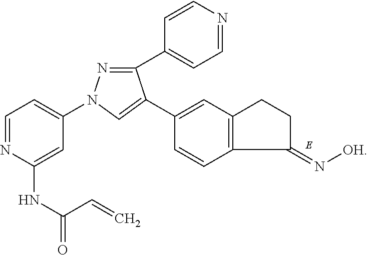 [SMILES: C=CC(=O)Nc1cc(-n2cc(-c3ccc4c(c3)CC/C4=N\O)c(-c3ccncc3)n2)ccn1]

### DETAILED DESCRIPTION OF THE INVENTION

### 2. Compounds and Definitions

Compounds of this invention include those described generally for formula IB and ID above, and are further illustrated by the classes, subclasses, and species disclosed herein. It will be appreciated that preferred subsets described for each variable herein can be used for any of the structural subsets as well. As used herein, the following definitions shall apply unless otherwise indicated.

As described herein, compounds of the invention may be optionally substituted with one or more substituents, such as are illustrated generally above, or as exemplified by particular classes, subclasses, and species of the invention. It will be appreciated that the phrase “optionally substituted” is used interchangeably with the phrase “substituted or unsubstituted.” In general, the term “substituted”, whether preceded by the term “optionally” or not, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which hydrogen atom can be replaced with the radical of a suitable substituent. Unless otherwise indicated, an “optionally substituted” group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

A stable compound or chemically feasible compound is one in which the chemical structure is not substantially altered when kept at a temperature from about −80° C. to about +40°, in the absence of moisture or other chemically reactive conditions, for at least a week, or a compound which maintains its integrity long enough to be useful for therapeutic or prophylactic administration to a patient.

The phrase “one or more substituents”, as used herein, refers to a number of substituents that equals from one to the maximum number of substituents possible based on the number of available bonding sites, provided that the above conditions of stability and chemical feasibility are met.

As used herein, the term “independently selected” means that the same or different values may be selected for multiple instances of a given variable in a single compound.

As used herein, “a 3-7-membered saturated, partially unsaturated, or aromatic monocyclic ring having 0-3 heteroatoms independently selected from nitrogen, oxygen, or sulfur, or an 8-10-membered partially unsaturated, or aromatic bicyclic ring system having 0-5 heteroatoms independently selected from nitrogen, oxygen, or sulfur” includes cycloaliphatic, heterocyclic, aryl and heteroaryl rings.

As used herein, the term “aromatic” includes aryl and heteroaryl groups as described generally below and herein.

The term “aliphatic” or “aliphatic group”, as used herein, means an optionally substituted straight-chain or branched C₁₋₁₂ hydrocarbon, or a cyclic C₁₋₁₂ hydrocarbon which is completely saturated or which contains one or more units of unsaturation, but which is not aromatic (also referred to herein as “carbocycle”, “cycloaliphatic”, “cycloalkyl”, or “cycloalkenyl”). For example, suitable aliphatic groups include optionally substituted linear, branched or cyclic alkyl, alkenyl, alkynyl groups and hybrids thereof, such as (cycloalkyl)alkyl, (cycloalkenyl)alkyl, or (cycloalkyl)alkenyl. Unless otherwise specified, in various embodiments, aliphatic groups have 1-12, 1-10, 1-8, 1-6, 1-4, 1-3, or 1-2 carbon atoms.

The term “alkyl”, used alone or as part of a larger moiety, refers to an optionally substituted straight or branched chain hydrocarbon group having 1-12, 1-10, 1-8, 1-6, 1-4, 1-3, or 1-2 carbon atoms.

The term “alkenyl”, used alone or as part of a larger moiety, refers to an optionally substituted straight or branched chain hydrocarbon group having at least one double bond and having 2-12, 2-10, 2-8, 2-6, 2-4, or 2-3 carbon atoms.

The term “alkynyl”, used alone or as part of a larger moiety, refers to an optionally substituted straight or branched chain hydrocarbon group having at least one triple bond and having 2-12, 2-10, 2-8, 2-6, 2-4, or 2-3 carbon atoms.

The terms “cycloaliphatic”, “carbocycle”, “carbocyclyl”, “carbocyclo”, or “carbocyclic”, used alone or as part of a larger moiety, refer to an optionally substituted saturated or partially unsaturated cyclic aliphatic ring system having from 3 to about 14 ring carbon atoms. In some embodiments, the cycloaliphatic group is an optionally substituted monocyclic hydrocarbon having 3-8 or 3-6 ring carbon atoms. Cycloaliphatic groups include, without limitation, optionally substituted cyclopropyl, cyclobutyl, cyclopentyl, cyclopentenyl, cyclohexyl, cyclohexenyl, cycloheptyl, cycloheptenyl, cyclooctyl, cyclooctenyl, or cyclooctadienyl. The terms “cycloaliphatic”, “carbocycle”, “carbocyclyl”, “carbocyclo”, or “carbocyclic” also include optionally substituted bridged or fused bicyclic rings having 6-12, 6-10, or 6-8 ring carbon atoms, wherein any individual ring in the bicyclic system has 3-8 ring carbon atoms.

The term “cycloalkyl” refers to an optionally substituted saturated ring system of about 3 to about 10 ring carbon atoms. Exemplary monocyclic cycloalkyl rings include cyclopropyl, cyclobutyl, cyclopentyl, cyclohexyl, and cycloheptyl.

The term “cycloalkenyl” refers to an optionally substituted non-aromatic monocyclic or multicyclic ring system containing at least one carbon-carbon double bond and having about 3 to about 10 carbon atoms. Exemplary monocyclic cycloalkenyl rings include cyclopentyl, cyclohexenyl, and cycloheptenyl.

The terms “haloaliphatic”, “haloalkyl”, “haloalkenyl” and “haloalkoxy” refer to an aliphatic, alkyl, alkenyl or alkoxy group, as the case may be, which is substituted with one or more halogen atoms. As used herein, the term “halogen” or “halo” means F, Cl, Br, or I. The term “fluoroaliphatic” refers to a haloaliphatic wherein the halogen is fluoro, including perfluorinated aliphatic groups. Examples of fluoroaliphatic groups include, without limitation, fluoromethyl, difluoromethyl, trifluoromethyl, 2-fluoroethyl, 2,2,2-trifluoroethyl, 1,1,2-trifluoroethyl, 1,2,2-trifluoroethyl, and pentafluoroethyl.

The term “heteroatom” refers to one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The terms “aryl” and “ar-”, used alone or as part of a larger moiety, e.g., “aralkyl”, “aralkoxy”, or “aryloxyalkyl”, refer to an optionally substituted C₆₋₁₄aromatic hydrocarbon moiety comprising one to three aromatic rings. Preferably, the aryl group is a C₆₋₁₀aryl group. Aryl groups include, without limitation, optionally substituted phenyl, naphthyl, or anthracenyl. The terms “aryl” and “ar-”, as used herein, also include groups in which an aryl ring is fused to one or more cycloaliphatic rings to form an optionally substituted cyclic structure such as a tetrahydronaphthyl, indenyl, or indanyl ring. The term “aryl” may be used interchangeably with the terms “aryl group”, “aryl ring”, and “aromatic ring”.

An “aralkyl” or “arylalkyl” group comprises an aryl group covalently attached to an alkyl group, either of which independently is optionally substituted. Preferably, the aralkyl group is C₆₋₁₀ arylC₁₋₆alkyl, including, without limitation, benzyl, phenethyl, and naphthylmethyl.

The terms “heteroaryl” and “heteroar-”, used alone or as part of a larger moiety, e.g., “heteroaralkyl”, or “heteroaralkoxy”, refer to groups having 5 to 14 ring atoms, preferably 5, 6, 9, or 10 ring atoms; having 6, 10, or 14 π electrons shared in a cyclic array; and having, in addition to carbon atoms, from one to five heteroatoms. A heteroaryl group may be mono-, bi-, tri-, or polycyclic, preferably mono-, bi-, or tricyclic, more preferably mono- or bicyclic. The term “heteroatom” refers to nitrogen, oxygen, or sulfur, and includes any oxidized form of nitrogen or sulfur, and any quaternized form of a basic nitrogen. For example, a nitrogen atom of a heteroaryl may be a basic nitrogen atom and may also be optionally oxidized to the corresponding N-oxide. When a heteroaryl is substituted by a hydroxy group, it also includes its corresponding tautomer. The terms “heteroaryl” and “heteroar-”, as used herein, also include groups in which a heteroaromatic ring is fused to one or more aryl, cycloaliphatic, or heterocycloaliphatic rings. Nonlimiting examples of heteroaryl groups include thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 3- to 8-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR⁺ (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, piperidinyl, decahydroquinolinyl, oxazolidinyl, piperazinyl, dioxanyl, dioxolanyl, diazepinyl, oxazepinyl, thiazepinyl, morpholinyl, and thiamorpholinyl. A heterocyclyl group may be mono-, bi-, tri-, or polycyclic, preferably mono-, bi-, or tricyclic, more preferably mono- or bicyclic. The term “heterocyclylalkyl” refers to an alkyl group substituted by a heterocyclyl, wherein the alkyl and heterocyclyl portions independently are optionally substituted. Additionally, a heterocyclic ring also includes groups in which the heterocyclic ring is fused to one or more aryl rings.

As used herein, the term “partially unsaturated” refers to a ring moiety that includes at least one double or triple bond between ring atoms. The term “partially unsaturated” is intended to encompass rings having multiple sites of unsaturation, but is not intended to include aromatic (e.g., aryl or heteroaryl) moieties, as herein defined.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. An optionally substituted alkylene chain is a polymethylene group in which one or more methylene hydrogen atoms is optionally replaced with a substituent. Suitable substituents include those described below for a substituted aliphatic group and also include those described in the specification herein. It will be appreciated that two substituents of the alkylene group may be taken together to form a ring system. In certain embodiments, two substituents can be taken together to form a 3-7-membered ring. The substituents can be on the same or different atoms.

An alkylene chain also can be optionally interrupted by a functional group. An alkylene chain is “interrupted” by a functional group when an internal methylene unit is interrupted by the functional group. Examples of suitable “interrupting functional groups” are described in the specification and claims herein.

For purposes of clarity, all bivalent groups described herein, including, e.g., the alkylene chain linkers described above, are intended to be read from left to right, with a corresponding left-to-right reading of the formula or structure in which the variable appears.

An aryl (including aralkyl, aralkoxy, aryloxyalkyl and the like) or heteroaryl (including heteroaralkyl and heteroarylalkoxy and the like) group may contain one or more substituents and thus may be “optionally substituted”. In addition to the substituents defined above and herein, suitable substituents on the unsaturated carbon atom of an aryl or heteroaryl group also include and are generally selected from -halo, —NO₂, —CN, —R⁺, —C(R⁺)═C(R⁺)₂, —C≡C—R⁺, —OR⁺, —SR^{o}, —S(O)R^{o}, —SO₂R^{o}, —SO₃R⁺, —SO₂N(R⁺)₂, —N(R⁺)₂, —NR⁺C(O)R⁺, —NR⁺C(S)R⁺, —NR⁺C(O)N(R⁺)₂, —NR⁺C(S)N(R⁺)₂, —N(R⁺)C(═NR⁺)—N(R⁺)₂, —N(R⁺)C(═NR⁺)—R^{o}, —NR⁺CO₂R⁺, —NR⁺SO₂R^{o}, —NR⁺SO₂N(R⁺)₂, —O—C(O)R⁺, —O—CO₂R⁺, —OC(O)N(R⁺)₂, —C(O)R⁺, —C(S)R^{o}, —CO₂R⁺, —C(O)—C(O)R⁺, —C(O)N(R⁺)₂, —C(S)N(R⁺)₂, —C(O)N(R⁺)—OR⁺, —C(O)N(R⁺)C(═NR⁺)—N(R⁺)₂, —N(R⁺)C(═NR⁺)—N(R⁺)—C(O)R⁺, —C(═NR⁺)—N(R⁺)₂, —C(═NR⁺)—OR⁺, —N(R⁺)—N(R⁺)₂, —C(═NR⁺)—N(R⁺)—OR⁺, —C(R^{o}═N—OR⁺, —P(O)(R⁺)₂, —P(O)(OR⁺)₂, —O—P(O)—OR⁺, and —P(O)(NR⁺)—N(R⁺)₂, wherein R⁺, independently, is hydrogen or an optionally substituted aliphatic, aryl, heteroaryl, cycloaliphatic, or heterocyclyl group, or two independent occurrences of R⁺ are taken together with their intervening atom(s) to form an optionally substituted 5-7-membered aryl, heteroaryl, cycloaliphatic, or heterocyclyl ring. Each R^{o} is an optionally substituted aliphatic, aryl, heteroaryl, cycloaliphatic, or heterocyclyl group.

An aliphatic or heteroaliphatic group, or a non-aromatic carbycyclic or heterocyclic ring may contain one or more substituents and thus may be “optionally substituted”. Unless otherwise defined above and herein, suitable substituents on the saturated carbon of an aliphatic or heteroaliphatic group, or of a non-aromatic carbocyclic or heterocyclic ring are selected from those listed above for the unsaturated carbon of an aryl or heteroaryl group and additionally include the following: ═O, ═S, ═C(R*)₂, ═N—N(R*)₂, ═N—OR*, ═N—NHC(O)R*, ═N—NHCO₂R^{o}═N—NHSO₂R^{o} or ═N—R* where R^{o} is defined above, and each R* is independently selected from hydrogen or an optionally substituted C₁₋₆ aliphatic group.

In addition to the substituents defined above and herein, optional substituents on the nitrogen of a non-aromatic heterocyclic ring also include and are generally selected from —R⁺, —N(R⁺)₂, —C(O)R⁺, —C(O)OR⁺, —C(O)C(O)R⁺, —C(O)CH₂C(O)R⁺, —S(O)₂R⁺, —S(O)₂N(R⁺)₂, —C(S)N(R⁺)₂, —C(═NH)—N(R⁺)₂, or —N(R⁺)S(O)₂R⁺; wherein each R⁺ is defined above. A ring nitrogen atom of a heteroaryl or non-aromatic heterocyclic ring also may be oxidized to form the corresponding N-hydroxy or N-oxide compound. A nonlimiting example of such a heteroaryl having an oxidized ring nitrogen atom is N-oxidopyridyl.

As detailed above, in some embodiments, two independent occurrences of R⁺ (or any other variable similarly defined in the specification and claims herein), are taken together with their intervening atom(s) to form a monocyclic or bicyclic ring selected from 3-13-membered cycloaliphatic, 3-12-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur.

Exemplary rings that are formed when two independent occurrences of R⁺ (or any other variable similarly defined in the specification and claims herein), are taken together with their intervening atom(s) include, but are not limited to the following: a) two independent occurrences of R⁺ (or any other variable similarly defined in the specification or claims herein) that are bound to the same atom and are taken together with that atom to form a ring, for example, N(R⁺)₂, where both occurrences of R⁺ are taken together with the nitrogen atom to form a piperidin-1-yl, piperazin-1-yl, or morpholin-4-yl group; and b) two independent occurrences of R⁺ (or any other variable similarly defined in the specification or claims herein) that are bound to different atoms and are taken together with both of those atoms to form a ring, for example where a phenyl group is substituted with two occurrences of OR⁺

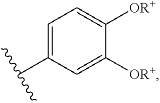

these two occurrences of R⁺ are taken together with the oxygen atoms to which they are bound to form a fused 6-membered oxygen containing ring:

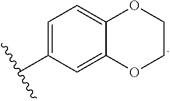 [SMILES: *c1ccc2c(c1)OCCO2]

It will be appreciated that a variety of other rings (e.g., spiro and bridged rings) can be formed when two independent occurrences of R⁺ (or any other variable similarly defined in the specification and claims herein) are taken together with their intervening atom(s) and that the examples detailed above are not intended to be limiting.

Exemplary rings that are formed when two independent occurrences of X₄ and X₅, X₆ and X₇, or X₇ and X₈; are taken together with their intervening atom(s) to form a fused group having 8 to 10 ring atoms include, but are not limited to the following:

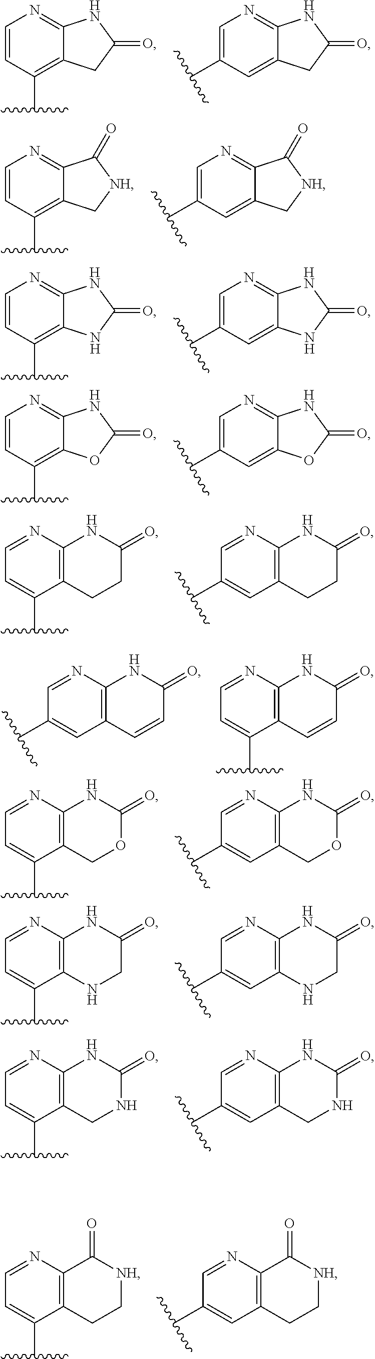

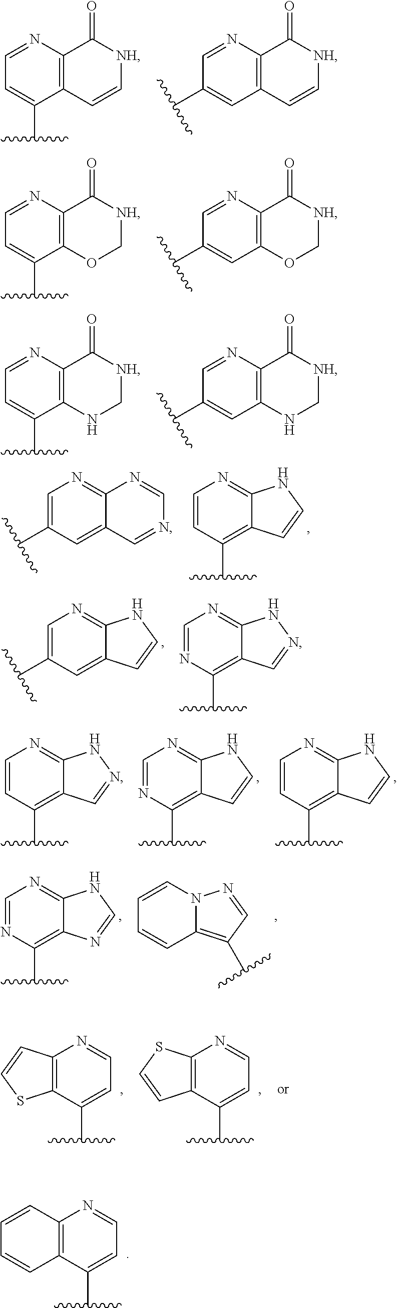

Exemplary rings that are formed when two independent occurrences of Y₁ and —NR⁹, Y₃ and —NR⁹, Y₄ and Y₅, or Y₆ and Y₇ are taken together with their intervening atom(s) to form a fused group having 8 to 10 ring atoms include, but are not limited to the following:

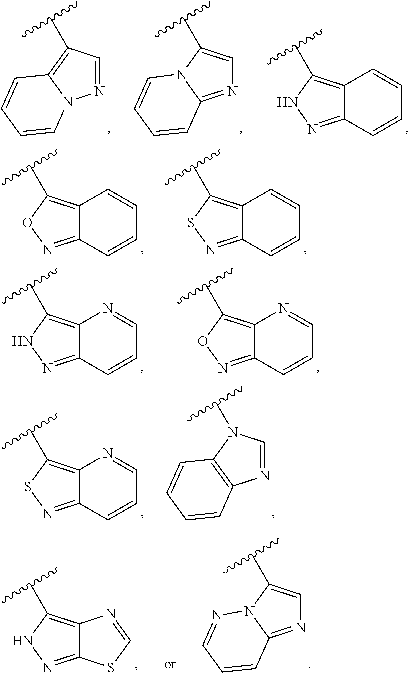

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures where there is a replacement of hydrogen by deuterium or tritium, or a replacement of a carbon by a ¹³C- or ¹⁴C-enriched carbon are within the scope of this invention. Such compounds are useful, as a nonlimiting example, as analytical tools or probes in biological assays.

It is to be understood that, when a disclosed compound has at least one chiral center, the present invention encompasses one enantiomer of inhibitor free from the corresponding optical isomer, racemic mixture of the inhibitor and mixtures enriched in one enantiomer relative to its corresponding optical isomer. When a mixture is enriched in one enantiomer relative to its optical isomers, the mixture contains, for example, an enantiomeric excess of at least 50%, 75%, 90%, 95% 99% or 99.5%.

The enantiomers of the present invention may be resolved by methods known to those skilled in the art, for example by formation of diastereoisomeric salts which may be separated, for example, by crystallization; formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. Where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

When a disclosed compound has at least two chiral centers, the present invention encompasses a diastereomer free of other diastereomers, a pair of diastereomers free from other diasteromeric pairs, mixtures of diasteromers, mixtures of diasteromeric pairs, mixtures of diasteromers in which one diastereomer is enriched relative to the other diastereomer(s) and mixtures of diasteromeric pairs in which one diastereomeric pair is enriched relative to the other diastereomeric pair(s). When a mixture is enriched in one diastereomer or diastereomeric pair(s) relative to the other diastereomers or diastereomeric pair(s), the mixture is enriched with the depicted or referenced diastereomer or diastereomeric pair(s) relative to other diastereomers or diastereomeric pair(s) for the compound, for example, by a molar excess of at least 50%, 75%, 90%, 95%, 99% or 99.5%.

The diastereoisomeric pairs may be separated by methods known to those skilled in the art, for example chromatography or crystallization and the individual enantiomers within each pair may be separated as described above. Specific procedures for chromatographically separating diastereomeric pairs of precursors used in the preparation of compounds disclosed herein are provided the examples herein.

### 3. Description of Exemplary Compounds

Other embodiments of the invention relate to a subgenus of the compounds of formula IB and ID, characterized by formula IIB:

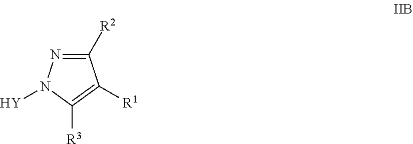

or a pharmaceutically acceptable salt thereof, where variables HY, R¹, R², and R³ are as defined above for formula ID.

Other embodiments of the invention relate to a subgenus of the compounds of formula IB and ID, characterized by formula IIC:

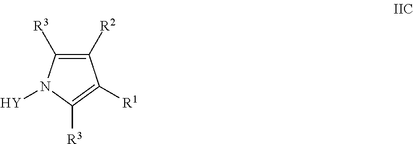

or a pharmaceutically acceptable salt thereof, where variables HY, R¹, R², and R³ are as defined above for formula ID.

Other embodiments of the invention relate to a subgenus of the compounds of formula IB, characterized by formula VB:

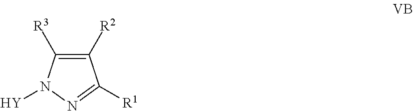

or a pharmaceutically acceptable salt thereof, where variables HY, R¹, R², and R³ are as defined above for formula IB.

In certain embodiments, for compounds of general formula IB, ID, IIB, VB, or IIC, R¹ is CY and

CY is

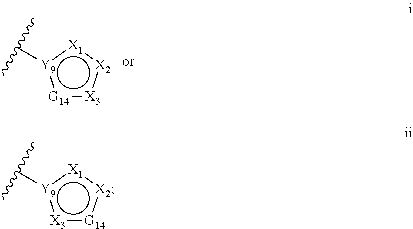

wherein:
X₁, X₂, and X₃, are each independently N, O, S, NR^{4′}, or CR⁷, provided that only one of
X₁, X₂, or X₃ may be O or S;
Y₉ is nitrogen or carbon;
G₁₄ is CR^{7′}, —N═ or —N^{4′}, wherein:
R^{4′} is independently hydrogen, —Z₂—R⁶, optionally substituted C₁₋₆ aliphatic, or optionally substituted 3-10-membered cycloaliphatic, wherein:
   Z₂ is selected from an optionally substituted C₁₋₃ alkylene chain, —S(O)—, —S(O)₂—, —C(O)—, —CO₂—, —C(O)NR^{4a}—, or —S(O)₂NR^{4a}—,
   R^{4a} is hydrogen or an optionally substituted C₁₋₄ aliphatic, and
   R⁶ is an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R⁷ and R^{7′} is independently hydrogen, —CN, halogen, —Z₃—R⁸, C₁₋₆ aliphatic, or 3-10-membered cycloaliphatic, wherein:
   Z₃ is selected from an optionally substituted C₁₋₃ alkylene chain, —O—, —N(R^{7a})—, —S—, —S(O)—, —S(O)₂—, —C(O)—, —CO₂—, —C(O)NR^{7a}—, —N(R^{7a})C(O)—, —N(R^{7a})CO₂—, —S(O)₂NR^{7a}—, —N(R^{7a})S(O)₂—, —OC(O)N(R^{7a})—, —N(R^{7a})C(O)NR^{7a}—, —N(R^{7a})S(O)₂N(R^{7a})—, or —OC(O)—;
   R^{7a} is hydrogen or an optionally substituted C₁₋₄ aliphatic, and
R⁸ is an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur.

In certain embodiments, for compounds of general formula IB, ID, IIB, VB, or IIC, R¹ is CY and

CY is

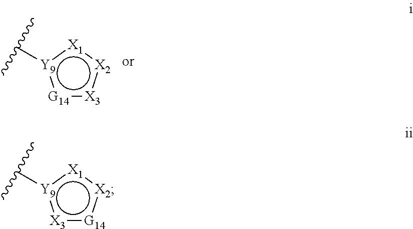

wherein:
X₁, X₂, and X₃, are each independently N, O, S, NR^{4′}, or CR⁷, provided that only one of
X₁, X₂, or X₃ may be O or S;
Y₉ is nitrogen or carbon;
G₁₄ is CR^{7′}, —N═ or —NR^{4′}—, wherein:
R^{4′} is independently hydrogen, —Z₂—R⁶, optionally substituted C₁₋₆ aliphatic, or optionally substituted 3-10-membered cycloaliphatic, wherein:
   Z₂ is selected from an optionally substituted C₁₋₃ alkylene chain, —S(O)—, —S(O)₂—, —C(O)—, —CO₂—, —C(O)NR^{4a}—, or —S(O)₂NR^{4a}—,
   R^{4a} is hydrogen or an optionally substituted C₁₋₄ aliphatic, and
   R⁶ is an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R⁷ and R^{7′} is independently hydrogen, —CN, halogen, —NH₂, —Z₃—R⁸, C₁₋₆ aliphatic, or 3-10-membered cycloaliphatic, wherein:
   Z₃ is selected from an optionally substituted C₁₋₃ alkylene chain, —O—, —N(R^{7a})—, —S—, —S(O)—, —S(O)₂—, —C(O)—, —CO₂—, —C(O)NR^{7a}—, —N(R^{7a})C(O)—, —N(R^{7a})CO₂—, —S(O)₂NR^{7a}—, —N(R^{7a})S(O)₂—, —OC(O)N(R^{7a})—, —N(R^{7a})C(O)NR^{7a}—, —N(R^{7a})S(O)₂N(R^{7a})—, or —OC(O)—;
   R^{7a} is hydrogen or an optionally substituted C₁₋₄ aliphatic, and
R⁸ is hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur.

In other embodiments, for compounds described directly above, CY is

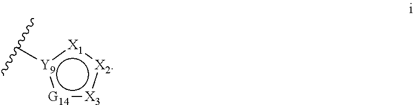

In still other embodiments for compounds of general formula IB, ID, IIB, VB, or IIC, Y₉ is carbon, X₁ is nitrogen, G₁₄ is N(R^{4′}), and X₂ and X₃, are CH.

In yet other embodiments, Y₉ is carbon, X₁ and X₃ are nitrogen, G₁₄ is N(R^{4′}), and X₂ is CH.

In other embodiments, Y₉ is carbon, X₁ and G₁₄ are nitrogen, X₃ is N(R^{4′}), and X₂ is CH.

In other embodiments, Y₉ is carbon, X₁ and X₂ are nitrogen, G₁₄ is N(R^{4′}), and X₃ is CH.

In other embodiments, Y₉ is carbon, G₁₄ is N(R^{4′}), X₃ is nitrogen, and X₁ and X₂ are CH.

In other embodiments, Y₉ is carbon, G₁₄ is nitrogen, X₃ is N(R^{4′}), and X₁ and X₂ are CH.

In other embodiments, Y₉ is carbon, X₃ is nitrogen, X₂ is N(R^{4′}), and X₁ and G₁₄ are CH.

In other embodiments, Y₉ is carbon, X₂ is nitrogen, G₁₄ is N(R^{4′}), and X₁ and X₃, are CH.

In other embodiments, Y₉ is carbon, X₂ is N(R^{4′}), G₁₄ is nitrogen, and X₁ and X₃, are CH.

In still other embodiments for compounds of general formula IB, ID, IIB, VB, or IIC, R¹ is Cy and Cy is an optionally substituted 6-membered aryl or heteroaryl ring.

In still other embodiments, R¹ is Cy and Cy is an optionally substituted 5- to 6-membered heteroaryl or heterocyclyl ring.

In yet other embodiments, R¹ is Cy and Cy is selected from:

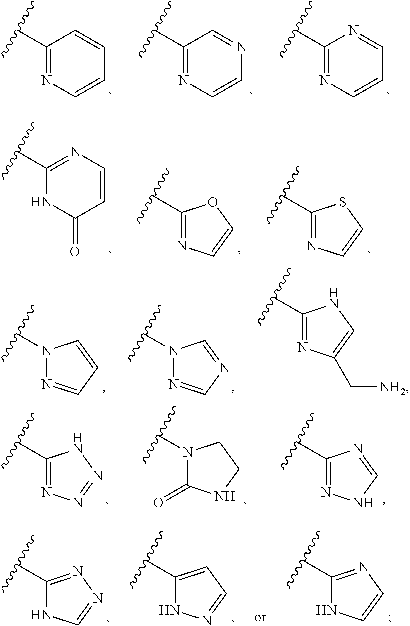

wherein R¹ is optionally further substituted with one or more occurrences of R⁷ or R^{4′}.

In other embodiments, R¹ is Cy, and Cy is selected from:

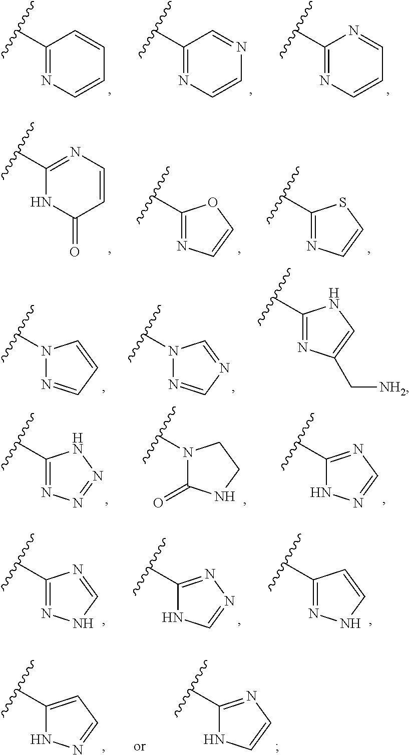

wherein Cy is optionally further substituted with one or more occurrences of R⁷ or R^{4′}.

In other embodiments, R¹ is Cy, and Cy is selected from:

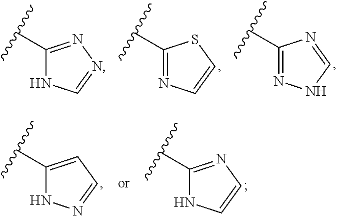

wherein R¹ is optionally further substituted with one or more occurrences of R⁷ or R^{4′}.

In other embodiments, R¹ is Cy, and Cy is selected from:

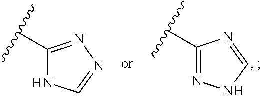 [SMILES: *c1nnc[nH]1 ; *c1nc[nH]n1]

In other embodiments, R¹ is Cy, and Cy is an optionally substituted 6-membered aryl ring.

In other embodiments, R¹ is —CON(R⁴)₂, —NHCOR⁴, or —COOR⁴.

In other embodiments, R¹ is —CON(R⁴)₂, —C(O)OR⁴, —NHCOR⁴, or CH₂OR⁴.

In any of the embodiments described above for R¹, other variables HY, R², R³, R¹⁰, R^{10′}, R¹¹, R^{10a}, R⁷, and R^{4′} are as defined in any one of the embodiments described herein.

In some embodiments, for compounds of general formula IB, ID, IIB, VB, or IIC, HY is selected from:

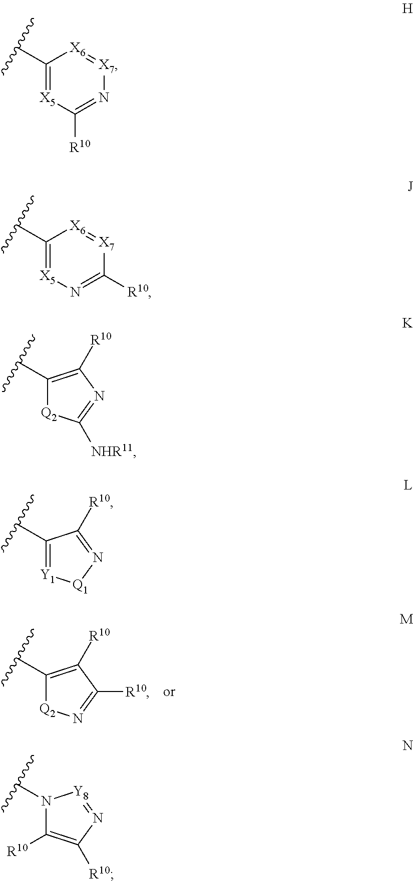

wherein each occurrence of X₅, X₆, and X₇ is independently —CR¹⁰, —CR^{10′} or N, provided no more than two occurrences of X₅, X₆, and X₇ are N;

each occurrence of Q₁ and Q₂ is independently S, O or —NR⁹;

each occurrence of Y₁ and Y₇ is independently —CR¹⁰;
or wherein two adjacent occurrences of X₆, and X₇, Y₁ and —NR⁹, or two adjacent occurrences of R¹⁰ taken together with the atoms to which they are bound, form an optionally substituted fused heteroaryl or heterocyclyl group having 8 to 10 ring atoms and having 1-5 heteroatoms independently selected from nitrogen, oxygen, or sulfur.

In some embodiments, for compounds of general formula IB, ID, IIB, VB, or IIC, HY is selected from:

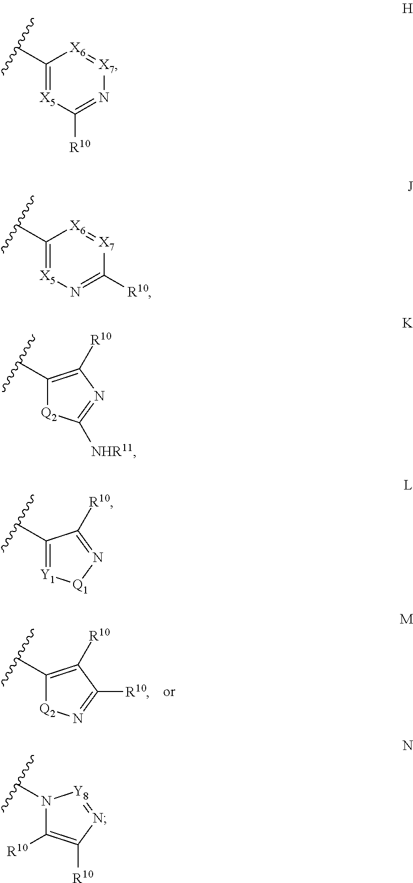

wherein each occurrence of X₅, X₆, and X₇ is independently —CR¹⁰, —CR^{10′} or N, provided no more than two occurrences of X₅, X₆, and X₇ are N;

each occurrence of Q₁ and Q₂ is independently S, O or —NR⁹;

each occurrence of Y₁ and Y₇ is independently —CR¹⁰;
or wherein two adjacent occurrences of X₆, and X₇, Y₁ and —NR⁹, or two adjacent occurrences of R¹⁰ taken together with the atoms to which they are bound, form an unsubstituted fused heteroaryl or heterocyclyl group having 8 to 10 ring atoms and having 1-5 heteroatoms independently selected from nitrogen, oxygen, or sulfur.

In yet other embodiments, HY is selected from:

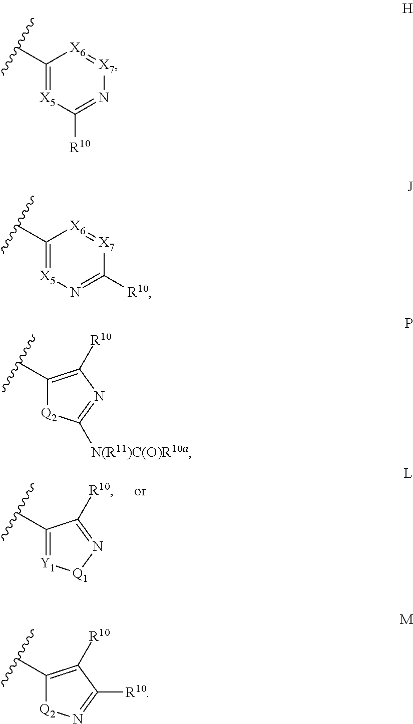

In yet other embodiments, HY is selected from:

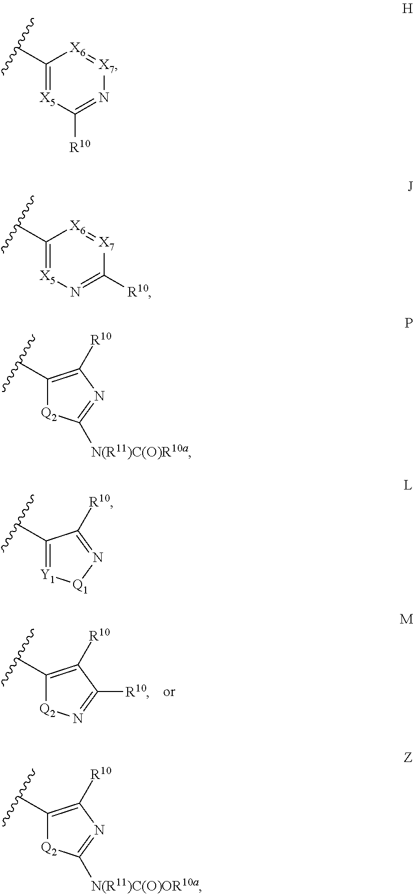

In some embodiments for compounds of formula IB, ID, IIB, VB, or IICHY is selected from:

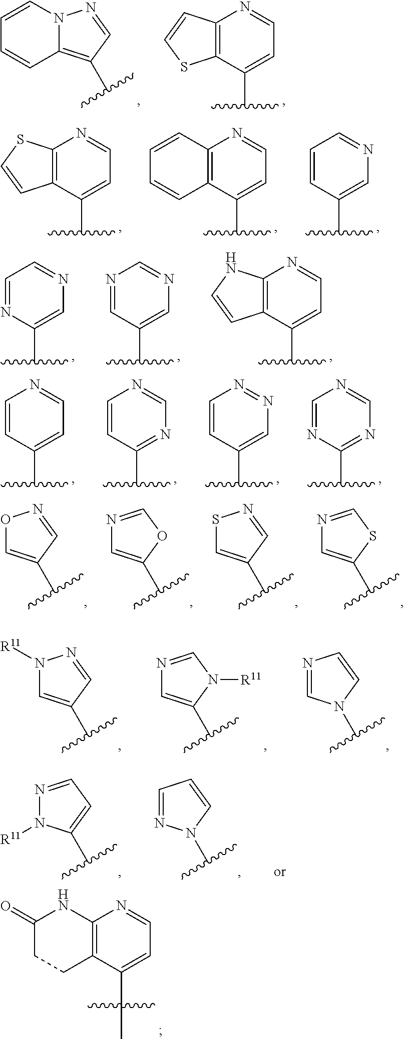

wherein each HY group is optionally additionally substituted with one or more occurrences of R¹⁰, and the dashed line represents a single bond or a double bond.

In some embodiments for compounds of formula IB, ID, IIB, VB, or IICHY is selected from:

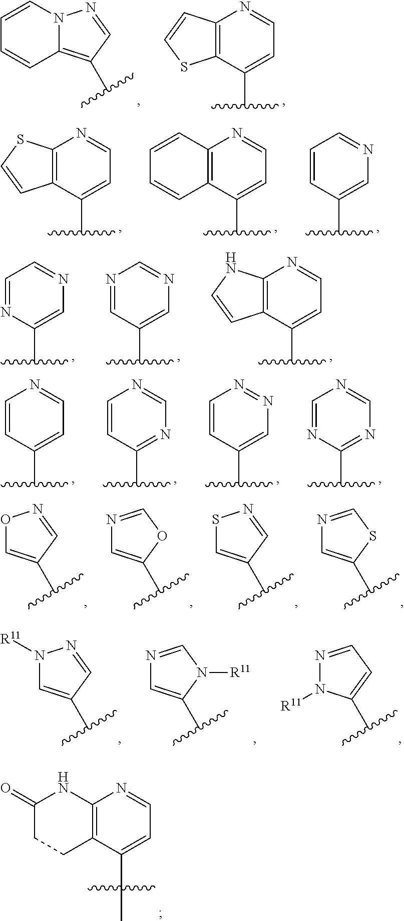

wherein each fused HY group is unsubstituted, and

each non-fused HY group is substituted with one or more occurrences of R¹⁰ or R^{10′}, and at least one occurrence of R¹⁰ or R^{10′} is —N(R¹¹)C(O)R^{10a}, —N(R¹¹)C(O)OR^{10a}, or —C(O)N(R¹¹)₂, and the dashed line represents a single bond or a double bond.

In some embodiments for compounds of general formula IB, ID, IIB, VB, or IIC, HY is selected from:

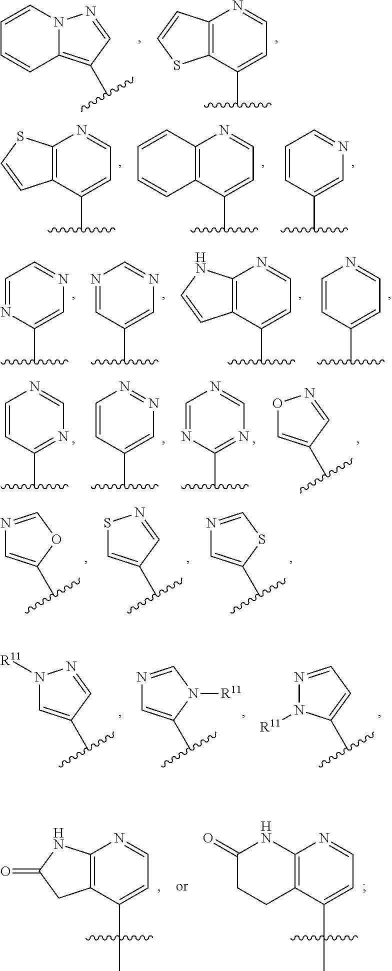

wherein each fused HY group is unsubstituted, and

each non-fused HY group is substituted with one or more occurrences of R¹⁰ or R^{10′}, and at least one occurrence of R¹⁰ or R^{10′} is —N(R¹¹)C(O)R^{10a}, —N(R¹¹)C(O)OR^{10a}, or —C(O)N(R¹¹)₂, and the dashed line represents a single bond or a double bond.

In some embodiments for compounds of general formula IB, ID, IIB, VB, or IIC, R^{10a} is C₁₋₆ aliphatic substituted with a 5-10-membered heteroaryl having 1-5 heteroatoms independently selected from nitrogen, oxygen, or sulfur.

In still other embodiments for compounds of general formula IB, ID, IIB, VB, or IIC, HY is selected from:

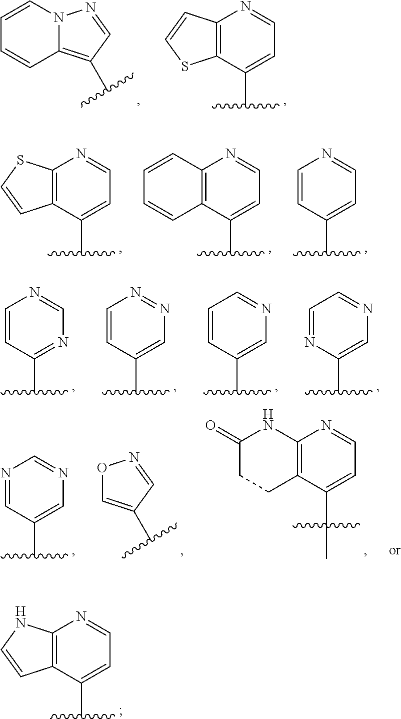

wherein each HY group is optionally additionally substituted with one or more occurrences of R¹⁰, and the dashed line in xviii represents a single bond or a double bond.

In still other embodiments, HY is selected from:

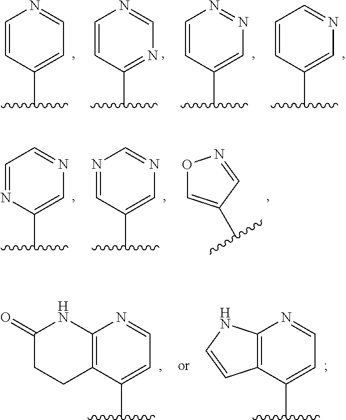

wherein each fused HY group is unsubstituted, and each non-fused HY group is substituted with one or more occurrences of R¹⁰ or R^{10′}, and at least one occurrence of R¹⁰ or R^{10′} is —N(R¹¹)C(O)R^{10a}, —N(R¹¹)C(O)OR^{10a}, or —C(O)N(R¹¹)₂, and the dashed line represents a single bond or a double bond.

In yet other embodiments, HY is

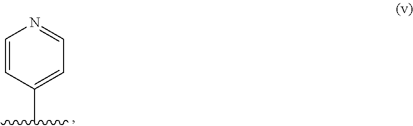 [SMILES: *c1ccncc1]

wherein HY is additionally optionally substituted with one or more occurrences of R¹⁰.

In yet other embodiments, HY is

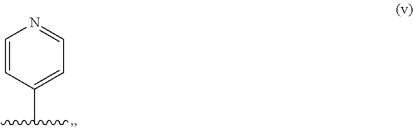 [SMILES: *c1ccncc1]

wherein HY is substituted with one or more occurrences of R¹⁰ or R^{10′}.

In still other embodiments, HY is

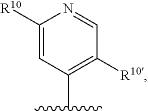

selected from wherein R^{10′} is hydrogen, methyl, chloro, bromo, fluoro, CN, CF₃, OR^{10a}, COR^{10a}, and R¹⁰ is NHCOR^{10a} or —NHC(O)OR^{10a}.

In still other embodiments, R^{10′} is hydrogen, methyl, or chloro, and R¹⁰ is —NHCOR^{10a} or —NHCOOR^{10a}.

In some embodiments, for compounds of general formula IB, ID, IIB, VB, or IIC, R^{10′} is hydrogen, methyl, or chloro, and R¹⁰ is —NHR¹¹, wherein R¹¹ is an optionally substituted group selected from 6-10-membered aryl, or 5-10-membered heteroaryl having 1-5 heteroatoms independently selected from nitrogen, oxygen, or sulfur.

In yet other embodiments, R^{10′} is hydrogen, methyl, or chloro.

In still other embodiments, R^{10′} is methyl, and R¹⁰ is —NHCOR^{10a}.

In still other embodiments, R¹⁰ is —NHR¹¹, wherein R¹¹ is an optionally substituted 5- to 10-membered heteroaryl having 1-5 heteroatoms independently selected from nitrogen, oxygen, or sulfur.

In other embodiments, R^{10a} is cyclopropyl, methyl, ethyl, or isopropyl.

In any of the embodiments described above for HY, other variables R¹, R², R³, R¹⁰, R^{10′}, R¹¹, R^{10a}, R⁷, and R^{4′} are as defined in any one of the embodiments described herein.

In some embodiments, for compounds of general formula IB, ID, IIB, VB, or IIC, R² is a 6-10-membered aryl or 5-10-membered heteroaryl having 1-5 heteroatoms independently selected from nitrogen, oxygen, or sulfur; optionally substituted with 1-3 occurrences of R^{2a}.

In other embodiments, R² is a phenyl or pyridyl group;

In other embodiments, R² is a phenyl group; optionally substituted with one or more independent occurrences of halogen, C₁₋₃ alkyl, —CN, C₁₋₃ haloalkyl, —(CH₂)ₚN(R^{12b})₂, —R^{12b}, —NHC(O)R^{12b}, —NHC(O)NHR^{12b}, —NHS(O)₂R^{12b}, —S(O)₂R^{12b}, —S(O)₂N(R^{12b})₂, C(O)OR^{12b}, —C(O)N(R^{12b})₂, or —C(O)R^{12b}; or

wherein two occurrences of R^{12b}, taken together with a nitrogen atom to which they are bound, form an optionally substituted 4-7-membered heterocyclyl ring having 0-1 additional heteroatoms selected from nitrogen, oxygen, or sulfur.

In other embodiments, R² is a phenyl group; optionally substituted with one or more independent occurrences of halogen, C₁₋₃ alkyl, —CN, C₁₋₃ haloalkyl, —(CH₂)ₚN(R^{12b})₂, —OR^{12b}, —NHC(O)R^{12b}, —NHC(O)NHR^{12b}, —NHS(O)₂R^{12b}, —S(O)₂R^{12c}, —S(O)₂N(R^{12b})₂, C(O)OR^{12b}, —C(O)N(R^{12b})₂, or —C(O)R^{12b}; wherein R^{12b} and R^{12c} are defined as described herein or

wherein two occurrences of R^{12b}, taken together with a nitrogen atom to which they are bound, form an optionally substituted 4-7-membered heterocyclyl ring having 0-1 additional heteroatoms selected from nitrogen, oxygen, or sulfur, and wherein p is 0 to 3.

In other embodiments, R² is a phenyl group; optionally substituted with 1 to 4 independent occurrences of halogen, C₁₋₃ alkyl, —CN, C₁₋₃ haloalkyl, —(CH₂)ₚN(R^{12b})₂, —OR^{12b}, —NHC(O)R^{12b}, —NHC(O)NHR^{12b}, —NHS(O)₂R^{12b}, —S(O)₂R^{12c}, —S(O)₂N(R^{12b})₂, —C(O)OR^{12b}, —C(O)N(R^{12b})₂, or —C(O)R^{12b}; wherein R^{12b} and R^{12c} are defined as described herein or

wherein two occurrences of R^{12b}, taken together with a nitrogen atom to which they are bound, form an optionally substituted 4-7-membered heterocyclyl ring having 0-1 additional heteroatoms selected from nitrogen, oxygen, or sulfur, and wherein p is 0 to 3.

In yet other embodiments, R² is a phenyl group; optionally substituted with one or more independent occurrences of halogen, C₁₋₃ alkyl, —CN, C₁₋₃ haloalkyl, —CH₂N(CH₃)₂, —OC₁₋₃ alkyl, —OC₁₋₃ haloalkyl, —NHC(O)C₁₋₃ alkyl, —NHC(O)NHC₁₋₃ alkyl, —NHS(O)₂C₁₋₃ alkyl, or —C(O)H.

In yet other embodiments, R² is a phenyl group substituted with 1 or 2 occurrences of halogen.

In still other embodiments, R² is a 3-10-membered cycloaliphatic, 4-10-membered heterocyclyl having 1-5 heteroatoms independently selected from nitrogen, oxygen, or sulfur.

In yet other embodiments, R² is an optionally substituted N-linked 3-, 4-, 5-, 6-, or 7-membered heterocyclyl ring, optionally substituted with one or more occurrences of R^{2a}.

In still other embodiments, R² is optionally substituted with one or more C₁₋₃ alkyl groups, —OR^{12b}, or —NR^{12b}.

In still other embodiments, R² is a C₁₋₆ aliphatic and each occurrence of R^{2a} is independently —C(O)OR^{12b}, —C(O)N(R^{12b})₂, —S(O)₂N(R^{12b})₂, —N(R^{12e})C(O)R^{12b}, or N(R^{12e})SO₂R^{12c}.

In still other embodiments, R² is a C₁₋₆ aliphatic, optionally substituted with halo, —N(R^{12b})₂, or a cyclopropyl ring, wherein each R^{12b} is independently selected from hydrogen, methyl, or ethyl, or wherein two R^{12b}, taken together with a nitrogen atom to which they are bound, form a pyrrolidinyl ring. In still other embodiments, R² is a C₁₋₃ aliphatic.

In still other embodiments, R² is halogen. In other embodiments, R² is hydrogen.

In any of the embodiments described above for R², other variables HY, R¹, R³, R¹⁰, R^{10′}, R^{10a}, R⁷, and R^{4′} are as defined in any one of the embodiments described herein.

In certain embodiments, for compounds of general formula IB, ID, IIB, VB, or IIC, R¹ is CY, —CON(R⁴)₂, —NHCOR⁴, or —COOR⁴; R² is optionally substituted aryl or heteroaryl; and HY is selected from

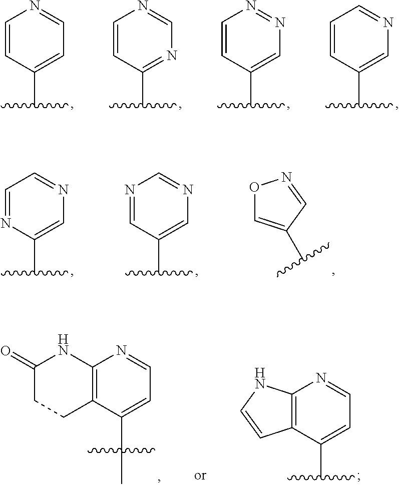

wherein each fused HY group is unsubstituted, and
each non-fused HY group is substituted with one or more occurrences of R¹⁰ or R^{10′}, and at least one occurrence of R¹⁰ or R^{10′} is —N(R¹¹)C(O)R^{10a} or —C(O)N(R¹¹)₂, and the dashed line represents a single bond or a double bond.

In certain embodiments, for compounds of general formula IB, ID, IIB, VB, or IIC R¹ is CY, —CON(^{R4})₂, —NHCO^{R4}, or —COO^{R4}; R² is an optionally substituted 6-10-membered aryl or 5-10-membered heteroaryl having 1-5 heteroatoms independently selected from nitrogen, oxygen, or sulfur; and HY is selected from

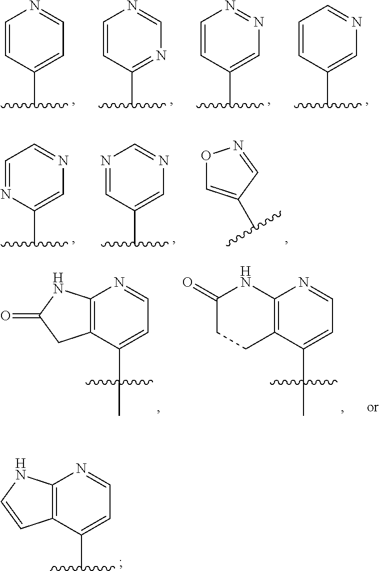

wherein each fused HY group is unsubstituted, and
each non-fused HY group is substituted with one or more occurrences of R¹⁰ or R^{10′}, and at least one occurrence of R¹⁰ or R^{10′} is —N(R¹¹)C(O)R^{10a} or —C(O)N(R¹¹)₂, and the dashed line represents a single bond or a double bond.

In still other embodiments, the compound has the formula IB and wherein R¹ is CY, —CON(R₄)₂, —NHCOR⁴, or —COOR⁴; R² is optionally substituted aryl or heteroaryl; and HY is selected from

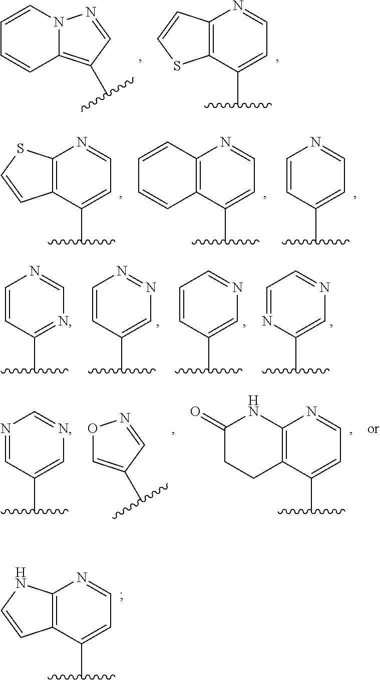

wherein each HY group is optionally additionally substituted with one or more occurrences of R¹⁰, and the dashed line represents a single bond or a double bond.

General Synthetic Methods and Intermediates:

The compounds of the present invention can be prepared by methods known to one of ordinary skill in the art and/or by reference to the schemes shown below and the synthetic examples that follow. Exemplary synthetic routes are set forth in the Schemes below, and in the Examples.

Examples of the solvent for the below-mentioned reactions include, but are not limited to, halogenated hydrocarbons such as dichloromethane, chloroform, carbon tetrachloride, 1,2-dichloroethane and the like, aromatic hydrocarbons such as benzene, toluene, xylene and the like, alcohols such as methanol, ethanol, isopropanol, tert-butanol, phenol and the like, ethers such as diethyl ether, tetrahydrofuran, dioxane, DME and the like, acetone, ACN, ethyl acetate, N,N-dimethylformamide, N,N-dimethylacetamide, 1-methyl-2-pyrrolidone, dimethyl sulfoxide, hexamethylphosphoramide, water or a mixed solvent thereof and the like.

One of ordinary skill in the art will recognize that numerous variations in reaction conditions including variations in solvent, reagents, catalysts, reaction temperatures and times are possible for each of the reactions described. Variation of order of synthetic steps and alternative synthetic routes are also possible.

In many cases, synthesis can be started from commercially available pyrazole analogs to prepare target compounds. In some cases, specially functionalized pyrazole/pyrrole analogs can be prepared by the procedures described in the Schemes below.

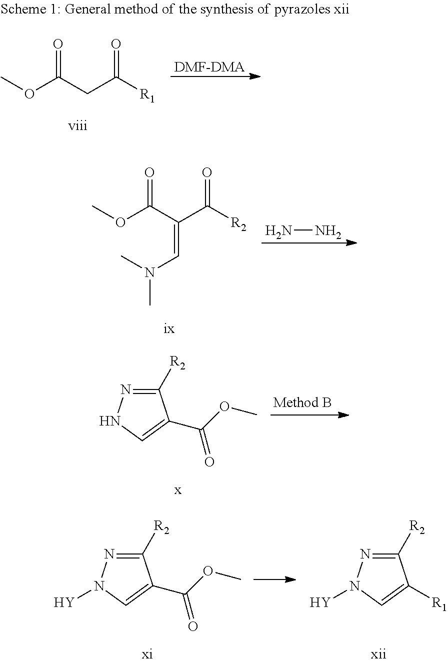

Scheme 1 describes a method of preparing substituted pyrazoles xii. Reaction of substituted β-keto esters viii with DMF-DMA is a method that can be used to prepare enamines ix, which can be cyclized to pyrazoles x by treatment with hydrazine. Pyrazoles x can be treated under conditions of Method B as a method of preparing esters xi. Method B can refer to the coupling of an aryl or heteroaryl halide with an amine under suitable conditions, for example Pd₂(dba)₃, Xantphos, Cs₂CO₃, in an appropriate solvent, such as dioxane, at elevated temperature or under microwave irradiation. Alternatively, Method B can refer to the oxidative coupling reaction of an aryl or heteroaryl halide, boronic acid, boronic ester, or stannane with an N—H containing compound in the presence or absence of oxygen and an appropriate copper species. Compounds xi can be elaborated to pyrazoles xii via the intermediate acids (obtained by hydrolysis of the ester of compounds xi under standard conditions) or by transformation of the esters xi to a variety of groups using standard methods.

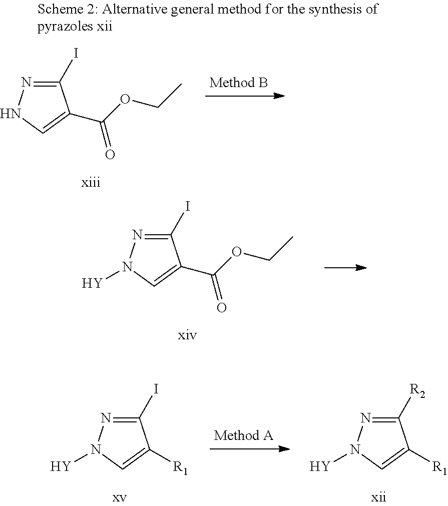

Scheme 2 describes an alternate method of preparing substituted pyrazoles xii. Reaction of iodopyrazole xiii under conditions of Method B can be used to prepare substituted pyrazoles xiv. Pyrazoles xiv can be elaborated to pyrazoles xv through a series of standard transformations as described for the preparation of compounds xii from compounds xi in Scheme 1. Pyrazoles xv can then be transformed into the desired pyrazoles xii following treatment under conditions of Method A. Method A can refer to the coupling reaction of an aryl or heteroaryl bromide with an appropriate aryl or heteroaryl stannane under suitable conditions, for example Pd(PPh₃)₄, CuI, LiCl in an appropriate solvent, such as dioxane at elevated temperature. Alternatively, Method A can refer to the coupling reaction of an aryl or heteroaryl halide with an appropriate boronic acid or boronic ester under suitable conditions, for example Pd(dppf)₂Cl₂, Na₂CO₃, in an appropriate solvent, such as DME, at elevated temperature or under microwave irradiation.

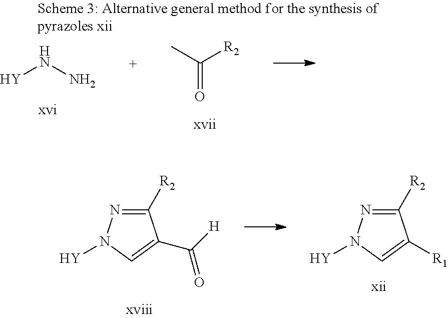

Scheme 3 describes another alternate method of preparing substituted pyrazoles xii. Heterocyclic hydrazines xvi can be condensed with methylketones xvii to give unsubstituted pyrazoles, which can be treated under Vilsmeier-Haack conditions, for example POCl₃ and DMF, to give 4-formylpyrazoles xviii. Pyrazoles xviii can be elaborated to pyrazoles xii following conversion of aldehydes xviii to the corresponding esters and then following a series of standard transformations as described for the preparation of compounds xii from compounds xi in Scheme 1.

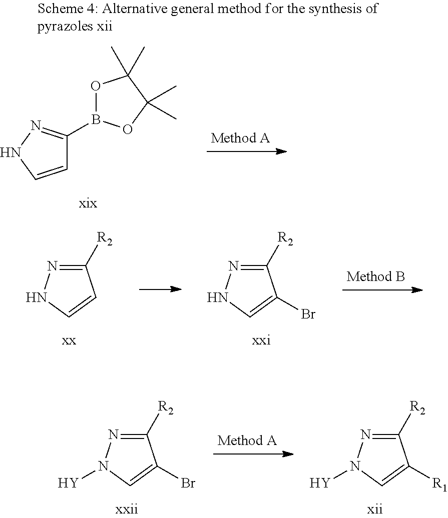

Scheme 4 describes another alternate method of preparing substituted pyrazoles xii. Pyrazoles xix can be transformed into pyrazoles xx following treatment under conditions of Method A. Treatment of compounds xx using a reagent such as NBS in a solvent such as DCM provides halogenated pyrazoles xxi. Reaction of bromopyrazole xxi under conditions of Method B can be used to prepare substituted pyrazoles xxii. Desired pyrazoles xii can be obtained from compounds xxii using conditions of Method A.

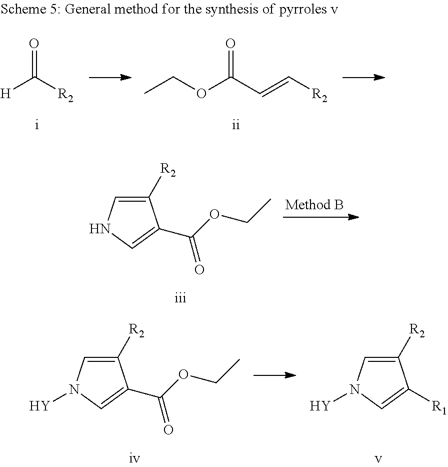

Scheme 5 describes a method of preparing substituted pyrroles v. Aldehydes i can be transformed to the corresponding α,β-unsaturated esters ii by reaction, for example, with sodium hydride and ethyl (diethoxyphosphoryl)acetate in an appropriate solvent, such as THF. Treatment of the resulting esters ii with an isocyanide, such as p-tolylsulfonylmethyl isocyanide, in an appropriate solvent, such as THF, at low temperature in the presence of a base such as sodium tert-butoxide is a method that can be used to prepare pyrroles iii. Substituted pyrroles iv can be prepared by treatment of iii under the conditions of Method B. Compounds iv can be elaborated to pyrroles v via the intermediate acids (obtained by hydrolysis of the esters iv under standard conditions) or by transformation of the esters iv to a variety of groups using standard methods.

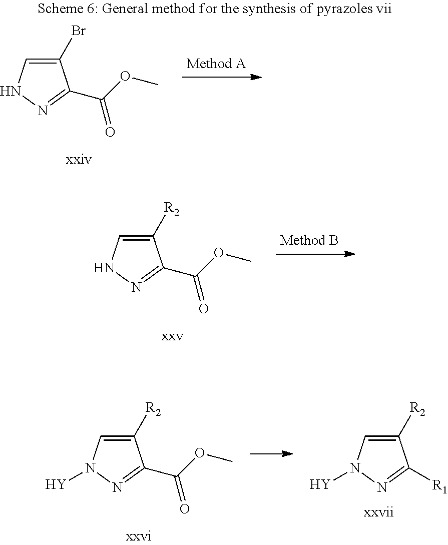

Scheme 6 describes a method of preparing substituted pyrazoles xxvii. Treatment of 4-bromo-1H-pyrazole-3-carboxylate (xxiv) under the conditions of Method A can be used to prepare pyrazoles xv. Method A can refer to the coupling reaction of an aryl or heteroaryl bromide with an appropriate aryl or heteroaryl stannane under suitable conditions, for example Pd(PPh₃)₄, CuI, LiCl in an appropriate solvent, such as dioxane at elevated temperature. Alternatively, Method A can refer to the coupling reaction of an aryl or heteroaryl halide with an appropriate boronic acid or boronic ester under suitable conditions, for example Pd(dppf)₂Cl₂, Na₂CO₃, in an appropriate solvent, such as DME, at elevated temperature or under microwave irradiation. Pyrazoles xxv can be treated under conditions of Method B as a method of preparing esters xxvi. Method B can refer to the coupling of an aryl or heteroaryl halide with an amine under suitable conditions, for example Pd₂(dba)₃, Xantphos, Cs₂CO₃, in an appropriate solvent, such as dioxane, at elevated temperature or under microwave irradiation. Alternatively, Method B can refer to the oxidative coupling reaction of an aryl or heteraryl halide, boronic acid, boronic ester, or stannane with an N—H containing compound in the presence or absence of oxygen and an appropriate copper species. Pyrazoles xxvi can be elaborated to pyrazoles xxvii via the intermediate acids (obtained by hydrolysis of the ester of compounds xxvi under standard conditions) or by transformation of the esters xxvi directly to a variety of groups using standard methods.

The compounds of the present invention can be prepared by methods known to one of ordinary skill in the art and/or by reference to the schemes shown below and the synthetic examples that follow.

### 4. Uses, Formulation and Administration

As discussed above, the present invention provides compounds that are useful as inhibitors of VPS34 and/or PI3K, and thus the present compounds are useful for treating proliferative, inflammatory, or cardiovascular disorders such as tumor and/or cancerous cell growth mediated by VPS34 and/or PI3K. In particular, the compounds are useful in the treatment of cancers in a subject, including, but not limited to, lung and bronchus, including non-small cell lung cancer (NSCLC), squamous lung cancer, brochioloalveolar carcinoma (BAC), adenocarcinoma of the lung, and small cell lung cancer (SCLC); prostate, including androgen-dependent and androgen-independent prostate cancer; breast, including metastatic breast cancer; pancreas; colon and rectum; thyroid; liver and intrahepatic bile duct; hepatocellular; gastric; endometrial; melanoma; kidney; and renal pelvis, urinary bladder; uterine corpus; uterine cervix; ovary, including progressive epithelial or primary peritoneal cancer; multiple myeloma; esophagus; acute myelogenous leukemia (AML); chronic myelogenous leukemia (CML), including accelerated CML and CML blast phase (CML-BP); lymphocytic leukemia; myeloid leukemia; acute lymphoblastic leukemia (ALL); chronic lymphocytic leukemia (CLL); Hodgkin's disease (HD); non-Hodgkin's lymphoma (NHL), including follicular lymphoma and mantle cell lymphoma; B-cell lymphoma, including diffuse large B-cell lymphoma (DLBCL); T-cell lymphoma; multiple myeloma (MM); amyloidosis; Waldenstrom's macroglobulinemia; myelodysplastic syndromes (MDS), including refractory anemia (RA), refractory anemia with ringed siderblasts (RARS), (refractory anemia with excess blasts (RAEB), and RAEB in transformation (RAEB-T); and myeloproliferative syndromes; brain, including glioma/glioblastoma, anaplastic oligodendroglioma, and adult anaplastic astrocytoma; neuroendocrine, including metastatic neuroendocrine tumors; head and neck, including, e.g., squamous cell carcinoma of the head and neck, and nasopharyngeal cancer; oral cavity; and pharynx; small intestine; bone; soft tissue sarcoma; and villous colon adenoma.

In some embodiments, compounds of the invention are suitable for the treatment of breast cancer, bladder cancer, colon cancer, glioma, glioblastoma, lung cancer, hepatocellular cancer, gastric cancer, melanoma, thyroid cancer, endometrial cancer, renal cancer, cervical cancer, pancreatic cancer, esophageal cancer, prostate cancer, brain cancer, or ovarian cancer.

In other embodiments, compounds of the invention are suitable for the treatment of inflammatory and cardiovascular disorders including, but not limited to, allergies/anaphylaxis, acute and chronic inflammation, rheumatoid arthritis; autoimmunity disorders, thrombosis, hypertension, cardiac hypertrophy, and heart failure.

Accordingly, in another aspect of the present invention, pharmaceutical compositions are provided, wherein these compositions comprise any of the compounds as described herein, and optionally comprise a pharmaceutically acceptable carrier, adjuvant or vehicle. In certain embodiments, these compositions optionally further comprise one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable prodrugs, salts, esters, salts of such esters, or any other adduct or derivative which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof. As used herein, the term “inhibitorily active metabolite or residue thereof” means that a metabolite or residue thereof is also an inhibitor of VPS34 and/or PI3K.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. This invention also envisions the quaternization of any basic nitrogen-containing groups of the compounds disclosed herein. Water or oil-soluble or dispersable products may be obtained by such quaternization. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

As described above, the pharmaceutically acceptable compositions of the present invention additionally comprise a pharmaceutically acceptable carrier, adjuvant, or vehicle, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylene-polyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

In yet another aspect, a method for treating a proliferative, inflammatory, or cardiovascular disorder is provided comprising administering an effective amount of a compound, or a pharmaceutical composition to a subject in need thereof. In certain embodiments of the present invention an “effective amount” of the compound or pharmaceutical composition is that amount effective for treating a proliferative, inflammatory, or cardiovascular disorder, or is that amount effective for treating cancer. In other embodiments, an “effective amount” of a compound is an amount which inhibits binding of PI3K and thereby blocks the resulting signaling cascades that lead to the abnormal activity of growth factors, receptor tyrosine kinases, protein serine/threonine kinases, G protein coupled receptors and phospholipid kinases and phosphatases.

The compounds and compositions, according to the method of the present invention, may be administered using any amount and any route of administration effective for treating the disease. The exact amount required will vary from subject to subject, depending on the species, age, and general condition of the subject, the severity of the disorder, the particular agent, its mode of administration, and the like. The compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disease being treated and the severity of the disease; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

The pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

While one or more of the inventive compounds may be used in an application of monotherapy to treat a disorder, disease or symptom, they also may be used in combination therapy, in which the use of an inventive compound or composition (therapeutic agent) is combined with the use of one or more other therapeutic agents for treating the same and/or other types of disorders, symptoms and diseases. Combination therapy includes administration of the therapeutic agents concurrently or sequentially. Alternatively, the therapeutic agents can be combined into one composition which is administered to the patient.

In one embodiment, the compounds of this invention are used in combination with other therapeutic agents, such as other inhibitors of VPS34 and/or PI3K. In some embodiments, a compound of the invention is administered in conjunction with a therapeutic agent selected from the group consisting of cytotoxic agents, radiotherapy, and immunotherapy. It is understood that other combinations may be undertaken while remaining within the scope of the invention.

Another aspect of the invention relates to inhibiting VPS34 and/or PI3K, activity in a biological sample or a patient, which method comprises administering to the patient, or contacting said biological sample with a compound of formula IB, ID, IIB, VB, or IIC, or a composition comprising said compound. The term “biological sample”, as used herein, generally includes in vivo, in vitro, and ex vivo materials, and also includes, without limitation, cell cultures or extracts thereof; biopsied material obtained from a mammal or extracts thereof; and blood, saliva, urine, feces, semen, tears, or other body fluids or extracts thereof.

Still another aspect of this invention is to provide a kit comprising separate containers in a single package, wherein the inventive pharmaceutical compounds, compositions and/or salts thereof are used in combination with pharmaceutically acceptable carriers to treat disorders, symptoms and diseases where VPS34 and/or PI3K kinase plays a role.

### Experimental Procedures

### I-A. Preparation of Certain Exemplary Compounds

Compounds (Shown in Table 1 below) were prepared using the general methods and specific examples described herein.

### Examples

Table 1 below depicts certain compounds represented by compounds of general formula IB, ID, and subsets IIB, VB, or IIC.

TABLE 1

| | |
|---|---|
| 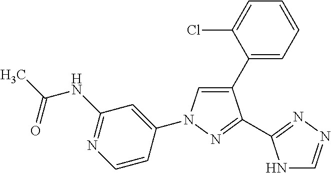 [SMILES: CC(=O)Nc1cc(-n2cc(-c3ccccc3Cl)c(-c3nnc[nH]3)n2)ccn1] | I-1 |
| 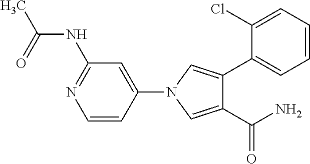 [SMILES: CC(=O)Nc1cc(-n2cc(C(N)=O)c(-c3ccccc3Cl)c2)ccn1] | I-2 |
| 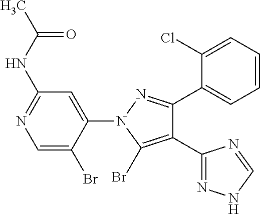 [SMILES: CC(=O)Nc1cc(-n2nc(-c3ccccc3Cl)c(-c3nc[nH]n3)c2Br)c(Br)cn1] | I-4 |
| 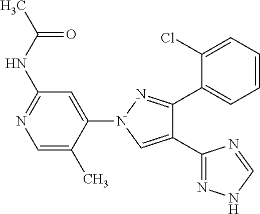 [SMILES: CC(=O)Nc1cc(-n2cc(-c3nc[nH]n3)c(-c3ccccc3Cl)n2)c(C)cn1] | I-5 |
| 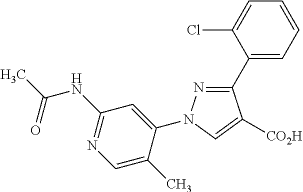 [SMILES: CC(=O)Nc1cc(-n2cc(C(=O)O)c(-c3ccccc3Cl)n2)c(C)cn1] | I-6 |
| 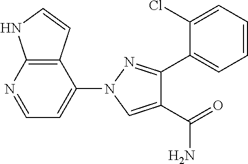 [SMILES: NC(=O)c1cn(-c2ccnc3[nH]ccc23)nc1-c1ccccc1Cl] | I-7 |
| 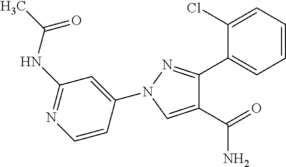 [SMILES: CC(=O)Nc1cc(-n2cc(C(N)=O)c(-c3ccccc3Cl)n2)ccn1] | I-8 |
| 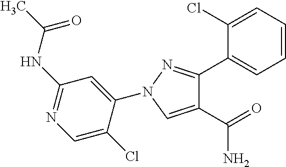 [SMILES: CC(=O)Nc1cc(-n2cc(C(N)=O)c(-c3ccccc3Cl)n2)c(Cl)cn1] | I-9 |
| 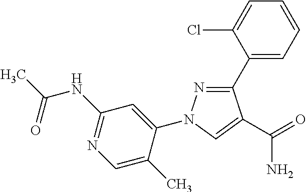 [SMILES: CC(=O)Nc1cc(-n2cc(C(N)=O)c(-c3ccccc3Cl)n2)c(C)cn1] | I-11 |
| 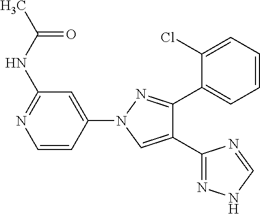 [SMILES: CC(=O)Nc1cc(-n2cc(-c3nc[nH]n3)c(-c3ccccc3Cl)n2)ccn1] | I-12 |
| 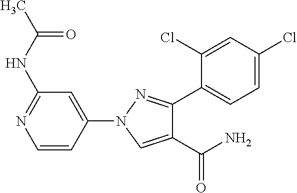 [SMILES: CC(=O)Nc1cc(-n2cc(C(N)=O)c(-c3ccc(Cl)cc3Cl)n2)ccn1] | I-13 |
| 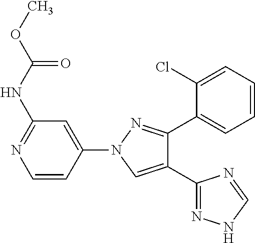 [SMILES: COC(=O)Nc1cc(-n2cc(-c3nc[nH]n3)c(-c3ccccc3Cl)n2)ccn1] | I-17 |
| 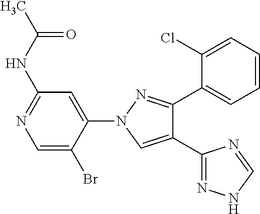 [SMILES: CC(=O)Nc1cc(-n2cc(-c3nc[nH]n3)c(-c3ccccc3Cl)n2)c(Br)cn1] | I-18 |
| 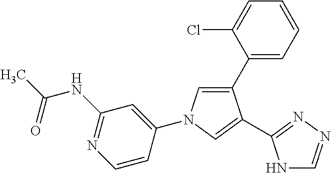 [SMILES: CC(=O)Nc1cc(-n2cc(-c3nnc[nH]3)c(-c3ccccc3Cl)c2)ccn1] | I-22 |
| 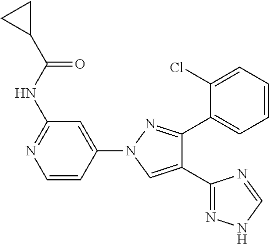 [SMILES: O=C(Nc1cc(-n2cc(-c3nc[nH]n3)c(-c3ccccc3Cl)n2)ccn1)C1CC1] | I-23 |
| 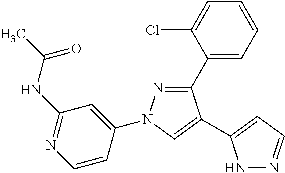 [SMILES: CC(=O)Nc1cc(-n2cc(-c3ccn[nH]3)c(-c3ccccc3Cl)n2)ccn1] | I-25 |
| 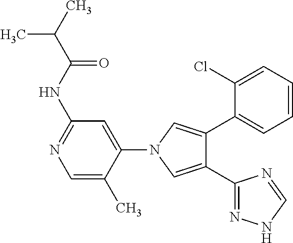 [SMILES: Cc1cnc(NC(=O)C(C)C)cc1-n1cc(-c2nc[nH]n2)c(-c2ccccc2Cl)c1] | I-26 |
| 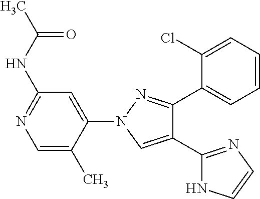 [SMILES: CC(=O)Nc1cc(-n2cc(-c3ncc[nH]3)c(-c3ccccc3Cl)n2)c(C)cn1] | I-27 |
| 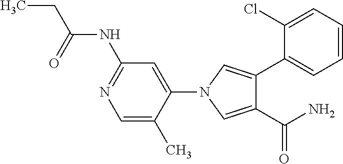 [SMILES: CCC(=O)Nc1cc(-n2cc(C(N)=O)c(-c3ccccc3Cl)c2)c(C)cn1] | I-28 |
| 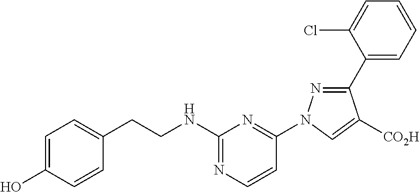 [SMILES: O=C(O)c1cn(-c2ccnc(NCCc3ccc(O)cc3)n2)nc1-c1ccccc1Cl] | I-29 |
| 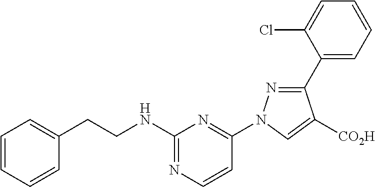 [SMILES: O=C(O)c1cn(-c2ccnc(NCCc3ccccc3)n2)nc1-c1ccccc1Cl] | I-30 |
| 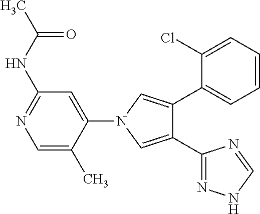 [SMILES: CC(=O)Nc1cc(-n2cc(-c3nc[nH]n3)c(-c3ccccc3Cl)c2)c(C)cn1] | I-31 |
| 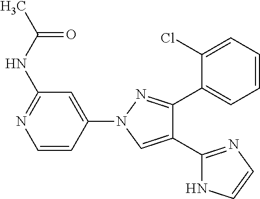 [SMILES: CC(=O)Nc1cc(-n2cc(-c3ncc[nH]3)c(-c3ccccc3Cl)n2)ccn1] | I-32 |
| 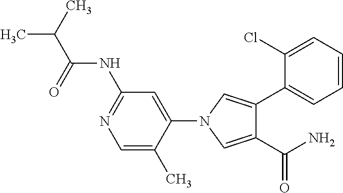 [SMILES: Cc1cnc(NC(=O)C(C)C)cc1-n1cc(C(N)=O)c(-c2ccccc2Cl)c1] | I-33 |
| 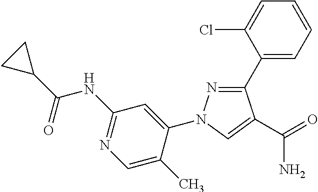 [SMILES: Cc1cnc(NC(=O)C2CC2)cc1-n1cc(C(N)=O)c(-c2ccccc2Cl)n1] | I-34 |
| 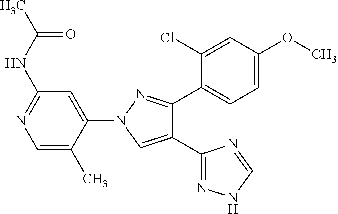 [SMILES: COc1ccc(-c2nn(-c3cc(NC(C)=O)ncc3C)cc2-c2nc[nH]n2)c(Cl)c1] | I-35 |
| 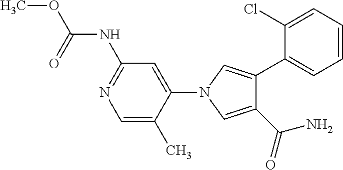 [SMILES: COC(=O)Nc1cc(-n2cc(C(N)=O)c(-c3ccccc3Cl)c2)c(C)cn1] | I-36 |
| 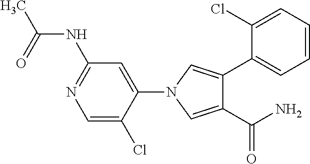 [SMILES: CC(=O)Nc1cc(-n2cc(C(N)=O)c(-c3ccccc3Cl)c2)c(Cl)cn1] | I-37 |
| 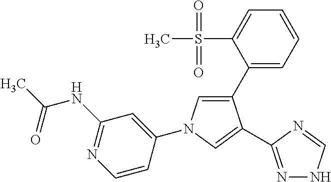 [SMILES: CC(=O)Nc1cc(-n2cc(-c3nc[nH]n3)c(-c3ccccc3S(C)(=O)=O)c2)ccn1] | I-38 |
| 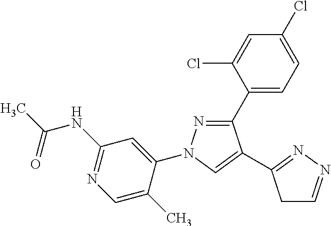 [SMILES: CC(=O)Nc1cc(-n2cc(C3=NN=CC3)c(-c3ccc(Cl)cc3Cl)n2)c(C)cn1] | I-39 |
| 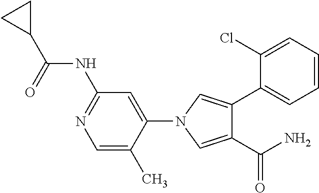 [SMILES: Cc1cnc(NC(=O)C2CC2)cc1-n1cc(C(N)=O)c(-c2ccccc2Cl)c1] | I-40 |
| 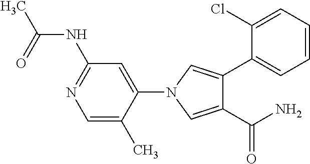 [SMILES: CC(=O)Nc1cc(-n2cc(C(N)=O)c(-c3ccccc3Cl)c2)c(C)cn1] | I-41 |
| 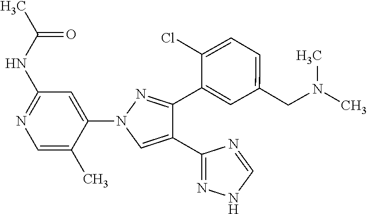 [SMILES: CC(=O)Nc1cc(-n2cc(-c3nc[nH]n3)c(-c3cc(CN(C)C)ccc3Cl)n2)c(C)cn1] | I-42 |
| 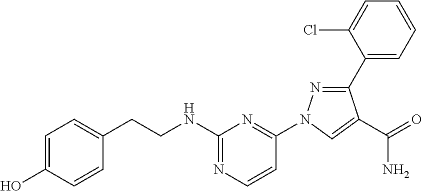 [SMILES: NC(=O)c1cn(-c2ccnc(NCCc3ccc(O)cc3)n2)nc1-c1ccccc1Cl] | I-43 |
| 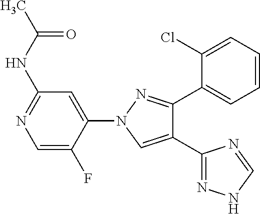 [SMILES: CC(=O)Nc1cc(-n2cc(-c3nc[nH]n3)c(-c3ccccc3Cl)n2)c(F)cn1] | I-44 |
| 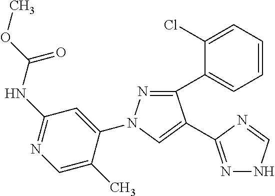 [SMILES: COC(=O)Nc1cc(-n2cc(-c3nc[nH]n3)c(-c3ccccc3Cl)n2)c(C)cn1] | I-45 |
| 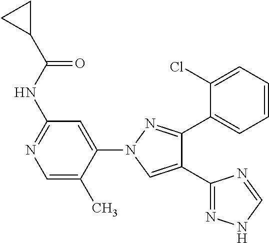 [SMILES: Cc1cnc(NC(=O)C2CC2)cc1-n1cc(-c2nc[nH]n2)c(-c2ccccc2Cl)n1] | I-46 |
| 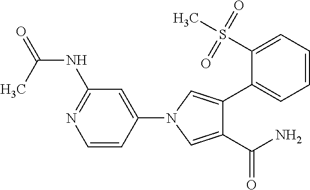 [SMILES: CC(=O)Nc1cc(-n2cc(C(N)=O)c(-c3ccccc3S(C)(=O)=O)c2)ccn1] | I-47 |
| 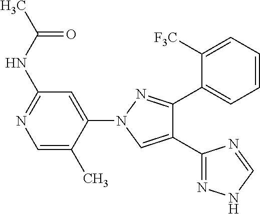 [SMILES: CC(=O)Nc1cc(-n2cc(-c3nc[nH]n3)c(-c3ccccc3C(F)(F)F)n2)c(C)cn1] | I-48 |
| 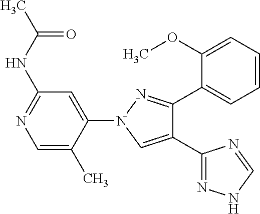 [SMILES: COc1ccccc1-c1nn(-c2cc(NC(C)=O)ncc2C)cc1-c1nc[nH]n1] | I-49 |
| 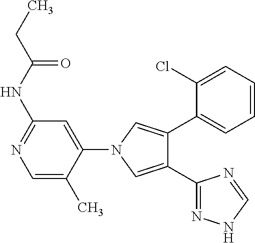 [SMILES: CCC(=O)Nc1cc(-n2cc(-c3nc[nH]n3)c(-c3ccccc3Cl)c2)c(C)cn1] | I-50 |
| 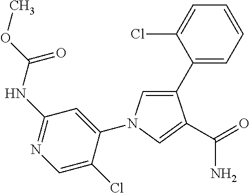 [SMILES: COC(=O)Nc1cc(-n2cc(C(N)=O)c(-c3ccccc3Cl)c2)c(Cl)cn1] | I-51 |
| 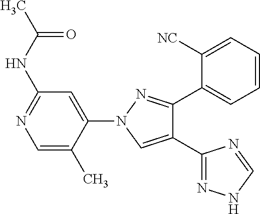 [SMILES: CC(=O)Nc1cc(-n2cc(-c3nc[nH]n3)c(-c3ccccc3C#N)n2)c(C)cn1] | I-52 |
| 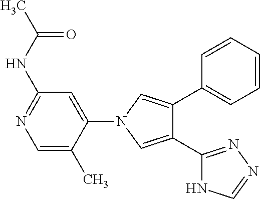 [SMILES: CC(=O)Nc1cc(-n2cc(-c3ccccc3)c(-c3nnc[nH]3)c2)c(C)cn1] | I-53 |
| 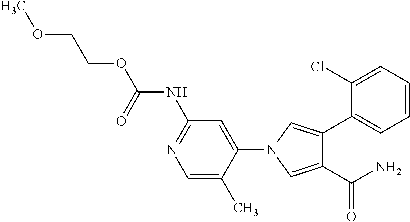 [SMILES: COCCOC(=O)Nc1cc(-n2cc(C(N)=O)c(-c3ccccc3Cl)c2)c(C)cn1] | I-54 |
| 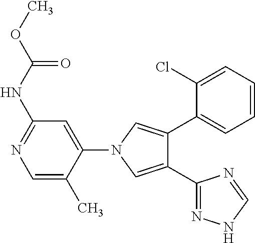 [SMILES: COC(=O)Nc1cc(-n2cc(-c3nc[nH]n3)c(-c3ccccc3Cl)c2)c(C)cn1] | I-55 |
| 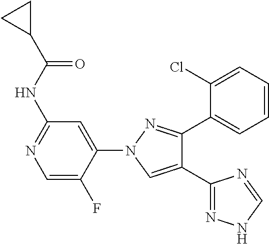 [SMILES: O=C(Nc1cc(-n2cc(-c3nc[nH]n3)c(-c3ccccc3Cl)n2)c(F)cn1)C1CC1] | I-56 |
| 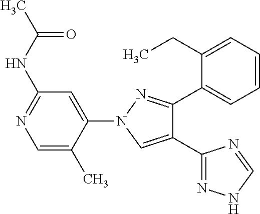 [SMILES: CCc1ccccc1-c1nn(-c2cc(NC(C)=O)ncc2C)cc1-c1nc[nH]n1] | I-57 |
| 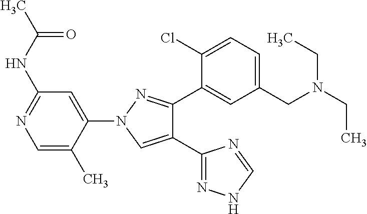 [SMILES: CCN(CC)Cc1ccc(Cl)c(-c2nn(-c3cc(NC(C)=O)ncc3C)cc2-c2nc[nH]n2)c1] | I-58 |
| 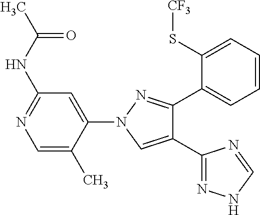 [SMILES: CC(=O)Nc1cc(-n2cc(-c3nc[nH]n3)c(-c3ccccc3SC(F)(F)F)n2)c(C)cn1] | I-59 |
| 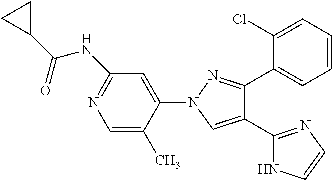 [SMILES: Cc1cnc(NC(=O)C2CC2)cc1-n1cc(-c2ncc[nH]2)c(-c2ccccc2Cl)n1] | I-60 |
| 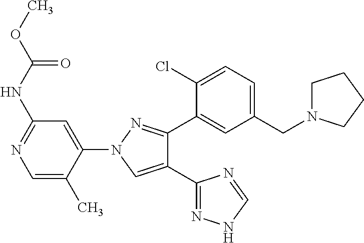 [SMILES: COC(=O)Nc1cc(-n2cc(-c3nc[nH]n3)c(-c3cc(CN4CCCC4)ccc3Cl)n2)c(C)cn1] | I-61 |
| 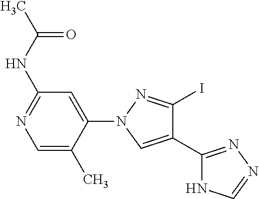 [SMILES: CC(=O)Nc1cc(-n2cc(-c3nnc[nH]3)c(I)n2)c(C)cn1] | I-62 |
| 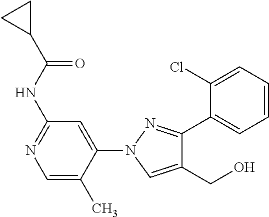 [SMILES: Cc1cnc(NC(=O)C2CC2)cc1-n1cc(CO)c(-c2ccccc2Cl)n1] | I-63 |
| 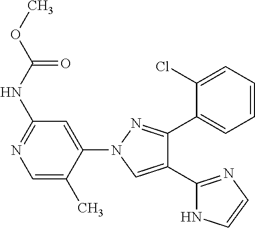 [SMILES: COC(=O)Nc1cc(-n2cc(-c3ncc[nH]3)c(-c3ccccc3Cl)n2)c(C)cn1] | I-64 |
| 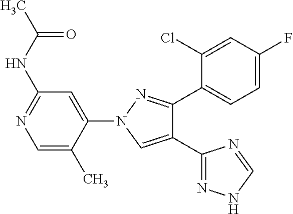 [SMILES: CC(=O)Nc1cc(-n2cc(-c3nc[nH]n3)c(-c3ccc(F)cc3Cl)n2)c(C)cn1] | I-65 |
| 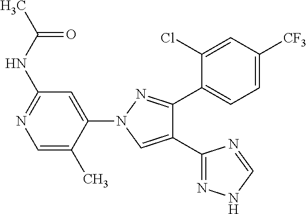 [SMILES: CC(=O)Nc1cc(-n2cc(-c3nc[nH]n3)c(-c3ccc(C(F)(F)F)cc3Cl)n2)c(C)cn1] | I-66 |
| 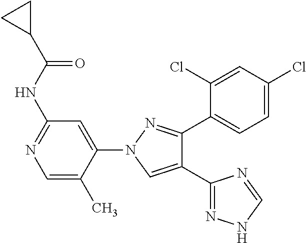 [SMILES: Cc1cnc(NC(=O)C2CC2)cc1-n1cc(-c2nc[nH]n2)c(-c2ccc(Cl)cc2Cl)n1] | I-67 |
| 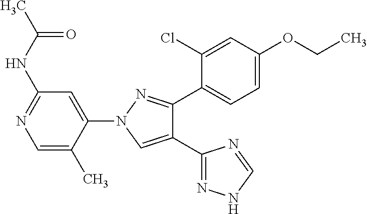 [SMILES: CCOc1ccc(-c2nn(-c3cc(NC(C)=O)ncc3C)cc2-c2nc[nH]n2)c(Cl)c1] | I-68 |
| 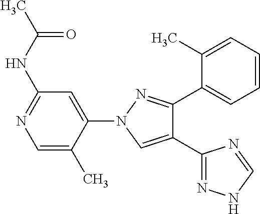 [SMILES: CC(=O)Nc1cc(-n2cc(-c3nc[nH]n3)c(-c3ccccc3C)n2)c(C)cn1] | I-69 |
| 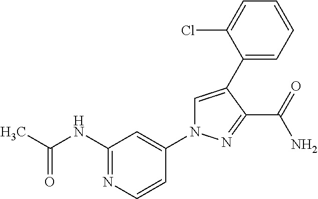 [SMILES: CC(=O)Nc1cc(-n2cc(-c3ccccc3Cl)c(C(N)=O)n2)ccn1] | I-20 |
| 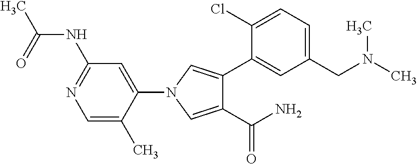 [SMILES: CC(=O)Nc1cc(-n2cc(C(N)=O)c(-c3cc(CN(C)C)ccc3Cl)c2)c(C)cn1] | I-70 |
| 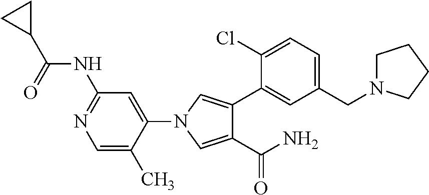 [SMILES: Cc1cnc(NC(=O)C2CC2)cc1-n1cc(C(N)=O)c(-c2cc(CN3CCCC3)ccc2Cl)c1] | I-71 |
| 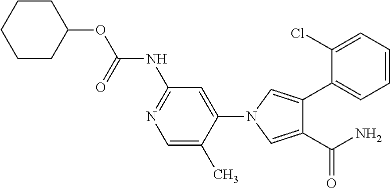 [SMILES: Cc1cnc(NC(=O)OC2CCCCC2)cc1-n1cc(C(N)=O)c(-c2ccccc2Cl)c1] | I-72 |
| 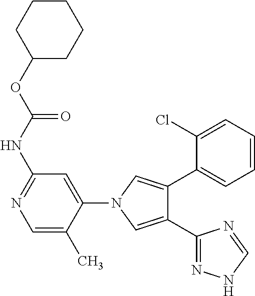 [SMILES: Cc1cnc(NC(=O)OC2CCCCC2)cc1-n1cc(-c2nc[nH]n2)c(-c2ccccc2Cl)c1] | I-73 |
| 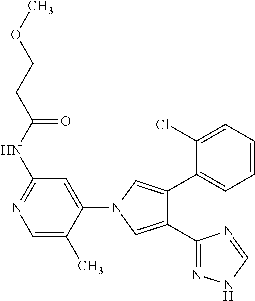 [SMILES: COCCC(=O)Nc1cc(-n2cc(-c3nc[nH]n3)c(-c3ccccc3Cl)c2)c(C)cn1] | I-74 |
| 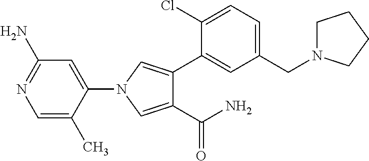 [SMILES: Cc1cnc(N)cc1-n1cc(C(N)=O)c(-c2cc(CN3CCCC3)ccc2Cl)c1] | I-75 |
| 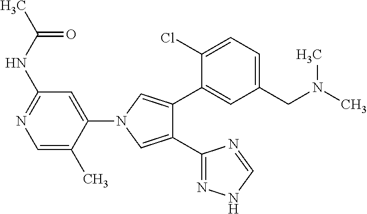 [SMILES: CC(=O)Nc1cc(-n2cc(-c3nc[nH]n3)c(-c3cc(CN(C)C)ccc3Cl)c2)c(C)cn1] | I-76 |
| 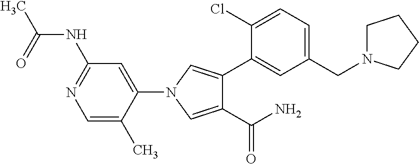 [SMILES: CC(=O)Nc1cc(-n2cc(C(N)=O)c(-c3cc(CN4CCCC4)ccc3Cl)c2)c(C)cn1] | I-77 |
| 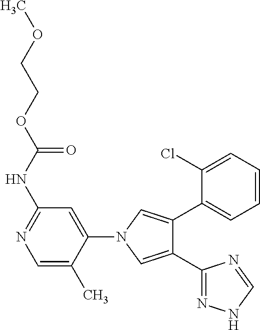 [SMILES: COCCOC(=O)Nc1cc(-n2cc(-c3nc[nH]n3)c(-c3ccccc3Cl)c2)c(C)cn1] | I-78 |
| 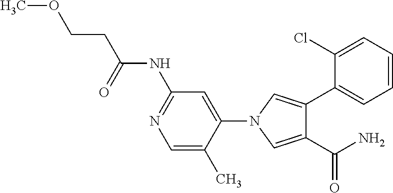 [SMILES: COCCC(=O)Nc1cc(-n2cc(C(N)=O)c(-c3ccccc3Cl)c2)c(C)cn1] | I-79 |
| 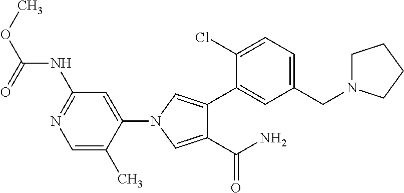 [SMILES: COC(=O)Nc1cc(-n2cc(C(N)=O)c(-c3cc(CN4CCCC4)ccc3Cl)c2)c(C)cn1] | I-80 |
| 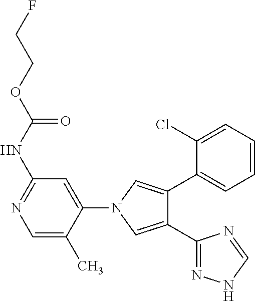 [SMILES: Cc1cnc(NC(=O)OCCF)cc1-n1cc(-c2nc[nH]n2)c(-c2ccccc2Cl)c1] | I-81 |
| 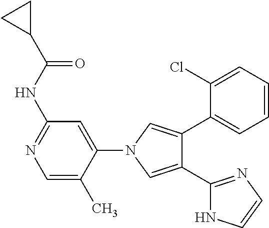 [SMILES: Cc1cnc(NC(=O)C2CC2)cc1-n1cc(-c2ncc[nH]2)c(-c2ccccc2Cl)c1] | I-82 |
| 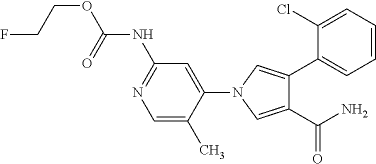 [SMILES: Cc1cnc(NC(=O)OCCF)cc1-n1cc(C(N)=O)c(-c2ccccc2Cl)c1] | I-83 |
| 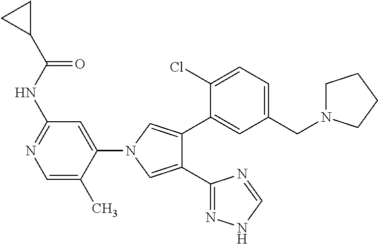 [SMILES: Cc1cnc(NC(=O)C2CC2)cc1-n1cc(-c2nc[nH]n2)c(-c2cc(CN3CCCC3)ccc2Cl)c1] | I-84 |
| 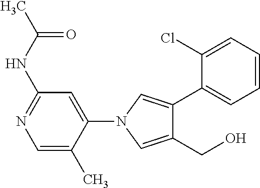 [SMILES: CC(=O)Nc1cc(-n2cc(CO)c(-c3ccccc3Cl)c2)c(C)cn1] | I-85 |
| 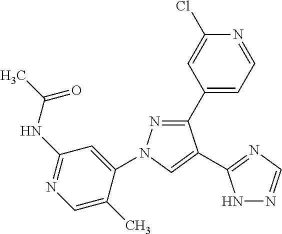 [SMILES: CC(=O)Nc1cc(-n2cc(-c3ncn[nH]3)c(-c3ccnc(Cl)c3)n2)c(C)cn1] | I-106 |
| 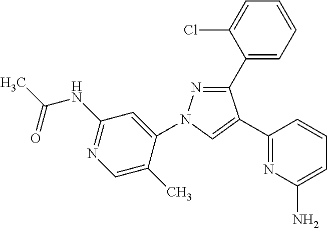 [SMILES: CC(=O)Nc1cc(-n2cc(-c3cccc(N)n3)c(-c3ccccc3Cl)n2)c(C)cn1] | I-107 |
| 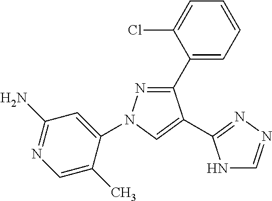 [SMILES: Cc1cnc(N)cc1-n1cc(-c2nnc[nH]2)c(-c2ccccc2Cl)n1] | I-108 |
| 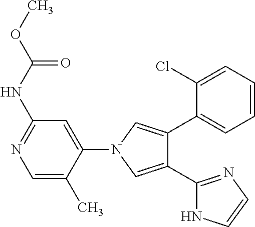 [SMILES: COC(=O)Nc1cc(-n2cc(-c3ncc[nH]3)c(-c3ccccc3Cl)c2)c(C)cn1] | I-109 |

The compounds of Table 1 above may also be identified by the following chemical names:

| Compound | Name |
|---|---|
| I-1 | N-{4-[4-(2-chlorophenyl)-3-(4H-1,2,4-triazol-3-yl)-1H-pyrazol-1-yljpyridin- |
| | 2-yl}acetamide |
| I-2 | 1-(2-acetamidopyridin-4-yl)-4-(2-chlorophenyl)-1H-pyrrole-3-carboxamide |
| I-4 | N-{5-bromo-4-[5-bromo-3-(2-chlorophenyl)-4-(1H-1,2,4-triazol-3-yl)-1H- |
| | pyrazol-1-yl]pyridin-2-yl}acetamide |
| I-5 | N-{4-[3-(2-chlorophenyl)-4-(1H-1,2,4-triazol-3-yl)-1H-pyrazol-1-yl]-5- |
| | methylpyridin-2-yl}acetamide |
| I-6 | 1-(2-acetamido-5-methylpyridin-4-yl)-3-(2-chlorophenyl)-1H-pyrazole-4- |
| | carboxylic acid |
| I-7 | 3-(2-chlorophenyl)-1-(1H-pyrrolo[2,3-b]pyridin-4-yl)-1H-pyrazole-4- |
| | carboxamide |
| I-8 | 1-(2-acetamidopyridin-4-yl)-3-(2-chlorophenyl)-1H-pyrazole-4-carboxamide |
| I-9 | 1-(2-acetamido-5-chloropyridin-4-yl)-3-(2-chlorophenyl)-1H-pyrazole-4- |
| | carboxamide |
| I-11 | 1-(2-acetamido-5-methylpyridin-4-yl)-3-(2-chlorophenyl)-1H-pyrazole-4- |
| | carboxamide |
| I-12 | N-{4-[3-(2-chlorophenyl)-4-(1H-1,2,4-triazol-3-yl)-1H-pyrazol-1-yl]pyridin- |
| | 2-yl}acetamide |
| I-13 | 1-(2-acetamidopyridin-4-yl)-3-(2,4-dichlorophenyl)-1H-pyrazole-4- |
| | carboxamide |
| I-17 | methyl {4-[3-(2-chlorophenyl)-4-(1H-1,2,4-triazol-3-yl)-1H-pyrazol-1- |
| | yl]pyridin-2-yl}carbamate |
| I-18 | N-{5-bromo-4-[3-(2-chlorophenyl)-4-(1H-1,2,4-triazol-3-yl)-1H-pyrazol-1- |
| | yl]pyridin-2-yl}acetamide |
| I-20 | 1-(2-acetamidopyridin-4-yl)-4-(2-chlorophenyl)-1H-pyrazole-3-carboxamide |
| I-22 | N-{4-[3-(2-chlorophenyl)-4-(4H-1,2,4-triazol-3-yl)-1H-pyrrol-1-yl]pyridin-2- |
| | yl}acetamide |
| I-23 | N-{4-[3-(2-chlorophenyl)-4-(1H-1,2,4-triazol-3-yl)-1H-pyrazol-1-yl]pyridin- |
| | 2-yl}cyclopropanecarboxamide |
| I-25 | N-{4-[3′-(2-chlorophenyl)-1′H,2H-3,4′-bipyrazol-1′-yl]pyridin-2-yl}acetamide |
| I-26 | N-{4-[3-(2-chlorophenyl)-4-(1H-1,2,4-triazol-3-yl)-1H-pyrrol-1-yl]-5- |
| | methylpyridin-2-yl}-2-methylpropanamide |
| I-27 | N-{4-[3-(2-chlorophenyl)-4-(1H-imidazol-2-yl)-1H-pyrazol-1-yl]-5- |
| | methylpyridin-2-yl}acetamide |
| I-28 | 4-(2-chlorophenyl)-1-[5-methyl-2-(propionylamino)pyridin-4-yl]-1H-pyrrole- |
| | 3-carboxamide |
| I-29 | 3-(2-chlorophenyl)-1-(2-{[2-(4-hydroxyphenyl)ethyl]amino}pyrimidin-4-yl)- |
| | 1H-pyrazole-4-carboxylic acid |
| I-30 | 3-(2-chlorophenyl)-1-{2-[(2-phenylethyl)amino]pyrimidin-4-yl}-1H-pyrazole- |
| | 4-carboxylic acid |
| I-31 | N-{4-[3-(2-chlorophenyl)-4-(1H-1,2,4-triazol-3-yl)-1H-pyrrol-1-yl]-5- |
| | methylpyridin-2-yl}acetamide |
| I-32 | N-{4-[3-(2-chlorophenyl)-4-(1H-imidazol-2-yl)-1H-pyrazol-1-yl]pyridin-2- |
| | yl}acetamide |
| I-33 | 4-(2-chlorophenyl)-1-[2-(isobutyrylamino)-5-methylpyridin-4-yl]-1H-pyrrole- |
| | 3-carboxamide |
| I-34 | 3-(2-chlorophenyl)-1-{2-[(cyclopropylcarbonyl)amino]-5-methylpyridin-4- |
| | yl}-1H-pyrazole-4-carboxamide |
| I-35 | N-{4-[3-(2-chloro-4-methoxyphenyl)-4-(1H-1,2,4-triazol-3-yl)-1H-pyrazol-1- |
| | yl]-5-methylpyridin-2-yl}acetamide |
| I-36 | methyl {4-[3-carbamoyl-4-(2-chlorophenyl)-1H-pyrrol-1-yl]-5-methylpyridin- |
| | 2-yl}carbamate |
| I-37 | 1-(2-acetamido-5-chloropyridin-4-yl)-4-(2-chlorophenyl)-1H-pyrrole-3- |
| | carboxamide |
| I-38 | N-(4-{3-[2-(methylsulfonyl)phenyl]-4-(1H-1,2,4-triazol-3-yl)-1H-pyrrol-1- |
| | yl}pyridin-2-yl)acetamide |
| I-39 | N-{4-[3-(2,4-dichlorophenyl)-4-(4H-1,2,4-triazol-3-yl)-1H-pyrazol-1-yl]-5- |
| | methylpyridin-2-yl}acetamide |
| I-40 | 4-(2-chlorophenyl)-1-{2-[(cyclopropylcarbonyl)amino]-5-methylpyridin-4- |
| | yl}-1H-pyrrole-3-carboxamide |
| I-41 | 1-(2-acetamido-5-methylpyridin-4-yl)-4-(2-chlorophenyl)-1H-pyrrole-3- |
| | carboxamide |
| I-42 | N-{4-[3-{2-chloro-5-[(dimethylamino)methyl]phenyl}-4-(1H-1,2,4-triazol-3- |
| | yl)-1H-pyrazol-1-yl]-5-methylpyridin-2-yl}acetamide |
| I-43 | 3-(2-chlorophenyl)-1-(2-{[2-(4-hydroxyphenyl)ethyl]amino}pyrimidin-4-yl)- |
| | 1H-pyrazole-4-carboxamide |
| I-44 | N-{4-[3-(2-chlorophenyl)-4-(1H-1,2,4-triazol-3-yl)-1H-pyrazol-1-yl]-5- |
| | fluoropyridin-2-yl}acetamide |
| I-45 | methyl {4-[3-(2-chlorophenyl)-4-(1H-1,2,4-triazol-3-yl)-1H-pyrazol-1-yl]-5- |
| | methylpyridin-2-yl}carbamate |
| I-46 | N-{4-[3-(2-chlorophenyl)-4-(1H-1,2,4-triazol-3-yl)-1H-pyrazol-1-yl]-5- |
| | methylpyridin-2-yl}cyclopropanecarboxamide |
| I-47 | 1-(2-acetamidopyridin-4-yl)-4-[2-(methylsulfonyl)phenyl]-1H-pyrrole-3- |
| | carboxamide |
| I-48 | N-(5-methyl-4-{4-(1H-1,2,4-triazol-3-yl)-3-[2-(trifluoromethyl)phenyl]-1H- |
| | pyrazol-1-yl}pyridin-2-yl)acetamide |
| I-49 | N-{4-[3-(2-methoxyphenyl)-4-(1H-1,2,4-triazol-3-yl)-1H-pyrazol-1-yl]-5- |
| | methylpyridin-2-yl}acetamide |
| I-50 | N-{4-[3-(2-chlorophenyl)-4-(1H-1,2,4-triazol-3-yl)-1H-pyrrol-1-yl]-5- |
| | methylpyridin-2-yl}propanamide |
| I-51 | methyl {4-[3-carbamoyl-4-(2-chlorophenyl)-1H-pyrrol-1-yl]-5-chloropyridin- |
| | 2-yl}carbamate |
| I-52 | N-{4-[3-(2-cyanophenyl)-4-(1H-1,2,4-triazol-3-yl)-1H-pyrazol-1-yl]-5- |
| | methylpyridin-2-yl}acetamide |
| I-53 | N-{5-methyl-4-[3-phenyl-4-(4H-1,2,4-triazol-3-yl)-1H-pyrrol-1-yl]pyridin-2- |
| | yl}acetamide |
| I-54 | 2-methoxyethyl {4-[3-carbamoyl-4-(2-chlorophenyl)-1H-pyrrol-1-yl]-5- |
| | methylpyridin-2-yl}carbamate |
| I-55 | methyl {4-[3-(2-chlorophenyl)-4-(1H-1,2,4-triazol-3-yl)-1H-pyrrol-1-yl]-5- |
| | methylpyridin-2-yl}carbamate |
| I-56 | N-{4-[3-(2-chlorophenyl)-4-(1H-1,2,4-triazol-3-yl)-1H-pyrazol-1-yl]-5- |
| | fluoropyridin-2-yl}cyclopropanecarboxamide |
| I-57 | N-{4-[3-(2-ethylphenyl)-4-(1H-1,2,4-triazol-3-yl)-1H-pyrazol-1-yl]-5- |
| | methylpyridin-2-yl}acetamide |
| I-58 | N-{4-[3-{2-chloro-5-[(diethylamino)methyl]phenyl}-4-(1H-1,2,4-triazol-3-yl)- |
| | 1H-pyrazol-1-yl]-5-methylpyridin-2-yl}acetamide |
| I-59 | N-{5-methyl-4-[4-(1H-1,2,4-triazol-3-yl)-3-{2- |
| | [(trifluoromethyl)sulfanyl]phenyl}-1H-pyrazol-1-yl]pyridin-2-yl}acetamide |
| I-60 | N-{4-[3-(2-chlorophenyl)-4-(1H-imidazol-2-yl)-1H-pyrazol-1-yl]-5- |
| | methylpyridin-2-yl}cyclopropanecarboxamide |
| I-61 | methyl (4-{3-[2-chloro-5-(pyrrolidin-1-ylmethyl)phenyl]-4-(1H-1,2,4-triazol- |
| | 3-yl)-1H-pyrazol-1-yl}-5-methylpyridin-2-yl)carbamate |
| I-62 | N-{4-[3-iodo-4-(4H-1,2,4-triazol-3-yl)-1H-pyrazol-1-yl]-5-methylpyridin-2- |
| | yl}acetamide |
| I-63 | N-{4-[3-(2-chlorophenyl)-4-(hydroxymethyl)-1H-pyrazol-1-yl]-5- |
| | methylpyridin-2-yl}cyclopropanecarboxamide |
| I-64 | methyl {4-[3-(2-chlorophenyl)-4-(1H-imidazol-2-yl)-1H-pyrazol-1-yl]-5- |
| | methylpyridin-2-yl}carbamate |
| I-65 | N-{4-[3-(2-chloro-4-fluorophenyl)-4-(1H-1,2,4-triazol-3-yl)-1H-pyrazol-1-yl]- |
| | 5-methylpyridin-2-yl}acetamide |
| I-66 | N-(4-{3-[2-chloro-4-(trifluoromethyl)phenyl]-4-(1H-1,2,4-triazol-3-yl)-1H- |
| | pyrazol-1-yl}-5-methylpyridin-2-yl)acetamide |
| I-67 | N-{4-[3-(2,4-dichlorophenyl)-4-(1H-1,2,4-triazol-3-yl)-1H-pyrazol-1-yl]-5- |
| | methylpyridin-2-yl}cyclopropanecarboxamide |
| I-68 | N-{4-[3-(2-chloro-4-ethoxyphenyl)-4-(1H-1,2,4-triazol-3-yl)-1H-pyrazol-1- |
| | yl]-5-methylpyridin-2-yl}acetamide |
| I-69 | N-{5-methyl-4-[3-(2-methylphenyl)-4-(1H-1,2,4-triazol-3-yl)-1H-pyrazol-1- |
| | yl]pyridin-2-yl}acetamide |
| I-70 | 1-(2-acetamido-5-methylpyridin-4-yl)-4-{2-chloro-5- |
| | [(dimethylamino)methyl]phenyl}-1H-pyrrole-3-carboxamide |
| I-71 | 4-[2-chloro-5-(pyrrolidin-1-ylmethyl)phenyl]-1-{2- |
| | [(cyclopropylcarbonyl)amino]-5-methylpyridin-4-yl}-1H-pyrrole-3- |
| | carboxamide |
| I-72 | cyclohexyl {4-[3-carbamoyl-4-(2-chlorophenyl)-1H-pyrrol-1-yl]-5- |
| | methylpyridin-2-yl}carbamate |
| I-73 | cyclohexyl {4-[3-(2-chlorophenyl)-4-(1H-1,2,4-triazol-3-yl)-1H-pyrrol-1-yl]- |
| | 5-methylpyridin-2-yl}carbamate |
| I-74 | N-{4-[3-(2-chlorophenyl)-4-(1H-1,2,4-triazol-3-yl)-1H-pyrrol-1-yl]-5- |
| | methylpyridin-2-yl}-3-methoxypropanamide |
| I-75 | 1-(2-amino-5-methylpyridin-4-yl)-4-[2-chloro-5-(pyrrolidin-1- |
| | ylmethyl)phenyl]-1H-pyrrole-3-carboxamide |
| I-76 | N-{4-[3-{2-chloro-5-[(dimethylamino)methyl]phenyl}-4-(1H-1,2,4-triazol-3- |
| | yl)-1H-pyrrol-1-yl]-5-methylpyridin-2-yl}acetamide |
| I-77 | 1-(2-acetamido-5-methylpyridin-4-yl)-4-[2-chloro-5-(pyrrolidin-1- |
| | ylmethyl)phenyl]-1H-pyrrole-3-carboxamide |
| I-78 | 2-methoxyethyl {4-[3-(2-chlorophenyl)-4-(1H-1,2,4-triazol-3-yl)-1H-pyrrol-1- |
| | yl]-5-methylpyridin-2-yl}carbamate |
| I-79 | 4-(2-chlorophenyl)-1-{2-[(3-methoxypropanoyl)amino]-5-methylpyridin-4- |
| | yl}-1H-pyrrole-3-carboxamide |
| I-80 | methyl (4-{3-carbamoyl-4-[2-chloro-5-(pyrrolidin-1-ylmethyl)phenyl]-1H- |
| | pyrrol-1-yl}-5-methylpyridin-2-yl)carbamate |
| I-81 | 2-fluoroethyl {4-[3-(2-chlorophenyl)-4-(1H-1,2,4-triazol-3-yl)-1H-pyrrol-1- |
| | yl]-5-methylpyridin-2-yl}carbamate |
| I-82 | N-{4-[3-(2-chlorophenyl)-4-(1H-imidazol-2-yl)-1H-pyrrol-1-yl]-5- |
| | methylpyridin-2-yl}cyclopropanecarboxamide |
| I-83 | 2-fluoroethyl {4-[3-carbamoyl-4-(2-chlorophenyl)-1H-pyrrol-1-yl]-5- |
| | methylpyridin-2-yl}carbamate |
| I-84 | N-(4-{3-[2-chloro-5-(pyrrolidin-1-ylmethyl)phenyl]-4-(1H-1,2,4-triazol-3-yl)- |
| | 1H-pyrrol-1-yl}-5-methylpyridin-2-yl)cyclopropanecarboxamide |
| I-85 | N-{4-[3-(2-chlorophenyl)-4-(hydroxymethyl)-1H-pyrrol-1-yl]-5- |
| | methylpyridin-2-yl}acetamide |
| I-106 | N-{4-[3-(2-chloropyridin-4-yl)-4-(1H-1,2,4-triazol-5-yl)-1H-pyrazol-1-yl]-5- |
| | methylpyridin-2-yl}acetamide |
| I-107 | N-{4-[4-(6-aminopyridin-2-yl)-3-(2-chlorophenyl)-1H-pyrazol-1-yl]-5- |
| | methylpyridin-2-yl}acetamide |
| I-108 | 4-[3-(2-chlorophenyl)-4-(4H-1,2,4-triazol-3-yl)-1H-pyrazol-1-yl]-5- |
| | methylpyridin-2-amine |
| I-109 | methyl {4-[3-(2-chlorophenyl)-4-(1H-imidazol-2-yl)- 1H-pyrrol-1-yl]-5- |
| | methylpyridin-2-yl}carbamate |

### DEFINITIONS

AA LCMS method using ammonium acetate
ACN acetonitrile
AcOH acetic acid
BOC tert-butoxycarbonyl

### C Celsius

dba dibenzylideneacetone
DBU 1,8-Diazabicyclo[5.4.0]undec-7-ene
DCE dichloroethane
DCM dichloromethane
DIEA diisopropylethylamine
DMAP 4-dimethylaminopyridine
DME 1,2-dimethoxyethane
DMF dimethylformamide
DMF-DMA dimethylformamide dimethylacetal
dppf 1, 1′-bis(diphenylphosphino)ferrocene
DMSO dimethylsulfoxide
EtOAc ethyl acetate
FA LCMS method using formic acid
h hours
HPLC high pressure liquid chromatography
HATU O-(7-azabenzotriazol-1-yl)-N,N,N′,N′-tetramethyluronium hexafluorophosphate
IC₅₀ inhibitory concentration 50%
KOH potassium hydroxide
LCMS liquid chromoatography mass spectrometry
m/z mass to charge
MeOH methanol
min minutes
MS mass spectrum

### NBS N-bromosuccinimide

### NCS N-chlorosuccinimide

PCC pyridinium chlorochromate
psi pounds per square inch
rt room temperature
SEM silylethoxymethyl
STAB sodium triacetoxyborohydride
TEA triethylamine
TFA trifluoroacetic acid
THF tetrahydrofuran
TBAF tetrabutylammoniumfluoride
TBTU O-(benzotriazol-1-yl)-N,N,N′,N′-tetramethyluronium tetrafluoroborate
TMS trimethylsilyl
Xantphos 4,5-bis(diphenylphosphino)-9,9-dimethylxanthene

Analytical LCMS Methods

LCMS spectra were recorded on a Hewlett-Packard HP1100 or Agilent 1100 Series LC system connected to a Micromass mass spectromteter using reverse phase C18 columns. Various gradients and run times were selected in order to best characterize the compounds. Mobile phases were based on ACN/water gradients and contained either 0.1% formic acid (methods indicated FA) or 10 mM ammonium acetate (methods indicated AA). One example of a solvent gradient that was used was 100% mobile phase A (mobile phase A=99% water+1% ACN+0.1% formic acid) to 100% mobile phase B (mobile phase B=95% ACN+5% water+0.1% formic acid) at a flow rate of 1 mL/min for a 16.5 min run.

One of ordinary skill in the art will recognize that modifications of the gradient, column length, and flow rate are possible and that some conditions may be suitable for compound characterization than others, depending on the chemical species being analyzed.

### Example 1

### Synthesis of Intermediate Stannanes and Boronic Esters

### N-[4-(4,4,5,5-tetramethyl-1,3,2-dioxaborolan-2-yl)pyridin-2-yl]acetamide

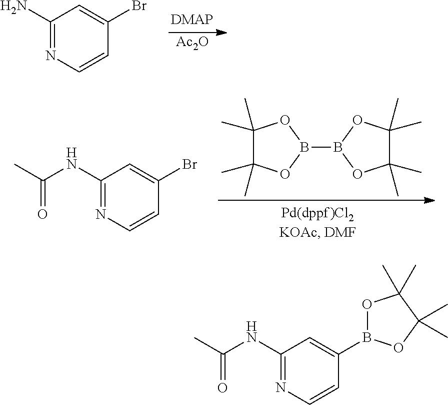

### Step 1: N-(4-bromopyridin-2-yl)acetamide

To a solution of 4-bromopyridin-2-amine (12.0 g, 69.4 mmol) in acetic anhydride (240 mL) was added DMAP (0.0847 g, 0.694 mmol). The reaction mixture was allowed to stir at 140° C. for 3 h and then allowed to cool to rt. Ice water was added and the pH of the mixture was adjusted to 8.5 by the addition of concentrated NH₄OH. The solid which precipitated was filtered, washed with cold water and hexanes, and dried to give N-(4-bromopyridin-2-yl)acetamide (13.3 g) as a white solid.

### Step 2: N-[4-(4,4,5,5-tetramethyl-1,3,2-dioxaborolan-2-yl)pyridin-2-yl]acetamide

To a mixture of N-(4-bromopyridin-2-yl)acetamide (17.2 g, 80 mmol, 1.0 equiv.), 4,4,4′,4′,5,5,5′,5′-octamethyl-2,2′-bi-1,3,2-dioxaborolane (26.4 g, 104 mmol), Pd(dppf)Cl₂ (11.7 g, 16 mmol) and KOAc (23.6 g, 240 mmol) under an atmosphere of nitrogen was added anhydrous DMF (1500 mL). The mixture was allowed to stir at 80° C. for 3.5 h. The solvent was removed and the residue was diluted with EtOAc (1000 mL). Activated carbon (100 g) was added. The slurry was heated at reflux for 5 min and then filtered. The organic solution was concentrated and the residue was re-crystallized from EtOAc to give N-[4-(4,4,5,5-tetramethyl-1,3,2-dioxaborolan-2-yl)pyridin-2-yl]acetamide (6.1 g, 29%) as a white solid. ¹H NMR (400 MHz, DMSO-d₆): δ 1.29 (s, 12H), 2.09 (s, 3H), 7.24 (dd, J=6.0, 1.2 Hz, 1H), 8.30-8.33 (m, 2H), 10.47 (br s, 1H).

Compounds in the following table were prepared from the appropriate starting materials using the procedures described above:

| | |
|---|---|
| 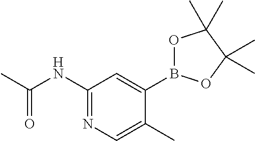 [SMILES: CC(=O)Nc1cc(B2OC(C)(C)C(C)(C)O2)c(C)cn1] | N-[5-methyl-4-(4,4,5,5-tetramethyl-1,3,2-dioxaborolan-2-y1)pyridin- 2-yl]acetamide |

### N-[5-methyl-4-(4,4,5,5-tetramethyl-1,3,2-dioxaborolan-2-yl)pyridin-2-yl]cyclopropanecarboxamide

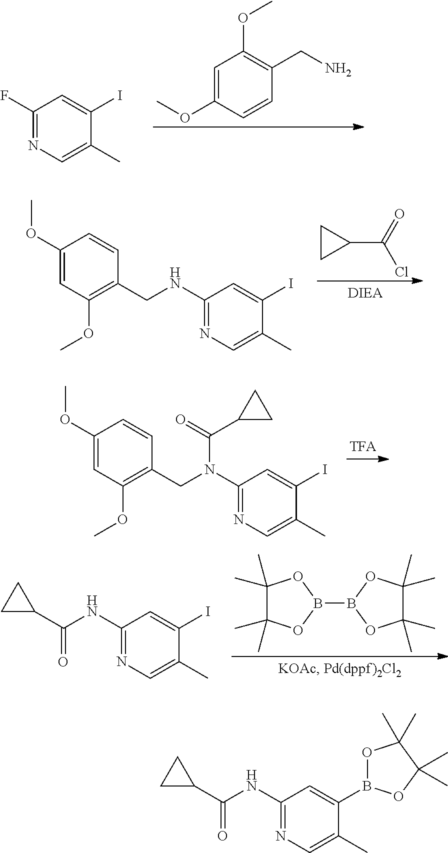

### Step 1: N-(2,4-dimethoxybenzyl)-4-iodo-5-methylpyridin-2-amine

A solution of 2-fluoro-4-iodo-5-methylpyridine (85 g, 340 mmol) in 1-(2,4-dimethoxyphenyl)methanamine (270 mL, 1.68 mol) was allowed to stir at 110° C. overnight. The reaction mixture was allowed to cool to rt and diluted with EtOAc. A precipitate formed and was filtered and then washed with EtOAc. The solid was purified further by column chromatography to give N-(2,4-dimethoxybenzyl)-4-iodo-5-methylpyridin-2-amine (138 g, 50%).

### Step 2: N-(2,4-dimethoxybenzyl)-N-(4-iodo-5-methylpyridin-2-yl)cyclopropanecarboxamide

To a solution of DIEA (76 mL, 440 mmol) in THF (1700 mL) was added N-(2,4-dimethoxybenzyl)-4-iodo-5-methylpyridin-2-amine (85 g, 220 mmol) and cyclopropanecarbonyl chloride (27.9 mL, 310 mmol). The reaction mixture was allowed to stir at 70° C. for 12 h and then concentrated. The residue was diluted with aqueous saturated ammonium chloride and extracted with DCM. The organic solutions were combined, dried over Na₂SO₄, filtered and concentrated to give N-(2,4-dimethoxybenzyl)-N-(4-iodo-5-methylpyridin-2-yl)cyclopropanecarboxamide (130 g, 80%) which was used in the next step without purification.

### Step 3: N-(4-iodo-5-methylpyridin-2-yl)cyclopropanecarboxamide

A solution of N-(2,4-dimethoxybenzyl)-N-(4-iodo-5-methylpyridin-2-yl)cyclopropanecarboxamide (65 g, 144 mmol) and TFA (833 mL, 4.13 mol) in DCM (850 mL) was allowed to stir at rt overnight. The reaction mixture was concentrated and the residue was redissolved in DCM. Aqueous sodium bicarbonate was added and the solution was extracted with DCM. The organic solutions were combined, dried over Na₂SO₄, filtered and concentrated. The residue was purified by column chromatography to give N-(4-iodo-5-methylpyridin-2-yl)cyclopropanecarboxamide (60 g, 70%).

### Step 4: N-[5-methyl-4-(4,4,5,5-tetramethyl-1,3,2-dioxaborolan-2-yl)pyridin-2-yl]cyclopropanecarboxamide

A mixture of N-(4-iodo-5-methylpyridin-2-yl)cyclopropanecarboxamide (20 g, 66 mmol). 4,4,4′,4′,5,5,5′,5′-octamethyl-2,2′-bi-1,3,2-dioxaborolane (33.6 g, 132 mmol) and potassium acetate (19.4 g, 198 mmol) in DMSO (200 mL) was degassed with nitrogen for 20 min. Pd(dppf)₂Cl₂ (5.4 g, 7 mmol) was added and the mixture was again degassed with nitrogen for 20 min. The reaction mixture was allowed to stir at 60° C. overnight and was then allowed to cool to rt and filtered. The filtrate was diluted with EtOAc and the solution was washed with water and brine. Activated charcoal was added to the organic solution and the mixture was heated at reflux for 3 h. The mixture was filtered and the filtrate was concentrated. The residue was taken up in tert-butyl dimethylether and the resulting solid was filtered. The filtrate was concentrated and the resulting solid was washed with petroleum ether to give pure N-[5-methyl-4-(4,4,5,5-tetramethyl-1,3,2-dioxaborolan-2-yl)pyridin-2-yl]cyclopropanecarboxamide (7.4 g, 37%).

### N-[5-methyl-4-(trimethylstannyl)pyridin-2-yl]acetamide

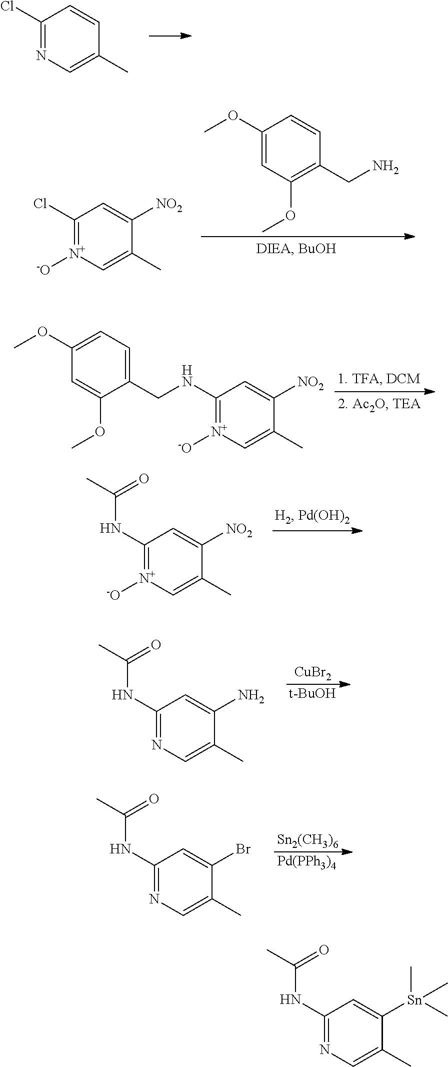

### Step 1: 2-chloro-5-methyl-4-nitropyridine 1-oxide

Hydrogen peroxide (17 mL) was added via addition funnel over 10 minutes to a solution of 2-chloro-5-methylpyridine (5.5 mL, 50 mmol) in acetic anhydride (17 mL). The reaction mixture was allowed to stir at rt overnight and then to stir at 60° C. for 30 h. Excess AcOH was removed under pressure and then the residue was added in small portions to concentrated sulfuric acid (10.3 mL). The resulting solution was added to a mixture of concentrated sulfuric acid (10.3 mL) and fuming nitric acid (17.2 mL) and allowed to stir at 100° C. After 1.5 h, the reaction mixture was poured onto ice. The solution was basified by the addition of solid ammonium carbonate until gas evolution ceased and a precipitate formed. The mixture was further basified with concentrated NH₄OH to a final pH of 11. After stirring for 1 h at rt, the mixture was filtered and 2-chloro-5-methyl-4-nitropyridine 1-oxide (6.25 g, 66%) was isolated as a yellow solid. LCMS (FA): m/z=189/191 (M+H).

### Step 2: N-(2,4-dimethoxybenzyl)-5-methyl-4-nitropyridin-2-amine 1-oxide

A mixture of 2-chloro-5-methyl-4-nitropyridine 1-oxide (1.1 g, 5.8 mmol), 1-(2,4-dimethoxyphenyl)methanamine (1.1 mL, 7.0 mmol), DIEA (2.0 mL, 11.6 mmol), and 1-butanol (9 mL) was heated at 120° C. under microwave irradiation for 8 h. The reaction mixture was allowed to cool to rt and was filtered. The resulting solid was washed with water (20 mL) and dried to give N-(2,4-dimethoxybenzyl)-5-methyl-4-nitropyridin-2-amine 1-oxide (1.2 g, 67%), which was used in the next step without further purification.

### Step 3: N-(5-methyl-4-nitro-1-oxidopyridin-2-yl)acetamide

A solution of N-(2,4-dimethoxybenzyl)-5-methyl-4-nitropyridin-2-amine 1-oxide (1.1 g, 3.5 mmol) in DCM (20 mL) and TFA (3 mL) was allowed to stir at rt for 4 h. The reaction mixture was concentrated and the residue was dissolved in DCM (20 mL). To this solution were added TEA (2.5 mL, 17.7 mmol) and acetic anhydride (0.4 g, 4.3 mmol). The reaction mixture was allowed to stir at rt overnight (usually 12 h, but again, probably not a big deal) and then filtered to give N-(5-methyl-4-nitro-1-oxidopyridin-2-yl)acetamide (0.73 g, 98%) which was used without further purification.

### Step 4: N-(4-amino-5-methylpyridin-2-yl)acetamide

A mixture of N-(5-methyl-4-nitro-1-oxidopyridin-2-yl)acetamide (3.1 g, 14.7 mmol) and Pd(OH)₂ (20% on carbon, 1.6 g) in MeOH (80 mL) was allowed to stir under 40 psi of hydrogen at rt for 6 days. The reaction mixture was then filtered over celite and the filter cake was washed with DCM. The filtrate was concentrated to give N-(4-amino-5-methylpyridin-2-yl)acetamide (2.1 g, 86%) which was used without further purification.

### Step 5: N-(4-bromo-5-methylpyridin-2-yl)acetamide

Copper(II) bromide (8.8 g, 39.5 mmol) was dissolved in ACN (85 mL). To this solution was added tert-butyl nitrite (4.1 mL, 34.2 mmol). The mixture was allowed to heat at 65° C. for 15 min, and then N-(4-amino-5-methylpyridin-2-yl)acetamide (4.4 g, 26.3 mmol) in ACN (40 mL) was added. The reaction mixture was allowed to continue to stir at 65° C. for 35 min. The reaction mixture was concentrated and 15% NH₄OH was added to the residue. The solution was extracted with EtOAc (3×150 mL). The organic solutions were combined and concentrated. The residue was purified by column chromatography to give N-(4-bromo-5-methylpyridin-2-yl)acetamide (2.56 g, 42%).

### Step 6: N-[5-methyl-4-(trimethylstannyl)pyridin-2-yl]acetamide

A mixture of N-(4-bromo-5-methylpyridin-2-yl)acetamide (2.56 g, 11.2 mmol), hexamethylditin (3.0 mL, 14.5 mmol) and tetrakis(triphenylphosphine) palladium(0) (0.65 g, 0.56 mmol) in 1,4-dioxane (42 mL) was allowed to stir at 95° C. for 4 h. The reaction mixture was allowed to cool to rt and then filtered over celite. The filtrate was concentrated and the residue was purified by column chromatography to give N-[5-methyl-4-(trimethylstannyl)pyridin-2-yl]acetamide (3.0 g, 86%). LCMS (FA): m/z=315.2 (M+H).

### Example 2

### Synthesis of Intermediate Heterocycles

### Methyl 3-(2-chlorophenyl)-1H-pyrazole-4-carboxylate

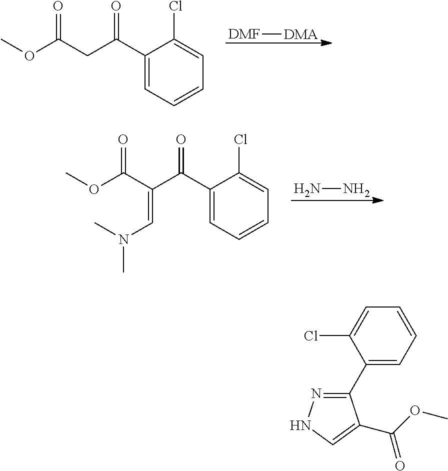

### Step 1: methyl 2-(2-chlorobenzoyl)-3-(dimethylamino)acrylate

A solution of methyl 3-(2-chlorophenyl)-3-oxopropanoate (5.00 g, 23.5 mmol) was dissolved in DMF-DMA (9.4 mL, 70 mmol). The reaction mixture was allowed to stir at rt for 2 h, at 60° C. for 1 h, and then allowed to cool to rt. Water was added and the mixture was extracted with EtOAc. The organic solutions were combined, dried over MgSO₄, filtered and concentrated to give methyl 2-(2-chlorobenzoyl)-3-(dimethylamino)acrylate (6.02 g, 96%), which was used without purification in the next step. LCMS (FA): m/z=268 (M+H).

### Step 2: methyl 3-(2-chlorophenyl)-1H-pyrazole-4-carboxylate

Methyl 2-(2-chlorobenzoyl)-3-(dimethylamino)acrylate (5.90 g, 22.0 mmol) was dissolved in AcOH (25 mL) and to this solution was added hydrazine hydrate (1.44 mL, 29.6 mmol). The reaction mixture was allowed to stir at rt overnight and then concentrated. The residue was dissolved in DCM and washed with aqueous saturated sodium bicarbonate. The organic solution was separated, dried over Na₂SO₄, filtered and concentrated. The resulting solid was dissolved in EtOAc and concentrated again to give methyl 3-(2-chlorophenyl)-1H-pyrazole-4-carboxylate (4.96 g, 95%), which was used without purification in the next step. LCMS (AA): m/z=237 (M+H).

### Example 3

### Synthesis of N-{4-[3-(2-chlorophenyl)-4-(1H-1,2,4-triazol-3-yl)-1H-pyrazol-1-yl]pyridin-2-yl}acetamide (I-12)

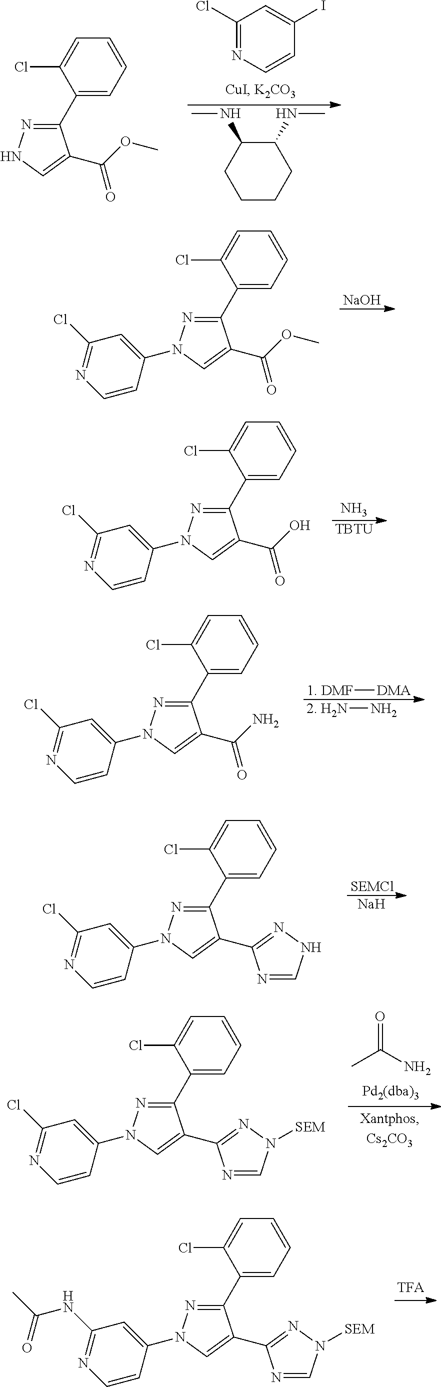

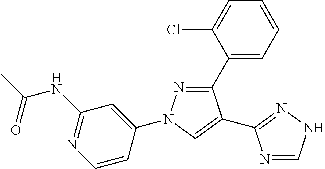 [SMILES: CC(=O)Nc1cc(-n2cc(-c3nc[nH]n3)c(-c3ccccc3Cl)n2)ccn1]

### Step 1: methyl 3-(2-chlorophenyl)-1-(2-chloropyridin-4-yl)-1H-pyrazole-4-carboxylate

A solution of 2-chloro-4-iodopyridine (10.0 g, 41.8 mmol), methyl 3-(2-chlorophenyl)-1H-pyrazole-4-carboxylate (1.65 g, 6.96 mmol), trans-1,2-bis(methylamino)cyclohexane (0.44 mL, 2.78 mmol), copper(I) iodide (0.073 g, 0.39 mmol), and potassium carbonate (5.77 g, 41.8 mmol) in 1,4-dioxane (56 mL) was allowed to stir at reflux for 3 h under an atmosphere of nitrogen. The reaction mixture was allowed to cool to rt, filtered through celite, washed with EtOAc, and concentrated. The residue was purified by column chromatography to give methyl 3-(2-chlorophenyl)-1-(2-chloropyridin-4-yl)-1H-pyrazole-4-carboxylate (1.74 g, 72%). LCMS (AA): m/z=348 (M+H).

### Step 2: 3-(2-chlorophenyl)-1-(2-chloropyridin-4-yl)-1H-pyrazole-4-carboxylic acid

To a solution of methyl 3-(2-chlorophenyl)-1-(2-chloropyridin-4-yl)-1H-pyrazole-4-carboxylate (0.97 g, 2.80 mmol) in THF (9 mL) was added aqueous sodium hydroxide (1M, 8.4 mL). The reaction mixture was allowed to stir at 80° C. overnight and then allowed to cool to rt. The THF was removed and the remaining solution was acidified with conc. HCl to pH=1. The mixture was extracted with EtOAc. The organic solution were combined, dried over Na₂SO₄, filtered and concentrated to give 3-(2-chlorophenyl)-1-(2-chloropyridin-4-yl)-1H-pyrazole-4-carboxylic acid (0.93 g, 99%), which was used in the next step without purification. LCMS (AA): m/z=334 (M+H).

### Step 3: 3-(2-chlorophenyl)-1-(2-chloropyridin-4-yl)-1H-pyrazole-4-carboxamide

To a solution of 3-(2-chlorophenyl)-1-(2-chloropyridin-4-yl)-1H-pyrazole-4-carboxylic acid (0.93 g, 2.8 mmol) in DCM (34 mL) were added TEA (3.0 mL, 21.2 mmol) and TBTU (3.57 g, 11.1 mmol). The reaction mixture was allowed to stir at rt for 15 min and then ammonia (0.5M in 1,4-dioxane, 30 mL) was added. The reaction mixture was allowed to stir at rt overnight and then diluted with water and extracted with DCM. The organic solutions were combined, dried over Na₂SO₄, filtered and concentrated. The residue was purified by column chromatography to give 3-(2-chlorophenyl)-1-(2-chloropyridin-4-yl)-1H-pyrazole-4-carboxamide (0.90 g, 97%). LCMS (FA): m/z=333 (M+H).

### Step 4: 2-chloro-4-[3-(2-chlorophenyl)-4-(1H-1,2,4-triazol-3-yl)-1H-pyrazol-1-yl]pyridine

To a suspension of 3-(2-chlorophenyl)-1-(2-chloropyridin-4-yl)-1H-pyrazole-4-carboxamide (1.50 g, 4.50 mmol) in anhydrous toluene (24 mL) was added DMF-DMA (1.77 mL, 13.3 mmol). The reaction mixture was allowed to stir at 50° C. for 2 h and then allowed to cool to rt and concentrated. The residue was dissolved in AcOH (17.7 mL) and hydrazine hydrate (1.08 mL, 22.1 mmol) was added. The reaction mixture was allowed to stir at rt for 3 h and then concentrated. The residue was azeotroped several times with toluene and then diluted with EtOAc and washed with saturated aqueous sodium bicarbonate. The organic solution was dried over Na₂SO₄, filtered and concentrated to give 2-chloro-4-[3-(2-chlorophenyl)-4-(1H-1,2,4-triazol-3-yl)-1H-pyrazol-1-yl]pyridine (1.46 g, 91%), which was used without purification in the next step. LCMS (FA): m/z=357 (M+H).

### Step 5: 2-chloro-4-[3-(2-chlorophenyl)-4-(1-{[2-(trimethylsilyl)ethoxy]methyl}-1H-1,2,4-triazol-3-yl)-1H-pyrazol-1-yl]pyridine

To a solution of 2-chloro-4-[3-(2-chlorophenyl)-4-(1H-1,2,4-triazol-3-yl)-1H-pyrazol-1-yl]pyridine (1.46 g, 4.09 mmol) in DMF (10 mL) was added NaH (60% in mineral oil, 0.245 g, 6.13 mmol) under an atmosphere of nitrogen at 0° C. The reaction mixture was allowed to stir at rt for 10 min and then cooled in an ice bath to 0° C. [β-(Trimethylsilyl)ethoxy]methyl chloride (1.33 mL, 7.53 mmol) in DMF (2.5 mL) was added and the reaction mixture was allowed to stir at 0° C. for 1.5 h. Water was added at 0° C. to quench the reaction and then the mixture was allowed to warm to rt. EtOAc was added and the aqueous solution was separated and further extracted with EtOAc. The organic solutions were combined, dried over Na₂SO₄, filtered and concentrated. The residue was purified by column chromatography to give the two separate isomers 2-chloro-4-[3-(2-chlorophenyl)-4-(1-{[2-(trimethylsilyl)ethoxy]methyl}-1H-1,2,4-triazol-3-yl)-1H-pyrazol-1-yl]pyridine and 2-chloro-4-[3-(2-chlorophenyl)-4-(4-{[2-(trimethylsilyl)ethoxy]methyl}-4H-1,2,4-triazol-3-yl)-1H-pyrazol-1-yl]pyridine (1.67 g total, 84%). LCMS (FA): m/z=333 (M+H).

### Step 6: N-{4-[3-(2-chlorophenyl)-4-(1-{[2-(trimethylsilyl)ethoxy]methyl}-1H-1,2,4-triazol-3-yl)-1H-pyrazol-1-yl]pyridin-2-yl}acetamide

To a solution of 2-chloro-4-[3-(2-chlorophenyl)-4-(1-{[2-(trimethylsilyl)ethoxy]methyl}-1H-1,2,4-triazol-3-yl)-1H-pyrazol-1-yl]pyridine (0.42 g, 0.85 mmol) in 1,4-dioxane (17 mL) were added acetamide (0.72 g, 12.2 mmol), tris(dibenzylideneacetone)dipalladium(0) (0.11 g, 0.12 mmol), Xantphos (0.21 g, 0.37 mmol) and cesium carbonate (1.19 g, 3.65 mmol). The reaction mixture was sealed in a vial and subjected to microwave irradiation at 130° C. for 60 min The reaction mixture was purified by column chromatography to give N-{4-[3-(2-chlorophenyl)-4-(1-{[2-(trimethylsilyl)ethoxy]methyl}-1H-1,2,4-triazol-3-yl)-1H-pyrazol-1-yl]pyridin-2-yl}acetamide (0.30 g, 70%). LCMS (AA): m/z=510 (M+H).

### Step 7: N-{4-[3-(2-chlorophenyl)-4-(1H-1,2,4-triazol-3-yl)-1H-pyrazol-1-yl]pyridin-2-yl}acetamide

To a solution of N-{4-[3-(2-chlorophenyl)-4-(1-{[2-(trimethylsilyl)ethoxy]methyl}-1H-1,2,4-triazol-3-yl)-1H-pyrazol-1-yl]pyridin-2-yl}acetamide (1.50 g, 2.9 mmol) in DCM (95 mL) was added TFA (28 mL). The reaction mixture was allowed to stir at rt overnight and then concentrated. The residue was purified by column chromatography to give N-{4-[3-(2-chlorophenyl)-4-(1H-1,2,4-triazol-3-yl)-1H-pyrazol-1-yl]pyridin-2-yl}acetamide (0.91 g, 82%). LCMS (AA): m/z=380 (M+H).

Compounds in the following table were prepared from the appropriate starting materials using the procedures described above:

| | |
|---|---|
| I-23 | LCMS (AA): m/z = 406 (M + H). |
| I-17 | LCMS (AA): m/z = 396 (M + H). |
| I-44 | LCMS (AA): m/z = 398 (M + H). |
| I-56 | LCMS (AA): m/z = 424 (M + H). |

### Example 4

### Synthesis of 1-(2-acetamidopyridin-4-yl)-3-(2-chlorophenyl)-1H-pyrazole-4-carboxamide (I-8)

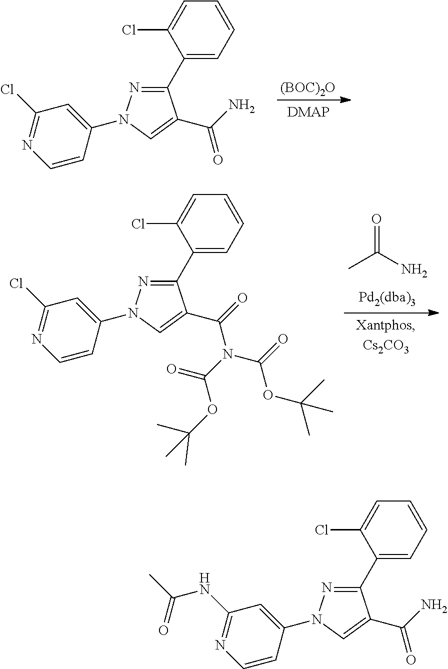

### Step 1: di-tert-butyl {[3-(2-chlorophenyl)-1-(2-chloropyridin-4-yl)-1H-pyrazol-4-yl]carbonyl}imidodicarbonate

To a solution of 3-(2-chlorophenyl)-1-(2-chloropyridin-4-yl)-1H-pyrazole-4-carboxamide (1.36 g, 4.08 mmol) in THF (27 mL) was added (BOC)₂O (4.01 g, 18.4 mmol) and DMAP (0.065 g, 0.531 mmol). The reaction mixture was allowed to stir at rt for 6 h and then diluted with EtOAc. The solution was washed with 1M HCl, saturated aqueous NaHCO₃ and brine, dried over Na₂SO₄, filtered and concentrated. The residue was purified by column chromatography to give di-tert-butyl {[3-(2-chlorophenyl)-1-(2-chloropyridin-4-yl)-1H-pyrazol-4-yl]carbonyl}imidodicarbonate (1.01 g, 46%). LCMS (FA): m/z=533 (M+H).

### Step 2: 1-(2-acetamidopyridin-4-yl)-3-(2-chlorophenyl)-1H-pyrazole-4-carboxamide

To a solution of di-tert-butyl {[3-(2-chlorophenyl)-1-(2-chloropyridin-4-yl)-1H-pyrazol-4-yl]carbonyl}imidodicarbonate (1.01 g, 1.89 mmol) in 1,4-dioxane (37 mL) were added acetamide (1.60 g, 27.0 mmol), tris(dibenzylideneacetone)dipalladium(0) (0.248 g, 0.270 mmol), Xantphos (0.470 g, 0.812 mmol) and cesium carbonate (2.64 g, 8.11 mmol). The reaction mixture was sealed in a vial and subjected to microwave irradiation at 130° C. for 60 min. The reaction mixture was filtered through celite, washed with EtOAc, and concentrated. The residue was purified by column chromatography to give 1-(2-acetamidopyridin-4-yl)-3-(2-chlorophenyl)-1H-pyrazole-4-carboxamide (0.070 g, 1%). LCMS (AA): m/z=356 (M+H).

Compounds in the following table were prepared from the appropriate starting materials using the procedures described above:

| | |
|---|---|
| I-13 | LCMS (AA): m/z = 390 (M + H). |

### Example 5

### Synthesis of 1-(2-acetamido-5-chloropyridin-4-yl)-3-(2-chlorophenyl)-1H-pyrazole-4-carboxamide (I-9)

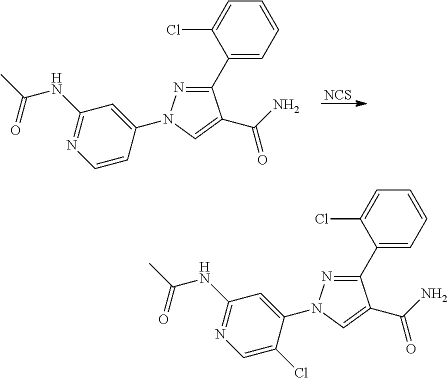 [SMILES: CC(=O)Nc1cc(-n2cc(C(N)=O)c(-c3ccccc3Cl)n2)ccn1 ; "NCS" ; CC(=O)Nc1cc(-n2cc(C(N)=O)c(-c3ccccc3Cl)n2)c(Cl)cn1]

To a solution of N-{4-[3-(2-chlorophenyl)-4-(1H-1,2,4-triazol-3-yl)-1H-pyrazol-1-yl]pyridin-2-yl}acetamide (0.031 g, 0.087 mmol) in ACN (0.2 mL) was added NCS (0.029 g, 0.218 mmol). The reaction was allowed to stir at 70° C. for 3.5 h and then allowed to cool to rt and concentrated. The residue was purified by column chromatography to give 1-(2-acetamido-5-chloropyridin-4-yl)-3-(2-chlorophenyl)-1H-pyrazole-4-carboxamide (0.010 g, 29%). LCMS (AA): m/z=390 (M+H).

Compounds in the following table were prepared from the appropriate starting materials using the procedures described above:

| | |
|---|---|
| I-4 | LCMS (AA): m/z = 538 (M + H). |
| I-18 | LCMS (AA): m/z = 460 (M + H). |

### Example 6

### Synthesis of N-{4-[3-(2-chlorophenyl)-4-(1H-1,2,4-triazol-3-yl)-1H-pyrazol-1-yl]-5-methylpyridin-2-yl}acetamide (I-5)

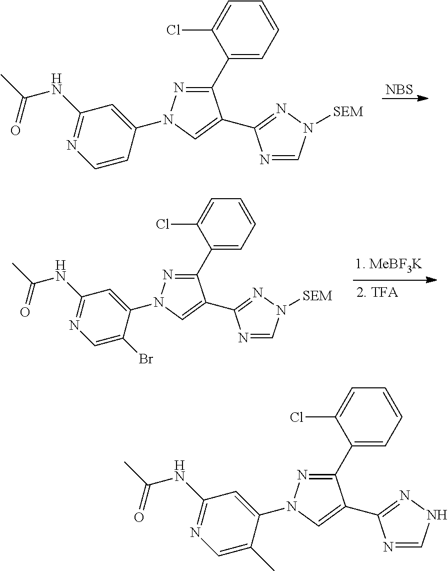

### Step 1: N-{5-bromo-4-[3-(2-chlorophenyl)-4-(1-{[2-(trimethylsilyl)ethoxy]methyl}-1H-1,2,4-triazol-3-yl)-1H-pyrazol-1-yl]pyridin-2-yl}acetamide

To a solution of N-{4-[3-(2-chlorophenyl)-4-(1-{[2-(trimethylsilyl)ethoxy]methyl}-1H-1,2,4-triazol-3-yl)-1H-pyrazol-1-yl]pyridin-2-yl}acetamide (0.345 g, 0.68 mmol) in DMF (7 mL) was added NBS (0.30 g, 1.7 mmol). The reaction mixture was allowed to stir at 85° C. for 1 h and then allowed to cool to rt and diluted with EtOAc. The mixture was washed with saturated aqueous NaHCO₃ and the organic solution was dried over Na₂SO₄, filtered and concentrated to give N-{5-bromo-4-[3-(2-chlorophenyl)-4-(1-{[2-(trimethylsilyl)ethoxy]methyl}-1H-1,2,4-triazol-3-yl)-1H-pyrazol-1-yl]pyridin-2-yl}acetamide (0.32 g, 81%), which was used without further purification. LCMS (AA): m/z=590 (M+H).

### Step 2: N-{4-[3-(2-chlorophenyl)-4-(1H-1,2,4-triazol-3-yl)-1H-pyrazol-1-yl]-5-methylpyridin-2-yl}acetamide

A solution of N-{5-bromo-4-[3-(2-chlorophenyl)-4-(1-{[2-(trimethylsilyl)ethoxy]methyl}-1H-1,2,4-triazol-3-yl)-1H-pyrazol-1-yl]pyridin-2-yl}acetamide (0.032 g, 0.54 mmol), potassium methyl trifluoroborate (0.33 g, 2.7 mmol), Pd(dppf)Cl₂ (0.067 g, 0.82 mmol) and sodium carbonate (1M in water, 1.1 mL) in DME (6.5 mL) was sealed in a vial and subjected to microwave irradiation at 150° C. for 2 h. The reaction mixture was diluted with EtOAc. The organic solution was separated, dried over Na₂SO₄, filtered and concentrated. The residue was redissolved in DCM (17 mL) and TFA (5.2 mL) was added. The reaction mixture was allowed to stir at rt overnight and then concentrated. The residue was purified by column chromatography to give N-{4-[3-(2-chlorophenyl)-4-(1H-1,2,4-triazol-3-yl)-1H-pyrazol-1-yl]-5-methylpyridin-2-yl}acetamide (0.034 g, 16%). LCMS (FA): m/z=394 (M+H).

### Example 7

### Alternative synthesis of N-{4-[3-(2-chlorophenyl)-4-(1H-1,2,4-triazol-3-yl)-1H-pyrazol-1-yl]-5-methylpyridin-2-yl}acetamide (I-5) and Synthesis of 1-(2-acetamido-5-methylpyridin-4-yl)-3-(2-chlorophenyl)-1H-pyrazole-4-carboxylic acid (I-6) and 1-(2-acetamido-5-methylpyridin-4-yl)-3-(2-chlorophenyl)-1H-pyrazole-4-carboxamide (I-11)

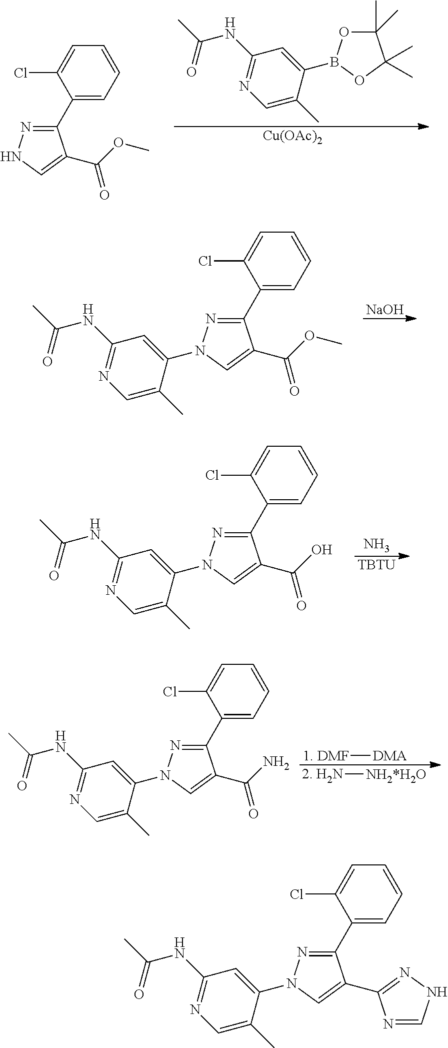

### Step 1: methyl 1-(2-acetamido-5-methylpyridin-4-yl)-3-(2-chlorophenyl)-1H-pyrazole-4-carboxylate

A mixture of methyl 3-(2-chlorophenyl)-1H-pyrazole-4-carboxylate (0.50 g, 2.11 mmol), N-[5-methyl-4-(4,4,5,5-tetramethyl-1,3,2-dioxaborolan-2-yl)pyridin-2-yl]acetamide (0.060 g, 0.22 mmol), cupric acetate (0.19 g, 0.11 mmol) and pyridine (0.34 mL, 4.23 mmol) in DMF (23 mL) was allowed to stir at 150° C. for 1 h. An additional 8 portions of N-[5-methyl-4-(4,4,5,5-tetramethyl-1,3,2-dioxaborolan-2-yl)pyridin-2-yl]acetamide (0.087 g, 0.32 mmol) were added evenly over the course of 5 h. The reaction mixture was allowed to stir for an additional 17 h and then concentrated. The residue was purified by column chromatography to give methyl 1-(2-acetamido-5-methylpyridin-4-yl)-3-(2-chlorophenyl)-1H-pyrazole-4-carboxylate (0.48 g, 60%). LCMS (FA): m/z=385 (M+H).

### Step 2: 1-(2-acetamido-5-methylpyridin-4-yl)-3-(2-chlorophenyl)-1H-pyrazole-4-carboxylic acid (I-6)

A mixture of methyl 1-(2-acetamido-5-methylpyridin-4-yl)-3-(2-chlorophenyl)-1H-pyrazole-4-carboxylate (0.34 g, 0.89 mmol) and sodium hydroxide (0.36 g, 8.9 mmol) in THF (17 mL) was allowed to stir at rt overnight and then concentrated. The residue was dissolved in water and the aqueous solution was washed with EtOAc. The aqueous solution was acidified with 1N HCl and then filtered. The solid was collected and dried to give 1-(2-acetamido-5-methylpyridin-4-yl)-3-(2-chlorophenyl)-1H-pyrazole-4-carboxylic acid (0.27 g, 82%). LCMS (FA): m/z=371 (M+H).

### Step 3: 1-(2-acetamido-5-methylpyridin-4-yl)-3-(2-chlorophenyl)-1H-pyrazole-4-carboxamide (I-11)

A mixture of 1-(2-acetamido-5-methylpyridin-4-yl)-3-(2-chlorophenyl)-1H-pyrazole-4-carboxylic acid (0.31 g, 0.84 mmol), TBTU (1.08 g, 3.36 mmol) and TEA (0.89 mL, 6.4 mmol) in DCM (10 mL) was allowed to stir at rt. To the reaction mixture was added ammonia (0.5 M in 1,4-dioxane, 8.9 mL). The reaction mixture was allowed to stir at rt overnight and then diluted with water and extracted with DCM. The organic solutions were combined, dried over MgSO₄, filtered and concentrated. The residue was purified by column chromatography to give 1-(2-acetamido-5-methylpyridin-4-yl)-3-(2-chlorophenyl)-1H-pyrazole-4-carboxamide (0.24 g, 77%). LCMS (FA): m/z=370 (M+H).

### Step 4: N-{4-[3-(2-chlorophenyl)-4-(1H-1,2,4-triazol-3-yl)-1H-pyrazol-1-yl]-5-methylpyridin-2-yl}acetamide (I-5)

A slurry of 1-(2-acetamido-5-methylpyridin-4-yl)-3-(2-chlorophenyl)-1H-pyrazole-4-carboxamide (0.24 g, 0.64 mmol) and DMF-DMA (0.25 mL, 1.91 mmol) in toluene (10 mL) was allowed to stir at 50° C. To this solution was added DMF (4 mL). The reaction mixture was allowed to stir overnight and then diluted with water and extracted with EtOAc. The organic solutions were combined, dried over MgSO₄, filtered and concentrated. The residue was dissolved in AcOH (5 mL) and hydrazine hydrate (0.15 mL, 3.2 mmol) was added. The reaction mixture was allowed to stir at rt for 2 h and was then concentrated. The residue was azeotroped several times with toluene and then dissolved in EtOAc. The organic solution was washed with aqueous saturated sodium bicarbonate, dried over MgSO₄, filtered and concentrated. The residue was purified by column chromatography to give N-{-4-[3-(2-chlorophenyl)-4-(1H-1,2,4-triazol-3-yl)-1H-pyrazol-1-yl]-5-methylpyridin-2-yl}acetamide (0.10 g, 40%). LCMS (FA): m/z=394 (M+H).

Compounds in the following table were prepared from the appropriate starting materials using the procedures described above:

| | |
|---|---|
| I-7 | LCMS (AA): m/z = 338 (M + H). |
| I-46 | LCMS (AA): m/z = 420 (M + H). |
| I-34 | LCMS (AA): m/z = 396 (M + H). |
| I-67 | LCMS (AA): m/z = 455 (M + H). |

### Example 8

### Synthesis of N-{4-[3-iodo-4-(1H-1,2,4-triazol-3-yl)-1H-pyrazol-1-yl]-5-methylpyridin-2-yl}acetamide (1-62) and N-{4-[3-(2-chloro-4-methoxyphenyl)-4-(1H-1,2,4-triazol-3-yl)-1H-pyrazol-1-yl]-5-methylpyridin-2-yl}acetamide (I-35)

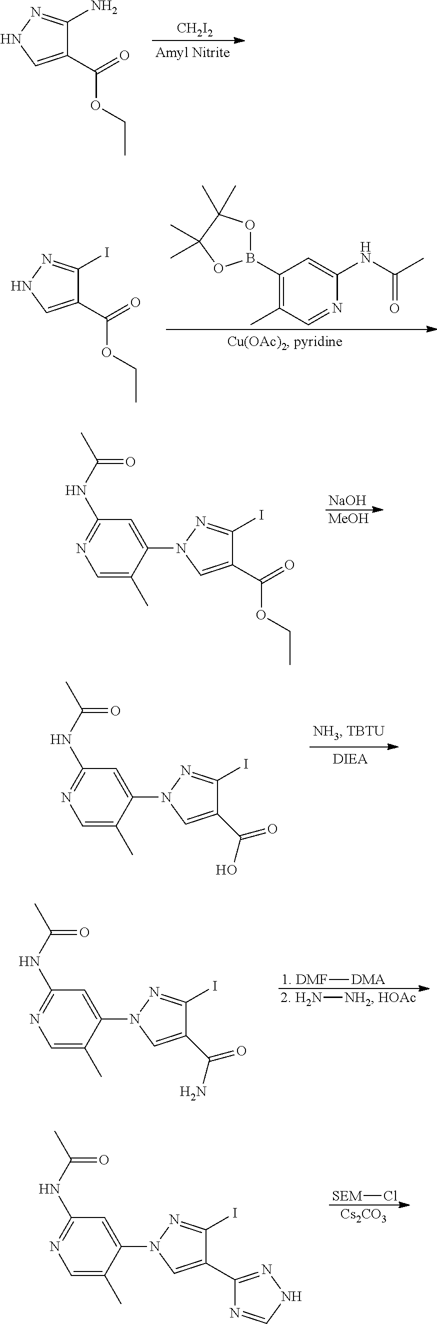

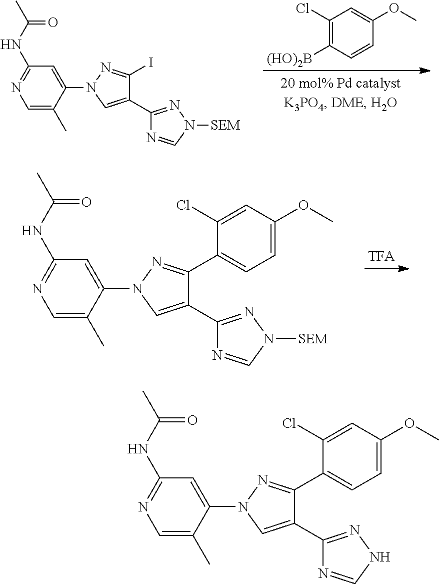 [SMILES: CC(=O)Nc1cc(-n2cc(-c3ncn(COCC[Si](C)(C)C)n3)c(I)n2)c(C)cn1 ; COc1ccc(B(O)O)c(Cl)c1 ; COc1ccc(-c2nn(-c3cc(NC(C)=O)ncc3C)cc2-c2ncn(COCC[Si](C)(C)C)n2)c(Cl)c1 ; COc1ccc(-c2nn(-c3cc(NC(C)=O)ncc3C)cc2-c2nc[nH]n2)c(Cl)c1]

### Step 1: ethyl 3-iodo-1H-pyrazole-4-carboxylate

To a suspension of ethyl 3-amino-1H-pyrazole-4-carboxylate (5 g, 0.032 mol) in diiodomethane (104 mL) was added amyl nitrite (38.8 mL, 2.75 mol) dropwise at −10° C. over a period of 30 min. The reaction mixture was allowed to warm to rt and then to heat and stir at 100° C. for 2 h. The reaction mixture was allowed to cool to room temperature and then concentrated. The residue was dissolved in EtOAc and washed with Na₂S₂O₃, 1M HCl, water and brine. The organic solution was dried over Na₂SO₄, filtered and concentrated. The crude product was prufied by column chromatography to give ethyl 3-iodo-1H-pyrazole-4-carboxylate (5.0 g, 56%).

### Step 2: ethyl 1-(2-acetamido-5-methylpyridin-4-yl)-3-iodo-1H-pyrazole-4-carboxylate

A mixture of ethyl 3-iodo-1H-pyrazole-4-carboxylate (2.0 g, 7.5 mmol), N-[5-methyl-4-(4,4,5,5-tetramethyl-1,3,2-dioxaborolan-2-yl)pyridin-2-yl]acetamide (3.0 g, 10.8 mmol), cupric acetate (2.0 g, 11.0 mmol) and pyridine (3 mL, 27.6 mmol) in THF (20 mL) was allowed to stir at reflux for 2 days. The reaction mixture was filtered and the solid was washed with EtOAc. The organic solutions were combined, washed with 10% aqueous ammonia and brine, dried over MgSO4, filtered and concentrated. The residue was purified by column chromatography to give ethyl 1-(2-acetamido-5-methylpyridin-4-yl)-3-iodo-1H-pyrazole-4-carboxylate (0.65 g, 21%).

### Step 3: 1-(2-acetamido-5-methylpyridin-4-yl)-3-iodo-1H-pyrazole-4-carboxylic acid

Ethyl 1-(2-acetamido-5-methylpyridin-4-yl)-3-iodo-1H-pyrazole-4-carboxylate (0.50 g, 1.2 mmol) was added to a mixture of THF (9 mL), MeOH (15 mL), and 1MNaOH (6.25 mL). The reaction mixture was allowed to stir at rt for 6 h and then 1M HCl (7.5 mL) was added. The mixture was concentrated to half volume and then filtered to give 1-(2-acetamido-5-methylpyridin-4-yl)-3-iodo-1H-pyrazole-4-carboxylic acid (0.42 g, 92%) which was used without purification in the next step.

### Step 4: 1-(2-acetamido-5-methylpyridin-4-yl)-3-iodo-1H-pyrazole-4-carboxamide

A mixture of 1-(2-acetamido-5-methylpyridin-4-yl)-3-iodo-1H-pyrazole-4-carboxylic acid (0.42 g, 1.1 mmol), TBTU (0.77 g, 2.4 mmol), DIEA (1.2 mL, 6.1 mmol) and ammonia (0.5M in 1,4-dioxane, 62.5 mL) in DCM (32 mL) was allowed to stir at rt overnight. The mixture was concentrated and water (22 mL) was added. The solid was filtered and purified by column chromatography to give 1-(2-acetamido-5-methylpyridin-4-yl)-3-iodo-1H-pyrazole-4-carboxamide (0.19 g, 45%).

### Step 5: N-{4-[3-iodo-4-(1H-1,2,4-triazol-3-yl)-1H-pyrazol-1-yl]-5-methylpyridin-2-yl}acetamide (I-62)

A suspension of 1-(2-acetamido-5-methylpyridin-4-yl)-3-iodo-1H-pyrazole-4-carboxamide (0.19 g, 0.50 mmol) in anhydrous toluene (5 mL) was sonicated for 15 min. To this suspension was added DMF-DMA (2.6 mL, 20.2 mmol). The reaction mixture was allowed to stir at 50° C. for 20 h. The reaction mixture was allowed to cool to rt and was concentrated. The residue was suspended in AcOH (6 mL) and hydrazine (0.07 mL, 2.4 mmol) was added dropwise. The reaction mixture was allowed to stir at 40° C. for 3 h. Water (10 mL) was added and the mixture was allowed to stir for 30 min at rt and then filtered. The solid was washed with water and dried to give N-{-4-[3-iodo-4-(1H-1,2,4-triazol-3-yl)-1H-pyrazol-1-yl]-5-methylpyridin-2-yl}acetamide (I-62) (0.08 g, 39%) which can be purified by column chromatography, but was is sufficiently pure to use in the next step without purification. LCMS (AA): m/z=410 (M+H).

### Step 6: N-{4-[3-iodo-4-(1-{[2-(trimethylsilyl)ethoxy)methyl}-1H-1,2,4-triazol-3-yl)-1H-pyrazol-1-yl]-5-methylpyridin-2-yl}acetamide

To a solution of N-{4-[3-iodo-4-(1H-1,2,4-triazol-3-yl)-1H-pyrazol-1-yl]-5-methylpyridin-2-yl}acetamide (0.50 g, 1.2 mmol) in DMF (9 mL) was added cesium carbonate (0.88 g, 2.7 mmol). The reaction mixture was allowed to stir at rt for 45 min and then [2-(chloromethoxy)ethyl](trimethyl)silane (0.48 mL, 2.7 mmol) was added. The reaction mixture was allowed to stir at rt for 90 min and was diluted with EtOAc. The solution was washed with brine and the organic solution was separated, dried over MgSO₄, filtered and concentrated. The residue was purified by column chromatography to give N-{4-[3-iodo-4-(1-{[2-(trimethylsilyl)ethoxy]methyl}-1H-1,2,4-triazol-3-yl)-1H-pyrazol-1-yl]-5-methylpyridin-2-yl}acetamide (0.15 g, 23%).

### Step 7: N-{4-[3-(2-chloro-4-methoxyphenyl)-4-(1H-1,2,4-triazol-3-yl)-1H-pyrazol-1-yl]-5-methylpyridin-2-yl}acetamide (I-35)

A mixture of N-{4-[3-iodo-4-(1-{[2-(trimethylsilyl)ethoxy]methyl}-1H-1,2,4-triazol-3-yl)-1H-pyrazol-1-yl]-5-methylpyridin-2-yl}acetamide (0.15 g, 0.29 mmol), (2-chloro-4-methoxyphenyl)boronic acid (0.11 g, 0.57 mmol), bis(di-tert-butyl(4-dimethylaminophenyl)phosphine)dichloropalladium(II) (0.040 g, 0.057 mmol) and potassium phosphate (0.18 g, 0.86 mmol) in DME (4 mL) and water (0.2 mL) was subjected to microwave irradiation for 30 min at 130° C. The reaction mixture was partitioned between EtOAc and saturated aqueous sodium bicarbonate. The organic solution was separated, dried over Na2SO4, filtered and concentrated to give N-{4-[3-(2-chloro-4-methoxyphenyl)-4-(1-{[2-(trimethylsilyl)ethoxy]methyl}-1H-1,2,4-triazol-3-yl)-1H-pyrazol-1-yl]-5-methylpyridin-2-yl}acetamide. The crude product was dissolved in DCM (10 mL) and TFA (1 mL) was added. The reaction mixture was allowed to stir at rt overnight and then diluted with toluene. The mixture was concentrated and the residue was redissolved in DCM. This solution was washed with saturated aqueous sodium bicarbonate, dried over MgSO₄, filtered and concentrated. The residue was purified by column chromatography to give N-{4-[3-(2-chloro-4-methoxyphenyl)-4-(1H-1,2,4-triazol-3-yl)-1H-pyrazol-1-yl]-5-methylpyridin-2-yl}acetamide (1-35) (0.054 g, 40%). LCMS (FA): m/z=424.4 (M+H).

Compounds in the following table were prepared from the appropriate starting materials using the procedures described above:

| | |
|---|---|
| I-52 | LCMS (FA): m/z = 385.4 (M + H). |
| I-48 | LCMS (FA): m/z = 428 (M + H). |
| I-39 | LCMS (AA): m/z = 428 (M + H). |
| I-49 | LCMS (AA): m/z = 390 (M + H). |
| I-57 | LCMS (AA): m/z = 388 (M + H). |
| I-66 | LCMS (AA): m/z = 462 (M + H). |
| I-65 | LCMS (AA): m/z = 412 (M + H). |
| I-59 | LCMS (AA): m/z = 460.2 (M + H). |
| I-69 | LCMS (AA): m/z = 374 (M + H). |
| I-68 | LCMS (AA): m/z = 438 (M + H). |
| I-106 | LCMS (FA): m/z = 395 (M + H). |

### Example 9

### Synthesis of N-{4-[3-(2-chlorophenyl)-4-(1H-1,2,4-triazol-3-yl)-1H-pyrrol-1-yl]-5-methylpyridin-2-yl}-2-methylpropanamide (I-25)

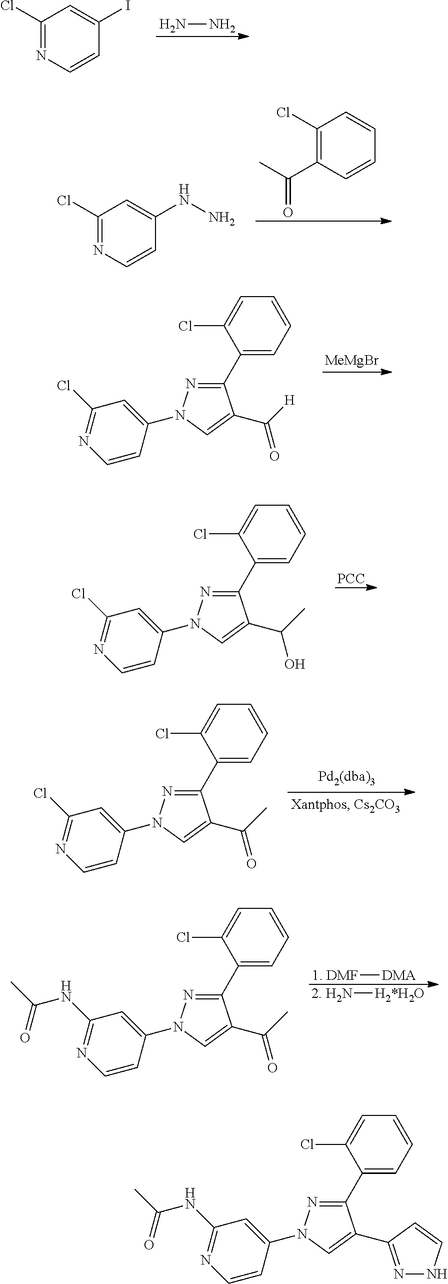

### Step 1: 2-chloro-4-hydrazinopyridine

To a solution of 2-chloro-4-iodopyridine (2.00 g, 8.35 mmol) in EtOH (38 mL) was added hydrazine hydrate (8 mL). The reaction mixture was allowed to stir at reflux overnight. After being allowed to cool to rt, the reaction mixture was poured into aqueous NaOH (1M, 100 mL) and was extracted several times with EtOAc. The organic solutions were combined, dried over Na₂SO₄, filtered and concentrated to give 2-chloro-4-hydrazinopyridine (1.16 g, 97%) which was used without purification in the next step. LCMS (AA): m/z=144 (M+H).

### Step 2: 3-(2-chlorophenyl)-1-(2-chloropyridin-4-yl)-1H-pyrazole-4-carbaldehyde

To a solution of 1-(2-chlorophenyl)ethanone (1.08 mL, 8.36 mmol) in AcOH (33 mL) was added 2-chloro-4-hydrazinopyridine (1.20 g, 8.36 mmol). The reaction mixture was allowed to stir at rt for 1 h and was then diluted with water. A white precipitate formed, and the mixture was extracted with EtOAc several times. The organic solutions were combined, washed with saturated aqueous NaHCO₃, dried over Na₂SO₄, filtered and concentrated. The residue was dissolved in DMF (15 mL) and added to a solution of phosphoryl chloride (1.56 mL, 16.7 mmol) in DMF (13 mL) which had been stirring at rt for 30 min. The reaction mixture was allowed to stir at 80° C. for 18 h and then at rt for 35 h. The mixture was diluted with water and extracted several times with DCM. The organic solutions were combined, dried over Na₂SO₄, filtered and concentrated. The residue was purified by column chromatography to give 3-(2-chlorophenyl)-1-(2-chloropyridin-4-yl)-1H-pyrazole-4-carbaldehyde (1.95 g, 73%). LCMS (AA): m/z=318 (M+H).

### Step 3: 1-[3-(2-chlorophenyl)-1-(2-chloropyridin-4-yl)-1H-pyrazol-4-yl]ethanol

A solution of 3-(2-chlorophenyl)-1-(2-chloropyridin-4-yl)-1H-pyrazole-4-carbaldehyde (0.50 g, 1.57 mmol) in THF (10 mL) was allowed to stir at 0° C. To this cooled solution was added methylmagnesium bromide (3.0 M in Et₂O, 0.66 mL, 1.96 mmol). The reaction mixture was allowed to stir at 0° C. for 1 h and was then quenched by the addition of 1M HCl. The solution was allowed to warm to rt and was then extracted several times with EtOAc. The organic solutions were combined, dried over Na₂SO₄, filtered and concentrated to give 1-[3-(2-chlorophenyl)-1-(2-chloropyridin-4-yl)-1H-pyrazol-4-yl]ethanol (0.24 g, 46%) which was used in the next step without purification. LCMS (AA): m/z=334 (M+H).

### Step 4: 1-[3-(2-chlorophenyl)-1-(2-chloropyridin-4-yl)-1H-pyrazol-4-yl]ethanone

To a solution of 1-[3-(2-chlorophenyl)-1-(2-chloropyridin-4-yl)-1H-pyrazol-4-yl]ethanol (024 g, 0.72 mmol) in DCM (5 mL) was added PCC (0.39 g, 1.80 mmol). The reaction mixture was allowed to stir at rt overnight and then diluted with Et₂O. The mixture was filtered through celite and the filtrate was concentrated. The residue was purified by column chromatography to give 1-[3-(2-chlorophenyl)-1-(2-chloropyridin-4-yl)-1H-pyrazol-4-yl]ethanone (0.086, 36%). LCMS (AA): m/z=332 (M+H).

### Step 5: N-{4-[4-acetyl-3-(2-chlorophenyl)-1H-pyrazol-1-yl]pyridin-2-yl}acetamide

A solution of 1-[3-(2-chlorophenyl)-1-(2-chloropyridin-4-yl)-1H-pyrazol-4-yl]ethanone (0.086 g, 0.26 mmol), acetamide (0.22 g, 3.70 mmol), tris(dibenzylideneacetone)dipalladium (0.034 g, 0.037 mmol), xantphos (0.064 g, 0.11 mmol) and cesium carbonate (0.36 g, 1.11 mmol) in 1,4-dioxane (4 mL) was sealed in a vial and subjected to microwave irradiation at 135° C. for 2 h. The reaction mixture was filtered through celite and washed with EtOAc. The filtrate was concentrated and the residue was purified by column chromatography to give N-{4-[4-acetyl-3-(2-chlorophenyl)-1H-pyrazol-1-yl]pyridin-2-yl}acetamide (0.091, 99%). LCMS (AA): m/z=355 (M+H).

### Step 6: N-{4-[3′-(2-chlorophenyl)-1H,1′H-3,4′-bipyrazol-1′-yl]pyridin-2-yl}acetamide (I-25)

To a solution of N-{4-[4-acetyl-3-(2-chlorophenyl)-1H-pyrazol-1-yl]pyridin-2-yl}acetamide (0.091 g, 0.26 mmol) in toluene (2.4 mL) was added DMF-DMA (0.17 mL, 1.28 mmol). The reaction mixture was allowed to stir at 100° C. for 6 h and then allowed to cool to rt and concentrated. The residue was redissolved in EtOH (1.6 mL) and to this solution was added hydrazine monohydrochloride (0.088 g, 1.28 mmol). The reaction mixture was allowed to stir at 60° C. overnight and was then allowed to cool to rt and concentrated. The residue was redissolved in DCM and water was added. The organic solution was separated and the aqueous solution was further extracted with DCM. The organic solutions were combined, dried over Na₂SO₄, filtered and concentrated. The residue was purified by column chromatography to give N-{4-[3′-(2-chlorophenyl)-1H,1′H-3,4′-bipyrazol-1′-yl]pyridin-2-yl}acetamide (I-25) (0.027 g, 28%). LCMS (AA): m/z=379 (M+H).

### Example 10

### Synthesis of 3-(2-chlorophenyl)-1-(2-{[2-(4-hydroxyphenyl)ethyl]amino}pyrimidin-4-yl)-1H-pyrazole-4-carboxamide (I-43)

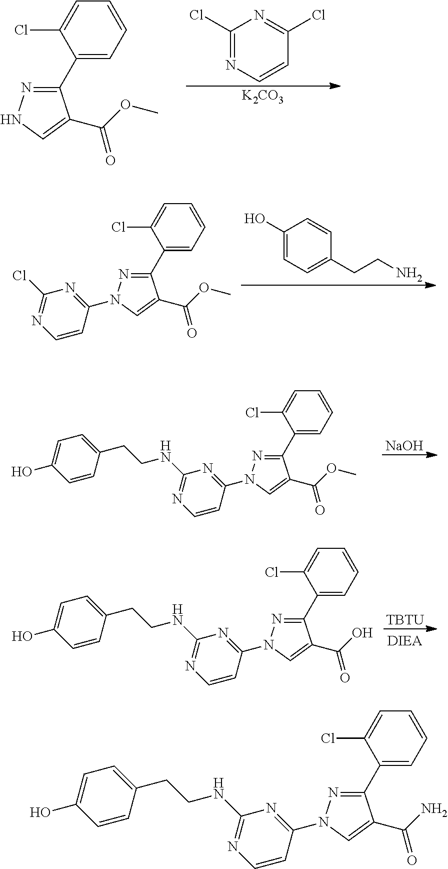

### Step 1: methyl 3-(2-chlorophenyl)-1-(2-chloropyrimidin-4-yl)-1H-pyrazole-4-carboxylate

To a slurry of potassium carbonate (0.95 g, 6.9 mmol) in ACN (65 mL) were added methyl 3-(2-chlorophenyl)-1H-pyrazole-4-carboxylate (0.65 g, 2.7 mmol) and 2,4-dichloropyrimidine (0.41 g, 2.7 mmol). The reaction mixture was allowed to stir at rt overnight and then concentrated. The residue was diluted with water and extracted with EtOAc. The organic solutions were combined, washed with water, dried over Na₂SO₄, filtered and concentrated. The residue was purified by column chromatography to give methyl 3-(2-chlorophenyl)-1-(2-chloropyrimidin-4-yl)-1H-pyrazole-4-carboxylate (0.50 g, 52%). LCMS (FA): m/z=349.4 (M+H).

### Step 2: methyl 3-(2-chlorophenyl)-1-(2-{[2-(4-hydroxyphenyl)ethyl]amino}pyrimidin-4-yl)-1H-pyrazole-4-carboxylate

A solution of 4-(2-aminoethyl)phenol (0.16 g, 1.15 mmol) and methyl 3-(2-chlorophenyl)-1-(2-chloropyrimidin-4-yl)-1H-pyrazole-4-carboxylate (0.18 g, 0.52 mmol) in ACN (5 mL) was allowed to stir at 80° C. overnight. The reaction mixture was allowed to cool to rt and was then diluted with DCM. The solution was washed several times with water and then concentrated to give methyl 3-(2-chlorophenyl)-1-(2-{[2-(4-hydroxyphenyl)ethyl]amino}pyrimidin-4-yl)-1H-pyrazole-4-carboxylate (0.24 g, 100%)). LCMS (FA): m/z=450.5 (M+H).

### Step 3: 3-(2-chlorophenyl)-1-(2-{[2-(4-hydroxyphenyl)ethyl]amino}pyrimidin-4-yl)-1H-pyrazole-4-carboxylic acid

A solution of methyl 3-(2-chlorophenyl)-1-(2-{[2-(4-hydroxyphenyl)ethyl]amino}pyrimidin-4-yl)-1H-pyrazole-4-carboxylate (0.24 g, 0.53 mmol) and sodium hydroxide (0.47 g, 1.17 mmol) in THF (0.7 mL and water (0.1 mL) was allowed to stir at rt overnight. The reaction mixture was concentrated to give 3-(2-chlorophenyl)-1-(2-{[2-(4-hydroxyphenyl)ethyl]amino}pyrimidin-4-yl)-1H-pyrazole-4-carboxylic acid (0.23 g, 99%) which was used in the next step without purification. LCMS (FA): m/z=436.6 (M+H).

### Step 4: 3-(2-chlorophenyl)-1-(2-{[2-(4-hydroxyphenyl)ethyl]amino}pyrimidin-4-yl)-1H-pyrazole-4-carboxamide (I-43)

To a solution of 3-(2-chlorophenyl)-1-(2-{[2-(4-hydroxyphenyl)ethyl]amino}pyrimidin-4-yl)-1H-pyrazole-4-carboxylic acid (0.25 g, 0.57 mmol) in DCM (20 mL) were added ammonia (0.5 M in 1,4-dioxane, 9.2 mL, 4.6 mmol), TBTU (0.37 g, 1.15 mmol) and DIEA (1.0 mL, 5.7 mmol). The reaction mixture was allowed to stir at rt overnight and then at 40° C. for 1 h. The reaction mixture was diluted with water and the pH was adjusted to 5.5-6 by the addition of 1N HCl. The solution was extracted with DCM. The organic solutions were combined, washed with water dried over Na₂SO₄, filtered and concentrated. The residue was purified by column chromatography to give 3-(2-chlorophenyl)-1-(2-{[2-(4-hydroxyphenyl)ethyl]amino}pyrimidin-4-yl)-1H-pyrazole-4-carboxamide (1-43) (0.13 g, 52%). LCMS (FA): m/z=435.5 (M+H).

Compounds in the following table were prepared from the appropriate starting materials using the procedures described above:

| | |
|---|---|
| I-30 | LCMS (FA): m/z = 420.4 (M + H). |
| I-29 | LCMS (FA): m/z = 436.5 (M + H). |

### Example 11

### Synthesis of N-{4-[3-(2-chlorophenyl)-4-(1H-imidazol-2-yl)-1H-pyrazol-1-yl]pyridin-2-yl}acetamide (I-32)

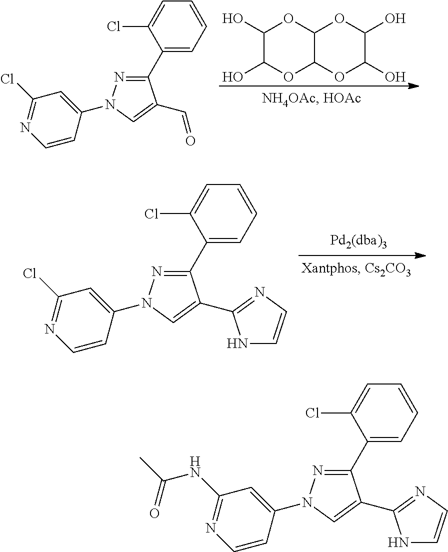

### Step 1: 2-chloro-4-[3-(2-chlorophenyl)-4-(1H-imidazol-2-yl)-1H-pyrazol-1-yl]pyridine

To a solution of 3-(2-chlorophenyl)-1-(2-chloropyridin-4-yl)-1H-pyrazole-4-carbaldehyde (0.50 g, 1.57 mmol) in MeOH (5.4 mL) was added hexahydro[1,4]dioxino[2,3-b][1,4]dioxine-2,3,6,7-tetrol (0.66 g, 3.14 mmol), ammonium acetate (0.73 g, 9.4 mmol) and AcOH (0.9 mL). The reaction mixture was allowed to stir at rt for 24 h and then carefully quenched with aqueous sodium bicarbonate solution. The mixture was extracted with EtOAc. The organic solutions were combined, dried over Na₂SO₄, filtered and concentrated. The residue was purified by column chromatography to give 2-chloro-4-[3-(2-chlorophenyl)-4-(1H-imidazol-2-yl)-1H-pyrazol-1-yl]pyridine (0.13 g, 24%). LCMS (AA): m/z=354 (M+H).

### Step 2: N-{4-[3-(2-chlorophenyl)-4-(1H-imidazol-2-yl)-1H-pyrazol-1-yl]pyridin-2-yl}acetamide (I-32)

A mixture of 2-chloro-4-[3-(2-chlorophenyl)-4-(1H-imidazol-2-yl)-1H-pyrazol-1-yl]pyridine (0.13 g, 0.37 mmol), acetamide (0.32 g, 5.33 mmol), tris(dibenzylideneacetone)dipalladium (0.049 g, 0.053 mmol), xantphos (0.093 g, 0.16 mmol) and cesium carbonate (0.52 g, 1.60 mmol) in 1,4-dioxane (7.3 mL) was sealed in a vial and subjected to microwave irradiation at 135° C. for 2 h. The reaction mixture was filtered through celite and washed with EtOAc. The filtrate was concentrated and the residue was redissolved in EtOAc and washed with water. The organic solutions were combined, dried over Na₂SO₄, filtered and concentrated. The residue was purified by column chromatography to give N-{4-[3-(2-chlorophenyl)-4-(1H-imidazol-2-yl)-1H-pyrazol-1-yl]pyridin-2-yl}acetamide (1-32) (0.065 g, 46%). LCMS (FA): m/z=379 (M+H).

Compounds in the following table were prepared from the appropriate starting materials using the procedures described above:

| | |
|---|---|
| I-109 | LCMS (AA): m/z = 408 (M + H). |

### Example 12

### Synthesis of N-{4-[3-{2-chloro-5-[(dimethylamino)methyl]phenyl}-4-(1H-1,2,4-triazol-3-yl)-1H-pyrazol-1-yl]-5-methylpyridin-2-yl}acetamide (I-42)

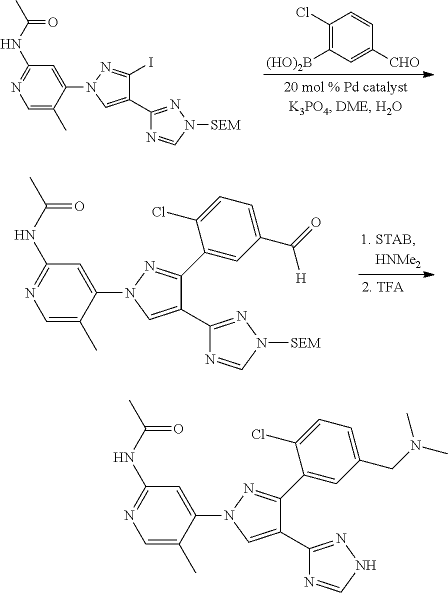 [SMILES: CC(=O)Nc1cc(-n2cc(-c3ncn(COCC[Si](C)(C)C)n3)c(I)n2)c(C)cn1 ; O=Cc1ccc(Cl)c(B(O)O)c1 ; CC(=O)Nc1cc(-n2cc(-c3ncn(COCC[Si](C)(C)C)n3)c(-c3cc(C=O)ccc3Cl)n2)c(C)cn1 ; CC(=O)Nc1cc(-n2cc(-c3nc[nH]n3)c(-c3cc(CN(C)C)ccc3Cl)n2)c(C)cn1]

### Step 1: N-{4-[3-(2-chloro-5-formylphenyl)-4-(1-{[2-(trimethylsilyl)ethoxy]methyl}-1H-1,2,4-triazol-3-yl)-1H-pyrazol-1-yl]-5-methylpyridin-2-yl}acetamide

A mixture of N-{4-[3-iodo-4-(1-{[2-(trimethylsilyl)ethoxy]methyl}-1H-1,2,4-triazol-3-yl)-1H-pyrazol-1-yl]-5-methylpyridin-2-yl}acetamide (0.30 g, 0.56 mmol), (2-chloro-5-formylphenyl)boronic acid (0.21 g, 1.11 mmol), bis(di-tert-butyl(4-dimethylaminophenyl)phosphine) dichloropalladium(II) (0.079 g, 0.11 mmol) and potassium phosphate (0.35 g, 1.67 mmol) in DME (8 mL) and water (0.4 mL) was allowed to stir at 110° C. in a sealed vessel for 45 min and was then diluted with aqueous sodium bicarbonate and extracted with EtOAc. The organic solutions were combined, dried over Na₂SO₄, filtered and concentrated. The residue was purified by column chromatography to give N-{4-[3-(2-chloro-5-formylphenyl)-4-(1-{[2-(trimethylsilyl)ethoxy]methyl}-1H-1,2,4-triazol-3-yl)-1H-pyrazol-1-yl]-5-methylpyridin-2-yl}acetamide (0.25 g, 81%). LCMS (AA): m/z=552 (M+H).

### Step 2: N-{4-[3-{2-chloro-5-[(dimethylamino)methyl]phenyl}-4-(1H-1,2,4-triazol-3-yl)-1H-pyrazol-1-yl]-5-methylpyridin-2-yl}acetamide

To a solution of N-{4-[3-(2-chloro-5-formylphenyl)-4-(1-{[2-(trimethylsilyl)ethoxy]methyl}-1H-1,2,4-triazol-3-yl)-1H-pyrazol-1-yl]-5-methylpyridin-2-yl}acetamide (0.25 g, 0.45 mmol) in DCM (7 mL) was added dimethyl amine (2.0 M in THF, 1.13 mL, 2.26 mmol) and sodium triacetoxyborohydride (0.29 g, 1.36 mmol). The reaction mixture was allowed to stir at rt for 7 h and then diluted with water and extracted with DCM. The organic solutions were combined, dried over Na₂SO₄, filtered and concentrated. The residue was dissolved in DCM (14.5 mL) and TFA (4.4 mL) was added. The reaction mixture was allowed to stir at rt overnight and then concentrated. The residue was dissolved in DCM, washed with aqueous saturated sodium bicarbonate solution, dried over Na₂SO₄, filtered and concentrated. The residue was purified by column chromatography to give N-{4-[3-{2-chloro-5-[(dimethylamino)methyl]phenyl}-4-(1H-1,2,4-triazol-3-yl)-1H-pyrazol-1-yl]-5-methylpyridin-2-yl}acetamide (I-42) (0.090 g, 44%). LCMS (AA): m/z=451 (M+H).

Compounds in the following table were prepared from the appropriate starting materials using the procedures described above:

| | |
|---|---|
| I-58 | LCMS (AA): m/z = 479 (M + H). |

### Example 13

### Synthesis of methyl {4-[3-(2-chlorophenyl)-4-(4H-1,2,4-triazol-3-yl)-1H-pyrazol-1-yl]-5-methylpyridin-2-yl}carbamate (I-45)

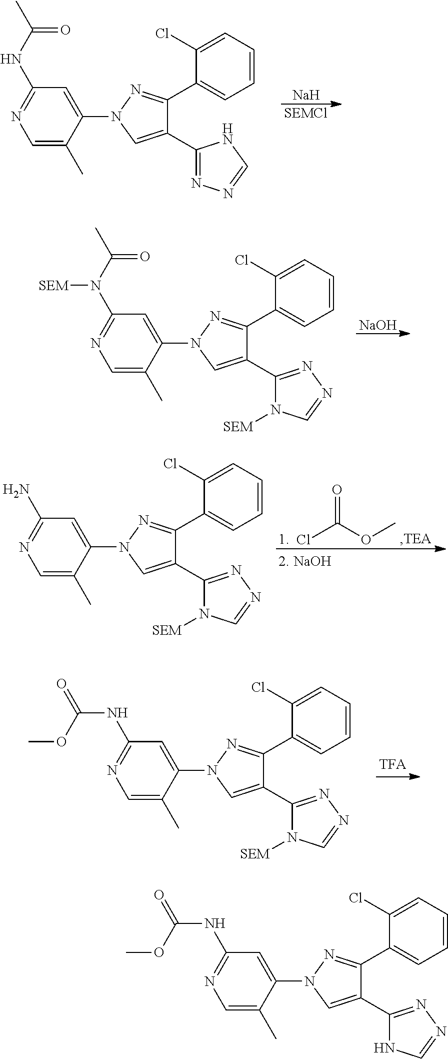

### Step 1: N-{4-[3-(2-chlorophenyl)-4-(4-{[2-(trimethylsilyl)ethoxy]methyl}-4H-1,2,4-triazol-3-yl)-1H-pyrazol-1-yl]-5-methylpyridin-2-yl}-N-{[2-(trimethylsilyl)ethoxy]methyl}acetamide

A solution of N-{4-[3-(2-chlorophenyl)-4-(4H-1,2,4-triazol-3-yl)-1H-pyrazol-1-yl]-5-methylpyridin-2-yl}acetamide (0.84 g, 2.13 mmol) in DMF (5.3 mL) was allowed to stir at 0° C. under an atmosphere of nitrogen. To this stirred solution was added NaH (60% in mineral oil, 0.13 g). The mixture was allowed to stir at rt for 5 min and then cooled to 0° C. To the cold solution was added [-(trimethylsilyl)ethoxy]methyl chloride (0.694 mL, 3.92 mmol) in DMF (1.3 mL). The reaction mixture was allowed to stir for 24 h and then quenched at 0° C. by the addition of water. The mixture was allowed to warm to rt and was then extracted with EtOAc. The organic solutions were combined, dried over Na₂SO₄, filtered and concentrated. The residue was purified by column chromatography to give N-{4-[3-(2-chlorophenyl)-4-(4-{[2-(trimethylsilyl)ethoxy]methyl}-4H-1,2,4-triazol-3-yl)-1H-pyrazol-1-yl]-5-methylpyridin-2-yl}-N-{[2-(trimethylsilyl)ethoxy]methyl}acetamide (0.23 g, 17%)). LCMS (FA): m/z=655 (M+H).

### Step 2: 4-[3-(2-chlorophenyl)-4-(4-{[2-(trimethylsilyl)ethoxy]methyl}-4H-1,2,4-triazol-3-yl)-1H-pyrazol-1-yl]-5-methylpyridin-2-amine

To a solution of N-{4-[3-(2-chlorophenyl)-4-(4-{[2-(trimethylsilyl)ethoxy]methyl}-4H-1,2,4-triazol-3-yl)-1H-pyrazol-1-yl]-5-methylpyridin-2-yl}-N-{[2-(trimethylsilyl)ethoxy]methyl}acetamide (0.23 g, 0.36 mmol) in EtOH (20 mL) was added sodium hydroxide (1M in water, 1.8 mL, 1.8 mmol). The reaction mixture was allowed to stir at reflux for 2 h and then allowed to cool to rt. The reaction mixture was concentrated and the residue was purified by column chromatography to give 4-[3-(2-chlorophenyl)-4-(4-{[2-(trimethylsilyl)ethoxy]methyl}-4H-1,2,4-triazol-3-yl)-1H-pyrazol-1-yl]-5-methylpyridin-2-amine (0.14 g, 82%). LCMS (FA): m/z=483 (M+H).

### Step 3: methyl {4-[3-(2-chlorophenyl)-4-(4-{[2-(trimethylsilyl)ethoxy)methyl}-4H-1,2,4-triazol-3-yl)-1H-pyrazol-1-yl]-5-methylpyridin-2-yl}carbamate

To a solution of 4-[3-(2-chlorophenyl)-4-(4-{[2-(trimethylsilyl)ethoxy]methyl}-4H-1,2,4-triazol-3-yl)-1H-pyrazol-1-yl]-5-methylpyridin-2-amine (0.14 g, 0.29 mmol) in DCM (4 mL) were added TEA (0.12 mL, 0.87 mmol) and methyl carbonochloridate (0.067 mL, 0.87 mmol). The reaction mixture was allowed to stir at rt for 6 h and was then concentrated. The residue was dissolved in MeOH (10 mL) and sodium hydroxide (1M in water, 2.0 mL, 2.0 mmol) was added. The reaction mixture was allowed to stir at rt for 1 h and was then diluted with water. The mixture was extracted with EtOAc and the organic solutions were combined, dried over Na₂SO₄, filtered and concentrated. The residue was purified by column chromatography to give methyl {4-[3-(2-chlorophenyl)-4-(4-{[2-(trimethylsilyl)ethoxy]methyl}-4H-1,2,4-triazol-3-yl)-1H-pyrazol-1-yl]-5-methylpyridin-2-yl}carbamate (0.082 g, 52%). LCMS (FA): m/z=540 (M+H).

### Step 4: methyl {4-[3-(2-chlorophenyl)-4-(4H-1,2,4-triazol-3-yl)-1H-pyrazol-1-yl]-5-methylpyridin-2-yl}carbamate (I-45)

To a solution of methyl {4-[3-(2-chlorophenyl)-4-(4-{[2-(trimethylsilyl)ethoxy]methyl}-4H-1,2,4-triazol-3-yl)-1H-pyrazol-1-yl]-5-methylpyridin-2-yl}carbamate (0.080 g, 0.15 mmol) in DCM (5 mL) was added TFA (1.4 mL). The reaction mixture was allowed to stir at rt overnight and then concentrated. The residue was purified by column chromatography to give methyl {4-[3-(2-chlorophenyl)-4-(4H-1,2,4-triazol-3-yl)-1H-pyrazol-1-yl]-5-methylpyridin-2-yl}carbamate (1-45) (0.025 g, 41%). LCMS (AA): m/z=410 (M+H).

Compounds in the following table were prepared from the appropriate starting materials using the procedures described above:

| | |
|---|---|
| I-108 | LCMS (FA): m/z = 352.2 (M + H). |

### Example 14

### Synthesis of N-{4-[3-(2-chlorophenyl)-4-(1H-imidazol-2-yl)-1H-pyrazol-1-yl]-5-methylpyridin-2-yl}acetamide (I-27)

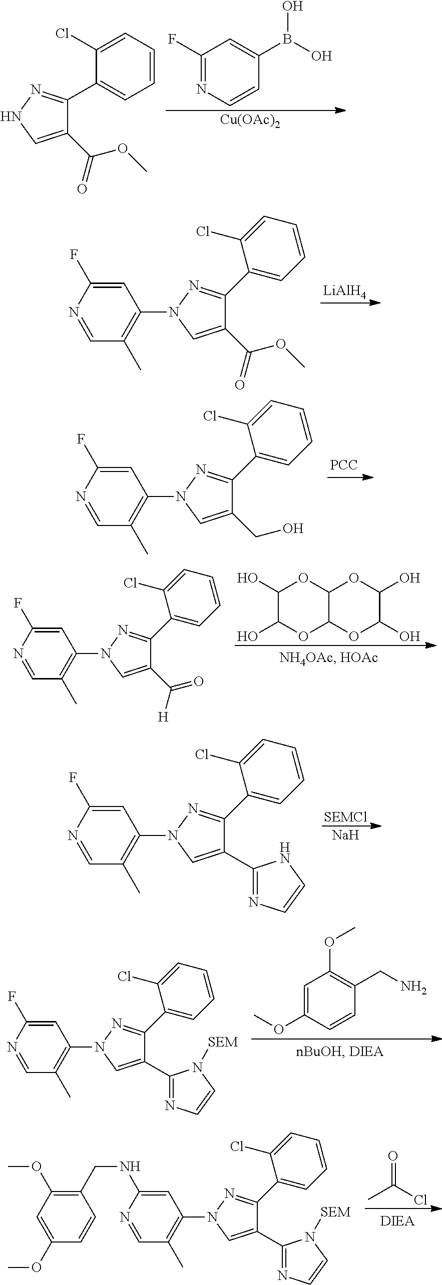

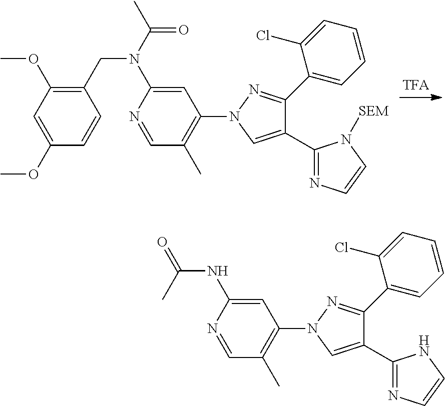 [SMILES: COc1ccc(CN(C(C)=O)c2cc(-n3cc(-c4nccn4COCC[Si](C)(C)C)c(-c4ccccc4Cl)n3)c(C)cn2)c(OC)c1 ; CC(=O)Nc1cc(-n2cc(-c3ncc[nH]3)c(-c3ccccc3Cl)n2)c(C)cn1]

### Step 1: methyl 3-(2-chlorophenyl)-1-(2-fluoro-5-methylpyridin-4-yl)-1H-pyrazole-4-carboxylate

A heterogeneous mixture of methyl 3-(2-chlorophenyl)-1H-pyrazole-4-carboxylate (0.50 g, 2.00 mmol), cupric acetate (0.58 g, 3.17 mmol), (2-fluoro-5-methylpyridin-4-yl)boronic acid (0.75 g, 4.80 mmol), activated 4A molecular sieves (125 mg) and pyridine (0.34 mL, 4.23 mmol) in DCM (50 mL) was allowed to stir at rt for 2 days. Additional (2-fluoro-5-methylpyridin-4-yl)boronic acid (0.18 g, 1.16 mmol) was added. The reaction mixture was allowed to stir for an additional 24 h and was then filtered. The solid was washed with MeOH and the filtrates were concentrated. The residue was purified by column chromatography to give methyl 3-(2-chlorophenyl)-1-(2-fluoro-5-methylpyridin-4-yl)-1H-pyrazole-4-carboxylate (0.33, 40%). LCMS (FA): m/z=346.4 (M+H).

### Step 2: [3-(2-chlorophenyl)-1-(2-fluoro-5-methylpyridin-4-yl)-1H-pyrazol-4-yl]methanol

A solution of methyl 3-(2-chlorophenyl)-1-(2-fluoro-5-methylpyridin-4-yl)-1H-pyrazole-4-carboxylate (0.063 g, 0.18 mmol) in THF (1 mL) was allowed to stir at 0° C. To this solution was added lithium tetrahydroaluminate (2.0 M in THF, 0.18 mL, 0.36 mmol) dropwise. The reaction mixture was allowed to stir at 0° C. for 1 h and was then quenched at this temperature by the slow addition of sodium sulfate decahydrate (1.0 g). The mixture was allowed to stir for 2 h and was then diluted with EtOAc and filtered. The solid was washed with EtOAc and then filtrate was concentrated to give [3-(2-chlorophenyl)-1-(2-fluoro-5-methylpyridin-4-yl)-1H-pyrazol-4-yl]methanol (0.063, 100%) which was used without purification in the next step. LCMS (FA): m/z=318.2 (M+H).

### Step 3: 3-(2-chlorophenyl)-1-(2-fluoro-5-methylpyridin-4-yl)-1H-pyrazole-4-carbaldehyde

To a solution of [3-(2-chlorophenyl)-1-(2-fluoro-5-methylpyridin-4-yl)-1H-pyrazol-4-yl]methanol (0.25 g, 0.79 mmol) in DCM (8 mL) was added PCC (0.20 g, 0.94 mmol). The reaction mixture was allowed to stir at rt for 2 h and then filtered through celite. The filter cake was washed with DCM and the filtrate was concentrated. The residue was purified by column chromatography to give 3-(2-chlorophenyl)-1-(2-fluoro-5-methylpyridin-4-yl)-1H-pyrazole-4-carbaldehyde (0.24, 95%). LCMS (FA): m/z=316.2 (M+H).

### Step 4: 4-[3-(2-chlorophenyl)-4-(1H-imidazol-2-yl)-1H-pyrazol-1-yl]-2-fluoro-5-methylpyridine

A slurry of 3-(2-chlorophenyl)-1-(2-fluoro-5-methylpyridin-4-yl)-1H-pyrazole-4-carbaldehyde (0.23 g, 0.73 mmol) in MeOH (2.5 mL) was allowed to stir at rt for 10 min. To this suspension was added AcOH (0.5 mL), hexahydro[1,4]dioxino[2,3-b][1,4]dioxine-2,3,6,7-tetrol (0.46 g, 2.20 mmol) and ammonium acetate (0.34 g, 4.39 mmol). The reaction mixture was allowed to stir at rt for 24 h and was then diluted with aqueous saturated sodium bicarbonate solution. The mixture was allowed to stir for 15 min and was then filtered. The solid was suspended in MeOH and filtered again. The filtrates were combined and concentrated. The residue was purified by column chromatography to give 4-[3-(2-chlorophenyl)-4-(1H-imidazol-2-yl)-1H-pyrazol-1-yl]-2-fluoro-5-methylpyridine (0.20, 73%). LCMS (FA): m/z=354.3 (M+H).

### Step 5: 4-[3-(2-chlorophenyl)-4-(1-{[2-(trimethylsilyl)ethoxy]methyl}-1H-imidazol-2-yl)-1H-pyrazol-1-yl]-2-fluoro-5-methylpyridine

To a solution of 4-[3-(2-chlorophenyl)-4-(1H-imidazol-2-yl)-1H-pyrazol-1-yl]-2-fluoro-5-methylpyridine (0.19 g, 0.53 mmol) in DMF (2 mL) under an atmosphere of argon was added sodium hydride (0.023 g, 0.59 mmol). The reaction mixture was allowed to stir at rt for 2 h and then [2-(chloromethoxy)ethyl]-(trimethyl)silane (0.10 mL, 0.59 mmol) was added dropwise. The reaction mixture was allowed to stir for 1 h at rt and was then diluted with water. The mixture was extracted with EtOAc. The organic solutions were combined, washed with brine, dried over Na₂SO₄, filtered and concentrated. The residue was purified by column chromatography to give 4-[3-(2-chlorophenyl)-4-(1-{[2-(trimethylsilyl)ethoxy]methyl}-1H-imidazol-2-yl)-1H-pyrazol-1-yl]-2-fluoro-5-methylpyridine (0.19, 73%). LCMS (FA): m/z=484.5 (M+H).

### Step 6: 4-[3-(2-chlorophenyl)-4-(1-{[2-(trimethylsilyl)ethoxy]methyl}-1H-imidazol-2-yl)-1H-pyrazol-1-yl]-N-(2,4-dimethoxybenzyl)-5-methylpyridin-2-amine

A mixture of 4-[3-(2-chlorophenyl)-4-(1-{[2-(trimethylsilyl)ethoxy]methyl}-1H-imidazol-2-yl)-1H-pyrazol-1-yl]-2-fluoro-5-methylpyridine (0.22 g, 0.46 mmol), 1-(2,4-dimethoxyphenyl)methanamine (0.69 mL, 4.61 mmol) and DIEA (0.24 mL, 1.38 mmol) in 1-butanol (7 mL) was sonicated in a sealed vial and then subjected to microwave irradiation at 165° C. for 4.5 h. The reaction mixture was concentrated and the residue was purified by column chromatography to give 4-[3-(2-chlorophenyl)-4-(1-{[2-(trimethylsilyl)ethoxy]methyl}-1H-imidazol-2-yl)-1H-pyrazol-1-yl]-N-(2,4-dimethoxybenzyl)-5-methylpyridin-2-amine (0.22, 75%). LCMS (FA): m/z=631.6 (M+H).

### Step 7: N-{4-[3-(2-chlorophenyl)-4-(1-{[2-(trimethylsilyl)ethoxy]methyl}-1H-imidazol-2-yl)-1H-pyrazol-1-yl]-5-methylpyridin-2-yl}-N-(2,4-dimethoxybenzyl)acetamide

To a solution of 4-[3-(2-chlorophenyl)-4-(1-{[2-(trimethylsilyl)ethoxy]methyl}-1H-imidazol-2-yl)-1H-pyrazol-1-yl]-N-(2,4-dimethoxybenzyl)-5-methylpyridin-2-amine (0.10 g, 0.17 mmol) in DCM (2.4 mL) was added DIEA (0.057 mL, 0.33 mmol). The reaction mixture was allowed to stir at 0° C. and then acetyl chloride (0.014 mL, 0.20 mmol) was added. The reaction mixture was allowed to stir at 0° C. for 1 h and was then diluted with EtOAc and water. The aqueous solution was separated and further extracted with EtOAc. The organic solutions were combined, washed with water and brine, dried over Na₂SO₄, filtered and concentrated. The residue was dissolved in MeOH (5 mL) and NaOH (1M in water, 1 mL) was added. The mixture was allowed to stir for 1 h and was then diluted with water. The mixture was extracted with EtOAc. The organic solutions were combined, washed with water, dried over Na₂SO₄, filtered and concentrated to give N-{4-[3-(2-chlorophenyl)-4-(1-{[2-(trimethylsilyl)ethoxy]methyl}-1H-imidazol-2-yl)-1H-pyrazol-1-yl]-5-methylpyridin-2-yl}-N-(2,4-dimethoxybenzyl)acetamide (0.090, 81%) which was used in the next step without purification. LCMS (FA): m/z=673.6 (M+H).

### Step 8: N-{4-[3-(2-chlorophenyl)-4-(1H-imidazol-2-yl)-1H-pyrazol-1-yl]-5-methylpyridin-2-yl}acetamide (I-27)

To a solution of N-{4-[3-(2-chlorophenyl)-4-(1-{[2-(trimethylsilyl)ethoxy]methyl}-1H-imidazol-2-yl)-1H-pyrazol-1-yl]-5-methylpyridin-2-yl}-N-(2,4-dimethoxybenzyl)acetamide (0.090 g, 0.10 mmol) in DCM (2 mL) was added TFA (0.40 mL, 5.18 mmol). The reaction mixture was allowed to stir at rt for 3 h and then additional TFA (0.2 mL) was added. The reaction mixture was allowed to stir overnight and was then concentrated. The residue was coevaporated several times with toluene and the residue was dissolved in DCM. The organic solution was washed with aqueous saturated sodium bicarbonate, dried over Na₂SO₄, filtered and concentrated. The residue was purified by column chromatography to give N-{4-[3-(2-chlorophenyl)-4-(1H-imidazol-2-yl)-1H-pyrazol-1-yl]-5-methylpyridin-2-yl}acetamide (I-27) (0.012, 23%). LCMS (FA): m/z=393.4 (M+H).

Compounds in the following table were prepared from the appropriate starting materials using the procedures described above:

| | |
|---|---|
| I-60 | LCMS (FA): m/z = 420.7 (M + H). |
| I-64 | LCMS (FA): m/z = 409.3 (M + H). |

### Example 15

### Synthesis of methyl (4-{3-[2-chloro-5-(pyrrolidin-1-ylmethyl)phenyl]-4-(1H-1,2,4-triazol-3-yl)-1H-pyrazol-1-yl}-5-methylpyridin-2-yl)carbamate (I-61)

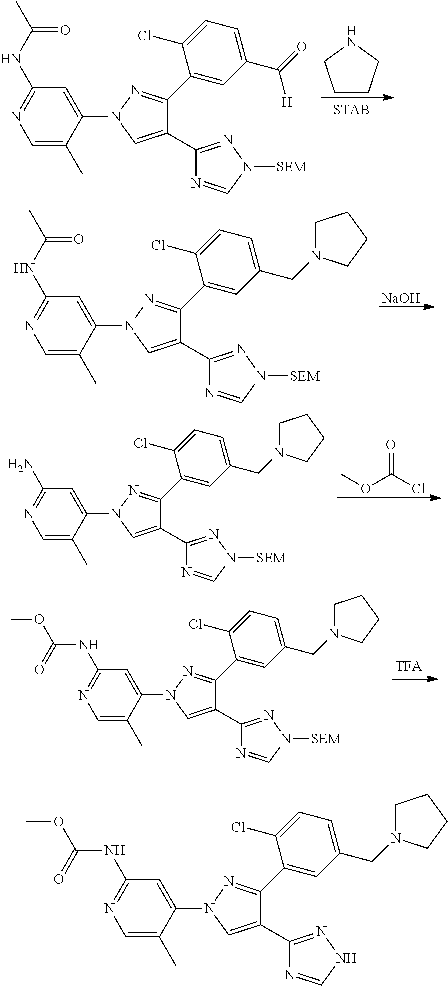

### Step 1: N-(4-{3-[2-chloro-5-(pyrrolidin-1-ylmethyl)phenyl]-4-(1-{[2-(trimethylsilyl)ethoxy]methyl}-1H-1,2,4-triazol-3-yl)-1H-pyrazol-1-yl}-5-methylpyridin-2-yl)acetamide

To a solution of N-{4-[3-(2-chloro-5-formylphenyl)-4-(1-{[2-(trimethylsilyl)ethoxy]methyl}-1H-1,2,4-triazol-3-yl)-1H-pyrazol-1-yl]-5-methylpyridin-2-yl}acetamide (0.20 g, 0.36 mmol) in DCM (6 mL) was added pyrrolidine (0.15 mL, 1.81 mmol) and sodium triacetoxyborohydride (0.23 g, 1.09 mmol). The reaction mixture was allowed to stir at rt overnight and then diluted with water and extracted with DCM. The organic solutions were combined, dried over Na₂SO₄, filtered and concentrated. The residue was purified by column chromatography to give N-(4-{3-[2-chloro-5-(pyrrolidin-1-ylmethyl)phenyl]-4-(1-{[2-(trimethylsilyl)ethoxy]methyl}-1H-1,2,4-triazol-3-yl)-1H-pyrazol-1-yl}-5-methylpyridin-2-yl)acetamide (0.18 g, 82%). LCMS (AA): m/z=607 (M+H).

### Step 2: 4-{3-[2-chloro-5-(pyrrolidin-1-ylmethyl)phenyl]-4-(1-{[2-(trimethylsilyl)ethoxy]methyl}-1H-1,2,4-triazol-3-yl)-1H-pyrazol-1-yl}-5-methylpyridin-2-amine

A solution of N-(4-{3-[2-chloro-5-(pyrrolidin-1-ylmethyl)phenyl]-4-(1-{[2-(trimethylsilyl)ethoxy]methyl}-1H-1,2,4-triazol-3-yl)-1H-pyrazol-1-yl}-5-methylpyridin-2-yl)acetamide (0.18 g, 0.30 mmol) in EtOH (17 mL) and 1N NaOH in water (1.5 mL) was allowed to stir at reflux for 4 h. The reaction mixture was allowed to cool to rt and was then diluted with water and extracted with EtOAc. The organic solutions were combined, dried over Na₂SO₄, filtered and concentrated to give 4-{3-[2-chloro-5-(pyrrolidin-1-ylmethyl)phenyl]-4-(1-{[2-(trimethylsilyl)ethoxy]methyl}-1H-1,2,4-triazol-3-yl)-1H-pyrazol-1-yl}-5-methylpyridin-2-amine (0.15 g, 87%) which was used in the next step without purification. LCMS (AA): m/z=566 (M+H).

### Step 3: methyl (4-{3-[2-chloro-5-(pyrrolidin-1-ylmethyl)phenyl]-4-(1-{[2-(trimethylsilyl)ethoxy]methyl}-1H-1,2,4-triazol-3-yl)-1H-pyrazol-1-yl}-5-methylpyridin-2-yl)carbamate

To a solution of 4-{3-[2-chloro-5-(pyrrolidin-1-ylmethyl)phenyl]-4-(1-{[2-(trimethylsilyl)ethoxy]methyl}-1H-1,2,4-triazol-3-yl)-1H-pyrazol-1-yl}-5-methylpyridin-2-amine (0.15 g, 0.26 mmol) in DCM (4 mL) under an atmosphere of nitrogen was added TEA (0.11 mL, 0.78 mmol) and methyl carbonochloridate (0.060 mL, 0.78 mmol). The reaction mixture was allowed to stir at rt for 4 h and was then concentrated. The residue was redissolved in MeOH (9 mL) and 1M NaOH in water (1.8 mL) was added. The reaction mixture was allowed to stir at rt for 1.5 h and was then diluted with water and extracted with EtOAc. The organic solutions were combined, dried over Na₂SO₄, filtered and concentrated. The residue was purified by column chromatography to give methyl (4-{3-[2-chloro-5-(pyrrolidin-1-ylmethyl)phenyl]-4-(1-{[2-(trimethylsilyl)ethoxy]methyl}-1H-1,2,4-triazol-3-yl)-1H-pyrazol-1-yl}-5-methylpyridin-2-yl)carbamate (0.039 g, 24%). LCMS (AA): m/z=623 (M+H).

### Step 4: methyl (4-{3-[2-chloro-5-(pyrrolidin-1-ylmethyl)phenyl]-4-(1H-1,2,4-triazol-3-yl)-1H-pyrazol-1-yl}-5-methylpyridin-2-yl)carbamate (I-61)

To a solution of methyl (4-{3-[2-chloro-5-(pyrrolidin-1-ylmethyl)phenyl]-4-(1-{[2-(trimethylsilyl)ethoxy]methyl}-1H-1,2,4-triazol-3-yl)-1H-pyrazol-1-yl}-5-methylpyridin-2-yl)carbamate (0.039 g, 0.63 mmol) in DCM (2 mL) was added TFA (0.60 mL, 7.82 mmol). The reaction mixture was allowed to stir at rt overnight and was then concentrated. The residue was purified by column chromatography to give methyl (4-{3-[2-chloro-5-(pyrrolidin-1-ylmethyl)phenyl]-4-(1H-1,2,4-triazol-3-yl)-1H-pyrazol-1-yl}-5-methylpyridin-2-yl)carbamate I-61 (0.027 g, 88%). LCMS (AA): m/z=493 (M+H).

### Example 16

### Synthesis of N-{4-[3-(2-chlorophenyl)-4-(hydroxymethyl)-1H-pyrazol-1-yl]-5-methylpyridin-2-yl}cyclopropanecarboxamide (I-63)

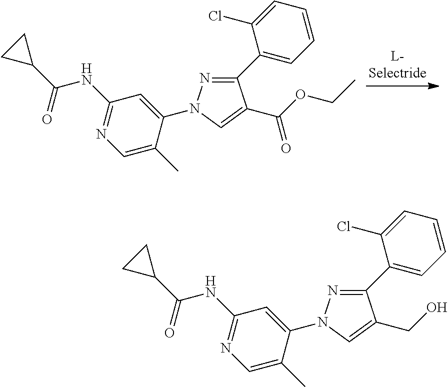 [SMILES: CCOC(=O)c1cn(-c2cc(NC(=O)C3CC3)ncc2C)nc1-c1ccccc1Cl ; Cc1cnc(NC(=O)C2CC2)cc1-n1cc(CO)c(-c2ccccc2Cl)n1]

To a solution of ethyl 3-(2-chlorophenyl)-1-{2-[(cyclopropylcarbonyl)amino]-5-methylpyridin-4-yl}-1H-pyrazole-4-carboxylate (0.025 g, 0.063 mmol) in THF (1.3 mL) was added slowly L-Selectride (1.0 M in THF, 0.61 mL). The reaction mixture was allowed to stir at rt until the starting material was consumed. The reaction mixture was diluted with EtOAc and water and allowed to stir at rt for 20 min. The mixture was extracted with EtOAc. The organic solutions were combined, dried over Na₂SO₄, filtered and concentrated. The residue was purified by column chromatography to give N-{4-[3-(2-chlorophenyl)-4-(hydroxymethyl)-1H-pyrazol-1-yl]-5-methylpyridin-2-yl}cyclopropanecarboxamide I-63 (0.018 g, 77%). LCMS (FA): m/z=383.3 (M+H).

### Example 17

### Synthesis of Intermediate Heterocycles

### Ethyl 4-(2-chlorophenyl)-1H-pyrrole-3-carboxylate

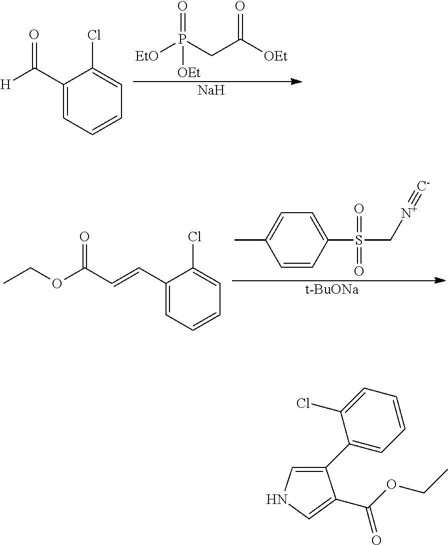

### Step 1: ethyl (2E)-3-(2-chlorophenyl)acrylate

To a stirred slurry of NaH (60% in mineral oil, 0.54 g, 1.34 mmol) in THF (60 mL) was added dropwise ethyl (diethoxyphosphoryl)acetate (2.44 mL, 12.3 mmol) at 0° C. The reaction mixture was allowed to stir for 15 min and then a solution of 2-chlorobenzaldehyde (1.60 g, 11.0 mmol) in THF (23 mL) was added dropwise. The reaction mixture was allowed to stir for 1 h and then quenched by the addition of aqueous saturated ammonium chloride. The mixture was extracted with EtOAc and the organic solutions were combined, dried over MgSO₄, filtered and concentrated. The residue was purified by column chromatography to give ethyl (2E)-3-(2-chlorophenyl)acrylate (1.76 g, 75%). LCMS (FA): m/z=211.3 (M+H).

### Step 2: ethyl 4-(2-chlorophenyl)-1H-pyrrole-3-carboxylate

To a mixture of ethyl (2E)-3-(2-chlorophenyl)acrylate (1.76 g, 8.4 mmol) and p-tolylsulfonylmethyl isocyanide (1.96 g, 10.0 mmol) in THF (10 mL) at −40° C. was added sodium tert-butoxide (0.80 g, 8.4 mmol) slowly. The reaction mixture was allowed to stir for 20 min at −40° C. and then to warm to rt and stir for 3 h. The reaction was quenched by the addition of aqueous saturated sodium bicarbonate and then extracted with EtOAc. The organic solutions were combined, washed with water, dried over Na₂SO₄, filtered and concentrated. The residue was purified by column chromatography to give ethyl 4-(2-chlorophenyl)-1H-pyrrole-3-carboxylate (1.12 g, 54%). LCMS (FA): m/z=249.9 (M+H).

Compounds in the following table were prepared from the appropriate starting materials using the procedures described above:

| | |
|---|---|
| 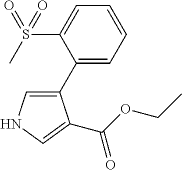 [SMILES: CCOC(=O)c1c[nH]cc1-c1ccccc1S(C)(=O)=O] | ethyl 4-[2-(methylsulfonyl)phenyl]-1H-pyrrole-3-carboxylate |

### Ethyl 4-{2-chloro-5-[(dimethylamino)methyl]phenyl}-1H-pyrrole-3-carboxylate

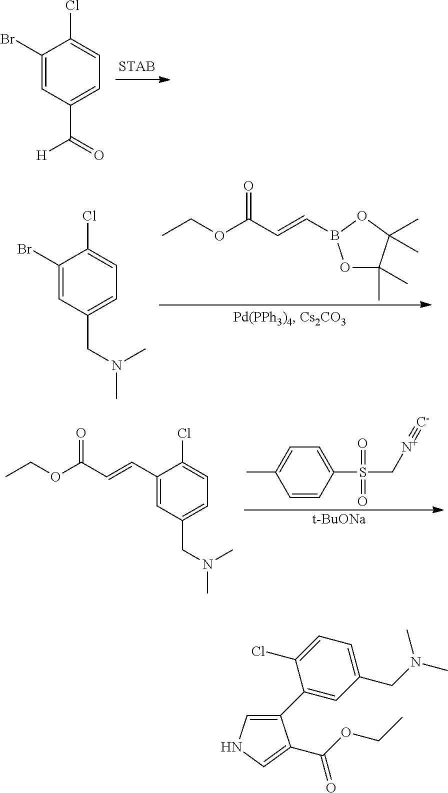

### Step 1: 1-(3-bromo-4-chlorophenyl)-N,N-dimethylmethanamine

To a solution of 3-bromo-4-chlorobenzaldehyde (1.9 g, 8.60 mmol) in DCM (60 mL) were added dimethylamine (2.0 M in THF, 21.6 mL) and STAB (5.50 g, 26.0 mmol). The reaction mixture was allowed to stir at rt overnight and then quenched by the addition of water. The mixture was extracted with DCM. The organic solutions were combined, washed with water, dried over Na₂SO₄, filtered and concentrated. The residue was purified by column chromatography to give 1-(3-bromo-4-chlorophenyl)-N,N-dimethylmethanamine (2.1 g, 98%). LCMS (FA): m/z=248.0 (M+H).

### Step 2: ethyl (2E)-3-{2-chloro-5-[(dimethylamino)methyl]phenyl}acrylate

A mixture of 1-(3-bromo-4-chlorophenyl)-N,N-dimethylmethanamine (0.54 g, 2.16 mmol), tetrakis(triphenylphosphine)palladium(0) (0.25 g, 0.22 mmol), cesium carbonate (2.12 g, 6.49 mmol), and ethyl (2E)-3-(4,4,5,5-tetramethyl-1,3,2-dioxaborolan-2-yl)acrylate (0.59 g, 2.60 mmol) in 1,4-dioxane (12 mL) and water (2.8 mL) were sealed in a vial and subjected to microwave irradiation at 105 oC for 45 min. The reaction mixture was diluted with water and EtOAc. The organic solution was separated and washed with brine, dried over Na₂SO₄, filtered and concentrated. The residue was purified by column chromatography to give ethyl (2E)-3-{2-chloro-5-[(dimethylamino)methyl]phenyl}acrylate (0.21 g, 35%). LCMS (FA): m/z=268.1 (M+H).

### Step 3: ethyl 4-{2-chloro-5-[(dimethylamino)methyl]phenyl}-1H-pyrrole-3-carboxylate

To a mixture of ethyl (2E)-3-{2-chloro-5-[(dimethylamino)methyl]phenyl}acrylate (0.21 g, 0.77 mmol) and p-tolylsulfonylmethyl isocyanide (0.21 g, 1.07 mmol) in THF (20 mL) at −40° C. was added sodium tert-butoxide (0.12 g, 1.07 mmol) slowly. The reaction mixture was allowed to stir for 20 min at −40° C. and then to warm to rt and stir for 3 h. The reaction was quenched by the addition of aqueous saturated sodium bicarbonate and then extracted with EtOAc. The organic solutions were combined, washed with water, dried over Na₂SO₄, filtered and concentrated. The residue was purified by column chromatography to give ethyl 4-{2-chloro-5-[(dimethylamino)methyl]phenyl}-1H-pyrrole-3-carboxylate (0.14 g, 60%). LCMS (FA): m/z=249.9 (M+H).

Compounds in the following table were prepared from the appropriate starting materials using the procedures described above:

| | |
|---|---|
| 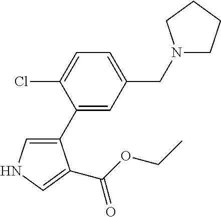 [SMILES: CCOC(=O)c1c[nH]cc1-c1cc(CN2CCCC2)ccc1Cl] | ethyl 4-[2-chloro-5-(pyrrolidin-1-ylmethyl)phenyl]- 1H-pyrrole-3-carboxylate |

### Example 18

### Synthesis of 1-(2-acetamidopyridin-4-yl)-4-(2-chlorophenyl)-1H-pyrrole-3-carboxamide (I-2)

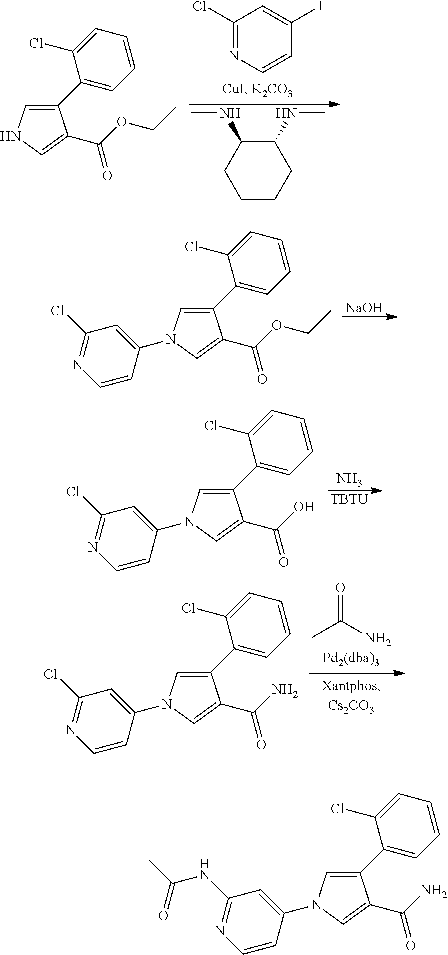

### Step 1: ethyl 4-(2-chlorophenyl)-1-(2-chloropyridin-4-yl)-1H-pyrrole-3-carboxylate

To a solution of copper(I) iodide (0.075 g, 0.39 mmol) in 1,4-dioxane (16 mL) were added 2-chloro-4-iodopyridine (2.83 g, 11.8 mmol), ethyl 4-(2-chlorophenyl)-1H-pyrrole-3-carboxylate (0.51 g, 2.1 mmol), and trans-N,N′-dimethylcyclohexane-1,2-diamine (0.12 mL, 0.79 mmol). The reaction mixture was allowed to stir for 1 h at 100° C. and then potassium carbonate (1.63 g, 11.8 mmol) was added. The reaction mixture was allowed to stir at 105° C. overnight and was then filtered through celite. The filter cake was washed with EtOAc. The filtrate was washed with concentrated ammonium hydroxide and brine. The organic solution was concentrated and purified by column chromatography to give ethyl 4-(2-chlorophenyl)-1-(2-chloropyridin-4-yl)-1H-pyrrole-3-carboxylate (0.28 g, 38%). LCMS (FA): m/z=360.9 (M+H).

### Step 2: 4-(2-chlorophenyl)-1-(2-chloropyridin-4-yl)-1H-pyrrole-3-carboxylic acid

A solution of ethyl 4-(2-chlorophenyl)-1-(2-chloropyridin-4-yl)-1H-pyrrole-3-carboxylate (0.34 g, 0.94 mmol) and sodium hydroxide (0.13 g, 3.2 mmol) in THF (14 mL) and water (24 mL) was allowed to stir at 50° C. overnight. The reaction mixture was concentrated to give 4-(2-chlorophenyl)-1-(2-chloropyridin-4-yl)-1H-pyrrole-3-carboxylic acid (0.31 g, 99%), which was used in the next step without purification. LCMS (FA): m/z=332.9 (M+H).

### Step 3: 4-(2-chlorophenyl)-1-(2-chloropyridin-4-yl)-1H-pyrrole-3-carboxamide

To a solution of 4-(2-chlorophenyl)-1-(2-chloropyridin-4-yl)-1H-pyrrole-3-carboxylic acid (3.7 g, 11.0 mmol) in DCM (300 mL) were added ammonia (0.5 M in 1,4-dioxane, 150 mL), TBTU (7.13 g, 22.2 mmol) and DIEA (19.3 mL, 111.0 mmol). The reaction mixture was allowed to stir at rt overnight and then diluted with water. The solution was extracted with DCM. The organic solutions were combined, washed with water, dried over Na₂SO₄, filtered and concentrated. The residue was purified by column chromatography to give 4-(2-chlorophenyl)-1-(2-chloropyridin-4-yl)-1H-pyrrole-3-carboxamide (2.9 g, 79%). LCMS (FA): m/z=332.4 (M+H).

### Step 4: 1-(2-acetamidopyridin-4-yl)-4-(2-chlorophenyl)-1H-pyrrole-3-carboxamide

To a solution of 4-(2-chlorophenyl)-1-(2-chloropyridin-4-yl)-1H-pyrrole-3-carboxamide (0.46 g, 0.41 mmol) in 1,4-dioxane (3.2 mL) were added acetamide (0.16 g, 2.76 mmol), tris(dibenzylideneacetone)dipalladium (0.018 g, 0.020 mmol), xantphos (0.034 g, 0.059 mmol) and cesium carbonate (0.23 g, 0.69 mmol). The reaction mixture was sealed in a vial and subjected to microwave irradiation at 130° C. for 1 h. The reaction mixture was diluted with water and extracted with EtOAc. The organic solutions were combined, washed with water, dried over Na₂SO₄, filtered and concentrated. The residue was purified by column chromatography to give 1-(2-acetamidopyridin-4-yl)-4-(2-chlorophenyl)-1H-pyrrole-3-carboxamide (0.015 g, 30%). LCMS (FA): m/z=355.5 (M+H).

Compounds in the following table were prepared from the appropriate starting materials using the procedures described above:

| | |
|---|---|
| I-47 | LCMS (FA): m/z = 399.9 (M + H). |

### Example 19

### Synthesis of N-{4-[3-(2-chlorophenyl)-4-(1H-1,2,4-triazol-3-yl)-1H-pyrrol-1-yl]pyridin-2-yl}acetamide (I-22)

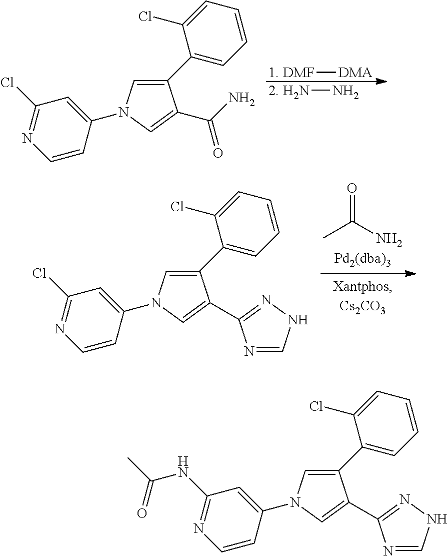

### Step 1: 2-chloro-4-[3-(2-chlorophenyl)-4-(1H-1,2,4-triazol-3-yl)-1H-pyrrol-1-yl]pyridine

To a suspension of 4-(2-chlorophenyl)-1-(2-chloropyridin-4-yl)-1H-pyrrole-3-carboxamide (0.20 g, 0.59 mmol) in anhydrous toluene (3 mL) was added DMF-DMA (0.23 mL, 1.75 mmol). The reaction mixture was allowed to stir for 2 h at 50° C. The reaction mixture was concentrated and the residue was dissolved in AcOH (2.3 mL) and hydrazine hydrate (0.14 mL, 2.90 mmol) was added. The reaction mixture was allowed to stir at rt for 1 h and was concentrated. The residue was azeotroped several times with toluene and then diluted with EtOAc. The solution was washed with aqueous saturated sodium bicarbonate, dried over Na₂SO₄, filtered and concentrated. The residue was purified by column chromatography to give 2-chloro-4-[3-(2-chlorophenyl)-4-(1H-1,2,4-triazol-3-yl)-1H-pyrrol-1-yl]pyridine (0.15 g, 71%). LCMS (FA): m/z=355.9 (M+H).

### Step 2: N-{4-[3-(2-chlorophenyl)-4-(1H-1,2,4-triazol-3-yl)-1H-pyrrol-1-yl]pyridin-2-yl}acetamide

To a solution of 2-chloro-4-[3-(2-chlorophenyl)-4-(1H-1,2,4-triazol-3-yl)-1H-pyrrol-1-yl]pyridine (0.16 g, 0.44 mmol) in 1,4-dioxane (4.9 mL) were added acetamide (0.39 g, 6.53 mmol), tris(dibenzylideneacetone)dipalladium (0.0.56 g, 0.061 mmol), xantphos (0.11 g, 0.18 mmol) and cesium carbonate (0.71 g, 2.18 mmol). The reaction mixture was sealed in a vial and subjected to microwave irradiation at 150° C. for 1 h. The reaction mixture was diluted with water and extracted with EtOAc. The organic solutions were combined, washed with water, dried over Na₂SO₄, filtered and concentrated. The residue was purified by column chromatography to give N-{4-[3-(2-chlorophenyl)-4-(1H-1,2,4-triazol-3-yl)-1H-pyrrol-1-yl]pyridin-2-yl}acetamide (0.024 g, 14%). LCMS (FA): m/z=379.5 (M+H).

Compounds in the following table were prepared from the appropriate starting materials using the procedures described above:

| | |
|---|---|
| I-38 | LCMS (FA): m/z = 423.1 (M + H). |

### Example 20

### Synthesis of 4-(2-chlorophenyl)-1-[2-(isobutyrylamino)-5-methylpyridin-4-yl]-1H-pyrrole-3-carboxamide (I-33) and N-{4-[3-(2-chlorophenyl)-4-(1H-1,2,4-triazol-3-yl)-1H-pyrrol-1-yl]-5-methylpyridin-2-yl}-2-methylpropanamide (I-26)

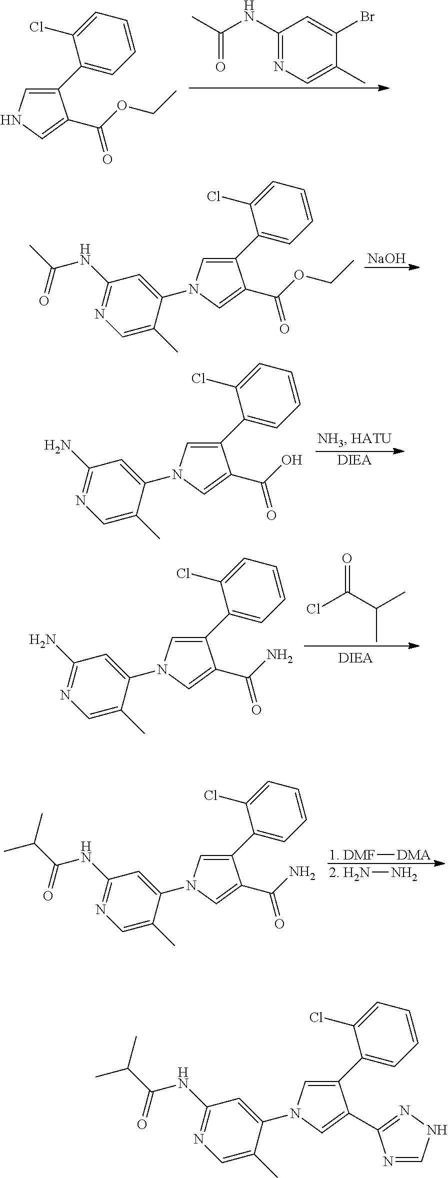

### Step 1: ethyl 1-(2-acetamido-5-methylpyridin-4-yl)-4-(2-chlorophenyl)-1H-pyrrole-3-carboxylate

To a mixture of ethyl 4-(2-chlorophenyl)-1H-pyrrole-3-carboxylate (0.55 g, 2.2 mmol), N-(4-bromo-5-methylpyridin-2-yl)acetamide (1.01 g, 4.40 mmol), trans-1,2-bis(methylamino)cyclohexane (0.13 mL, 0.85 mmol), and copper(I) iodide (0.081 g, 0.42 mmol) in 1,4-dioxane (15 mL) was added potassium carbonate (1.22 g, 8.81 mmol). The reaction mixture was allowed to stir at 105° C. in a sealed vial for 48 h. The reaction mixture was allowed to cool to rt, diluted with EtOAc, and then washed with brine and 20% aqueous ammonia. The organic solution was concentrated and the residue was purified by column chromatography to give ethyl 1-(2-acetamido-5-methylpyridin-4-yl)-4-(2-chlorophenyl)-1H-pyrrole-3-carboxylate (0.70 g, 80%). LCMS (FA): m/z=398.3 (M+H).

### Step 2: 1-(2-amino-5-methylpyridin-4-yl)-4-(2-chlorophenyl)-1H-pyrrole-3-carboxylic acid

A mixture of ethyl 1-(2-acetamido-5-methylpyridin-4-yl)-4-(2-chlorophenyl)-1H-pyrrole-3-carboxylate (0.20 g, 0.50 mmol) and sodium hydroxide (0.10 g, 2.51 mmol) in THF (10 mL), MeOH (10 mL) and water (10 mL) was allowed to stir at 50° C. for 4 days. The reaction mixture was concentrated to give 1-(2-amino-5-methylpyridin-4-yl)-4-(2-chlorophenyl)-1H-pyrrole-3-carboxylic acid (0.17 g, 100%). LCMS (FA): m/z=328.0 (M+H).

### Step 3: 1-(2-amino-5-methylpyridin-4-yl)-4-(2-chlorophenyl)-1H-pyrrole-3-carboxamide

A mixture of 1-(2-amino-5-methylpyridin-4-yl)-4-(2-chlorophenyl)-1H-pyrrole-3-carboxylic acid (0.60 g, 2.0 mmol), ammonia (0.5 M in 1,4-dioxane, 73.2 mL, 36.6 mmol), HATU (1.39 g, 3.66 mmol) and DIEA (3.19 mL, 18.3 mmol) in DCM (50 mL) was allowed to stir at rt for 3 h. The reaction mixture was diluted with water and extracted with DCM. The organic solutions were combined, washed with water, dried over Na₂SO₄, filtered and concentrated. The residue was purified by column chromatography to give 1-(2-amino-5-methylpyridin-4-yl)-4-(2-chlorophenyl)-1H-pyrrole-3-carboxamide (0.56 g, 90%). LCMS (FA): m/z=327.2 (M+H).

### Step 4: 4-(2-chlorophenyl)-1-[2-(isobutyrylamino)-5-methylpyridin-4-yl]-1H-pyrrole-3-carboxamide (I-33)

To a solution of 1-(2-amino-5-methylpyridin-4-yl)-4-(2-chlorophenyl)-1H-pyrrole-3-carboxamide (0.14 g, 0.43 mmol) in DCM (4.25 mL) were added DIEA (0.14 mL, 0.86 mmol) and 2-methylpropanoyl chloride (0.090 mL, 0.86 mmol) at 0° C. The reaction mixture was allowed to stir for 1 h at this temperature and then concentrated. The residue was redissolved in MeOH (5 mL) and NaOH (1N, 1 mL) was added. The mixture was allowed to stir at rt for 1 h and then concentrated. The residue was dissolved in DCM and purified by column chromatography to give 4-(2-chlorophenyl)-1-[2-(isobutyrylamino)-5-methylpyridin-4-yl]-1H-pyrrole-3-carboxamide (1-33) (0.13 g, 76%). LCMS (FA): m/z=397.4 (M+H).

### Step 5: N-{4-[3-(2-chlorophenyl)-4-(1H-1,2,4-triazol-3-yl)-1H-pyrrol-1-yl]-5-methylpyridin-2-yl}-2-methylpropanamide (I-26)

To a suspension of 4-(2-chlorophenyl)-1-[2-(isobutyrylamino)-5-methylpyridin-4-yl]-1H-pyrrole-3-carb oxamide (0.11 g, 0.28 mmol) in anhydrous toluene (12.5 mL) was added DMF-DMA (4 mL). The reaction mixture was allowed to stir for 2 h at 50° C. The reaction mixture was concentrated and the residue was dissolved in AcOH (1.1 mL) and hydrazine hydrate (0.068 mL, 1.39 mmol) was added. The reaction mixture was allowed to stir at rt for 1 h and was concentrated. The residue was azeotroped several times with toluene and then diluted with EtOAc. The solution was washed with aqueous saturated sodium bicarbonate, dried over Na₂SO₄, filtered and concentrated. The residue was triturated with ether and hexane. The resulting solid was washed with water and hexane to give N-{-4-[3-(2-chlorophenyl)-4-(1H-1,2,4-triazol-3-yl)-1H-pyrrol-1-yl]-5-methylpyridin-2-yl}-2-methylpropanamide (I-26) (0.080 g, 67%). LCMS (FA): m/z=421.2 (M+H).

Compounds in the following table were prepared from the appropriate starting materials using the procedures described above:

| | |
|---|---|
| I-28 | LCMS (FA): m/z = 383.3 (M + H). |
| I-40 | LCMS (FA): m/z = 395.2 (M + H). |
| I-41 | LCMS (FA): m/z = 369.5 (M + H). |
| I-50 | LCMS (FA): m/z = 407.3 (M + H). |
| I-36 | LCMS (FA): m/z = 385.2 (M + H). |
| I-53 | LCMS (FA): m/z = 359.5 (M + H). |
| I-55 | LCMS (FA): m/z = 409.2 (M + H). |
| I-54 | LCMS (FA): m/z = 429.1 (M + H). |
| I-79 | LCMS (FA): m/z = 413.4 (M + H). |
| I-72 | LCMS (FA): m/z = 453.4 (M + H). |
| I-78 | LCMS (FA): m/z = 453.1 (M + H). |
| I-74 | LCMS (FA): m/z = 437.2 (M + H). |
| I-83 | LCMS (FA): m/z = 417.3 (M + H). |
| I-81 | LCMS (FA): m/z = 441.1 (M + H). |
| I-73 | LCMS (FA): m/z = 477.1 (M + H). |
| I-70 | LCMS (FA): m/z = 426.3 (M + H). |
| I-75 | LCMS (FA): m/z = 410.2 (M + H). |
| I-76 | LCMS (FA): m/z = 450.3 (M + H). |
| I-71 | LCMS (FA): m/z = 478.3 (M + H). |
| I-84 | LCMS (FA): m/z = 502.3 (M + H). |
| I-80 | LCMS (FA): m/z = 468.1 (M + H). |
| I-77 | LCMS (FA): m/z = 452.2 (M + H). |
| I-31 | LCMS (FA): m/z = 393.2 (M + H). |

### Example 21

### Synthesis of 1-(2-acetamido-5-chloropyridin-4-yl)-4-(2-chlorophenyl)-1H-pyrrole-3-carboxamide (I-37) and methyl {4-[3-carbamoyl-4-(2-chlorophenyl)-1H-pyrrol-1-yl]-5-chloropyridin-2-yl}carbamate (I-51)

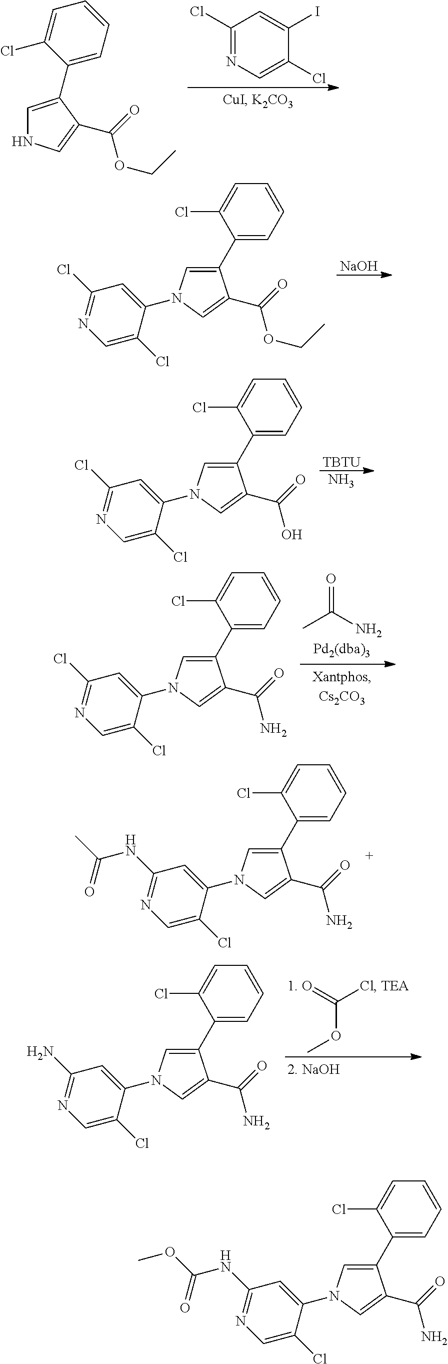

### Step 1: ethyl 4-(2-chlorophenyl)-1-(2,5-dichloropyridin-4-yl)-1H-pyrrole-3-carboxylate

To a mixture of copper(I) iodide (0.011 g, 0.060 mmol) in 1,4-dioxane (10 mL) were added 2,5-dichloro-4-iodopyridine (0.49 g, 1.80 mmol), ethyl 4-(2-chlorophenyl)-1H-pyrrole-3-carboxylate (0.078 g, 0.31 mmol), trans-1,2-bis(methylamino)cyclohexane (0.019 mL, 0.12 mmol) and potassium carbonate (0.25 g, 1.80 mmol). The reaction mixture was allowed to stir at 105° C. in a sealed vessel overnight and then allowed to cool to rt. The contents were filtered through celite and washed with EtOAc. The filtrate was washed with ammonium hydroxide and brine and then concentrated. The reside was purified by column chromatography to give ethyl 4-(2-chlorophenyl)-1-(2,5-dichloropyridin-4-yl)-1H-pyrrole-3-carboxylate (0.048 g, 39%). LCMS (FA): m/z=397.3 (M+H).

### Step 2: 4-(2-chlorophenyl)-1-(2,5-dichloropyridin-4-yl)-1H-pyrrole-3-carboxylic acid

A solution of ethyl 4-(2-chlorophenyl)-1-(2,5-dichloropyridin-4-yl)-1H-pyrrole-3-carboxylate (0.13 g, 0.34 mmol) and sodium hydroxide (0.027 g, 0.68 mmol) in water (10 mL), MeOH (10 mL) and THF (10 mL) was allowed to stir at 70° C. overnight. The reaction mixture was concentrated to give 4-(2-chlorophenyl)-1-(2,5-dichloropyridin-4-yl)-1H-pyrrole-3-carboxylic acid (0.12 g, 99%) which was used without purification in the next step. LCMS (FA): m/z=367.3 (M+H).

### Step 3: 4-(2-chlorophenyl)-1-(2,5-dichloropyridin-4-yl)-1H-pyrrole-3-carboxamide

A mixture of 4-(2-chlorophenyl)-1-(2,5-dichloropyridin-4-yl)-1H-pyrrole-3-carboxylic acid (0.13 g. 0.34 mmol), ammonia (0.5M in 1,4-dioxane, 5.4 mL, 2.7 mmol), TBTU (0.22 g, 0.68 mmol) and DIEA (0.6 mL, 3.4 mmol) in DCM (10 mL) was allowed to stir at rt overnight. The reaction mixture was diluted with water and extracted with DCM. The organic solutions were combined, washed with water, dried over Na₂SO₄, filtered and concentrated. The residue was purified by column chromatography to give 4-(2-chlorophenyl)-1-(2,5-dichloropyridin-4-yl)-1H-pyrrole-3-carboxamide (0.042 g, 34%). LCMS (FA): m/z=366.4 (M+H).

### Step 4: 1-(2-acetamido-5-chloropyridin-4-yl)-4-(2-chlorophenyl)-1H-pyrrole-3-carboxamide (I-37) and 1-(2-amino-5-chloropyridin-4-yl)-4-(2-chlorophenyl)-1H-pyrrole-3-carboxamide

To a solution of 4-(2-chlorophenyl)-1-(2,5-dichloropyridin-4-yl)-1H-pyrrole-3-carboxamide (0.051 g, 0.14 mmol) in 1,4-dioxane (3.2 mL) were added acetamide (0.12 g, 2.08 mmol), tris(dibenzylideneacetone)dipalladium (0.018 g, 0.020 mmol), xantphos (0.034 g, 0.59 mmol) and cesium carbonate (0.23 g, 0.69 mmol). The reaction mixture was sealed in a vial and subjected to microwave irradiation at 130° C. for 1 h. The reaction mixture was diluted with water and extracted with EtOAc. The organic solutions were combined, washed with water, dried over Na₂SO₄, filtered and concentrated. The residue was purified by column chromatography to give 1-(2-acetamido-5-chloropyridin-4-yl)-4-(2-chlorophenyl)-1H-pyrrole-3-carboxamide (I-37) (0.003 g, 5%) and 1-(2-amino-5-chloropyridin-4-yl)-4-(2-chlorophenyl)-1H-pyrrole-3-carboxamide (0.005 g, 10%). LCMS (FA): m/z=389.5 (M+H) and LCMS (FA): m/z=347.5 (M+H), respectively.

### Step 5: methyl {4-[3-carbamoyl-4-(2-chlorophenyl)-1H-pyrrol-1-yl]-5-chloropyridin-2-yl}carbamate (I-51)

To a stirred solution of 1-(2-amino-5-chloropyridin-4-yl)-4-(2-chlorophenyl)-1H-pyrrole-3-carboxamide (0.005 g, 0.043 mmol) and TEA (0.006 mL, 0.043 mmol) in DCM (2 mL) was added methyl carbonochloridate (0.003 mL, 0.043 mmol) slowly at 0° C. The reaction mixture was allowed to warm to rt and stir for 1 h and then concentrated. The residue was redissolved in MeOH (0.5 mL) and 1M NaOH (0.1 mL) was added. The reaction mixture was allowed to stir at rt for 1 h and then concentrated. The residue was purified by column chromatography to give methyl {4-[3-carbamoyl-4-(2-chlorophenyl)-1H-pyrrol-1-yl]-5-chloropyridin-2-yl}carbamate (I-51) (0.004 g, 60%). LCMS (FA): m/z=405.5 (M+H).

### Example 22

### Synthesis of N-{4-[3-(2-chlorophenyl)-4-(1H-imidazol-2-yl)-1H-pyrrol-1-yl]-5-methylpyridin-2-yl}cyclopropanecarboxamide (I-82)

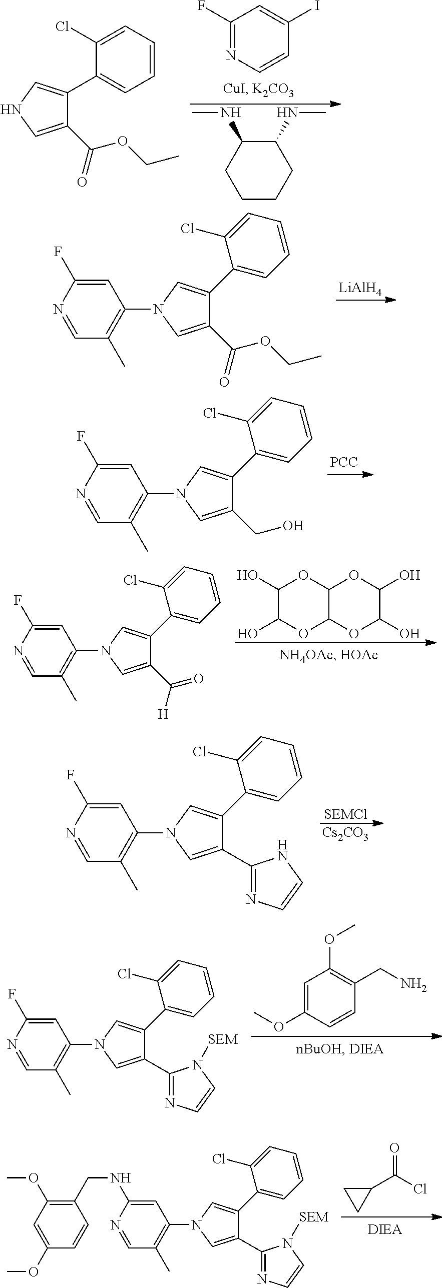

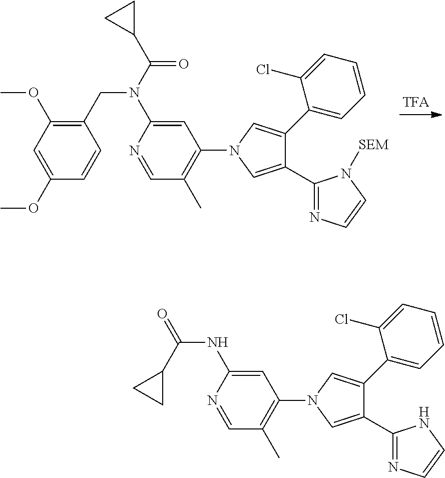 [SMILES: COc1ccc(CN(C(=O)C2CC2)c2cc(-n3cc(-c4ccccc4Cl)c(-c4nccn4COCC[Si](C)(C)C)c3)c(C)cn2)c(OC)c1 ; Cc1cnc(NC(=O)C2CC2)cc1-n1cc(-c2ncc[nH]2)c(-c2ccccc2Cl)c1]

### Step 1: ethyl 4-(2-chlorophenyl)-1-(2-fluoro-5-methylpyridin-4-yl)-1H-pyrrole-3-carboxylate

To a slurry of copper(I) iodide (0.14 g, 0.73 mmol) and potassium carbonate (3.03 g, 21.9 mmol) in 1,4-dioxane (15 mL) were added 2-fluoro-4-iodo-5-methylpyridine (5.03 g, 21.2 mmol), ethyl 4-(2-chlorophenyl)-1H-pyrrole-3-carboxylate (0.95 g, 3.8 mmol), and trans-N,N′-dimethylcyclohexane-1,2-diamine (0.23 mL, 1.46 mmol). The reaction mixture was allowed to stir in a sealed vial for 48 h at 105° C. and then allowed to cool to rt. The mixture was diluted with EtOAc and washed with brine. The organic solutions were combined, concentrated, and purified by column chromatography to give ethyl 4-(2-chlorophenyl)-1-(2-fluoro-5-methylpyridin-4-yl)-1H-pyrrole-3-carboxylate (1.2 g, 88%). LCMS (FA): m/z=359.3 (M+H).

### Step 2: [4-(2-chlorophenyl)-1-(2-fluoro-5-methylpyridin-4-yl)-1H-pyrrol-3-yl]methanol

A solution of ethyl 4-(2-chlorophenyl)-1-(2-fluoro-5-methylpyridin-4-yl)-1H-pyrrole-3-carboxylate (1.95 g, 5.43 mmol) in THF (28 mL) was allowed to stir at 0° C. under an atmosphere of nitrogen. To this solution was added lithium tetrahydroaluminate (1.0 M in diethyl ether, 10.9 mL, 10.9 mmol) dropwise. The reaction mixture was allowed to stir at 0° C. for 2 h and was then quenched at this temperature by the slow addition of sodium sulfate decahydrate (7.0 g). The mixture was allowed to stir for 3 h and warm to rt and was then diluted with EtOAc and filtered. The solid was washed with EtOAc and then filtrate was concentrated to give [4-(2-chlorophenyl)-1-(2-fluoro-5-methylpyridin-4-yl)-1H-pyrrol-3-yl]methanol (1.54 g, 89%) which was used in the next step without purificiation. LCMS (AA): m/z=317 (M+H).

### Step 3: 4-(2-chlorophenyl)-1-(2-fluoro-5-methylpyridin-4-yl)-1H-pyrrole-3-carbaldehyde

To a solution of [4-(2-chlorophenyl)-1-(2-fluoro-5-methylpyridin-4-yl)-1H-pyrrol-3-yl]methanol (1.59 g, 5.02 mmol) in DCM (25 mL) was added celite (0.75 g) and PCC (1.35 g, 6.27 mmol). The reaction mixture was allowed to stir at rt for 1 h and was then diluted with diethyl ether and filtered through silica gel. The filter cake was washed with diethyl ether and the filtrate was concentrated. The residue was purified by column chromatography to give 4-(2-chlorophenyl)-1-(2-fluoro-5-methylpyridin-4-yl)-1H-pyrrole-3-carbaldehyde (0.98, 62%). LCMS (AA): m/z=315 (M+H).

### Step 4: 4-[3-(2-chlorophenyl)-4-(1H-imidazol-2-yl)-1H-pyrrol-1-yl]-2-fluoro-5-methylpyridine

To a solution of 4-(2-chlorophenyl)-1-(2-fluoro-5-methylpyridin-4-yl)-1H-pyrrole-3-carbaldehyde (1.16 g, 3.68 mmol) in MeOH (13 mL) were added hexahydro[1,4]dioxino[2,3-b][1,4]dioxine-2,3,6,7-tetrol (1.55 g, 7.38 mmol), ammonium acetate (1.70 g, 22.1 mmol), and AcOH (2.10 mmol). The reaction mixture was allowed to stir at rt for 24 h and was then allowed to stir at 45° C. for 8 h. The reaction mixture was allowed to cool to rt overnight and was then carefully diluted with aqueous saturated sodium bicarbonate solution. The mixture was extracted with EtOAc. The organic solutions were combined, washed with water, dried over Na₂SO₄, filtered and concentrated. The residue was purified by column chromatography to give 4-[3-(2-chlorophenyl)-4-(1H-imidazol-2-yl)-1H-pyrrol-1-yl]-2-fluoro-5-methylpyridine (0.79 g, 61%). LCMS (AA): m/z=355 (M+H).

### Step 5: 4-[3-(2-chlorophenyl)-4-(1-{[2-(trimethylsilyl)ethoxy]methyl}-1H-imidazol-2-yl)-1H-pyrrol-1-yl]-2-fluoro-5-methylpyridine

To a solution of 4-[3-(2-chlorophenyl)-4-(1H-imidazol-2-yl)-1H-pyrrol-1-yl]-2-fluoro-5-methylpyridine (0.43 g, 1.22 mmol) in DMF (4 mL) was added cesium carbonate (0.40 g, 1.21 mmol). The reaction mixture was allowed to stir at rt for 45 min and then [2-(chloromethoxy)ethyl]-(trimethyl)silane (0.22 mL, 1.22 mmol) was added. The reaction mixture was allowed to stir for 1 h at rt and was then diluted with EtOAc. The organic solutions was filtered and concentrated. The residue was purified by column chromatography to give 4-[3-(2-chlorophenyl)-4-(1-{[2-(trimethylsilyl)ethoxy]-methyl}-1H-imidazol-2-yl)-1H-pyrrol-1-yl]-2-fluoro-5-methylpyridine (0.15 g, 25%). LCMS (AA): m/z=483 (M+H).

### Step 6: 4-[3-(2-chlorophenyl)-4-(1-{[2-(trimethylsilyl)ethoxy]methyl}-1H-imidazol-2-yl)-1H-pyrrol-1-yl]-N-(2,4-dimethoxybenzyl)-5-methylpyridin-2-amine

To a solution of 4-[3-(2-chlorophenyl)-4-(1-{[2-(trimethylsilyl)ethoxy]-methyl}-1H-imidazol-2-yl)-1H-pyrrol-1-yl]-2-fluoro-5-methylpyridine (0.15 g, 0.30 mmol) in 1-butanol (5 mL) were added 1-(2,4-dimethoxyphenyl)-methanamine (0.45 mL, 3.02 mmol) and DIEA (0.16 mL, 0.91 mmol). The reaction mixture was subjected to microwave irradiation at 175° C. for 6 h and was then concentrated. The residue was purified by column chromatography to give 4-[3-(2-chlorophenyl)-4-(1-{[2-(trimethylsilyl)ethoxy]methyl}-1H-imidazol-2-yl)-1H-pyrrol-1-yl]-N-(2,4-dimethoxybenzyl)-5-methylpyridin-2-amine (0.15 g, 78%). LCMS (AA): m/z=630 (M+H).

### Step 7: N-{4-[3-(2-chlorophenyl)-4-(1-{[2-(trimethylsilyl)ethoxy]methyl}-1H-imidazol-2-yl)-1H-pyrrol-1-yl]-5-methylpyridin-2-yl}-N-(2,4-dimethoxybenzyl)cyclopropanecarboxamide

To a solution of 4-[3-(2-chlorophenyl)-4-(1-{[2-(trimethylsilyl)ethoxy]methyl}-1H-imidazol-2-yl)-1H-pyrrol-1-yl]-N-(2,4-dimethoxybenzyl)-5-methylpyridin-2-amine (0.15 g, 0.23 mmol) in DCM (3.4 mL) under an atmosphere of nitrogen at 0° C. was added DIEA (0.12 mL, 0.70 mmol) and then cyclopropanecarbonyl chloride (0.042 mL, 0.47 mmol) was added. The reaction mixture was allowed to stir at 0° C. for 1 h and was then diluted with water. The aqueous solution was separated and further extracted with DCM. The organic solutions were combined, washed with water and brine, dried over Na₂SO₄, filtered and concentrated. The residue was purified by column chromatography to give N-{4-[3-(2-chlorophenyl)-4-(1-{[2-(trimethylsilyl)ethoxy]methyl}-1H-imidazol-2-yl)-1H-pyrrol-1-yl]-5-methylpyridin-2-yl}-N-(2,4-dimethoxybenzyl)cyclopropanecarboxamide (0.12 g, 74%). LCMS (AA): m/z=699 (M+H).

### Step 8: N-{4-[3-(2-chlorophenyl)-4-(1H-imidazol-2-yl)-1H-pyrrol-1-yl]-5-methylpyridin-2-yl}cyclopropanecarboxamide

To a solution of N-{4-[3-(2-chlorophenyl)-4-(1-{[2-(trimethylsilyl)ethoxy]methyl}-1H-imidazol-2-yl)-1H-pyrrol-1-yl]-5-methylpyridin-2-yl}-N-(2,4-dimethoxybenzyl)cyclopropane-carboxamide (0.12 g, 0.17 mmol) in DCM (5 mL) was added TFA (1.67 mL, 21.7 mmol). The reaction mixture was allowed to stir at rt overnight and then additional TFA (1.5 mL) was added. The reaction mixture was allowed to stir overnight and was then concentrated. The residue was redissolved in TFA (2.5 mL) and allowed to stir at rt overnight. The reaction mixture was concentrated and the residue was redissolved in DCM. The organic solution was washed with aqueous saturated sodium bicarbonate, dried over Na₂SO₄, filtered and concentrated. The residue was purified by column chromatography to give N-{4-[3-(2-chlorophenyl)-4-(1H-imidazol-2-yl)-1H-pyrrol-1-yl]-5-methylpyridin-2-yl}cyclopropanecarboxamide (0.025, 34%). LCMS (AA): m/z=418 (M+H).

### Example 23

### Synthesis of N-{4-[3-(2-chlorophenyl)-4-(hydroxymethyl)-1H-pyrrol-1-yl]-5-methylpyridin-2-yl}acetamide (I-85)

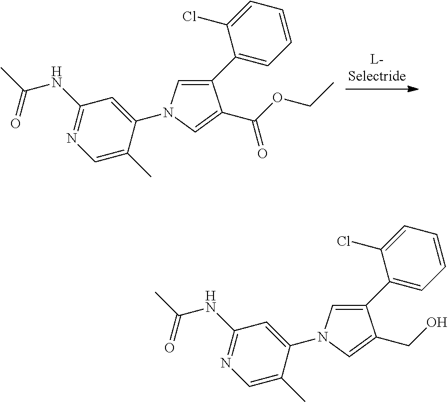

To a solution of ethyl 1-(2-acetamido-5-methylpyridin-4-yl)-4-(2-chlorophenyl)-1H-pyrrole-3-carboxylate (0.25 g, 0.63 mmol) in THF (14 mL) was added slowly L-Selectride (1.0 M in THF, 3.1 mL). The reaction mixture was allowed to stir at rt until the starting material was consumed. The reaction mixture was diluted with water and extracted with EtOAc. The organic solutions were combined, dried over Na₂SO₄, filtered and concentrated. The residue was purified by column chromatography to give N-{4-[3-(2-chlorophenyl)-4-(hydroxymethyl)-1H-pyrrol-1-yl]-5-methylpyridin-2-yl}acetamide (0.13 g, 58%). LCMS (FA): m/z=356.2 (M+H).

### Example 24

### Synthesis of N-{4-[4-(6-aminopyridin-2-yl)-3-(2-chlorophenyl)-1H-pyrazol-1-yl]-5-methylpyridin-2-yl}acetamide (I-107)

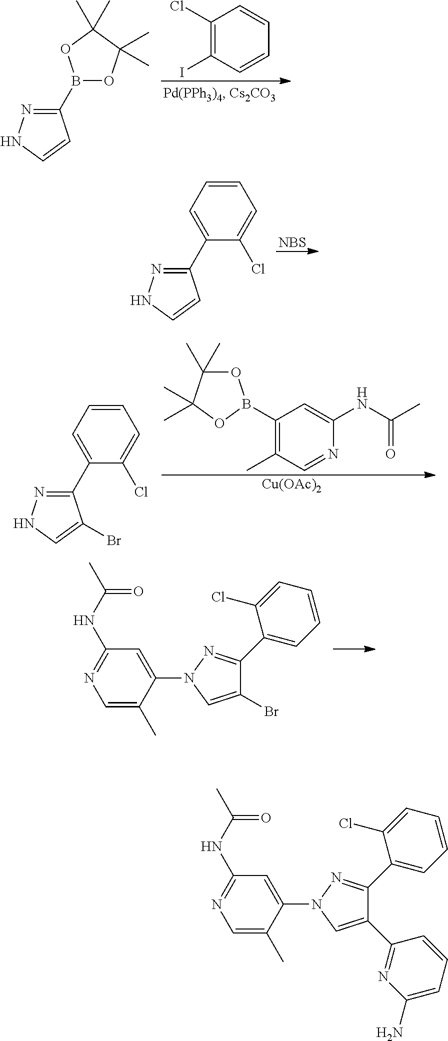

### Step 1: 3-(2-Chlorophenyl)-1H-pyrazole

A mixture of 1-chloro-2-iodobenzene (0.67 g, 2.8 mmol), 3-(4,4,5,5-tetramethyl-1,3,2-dioxaborolan-2-yl)-1H-pyrazole (1.1 g, 5.6 mmol), tetrakis(triphenylphosphine)palladium(0) (0.65 g, 0.56 mmol), and cesium carbonate (4.6 g, 14 mmol) in 1,4-dioxane (18 mL) and water (2.0 mL) were sealed in a vial and subjected to microwave irradiation at 150° C. for 50 min. The organic solution was separated and concentrated. The residue was purified by column chromatography to give 3-(2-chlorophenyl)-1H-pyrazole (0.50 g, 99%). LCMS (FA): m/z=179 (M+H).

### Step 2: 4-Bromo-3-(2-chlorophenyl)-1H-pyrazole

To a solution of 3-(2-chlorophenyl)-1H-pyrazole (1.2 g, 6.5 mmol) in DCM (42 mL) was added NBS (1.2 g, 6.5 mmol). The reaction was allowed to stir at rt for 50 min. The reaction mixture was then treated with saturated aqueous sodium thiosulfate and extracted with DCM. The organic solutions were combined, washed with brine, dried over Na₂SO₄, filtered and concentrated. The residue was purified by column chromatography to give 4-bromo-3-(2-chlorophenyl)-1H-pyrazole (1.7 g, 99%). LCMS (FA): m/z=257 (M+H).

### Step 3: N-(4-(4-bromo-3-(2-chlorophenyl)-1H-pyrazol-1-yl)-5-methylpyridin-2-yl)acetamide

A mixture of 4-bromo-3-(2-chlorophenyl)-1H-pyrazole (1.07 g, 4.16 mmol), N-[5-methyl-4-(4,4,5,5-tetramethyl-1,3,2-dioxaborolan-2-yl)pyridin-2-yl]acetamide (2.16 g, 7.82 mmol), cupric acetate (0.96 g, 5.3 mmol) and pyridine (1.67 mL, 20.6 mmol) in THF (11 mL) was allowed to stir at 70° C. overnight. An additional portion of 4-bromo-3-(2-chlorophenyl)-1H-pyrazole (1.0 g, 4.1 mmol) and cupric acetate (0.36 g, 2.0 mmol) were added and the reaction was again heated at 70° C. overnight. The reaction mixture was then filtered and the solid washed with EtOAc. The organic solution was washed with 10% aqueous ammonia solution followed by brine, dried over MgSO₄, filtered and concentrated. The residue was purified by column chromatography to give N-(4-(4-bromo-3-(2-chlorophenyl)-1H-pyrazol-1-yl)-5-methylpyridin-2-yl)acetamide (0.94 g, 56%). LCMS (FA): m/z=405 (M+H).

### Step 4: N-{4-[4-(6-aminopyridin-2-yl)-3-(2-chlorophenyl)-1H-pyrazol-1-yl]-5-methylpyridin-2-yl}acetamide (I-107)

A mixture of N-(4-(4-bromo-3-(2-chlorophenyl)-1H-pyrazol-1-yl)-5-methylpyridin-2-yl)acetamide (0.15 g, 0.37 mmol), imidodicarbonic acid, 2-[6-(4,4,5,5-tetramethyl-1,3,2-dioxaborolan-2-yl)-2-pyridinyl]-, 1,3-bis(1,1-dimethylethyl) ester (0.16 g, 0.37 mmol), tetrakis(triphenylphosphine)palladium(0) (0.043 g, 0.037 mmol), and cesium carbonate (0.36 g, 1.1 mmol) in 1,4-dioxane (1.8 mL) and water (0.4 mL) were sealed in a vial and subjected to microwave irradiation at 150° C. for 30 min. The reaction mixture was diluted with water and extracted with EtOAc. The organic solutions were combined, dried over Na₂SO₄, filtered and concentrated. The crude product was dissolved in DCM (10 mL) and TFA (1 mL) was added. The reaction mixture was allowed to stir at rt for 4 h. The mixture was concentrated and the residue was purified by column chromatography to give N-{4-[4-(6-aminopyridin-2-yl)-3-(2-chlorophenyl)-1H-pyrazol-1-yl]-5-methylpyridin-2-yl}acetamide (1-107) (0.035 g, 20%). LCMS (FA): m/z=419.4 (M+H).

### Example 25

### Synthesis of Intermediate Heterocycles

### Methyl 4-(2-chlorophenyl)-1H-pyrazole-3-carboxylate

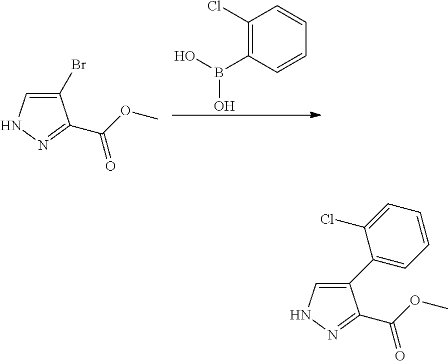

A mixture of methyl 4-bromo-1H-pyrazole-3-carboxylate (0.10 g, 0.50 mmol), (2-chlorophenyl)boronic acid (0.39 g, 2.5 mmol), tetrakis(triphenylphosphine)palladium(0) (0.056 g, 0.049 mmol) and cesium carbonate (0.54 g, 1.6 mmol) in 1,4-dioxane (2.6 mL) and water (0.6 mL) was sealed in a vial and subjected to microwave irradiation for 40 min at 150° C. The reaction mixture was diluted with water and extracted with DCM. The organic layer was separated, washed with water, dried over Na₂SO₄, filtered and concentrated. The residue was purified by column chromatography to give methyl 4-(2-chlorophenyl)-1H-pyrazole-3-carboxylate (0.064 g, 60%). LCMS (FA): m/z=236.9 (M+H).

### Example 26

### Synthesis of N-{4-[4-(2-chlorophenyl)-3-(1H-1,2,4-triazol-3-yl)-1H-pyrazol-1-yl]pyridin-2-yl}acetamide (I-1)

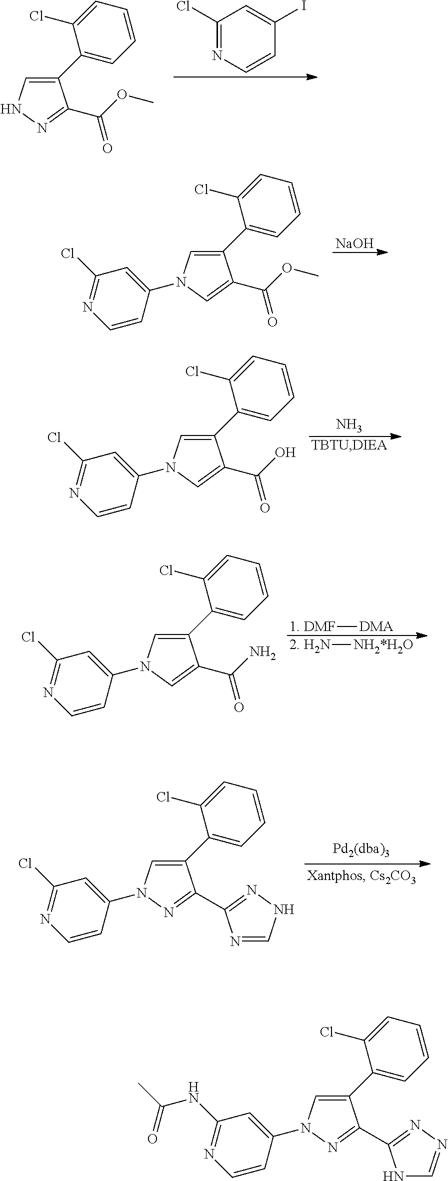

### Step 1: methyl 4-(2-chlorophenyl)-1-(2-chloropyridin-4-yl)-1H-pyrazole-3-carboxylate

To a mixture of copper(I) iodide (0.073 g, 0.39 mmol) in 1,4-dioxane (15 mL) were added 2-chloro-4-iodopyridine (2.77 g, 11.6 mmol), methyl 4-(2-chlorophenyl)-1H-pyrazole-3-carboxylate (0.46 g, 1.9 mmol), and trans-1,2-bis(methylamino)cyclohexane (0.12 mL, 0.77 mmol). The reaction mixture was allowed to stir at 100° C. for 1 h and then potassium carbonate (1.60 g, 11.6 mmol) was added. The reaction mixture was allowed to stir at 105° C. for 4 h and then filtered through celite. The filter cake was washed with EtOAc and the organic solutions were collected, washed with concentrated NH₄OH and brine, and then concentrated. The residue was purified by column chromatography to give methyl 4-(2-chlorophenyl)-1-(2-chloropyridin-4-yl)-1H-pyrazole-3-carboxylate (0.54 g, 80%). LCMS (FA): m/z=348.3 (M+H).

### Step 2: 4-(2-chlorophenyl)-1-(2-chloropyridin-4-yl)-1H-pyrazole-3-carboxylic acid

Methyl 4-(2-chlorophenyl)-1-(2-chloropyridin-4-yl)-1H-pyrazole-3-carb oxylate (0.069 g, 0.20 mmol) and sodium hydroxide (0.027 g, 0.68 mmol) were added to a mixture of THF (3 mL) and water (5 mL). The reaction mixture was allowed to stir at rt overnight and then concentrated to give 4-(2-chlorophenyl)-1-(2-chloropyridin-4-yl)-1H-pyrazole-3-carboxylic acid (0.060 g, 90%), which was used in the next step without purification. LCMS (FA): m/z=334.3 (M+H).

### Step 3: 4-(2-chlorophenyl)-1-(2-chloropyridin-4-yl)-1H-pyrazole-3-carboxamide

A mixture of 4-(2-chlorophenyl)-1-(2-chloropyridin-4-yl)-1H-pyrazole-3-carboxylic acid (0.52 g, 1.6 mmol), TBTU (1.00 g, 3.11 mmol), DIEA (2.7 mL, 15.6 mmol) and ammonia (0.5 M in 1,4-dioxane, 25 mL) in DCM (40 mL) was allowed to stir at rt overnight. The reaction mixture was diluted with water and extracted with DCM. The organic solutions were combined, washed with water, dried over Na₂SO₄, filtered and concentrated. The residue was purified by column chromatography to give 4-(2-chlorophenyl)-1-(2-chloropyridin-4-yl)-1H-pyrazole-3-carboxamide (0.40 g, 77%). LCMS (FA): m/z=333.3 (M+H).

### Step 4: 2-chloro-4-[4-(2-chlorophenyl)-3-(1H-1,2,4-triazol-3-yl)-1H-pyrazol-1-yl]pyridine

To a suspension of 4-(2-chlorophenyl)-1-(2-chloropyridin-4-yl)-1H-pyrazole-3-carboxamide (0.40 g, 1.20 mmol) in anhydrous toluene (6.5 mL) was added DMF-DMA (0.47 mL, 3.6 mmol). The reaction mixture was allowed to stir at 50° C. for 2 h and then allowed to cool to rt. The mixture was concentrated and the residue was dissolved in AcOH (4.7 mL) and hydrazine hydrate (0.29 mL, 5.9 mmol) was added. The reaction mixture was allowed to stir at rt for 1 h and then concentrated. The mixture was azeotroped two times with toluene. The residue was diluted with EtOAc and washed with aqueous saturated sodium bicarbonate. The organic solution was dried over Na₂SO₄, filtered and concentrated. The residue was purified by column chromatography to give 2-chloro-4-[4-(2-chlorophenyl)-3-(1H-1,2,4-triazol-3-yl)-1H-pyrazol-1-yl]pyridine (0.086 g, 20%). LCMS (FA): m/z=357.3 (M+H).

### Step 5: N-{4-[4-(2-chlorophenyl)-3-(1H-1,2,4-triazol-3-yl)-1H-pyrazol-1-yl]pyridin-2-yl}acetamide

To a solution of 2-chloro-4-[4-(2-chlorophenyl)-3-(1H-1,2,4-triazol-3-yl)-1H-pyrazol-1-yl]pyridine (0.046 g, 0.13 mmol) in 1,4-dioxane (1.5 mL) were added acetamide (0.11 g, 1.9 mmol), tris(dibenzylideneacetone)dipalladium(0) (0.017 g, 0.018 mmol), Xantphos (0.031 g, 0.054 mmol) and cesium carbonate (0.21 g, 0.64 mmol). The reaction mixture was sealed in a vial and subjected to microwave irradiation at 150° C. for 60 min. The reaction mixture was diluted with water and extracted with EtOAc. The organic solutions were combined, washed with water, dried over Na₂SO₄, filtered and concentrated. The residue was purified by column chromatography to give N-{4-[4-(2-chlorophenyl)-3-(1H-1,2,4-triazol-3-yl)-1H-pyrazol-1-yl]pyridin-2-yl}acetamide (0.012 g, 24%). LCMS (FA): m/z=380.6 (M+H).

Compounds in the following table were prepared from the appropriate starting materials using the procedures described above:

| | |
|---|---|
| I-20 | LCMS (AA): m/z = 356.3 (M + H). |

Biological Data:

VPS34 Enzyme Assays

Cloning, Expression, and Purification of VPS34

VPS34 (accession number GB:BCO33004) was cloned into pDEST20-Thombin as N-terminal GST tagged fusion proteins using the Gateway system (Invitrogen, catalog#11804-013). The sequences were verified before recombinant protein expression using the Baculovirus Expression System with Gateway® Technology.

For expression VPS34 was infected at 1MOI in SF9 cells and harvested 72 hours post infection.

For purification, VPS34 is purified by Glutathione Sepharose 4 Fast Flow (GE Healthcare #17-5132-03) followed by HiTrap Q (GE Heₐₗthcare #17-1153-01).

VPS34 Assay Conditions

Human VPS34 Enzyme Assay Method

100 nL compounds in DMSO are added to wells of a 384 well microtitre plate (Greiner 780076). At room temperature: 5 ul VPS34 reaction buffer (Invitrogen Assay Buffer Q (diluted 1 in 5 with nanopure water) plus 2 mM DTT and 2 mM MnCl₂) containing ATP (20 uM, Promega) and 200 uM PI-PS substrate (Invitrogen PV5122) is added followed immediately by 5 ul VPS34 reaction buffer (as above) containing VPS34 (5 nM, Millennium Protein Sciences Group) and the mixture is incubated with shaking at room temperature for 1 hour. Then 5 ul VPS34 stop-detect mix (as per Invitrogen Adapta Assay kit (PV5009) instructions (contains kinase quench buffer, TR-FRET buffer, Adapta Eu anti-ADP antibody and Alexa Fluor 647 ADP tracer)) is added to quench the reaction. The plates are then incubated for 30 minutes at room temperature with shaking and then read on a BMG PheraStar Plus reader.

For the assay methods described above, test compound percent inhibition, at various concentrations, is calculated relative to control (DMSO and EDTA) treated samples. Compound concentration versus percent inhibition curves are fitted to generate IC₅₀ values. One skilled in the art will appreciate that the values generated either as percentage inhibition at a single concentration or IC₅₀ values are subject to experimental variation.

Vps34 Cell Assays

1) FYVE Domain Redistribution Assay

The FYVE domain redistribution assay monitors translocation of EGFP-2×FYVE from its initial location bound to (PtdIns(3)P) in early endosomes to the cytoplasm in response to test compounds. Recombinant U2OS cells stable expressing the FYVE finger from the human homologue of the hepatocyte growth factor-regulated tyrosine kinase substrate Hrs, duplicated in tandem (GenBank Acc. NM_{—}004712) and fused to the C-terminus of enhanced green fluorescent protein (EGFP). U2OS cells are adherent epithelial cells derived from human osteosarcoma. Expression of EGFP-2X-FYVE is controlled by a standard CMV promoter and continous expression is maintained by addition of geneticin to the culture medium. Localization of the fusion protein within the cells is imaged on the Evotec Technologies OPERA Confocal Imager and Integrated Spot Signal Per Cellular Signal is quantified using Acapella software. Using this information, IC50 values for inhibitors can be determined

U2OS EGFP-2XFYVE cells are propagated in Dulbecco's Modified Eagle Media High glucose (D-MEM) (Invitrogen cat. 11995) containing 10% Fetal Bovine Serum (HyClone cat. SH30071.02) and 0.5 mg/ml Geneticin (Invitrogen) and kept in a humidified chamber at 37° C. with 5% CO₂. 8×10³ cells are cultured in 100 μl of media per well in tissue culture-treated black-walled, clear bottom Optilux 96-well plates (BD Biosciences) for 16-24 hours.

Prior to addition of compounds, cell media is removed and replaced with 75 μl of fresh media. Test compounds in DMSO are diluted 1:100 in media. The diluted test compounds are added to the cells (25 μl per well) in 3-fold dilutions with a final concentration range of 0.0015 to 10 μM. The cells are incubated for 30 minutes in a humidified chamber at 37° C. with 5% CO₂ Immediately following compound incubation, all liquid is removed from the wells and cells are fixed with 4% paraformaldehyde in PBS (75 μl per well) for 15 minutes at room temperature. The paraformaldehyde solution is removed from wells and washed once with PBS (100 μl per well). The PBS is removed and cells are incubated with DRAQ5 Nuclear Dye (Alexis/Biostatus) (85 μl per well). The plates are covered with Flash Plate plastic adhesive foil and imaged on the Evotec Technologies OPERA Confocal Imager Opera after at least a 30 minute incubation. Concentration curves are generated by calculating the Integrated Spot Intensity Per Cellular Signal decrease in test-compound treated samples relative to DMSO-treated controls and a 100% control inhibitor, and percentage inhibition values at a single concentration or growth inhibition (IC₅₀) values are determined from the curves. One skilled in the art will appreciate that the values generated either as percentage inhibition at a single concentration or IC₅₀ values are subject to experimental variation.

PI3K Enzyme Assays

Cloning, Expression, and Purification of PI3Ks

The catalytic subunits of PI3Ks are cloned into either pDEST8(p110 alpha) or pDEST10(p110beta, p110delta, and p110gamma) as N-terminal His tagged fusion proteins using the Gateway system (Invitrogen, catalog#11804-010 for pDEST8 and 11806-015 for pDEST10). The sequences are verified before recombinant protein expression using the Baculovirus Expression System with Gateway® Technology. The accession numbers for the subunits are as follows:

p110 alpha (GB:U79143)
p110beta (GB:S67334)
p110delta (GB: U86453)
p110gamma (GB: X83368)

The regulatory subunits of PI3Ks are cloned into pDEST8 as un-tagged protein using the Gateway system (Catalog#11804-010). The sequences are verified before recombinant protein expression using the Baculovirus Expression System with Gateway® Technology. The accession numbers for the subunits are as following:

p85 alpha (GB: BC030815)
p101(GB: AB028925)
VPS34 is cloned into pDEST20-Thombin as N-terminal GST tagged fusion proteins using the Gateway system (Invitrogen, catalog#11804-013). The sequences are verified before recombinant protein expression using the Baculovirus Expression System with Gateway® Technology.

For expression of the p110 complexes, the p85 (MOI of 4) is co-infected with p110 alpha, beta, and delta respectively (1MOI) in SF9 cells and harvested at 60 hours post co-infection. P110 gamma was infected at 1 MOI and harvested at 60 hours post infection.

For purification, PI3Ks are purified by Ni-NTA Agarose (Qiagen #30250) followed by Mono Q 10/100 GL (Ge Healthcare #17-5167-01). VPS34 is purified by Glutathione Sepharose 4 Fast Flow (GE Healthcare #17-5132-03) followed by HiTrap Q (GE Healthcare #17-1153-01).

PI3K Assay Conditions

1) Human PI3Kα Enzyme Assay Method

0.5 uL compounds in DMSO are added to wells of a 384 well microtitre plate (Corning 3575). At room temperature: 10 ul PI3K reaction buffer (50 mM Hepes, 5 mM DTT, 150 mM NaCl, 10 mM beta-glycerophosphate, 10 mM MgCl2, 0.25 mM sodium cholate and 0.001% CHAPS, pH 7.00) containing ATP (25 uM, Promega) is added followed immediately by 10 ul PI3K reaction buffer containing di-C8 PI(4,5)P2 (3.5 uM, CellSignals) and PI3Kalpha (0.4875 nM, Millennium Protein Sciences Group) and the mixture is incubated with shaking at room temperature for 30 minutes. Then 5 ul PI3K stop mix (50 mM Hepes, 5 mM DTT, 150 mM NaCl, 0.01% Tween-20, 15 mM EDTA and 25 nM biotin-PI(3,4,5)P3 (Echelon) is added to quench the reaction followed immediately by addition of 5 ul HTRF detection mix (50 mM Hepes, 5 mM DTT, 150 mM NaCl, 0.01% Tween-20, 40 mM KF, 10 nM GST:GRP-1 PH domain (Millennium Protein Sciences Group), 15 nM Streptavidin-XL (CisBio) and 0.375 nM anti-GST Eu++ antibody (CisBio) at pH 7.00). The plates are then incubated for 1 hour at room temperature with shaking and then read on a BMG PheraStar Plus reader.

2) Human PI3K beta, delta and gamma isoforms are tested using the procedure described for PI3K alpha above but with the following changes: PI3K beta (5.25 nM), PI3K delta (0.75 nM) and PI3K gamma (5 nM). All isoforms supplied by Millennium Protein Science Group.

3) VPS34 is assayed using Adapta™ Universal Kinase Assay Kit (Invitrogen).

For the assay methods described above, test compound percent inhibition, at various concentrations, is calculated relative to control (DMSO and EDTA) treated samples. Compound concentration versus percent inhibition curves are fitted to generate IC₅₀ values. One skilled in the art will appreciate that the values generated either as percentage inhibition at a single concentration or IC₅₀ values are subject to experimental variation.

PI3K Cell Assays

1) In-Cell Western Assay

The pSer473 AKT LI-COR In-Cell Western Assay is a quantitative immunofluorescent assay that measures phosphorylation of serine 473 AKT (pSer473 AKT) in WM266.4 and SKOV3 tumor cell lines grown in cell culture.

WM266.4 cells are propagated in Minimum Essential Media (MEM) (Invitrogen) containing L-glutamine, 10% Fetal Bovine Serum, 1 mM MEM Sodium Pyruvate, and 0.1 mM MEM Non-Essential Amino Acids and SKOV3 cells are propagated in McCoy's 5A Media (modified) (Invitrogen) containing L-Glutamine and 10% Fetal Bovine Serum. Both cell lines are kept in a humidified chamber at 37° C. with 5% CO₂. For the pSer473 AKT LI-COR In-Cell Western Assay, 1.5×10⁴ WM266.4 and 1.5×10⁴ SKOV3 cells are cultured in 100 μl of media per well in tissue culture-treated black-walled, clear bottom Optilux 96-well plates (BD Biosciences) for 16-20 hours. Prior to addition of compounds, cell media is removed and replaced with 75 μl of fresh media. Test compounds in DMSO are diluted 1:100 in media. The diluted test compounds are added to the cells (25 μl per well) in 3-fold dilutions with a final concentration range of 0.0015 to 10 μM. The cells are incubated for 2 hours in a humidified chamber at 37° C. with 5% CO₂. Immediately following compound incubation, all liquid is removed from the wells and cells are fixed with 4% paraformaldehyde in PBS (150 μl per well) for 20 minutes at room temperature. The paraformaldehyde solution is removed from wells and the cells are permeabilized with 200 μl 0.1% Triton X-100 in PBS per well for 10 min×3 at room temperature. After removal of PBS+0.1% Triton X-100, 150 μl Odyssey blocking buffer (LI-COR Biosciences) is added to each well and plates are incubated at room temperature for 1.5 h. Blocking buffer is removed from the wells and primary antibodies (Phospho-AKT (Ser473) (D9E) XP™ Rabbit mAb and AKT (pan) (40D4) Mouse mAb, Cell Signaling Technology) diluted in Odyssey blocking buffer are added (50 μl per well). Plates are incubated at 4° C. overnight. The cells are washed for 20 min×3 with PBS+0.1% Tween-20 (200 μl per well). Secondary antibodies (IRDye 680 Goat anti-Rabbit IgG (H+ L) and IRDye 800CW Goat anti-Mouse IgG (H+L), LI-COR Biosciences) are diluted in Odyssey blocking buffer and added to wells (50 μl per well) followed by a 1 h incubation at room temperature, protected from light. Cells are washed for 20 min×3 with PBS+0.1% Tween-20 (200 μl per well). Wash buffer is completely removed from wells after last wash, plates are protected from light until scanned and analyzed with the Odyssey Infrared Imaging System (LI-COR Biosciences). Both pS473 AKT and AKT are simultaneously visualized with the 680 nm fluorophore indicated by a red color and the 800 nm fluorophore indicated by a green color. Relative fluorescence units derived from the scans allow for quantitative analyses of both labeled proteins and the ratio of pS473 AKT to AKT is calculated. Concentration response curves are generated by plotting the average ratios of PI3K inhibitor-treated samples relative to DMSO-treated controls to determine percent change in expression of pS473 AKT, and percentage inhibition values at a single concentration or growth inhibition (IC₅₀) values are determined from those curves. One skilled in the art will appreciate that the values generated either as percentage inhibition at a single concentration or IC₅₀ values are subject to experimental variation.

In some embodiments, compounds of the invention inhibit VPS34 at a 1.11 μM concentration with the percent inhibition as shown in the table below. In certain embodiments, compounds of the invention inhibit VPS34 with the IC₅₀ values shown in the table below. In certain embodiments, compounds of the invention that inhibit VPS34 have an IC₅₀ value A) less than 50 nM. In certain embodiments, compounds of the invention inhibit VPS34 have an IC₅₀ value B) 50 nM-less than 150 nM. C) 150 nM-less than 1 uM, D) 1 uM to 5 uM.

| | VPS34 | |
|---|---|---|
| | Percent | |
| Compound | Inhibition | IC₅₀ |
| I-23 | >99 | A |
| I-2 | >99 | A |
| I-7 | 88 | C |
| I-6 | 97 | C |
| I-70 | 20 | D |
| I-22 | >99 | A |
| I-4 | 18 | D |
| I-18 | 89 | C |
| I-5 | >99 | A |
| I-42 | >99 | A |
| I-55 | >99 | A |
| I-27 | >99 | A |
| I-35 | >99 | A |
| I-26 | 78 | C |
| I-33 | 42 | D |
| I-28 | >99 | A |
| I-40 | >99 | A |
| I-44 | >99 | A |
| I-38 | >99 | B |
| I-47 | 64 | C |
| I-41 | 78 | B |
| I-37 | 88 | C |
| I-34 | 92 | C |
| I-32 | >99 | A |
| I-29 | 44 | D |
| I-64 | >99 | A |
| I-69 | >99 | B |
| I-62 | 59 | C |
| I-57 | 94 | C |
| I-59 | 75 | C |
| I-65 | >99 | B |
| I-77 | 79 | B |
| I-84 | >99 | A |
| I-71 | >99 | C |
| I-75 | 15 | D |
| I-82 | >99 | B |
| I-17 | >99 | A |
| I-50 | >99 | A |
| I-106 | 54 | C |
| I-107 | 54 | C |
| I-20 | 29 | D |
| I-63 | 62 | C |
| I-11 | 96 | C |
| I-12 | >99 | A |
| I-9 | >99 | C |
| I-8 | >99 | B |
| I-13 | >99 | B |
| I-54 | >99 | B |
| I-49 | 56 | C |
| I-52 | >99 | B |
| I-53 | 81 | C |
| I-48 | >99 | B |
| I-56 | >99 | A |
| I-39 | >99 | A |
| I-31 | >99 | A |
| I-45 | >99 | B |
| I-51 | 85 | B |
| I-46 | >99 | A |
| I-25 | >99 | A |
| I-43 | 27 | D |
| I-30 | 14 | D |
| I-68 | >99 | B |
| I-58 | >99 | B |
| I-61 | >99 | B |
| I-66 | 89 | C |
| I-67 | >99 | B |
| I-60 | >99 | A |
| I-80 | >99 | B |
| I-85 | 93 | C |
| I-76 | 23 | D |
| I-79 | >99 | B |
| I-73 | >99 | B |
| I-83 | >99 | B |
| I-78 | >99 | A |
| I-81 | >99 | A |
| I-74 | >99 | B |
| I-72 | 71 | C |
| I-36 | >99 | A |
| I-108 | 10 | D |
| I-109 | 97 | A |
| I-1 | 98 | C |

IC₅₀: A) less than 50 nM; B) 50 nM-less than 150 uM, C) 150 nM-less than 1 uM, and D) 1 uM-5 uM

While we have described a number of embodiments of this invention, it is apparent that our basic examples may be altered to provide other embodiments, which utilize the compounds and methods of this invention. Therefore, it will be appreciated that the scope of this invention is to be defined by the appended claims rather than by the specific embodiments, which have been represented by way of example.

1. A compound of formula ID: 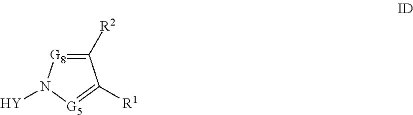
or a pharmaceutically acceptable salt thereof are provided, wherein:
both of G₅ and G₈ are CR³, or one of G₅ and G₈ is N and the other is CR³;
when one of G₅ or G₈ is N, R³ is hydrogen, —CN, halogen, —Z—R⁵, or an optionally substituted group selected from C₁₋₆ aliphatic and 3- to 10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
R^{3a} is hydrogen or an optionally substituted C₁₋₄ aliphatic, and
R⁵ is hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
when G₅ and G₈ are both CR³, each occurrence of R³ is independently hydrogen, CN, or an optionally substituted C₁₋₃ aliphatic;
R¹ is —CN, —C(O)N(R⁴)₂, —C(O)OR⁴, —C(NR⁴)N(R⁴)₂, —NHCOR⁴, —NHSO₂R⁴, —NHCON(R⁴)₂, —NHCOOR⁴, —NHSO₂N(R⁴)₂, —CH₂OR⁴, —CH₂N(R⁴)₂, —CH₂NHC(O)R⁴, —SO₂NR⁴₂, —CONHC(═NH)N(R⁴)₂, —NHSO₂OR⁴, or CY, wherein CY is an optionally substituted group selected from a 3- to −7-membered cycloaliphatic; a 4- to 10-membered heterocyclyl having 1-5 heteroatoms independently selected from nitrogen, oxygen, or sulfur; a 6-10 membered aryl, or 5- to 10-membered heteroaryl having 1-5 heteroatoms independently selected from nitrogen, oxygen, or sulfur; wherein:
each R⁴ is independently selected from hydrogen, —OH, or an optionally substituted group selected from C₁₋₆ aliphatic, 3- to 10-membered cycloaliphatic, 6- to 10-membered aryl, or 5- to 10-membered heteroaryl having 1-5 heteroatoms independently selected from nitrogen, oxygen, or sulfur; or
R⁴ is —Z₂—R⁶ wherein:
Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
R^{4a} is hydrogen or an optionally substituted C₁₋₄ aliphatic, and
R⁶ is hydrogen, —NH₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur; or
two occurrences of R⁴, taken together with a nitrogen atom to which they are bound, form an optionally substituted 4- to −7-membered heterocyclyl ring having 0-1 additional heteroatoms independently selected from nitrogen, oxygen, or sulfur;
R² is hydrogen, halo or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, wherein R² is optionally substituted with 1-4 occurrences of R^{2a}, wherein each occurrence of R^{a} is independently —R^{12a}, -T₂-R^{12d}, -T₂-R^{12a}, or —V₂-T₂-R^{12d}, and:
each occurrence of R^{12a} is independently halogen, —CN, —NO₂, —R^{12c}, —N(R¹²)₂, —OR^{12b}, —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})SO₂R^{12c}, —N(R^{12e})C(O)OR^{12b}, —N(R^{12e})C(O)N(R¹²ⁿ)₂, or —N(R^{12e})SO₂N(R^{12b})₂, or an optionally substituted C₁-C₆ aliphatic or C₁-C₆haloaliphatic;
each occurrence of R^{12b} is independently hydrogen or an optionally substituted group selected from C₁-C₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or two occurrences of R^{12b}, taken together with a nitrogen atom to which they are bound, form an optionally substituted 4- to −7-membered heterocyclyl ring having 0-1 additional heteroatoms selected from nitrogen, oxygen, or sulfur;
each occurrence of R^{12c} is independently hydrogen or an optionally substituted group selected from C₁-C₆ aliphatic, C₁-C₆ haloaliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12d} is independently hydrogen or an optionally substituted group selected from 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, N(R^{12e})C(O)—, —N(R^{12e})SO₂N(R^{12e})C(O)O—, —N(R^{12e})C(O)N(R^{12e})—, N(R^{12e})SO₂N(R^{12e})—, —OC(O)—, or —C(O)N(R^{12e})—O—; and
T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3- to −7 membered cycloaliphatic or heterocyclyl ring, wherein R¹³ is hydrogen or an optionally substituted C₁₋₄ aliphatic group; and
HY is a group selected from: 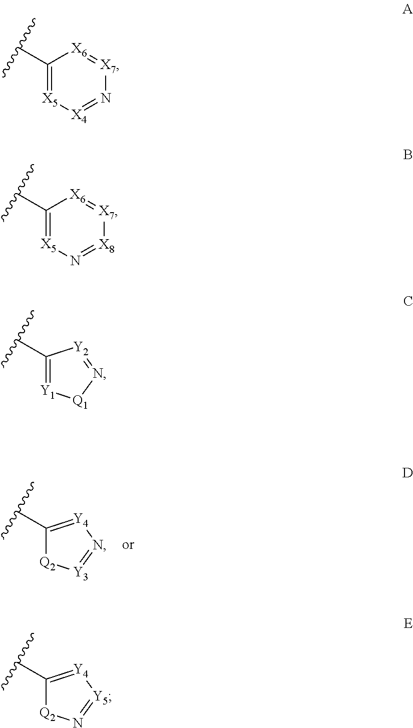
wherein
each occurrence of X₄, X₅, X₆, X₇, and X₈ is independently —CR¹⁰, —CR^{10′} or N, provided no more than two occurrences of X₄, X₅, X₆, X₇, and X₈ is N;
each occurrence of Y₁, Y₂, Y₃, Y₄, and Y₅ is —CR¹⁰;
each occurrence of Q₁ and Q₂ is independently S, O or —NR⁹;
two adjacent occurrences of X₄ and X₅, X₆ and X₇, X₇ and X₈, Y₁ and —NR⁹, Y₃ and —NR⁹, or Y₄ and Y₅, may be taken together with the atom to which they are bound, to form an unsubstituted fused group having 8 to 10 ring atoms selected from an aryl group, or a heteroaryl group having 1-5 heteroatoms independently selected from nitrogen, oxygen, or sulfur;
each occurrence of R¹⁰ or R^{10′} is independently —R^{10b}, —V₁—R^{10c}, -T₁-R^{10b}, or —V₁-T₁-R^{10b}, wherein:
V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
each occurrence of R^{10a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, —N(R^{10a})C(O)N(R^{10a})—, —N(R^{10a})S(O)₂N(R^{10a})—, —OC(O)—, or —C(O)N(R¹¹)—O— or wherein T₁ forms part of an optionally substituted 3- to −7 membered cycloaliphatic or heterocyclyl ring;
each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{10c} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or
R^{10a} and R^{10b}, taken together with a nitrogen atom to which they are bound, form an optionally substituted 4- to −7-membered heterocyclyl ring having 0-1 additional heteroatoms independently selected from nitrogen, oxygen, or sulfur;
each occurrence of R¹¹ is independently hydrogen, —C(O)R^{11a}, —CO₂R^{11a}, —C(O)N(R^{11a})₂, —C(O)N(R^{11a})—OR^{11a}, —SO₂R^{11a}, —SO₂N(R^{11a})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
wherein each occurrence of R^{11a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
wherein each occurrence of R^{9a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
wherein each occurrence of R^{9b} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3- to 6-membered heterocyclyl having 1-5 heteroatoms independently selected from nitrogen, oxygen, or sulfur, or 5- to 10-membered heteroaryl having 1-5 heteroatoms independently selected from nitrogen, oxygen, or sulfur; and
provided that when HY is a non-fused group then HY is substituted with at least one occurrence of R¹⁰ or R^{10′}, wherein R¹⁰ or R^{10′} is:
—N(R¹¹)C(O)R^{10a}, —C(O)N(R¹¹)₂), r —NR¹¹C(O)OR^{10a}; or
—V₁-T₁-R^{10b}, wherein V₁ is —NR¹¹—, T₁ is a C₁-C₃ alkylene chain, and R^{10b} is an optionally substituted 6- to 10-membered aryl ring or a 5- to 10-membered heteroaryl ring having 1-5 heteroatoms independently selected from nitrogen, oxygen, or sulfur, or V₁ is —NR¹¹C(O)NR¹¹—, T₁ is a C₁-C₃ alkylene chain, and R^{10b} is —OR^{10a}; or
—V₁—R^{10c}, wherein V₁ is —NR¹¹—, and R^{10c} is a 5- to 10-membered heteroaryl ring having 1-5 heteroatoms independently selected from nitrogen, oxygen, or sulfur; and
provided that:
a) for compounds where -G₅-G₆-G₇-G₈-G₉ is —CR³═C—C═N—N or —CR³═C—C═CR³N:
(i) when G₈ is N, R² is methyl, and HY is 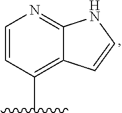 [SMILES: *c1ccnc2[nH]ccc12]
then R¹ is not an optionally substituted phenyl;
(ii) when G₈ is CH, then HY is not 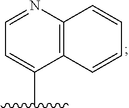 [SMILES: *c1ccnc2ccccc12]
(iii) when G₈ is CH, R² is hydrogen, and HY is 3-pyridyl, then HY is not substituted with 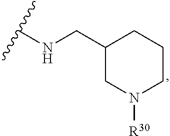
wherein R³⁰ is hydrogen, or —CO₂-tert-butyl;
(iv) provided that for compounds where G₈ is N and R² is hydrogen:
aa) when HY is 4-pyridyl, then R¹ is not —CO₂H;
bb) HY is not substituted with: 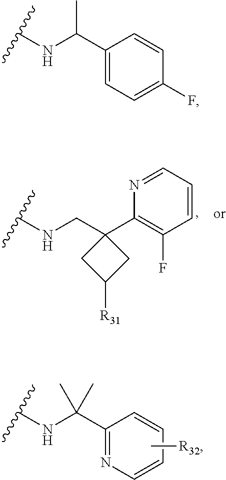
wherein R³¹ is hydrogen or fluoro;
R³² is fluoro, chloro, or —OCHF₂;
cc) when G₅ is C—R³, and R³ is —CH₃ or —NH₂, then R¹ is not —CO₂Et; and
b) for compounds where -G₅-G₆-G₇-G₈-G₉ is —N═C—C═CR³—N:
(i) when R³ is 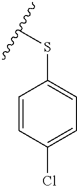 [SMILES: *Sc1ccc(Cl)cc1]
and R² is H, then R¹ is not 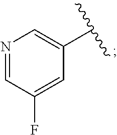 [SMILES: *c1cncc(F)c1]
(ii) when R² is methyl or hydrogen and R³ is hydrogen, then HY is not 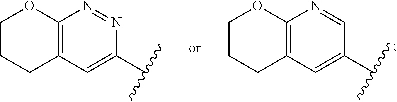 [SMILES: *c1cc2c(nn1)OCCC2 ; *c1cnc2c(c1)CCCO2]
(iii) when R² and R³ are both hydrogen then HY is not 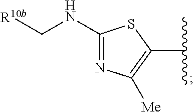
(iv) when R² is hydrogen and R³ is —CF₃, then R¹ is not optionally substituted 3-pyridinyl, 1,6-dihydro-6-oxo-3-pyridinyl,tetrahydro-2H-pyran-4-yl or thiazolyl;
(v) when R² is hydrogen and R³ is —CF₃ or —NH₂, then HY is not 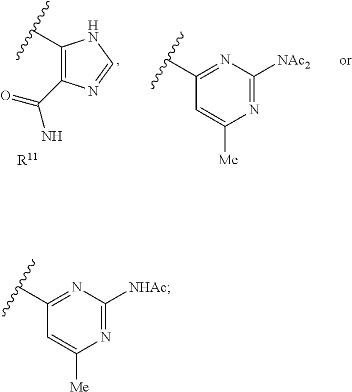
(vi) when R² and R³ are both hydrogen and HY is 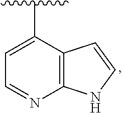 [SMILES: *c1ccnc2[nH]ccc12]
then R¹ is not an optionally substituted phenyl ring;
(vii) when R¹ is unsubstituted thiazolyl, then HY is not substituted with —CH₂CH₂OH or —CH₂CH₂OSiMe₂t-Bu;
(viii) when R³ is —SCH₃, and R² is hydrogen, then R¹ is not substituted phenyl;
(ix) when R¹ is —CO₂R⁴, R² is hydrogen, and HY is 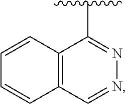 [SMILES: *c1nncc2ccccc12]
then R³ is not —CR¹⁰¹═CHR¹⁰² where R¹⁰¹ is hydrogen, methyl, or phenyl and R¹⁰² is an optionally substituted ring; and
c) provided that the compound is other than:

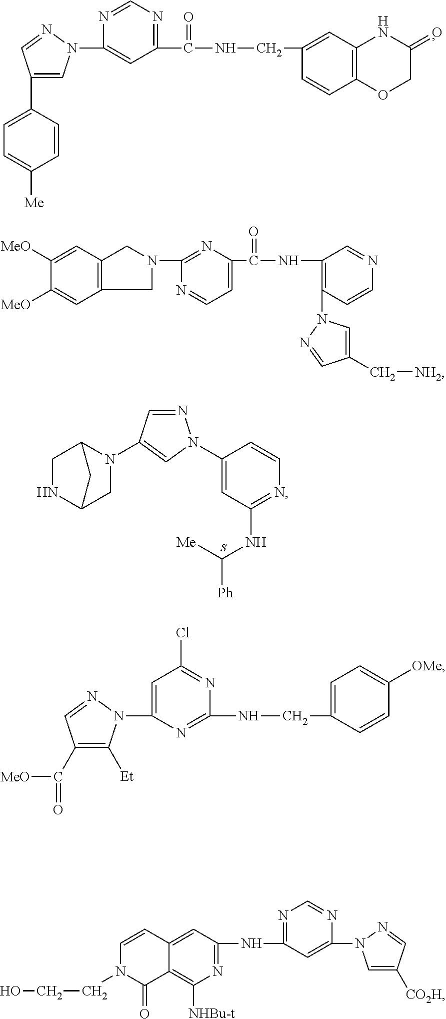

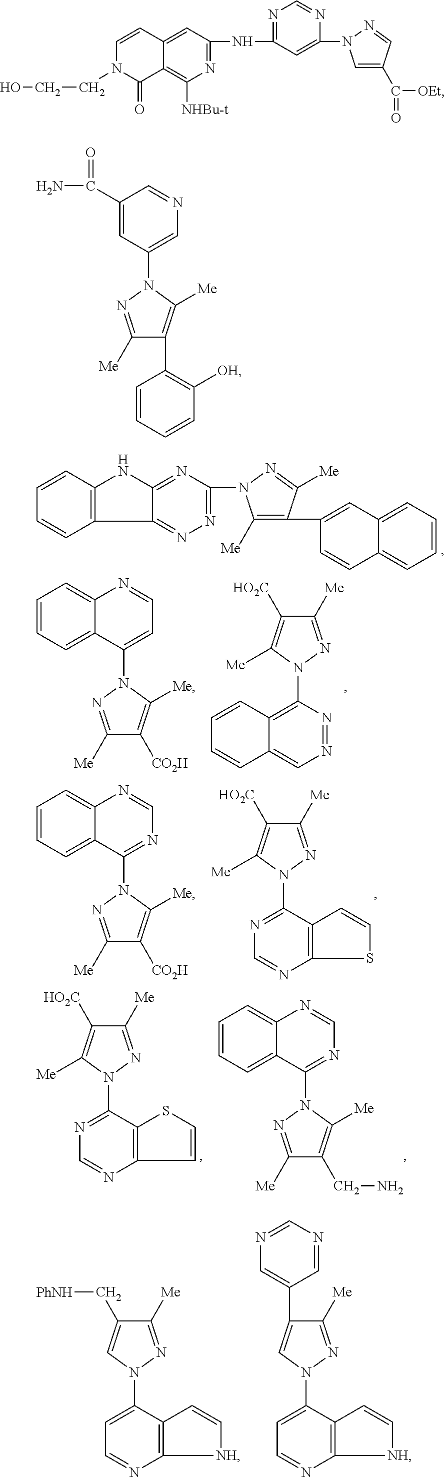

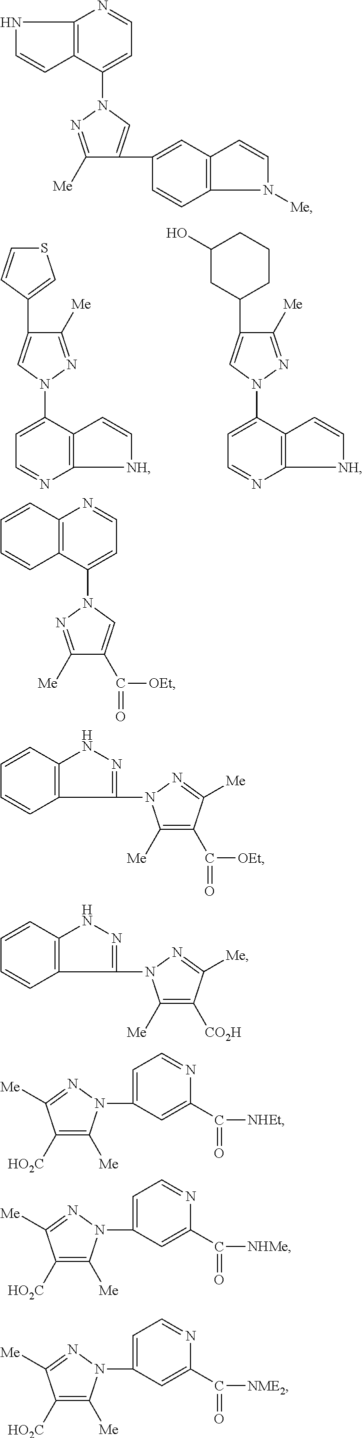

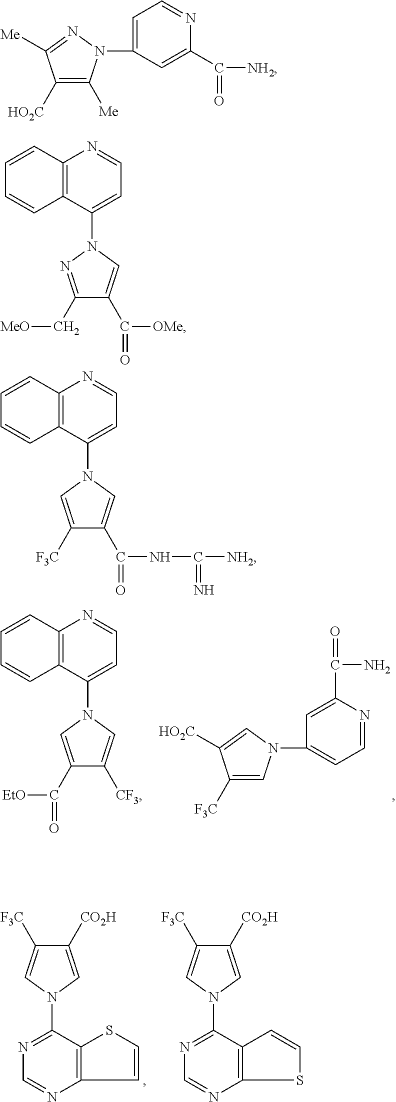

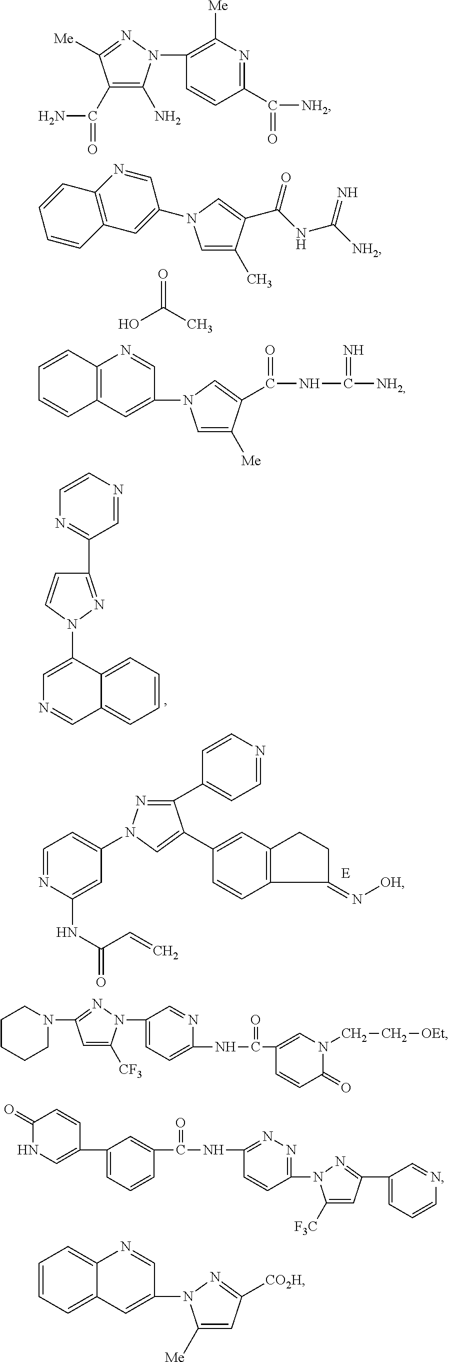

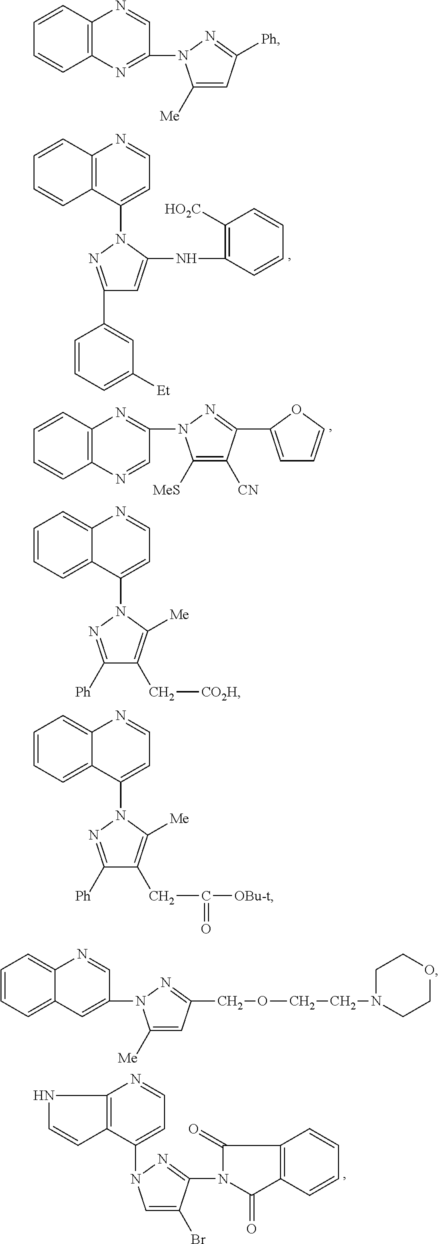

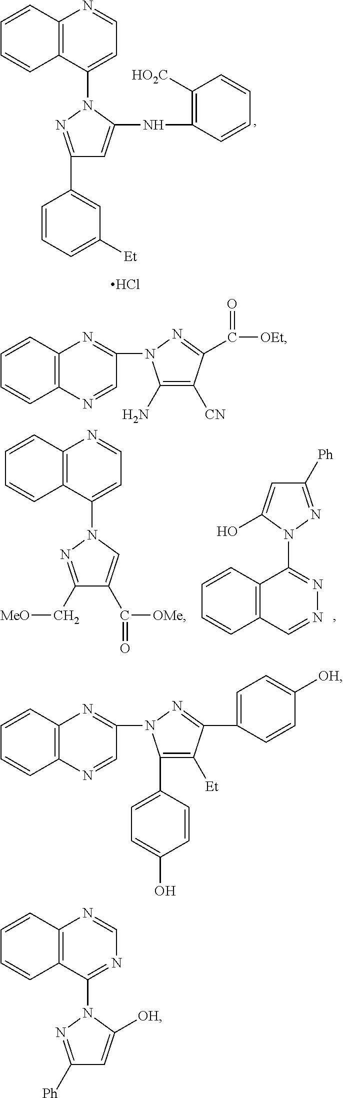

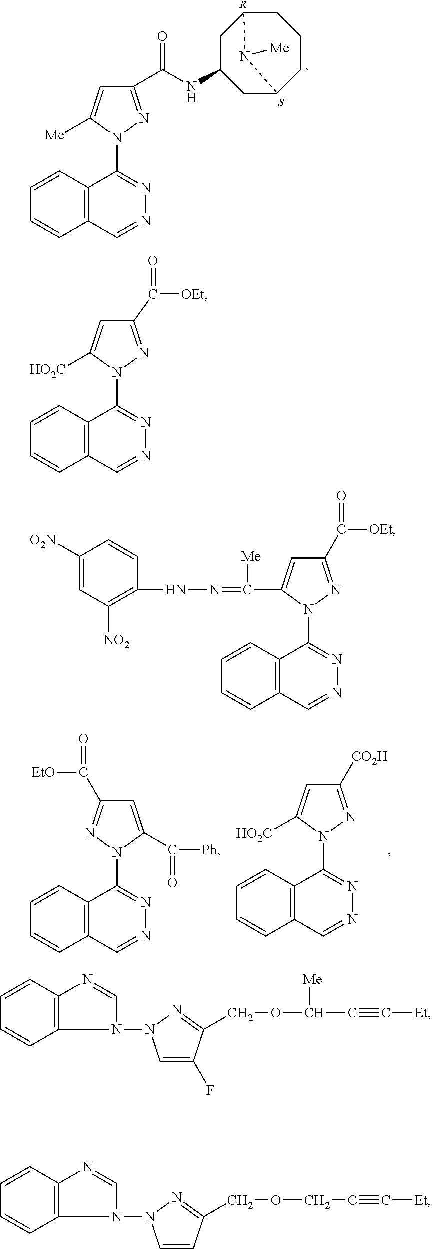

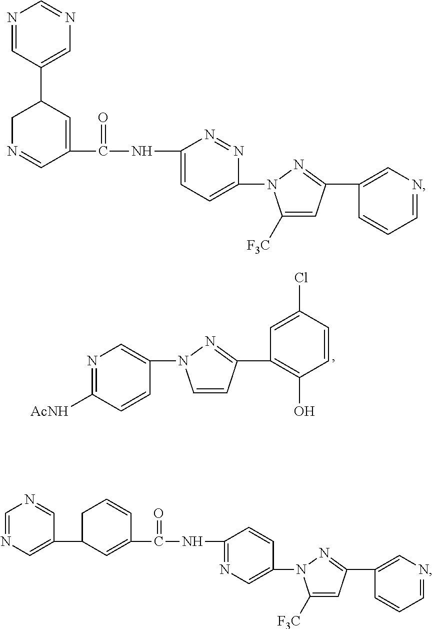

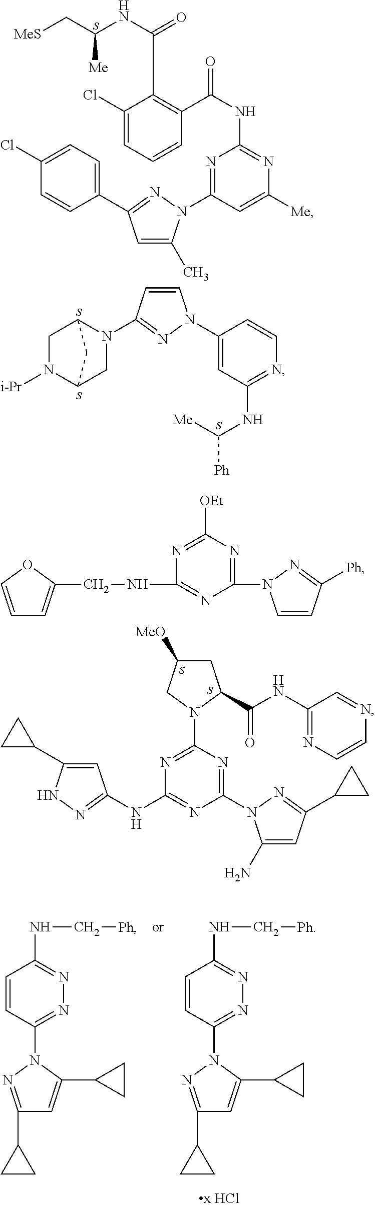

2. The compound of claim 1, wherein the comnound is of formula ID: 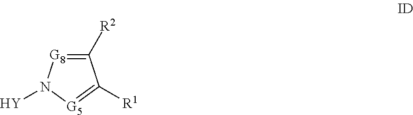
or a pharmaceutically acceptable salt thereof, wherein:
G₅ is CR³;
G₈ is N or CR³;
when G₈ is N, R³ is hydrogen, —CN, halogen, —Z—R⁵, or an optionally substituted group selected from C₁₋₆ aliphatic and 3- to 10-membered cycloaliphatic, wherein:
Z is selected from an optionally substituted C₁₋₃ alkylene chain, —O—, —N(R^{3a})—, —S—, —S(O)—, —S(O)₂—, —C(O)—, —CO₂—, —C(O)NR^{3a}—, —N(R^{3a})C(O)—, —N(R^{3a})CO₂—, —S(O)₂NR^{3a}—, —N(R^{3a})S(O)₂—, —OC(O)N(R^{3a})—, —N(R^{3a})C(O)NR^{3a}—, —N(R^{3a})S(O)₂N(R^{3a})—, or —OC(O)—;
R^{3a} is hydrogen or an optionally substituted C₁₋₄ aliphatic, and
R⁵ is hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
when G₈ is CR³, each occurrence of R³ is independently hydrogen, CN, or an optionally substituted C₁₋₃ aliphatic;
R¹ is —CN, —C(O)N(R⁴)₂, —C(O)OR⁴, —C(NR⁴)N(R⁴)₂, —NHCOR⁴, —NHSO₂R⁴, —NHCON(R⁴)₂, —NHCOOR⁴, —NHSO₂N(R⁴)₂, —CH₂OR⁴, —CH₂N(R⁴)₂, —CH₂NHC(O)R⁴, —SO₂NR⁴₂, —CONHC(═NH)N(R⁴)₂, —NHSO₂OR⁴, or CY, wherein CY is an optionally substituted group selected from a 3- to −7-membered cycloaliphatic; a 4- to 10-membered heterocyclyl having 1-5 heteroatoms independently selected from nitrogen, oxygen, or sulfur; a 6- to 10-membered aryl, or 5- to 10-membered heteroaryl having 1-5 heteroatoms independently selected from nitrogen, oxygen, or sulfur; wherein:
each R⁴ is independently selected from hydrogen, —OH, or an optionally substituted group selected from C₁₋₆ aliphatic, 3- to 10-membered cycloaliphatic, 6- to 10-membered aryl, or 5- to 10-membered heteroaryl having 1-5 heteroatoms independently selected from nitrogen, oxygen, or sulfur; or
R⁴ is —Z₂—R⁶ wherein:
Z₂ is selected from an optionally substituted C₁₋₃ alkylene chain, —S(O)—, —S(O)₂—, —C(O)—, —CO₂—, —C(O)NR^{4a}—, —C(NH)—, or —S(O)₂NR^{4a}—,
R^{4a} is hydrogen or an optionally substituted C₁₋₄ aliphatic, and
R⁶ is hydrogen, —NH₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur; or
two occurrences of R⁴, taken together with a nitrogen atom to which they are bound, form an optionally substituted 4- to −7-membered heterocyclyl ring having 0-1 additional heteroatoms independently selected from nitrogen, oxygen, or sulfur;
R² is halo or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, wherein R² is optionally substituted with 1-4 occurrences of R^{2a}, wherein each occurrence of R^{2a} is independently —R^{12a}, -T₂-R^{12d}, -T₂-R^{12a}, or —V₂-T₂-R^{12d}, and:
each occurrence of R^{12a} is independently halogen, —CN, —NO₂, —R^{12c}, —N(R^{12b})₂, —OR^{12b}), —SR^{12c}, —S(O)₂R^{12c}, —C(O)R^{12b}, —C(O)OR^{12b}, —C(O)N(R^{12b})₂, —S(O)₂N(R^{12b})₂, —OC(O)N(R^{12b})₂, —N(R^{12e})C(O)R^{12b}, —N(R^{12e})SO₂R^{12c}, —N(R^{12e})C(O)OR^{12b}, —N(R^{12e})C(O)N(R^{12b})₂, or —N(R^{12e})SO₂N(R^{12b})₂, or an optionally substituted C₁-C₆ aliphatic or C₁-C₆haloaliphatic;
each occurrence of R^{12b} is independently hydrogen or an optionally substituted group selected from C₁-C₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or two occurrences of R^{12b}, taken together with a nitrogen atom to which they are bound, form an optionally substituted 4- to −7-membered heterocyclyl ring having 0-1 additional heteroatoms selected from nitrogen, oxygen, or sulfur;
each occurrence of R^{12c} is independently hydrogen or an optionally substituted group selected from C₁-C₆ aliphatic, C₁-C₆ haloaliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12d} is independently hydrogen or an optionally substituted group selected from 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{12e} is independently hydrogen or an optionally substituted C₁₋₆ aliphatic group;
each occurrence of V₂ is independently —N(R^{12e})—, —O—, —S—, —S(O)—, —S(O)₂—, —C(O)—, —C(O)O—, —C(O)N(R^{12e})—, —S(O)₂N(R^{12e})—, —OC(O)N(R^{12e})—, N(R^{12e})C(O)—, —N(R^{12e})SO₂N(R^{12e})C(O)O—, —N(R^{12e})C(O)N(R^{12e})—, —N(R^{12e})SO₂N(R^{12e})—, —OC(O)—, or —C(O)N(R^{12e})—O—; and
T₂ is an optionally substituted C₁-C₆ alkylene chain wherein the alkylene chain optionally is interrupted by —N(R¹³)—, —O—, —S—, —S(O)—, —S(O)₂—, —C(O)—, —C(O)O—, —C(O)N(R¹³)—, —S(O)₂N(R¹³)—, —OC(O)N(R¹³)—, —N(R¹³)C(O)—, —N(R¹³)SO₂—, —N(R¹³)C(O)O—, —N(R¹³)C(O)N(R¹³)—, —N(R¹³)S(O)₂N(R¹³)—, —OC(O)—, or —C(O)N(R¹³)—O— or wherein T₂ or a portion thereof optionally forms part of an optionally substituted 3- to −7 membered cycloaliphatic or heterocyclyl ring, wherein R¹³ is hydrogen or an optionally substituted C₁₋₄ aliphatic group; and
HY is a group selected from: 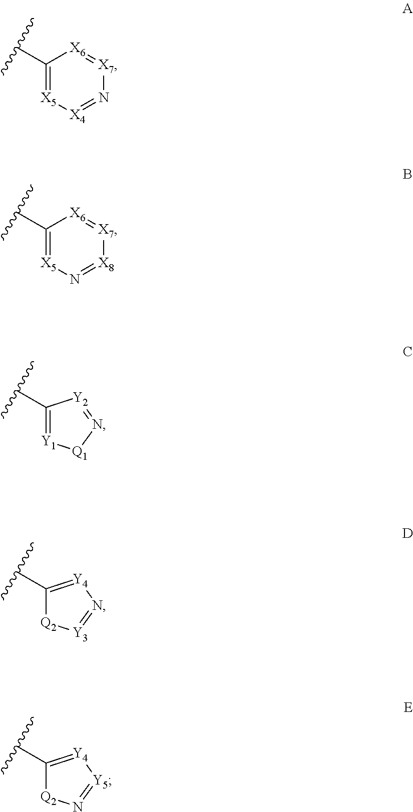
wherein
each occurrence of X₄, X₅, X₆, X₇, and X₈ is independently —CR¹⁰, —CR^{10′} or N, provided no more than two occurrences of X₄, X₅, X₆, X₇, and X₈ is N;
each occurrence of Y₁, Y₂, Y₃, Y₄, and Y₅ is —CR¹⁰;
each occurrence of Q₁ and Q₂ is independently S, O or —NR⁹;
two adjacent occurrences of X₄ and X₅, X₆ and X₇, X₇ and X₈, Y₁ and —NR⁹, Y₃ and —NR⁹, or Y₄ and Y₅, may be taken together with the atoms to which they are bound, to form an unsubstituted fused heteroaryl or heterocyclyl group having 8 to 10 ring atoms and having 1-5 heteroatoms independently selected from nitrogen, oxygen, or sulfur;
each occurrence of R¹⁰ or R^{10′} is independently —R^{10b}, —V₁—R^{10c}, -T₁-R^{10b}, or —V₁-T₁-R^{10b}, wherein:
V₁ is —NR¹¹—, —NR¹¹—C(O)—, —NR¹¹—C(S)—, —NR¹¹—C(NR¹¹)—, —NR¹¹C(O)O—, —NR¹¹C(O)NR¹¹—, —NR¹¹C(O)S—, —NR¹¹C(S)O—, —NR¹¹C(S)NR¹¹—, —NR¹¹C(S)S—, —NR¹¹C(NR¹¹)O—, —NR¹¹C(NR¹¹)NR¹¹—, —NR¹¹S(O)₂—, —NR¹¹S(O)₂NR¹¹—, —C(O)—, —CO₂—, —C(O)NR¹¹—, —C(O)NR¹¹O—, —SO₂—, or —SO₂NR¹¹—;
each occurrence of R^{10a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
T₁ is an optionally substituted C₁-C₆ alkylene chain wherein the alkylene chain optionally is interrupted by —N(R¹¹)—, —O—, —S—, —S(O)—, —S(O)₂—, —C(O)—, —C(O)O—, —C(O)N(R¹¹)—, —S(O)₂N(R¹¹)—, —OC(O)N(R¹¹)—, —N(R¹¹)C(O)—, —N(R¹¹)SO₂—, —N(R^{11a})C(O)O—, N(R^{10a})C(O)N(R^{10a}), —N(R^{10a})S(O)₂N(R^{10a})—, —OC(O)—, or —C(O)N(R¹¹)—O— or wherein T₁ forms part of an optionally substituted 3- to −7 membered cycloaliphatic or heterocyclyl ring;
each occurrence of R^{10b} is independently hydrogen, halogen, —CN, —NO₂, —N(R¹¹)₂, —OR^{10a}, —SR^{10a}, —S(O)₂R^{10a}, —C(O)R^{10a}, —C(O)OR^{10a}, —C(O)N(R¹¹)₂, —S(O)₂N(R¹¹)₂, —OC(O)N(R¹¹)₂, —N(R¹¹)C(O)R^{10a}, —N(R¹¹)SO₂R^{10a}, —N(R¹¹)C(O)OR^{10a}, —N(R¹¹)C(O)N(R¹¹)₂, or —N(R¹¹)SO₂N(R¹¹)₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R^{10c} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur, or
R^{10a} and R^{10b}, taken together with a nitrogen atom to which they are bound, form an optionally substituted 4- to −7-membered heterocyclyl ring having 0-1 additional heteroatoms independently selected from nitrogen, oxygen, or sulfur;
each occurrence of R¹¹ is independently hydrogen, —C(O)R^{11a}, —CO₂R^{11a}, —C(O)N(R^{11a})₂, —C(O)N(R^{11a})—OR¹¹, —SO₂R^{11a}, —SO₂N(R^{11a})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
wherein each occurrence of R^{11a} is independently hydrogen or an optionally substituted group selected from C₁₋₆aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteroatoms independently selected from nitrogen, oxygen, or sulfur;
each occurrence of R⁹ is independently hydrogen, —C(O)R^{9a}, —CO₂R^{9a}, —C(O)N(R^{9b})₂, —SO₂R^{9a}, —SO₂N(R^{9b})₂, or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
wherein each occurrence of R^{9a} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur;
wherein each occurrence of R^{9b} is independently hydrogen or an optionally substituted group selected from C₁₋₆ aliphatic, 3- to 10-membered cycloaliphatic, 4- to 10-membered heterocyclyl having 1-5 heteroatoms independently selected from nitrogen, oxygen, or sulfur, 6- to 10-membered aryl, or 5- to 10-membered heteroaryl having 1-5 heteratoms independently selected from nitrogen, oxygen, or sulfur; or two occurrences of R^{9b}, taken together with the nitrogen atom to which they are bound, form an optionally substituted group selected from 3- to 6-membered heterocyclyl having 1-5 heteroatoms independently selected from nitrogen, oxygen, or sulfur, or 5- to 10-membered heteroaryl having 1-5 heteroatoms independently selected from nitrogen, oxygen, or sulfur; and
provided that when HY is a non-fused group then HY is substituted with at least one occurrence of R¹⁰ or R^{10′}, wherein R¹⁰ or R^{10′} is:
—N(R¹¹)C(O)R^{10a}, —C(O)N(R¹¹)₂, or —NR¹¹C(O)OR^{10a}, or
—V₁-T₁-R^{10b}, wherein V₁ is —NR¹¹—, T₁ is a C₁-C₃ alkylene chain, and R^{10b} is an optionally substituted 6- to 10-membered aryl ring or a 5- to 10-membered heteroaryl ring having 1-5 heteroatoms independently selected from nitrogen, oxygen, or sulfur, or V₁ is —NR¹¹C(O)NR¹¹—, T₁ is a C₁-C₃ alkylene chain, and R^{10b} is —OR^{10a}; or
—V₁—R^{10c}, wherein V₁ is —NR¹¹—, and R^{10c} is a 5- to 10-membered heteroaryl ring having 1-5 heteroatoms independently selected from nitrogen, oxygen, or sulfur; and
provided that:
when G₈ is N, R² is methyl, and HY is 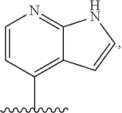 [SMILES: *c1ccnc2[nH]ccc12]
then R¹ is not an optionally substituted phenyl;
when G₈ is CH, then HY is not 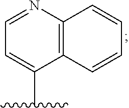 [SMILES: *c1ccnc2ccccc12]
and
provided that the compound is other than:

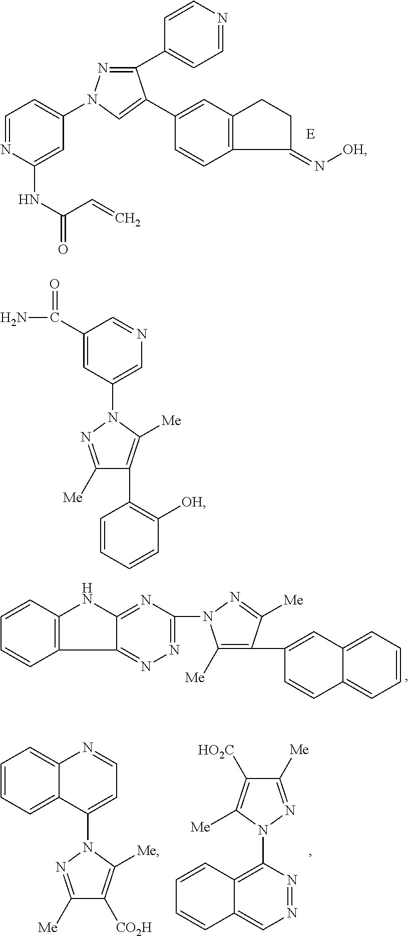

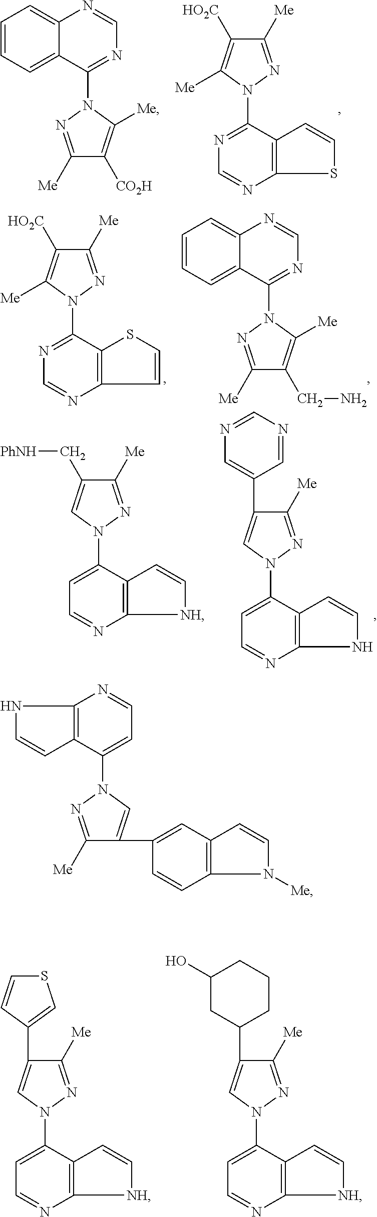

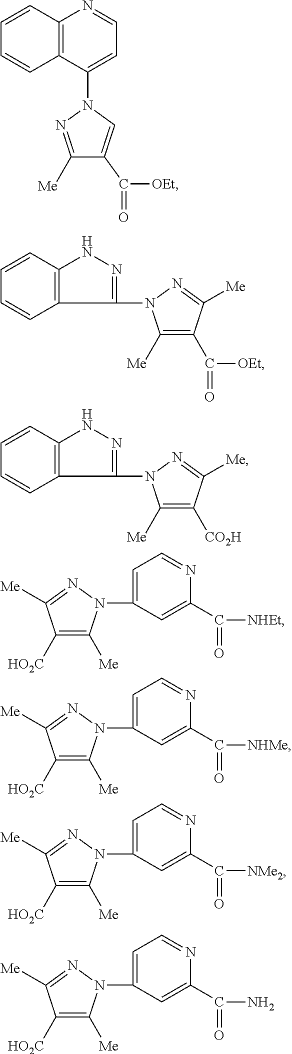

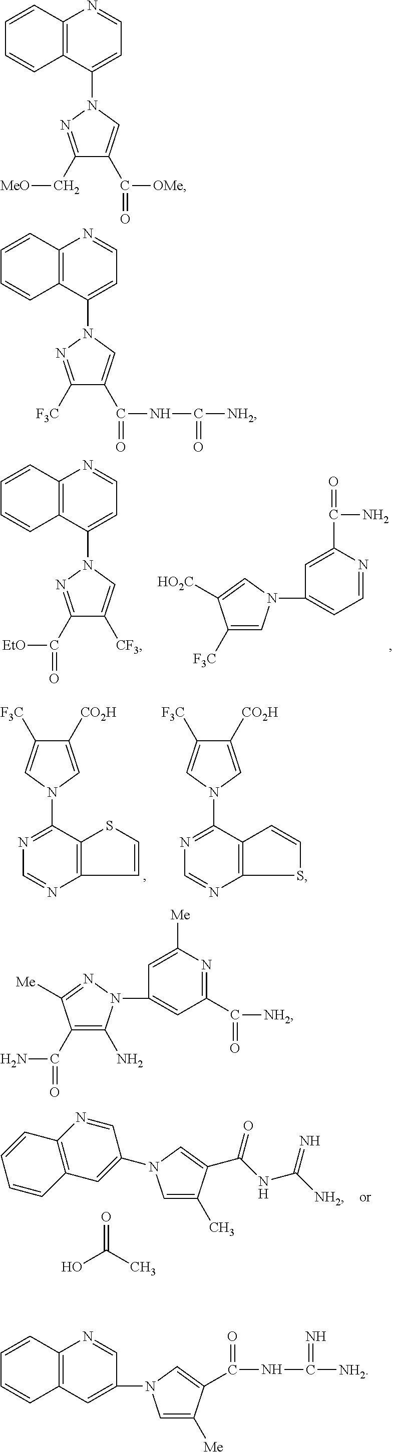

3. The compound of claim 2, provided that when HY is a non-fused group then HY is substituted with at least one occurrence of R¹⁰ or R^{10′}, wherein R¹⁰ or R^{10′} is:
—N(R¹¹)C(O)R^{10a}, —C(O)N(R¹¹)₂, or —NR¹¹C(O)OR^{10a}; or
—V₁-T₁-R^{10b}, wherein V₁ is —NR¹¹—, T₁ is a C₁-C₃ alkylene chain, and R^{10b} is an optionally substituted 6- to 10-membered aryl ring or a 5- to 10-membered heteroaryl ring having 1-5 heteroatoms independently selected from nitrogen, oxygen, or sulfur; or
—V₁—R^{10c}, wherein V₁ is —NR¹¹—, and R^{10c} is a 5- to 10-membered heteroaryl ring having 1-5 heteroatoms independently selected from nitrogen, oxygen, or sulfur.

4. The compound of claim 1, wherein R¹ is CY and CY is 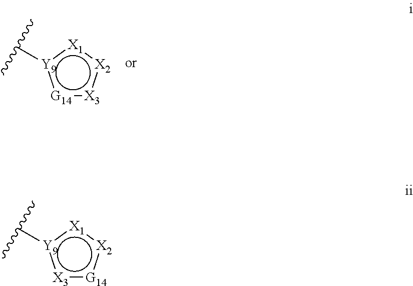
wherein:
X₁, X₂, and X₃, are each independently N, O, S, NR^{4′}, or CR⁷, provided that only one of
X₁, X₂, or X₃ may be O or S;
Y₉ is N or CR⁷;
G₁₄ is CR^{7′}, —N═ or —NR^{4′}—, wherein:
R^{4′} is independently hydrogen, —Z₂—R⁶, optionally substituted C₁₋₆ aliphatic, or optionally substituted 3-10-membered cycloaliphatic, wherein:
Z₂ is selected from an optionally substituted C₁₋₃ alkylene chain, —S(O)—, —S(O)₂—, —C(O)—, —CO₂—, —C(O)NR^{4a}—, or —S(O)₂NR^{4a}—,
R^{4a} is hydrogen or an optionally substituted C₁₋₄ aliphatic, and
R⁶ is hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur;
each occurrence of R⁷ and R^{7′} is independently hydrogen, —CN, halogen, —NH₂, —Z₃—R⁸, C₁₋₆ aliphatic, or 3-10-membered cycloaliphatic, wherein:
Z₃ is selected from an optionally substituted C₁₋₃ alkylene chain, —O—, —N(R^{7a})—, —S—, —S(O)—, —S(O)₂—, —C(O)—, —CO₂—, —C(O)NR^{7a}—, —N(R^{7a})C(O)—, —N(R^{7a})CO₂—, —S(O)₂NR^{7a}—, —N(R^{7a})S(O)₂—, —OC(O)N(R^{7a})—, —N(R^{7a})C(O)NR^{7a}—, —N(R^{7a})S(O)₂N(R^{7a})—, or —OC(O)—;
R^{7a} is hydrogen or an optionally substituted C₁₋₄ aliphatic, and
R⁸ is hydrogen or an optionally substituted group selected from C₁₋₆ aliphatic, 3-10-membered cycloaliphatic, 4-10-membered heterocyclyl having 1-5 heteroatoms independently selected from nitrogen, oxygen, or sulfur, 6-10-membered aryl, or 5-10-membered heteroaryl having 1-5 heteroatoms independently selected from nitrogen, oxygen, or sulfur.

5. The compound of claim 4, wherein CY is

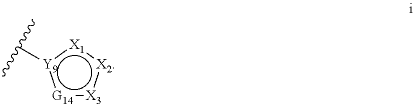

6. The compound of claim 5, wherein Y₉ is carbon, X₁ is nitrogen, G₁₄ is N(R^{4′}), and X₂ and X₃, are CH.

7. The compound of claim 5, wherein Y₉ is carbon, X₁ and X₃ are nitrogen, G₁₄ is N(R^{4′}), and X₂ is CH.

8. The compound of claim 5, wherein Y₉ is carbon, X₁ and G₁₄ are nitrogen, X₃ is N(R^{4′}), and X₂ is CH.

9. The compound of claim 5, wherein Y₉ is carbon, X₁ and X₂ are nitrogen, G₁₄ is N(R^{4′}), and X₃ is CH.

10. The compound of claim 5, wherein Y₉ is carbon, G₁₄ is N(R^{4′}), X₃ is nitrogen, and X₁ and X₂ CH.

11. The compound of claim 5, wherein Y₉ is carbon, G₁₄ is nitrogen, X₃ is N(R^{4′}), and X₁ and X₂ are CH.

12. The compound of claim 5, wherein Y₉ is carbon, X₃ is nitrogen, X₂ is N(R^{4′}), and X₁ and G₁₄ are CH.

13. The compound of claim 5, wherein Y₉ is carbon, X₂ is nitrogen, G₁₄ is N(R^{4′}), and X₁ and X₃, are CH.

14. The compound of claim 5, wherein Y₉ is carbon, X₂ is N(R^{4′}), G₁₄ is nitrogen, and X₁ and X₃, are CH.

15. The compound of claim 1, wherein R¹ is Cy, and Cy is an optionally substituted 5- to 6-membered heteroaryl or heterocyclyl ring.

16. The compound of claim 15, wherein Cy is selected from: 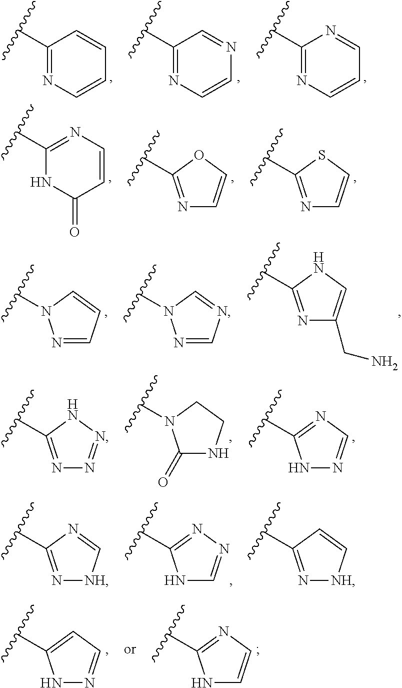
and Cy is optionally further substituted with one or more occurrences of R⁷ or R^{4′}.

17. The compound of claim 1, wherein R¹ is Cy, and Cy is an optionally substituted 6-membered aryl ring.

18. The compound of claim 1, wherein R¹ is —CON(R⁴)₂, —NHCOR⁴, or —COOR⁴.

19. The compound of claim 1, wherein HY is selected from:

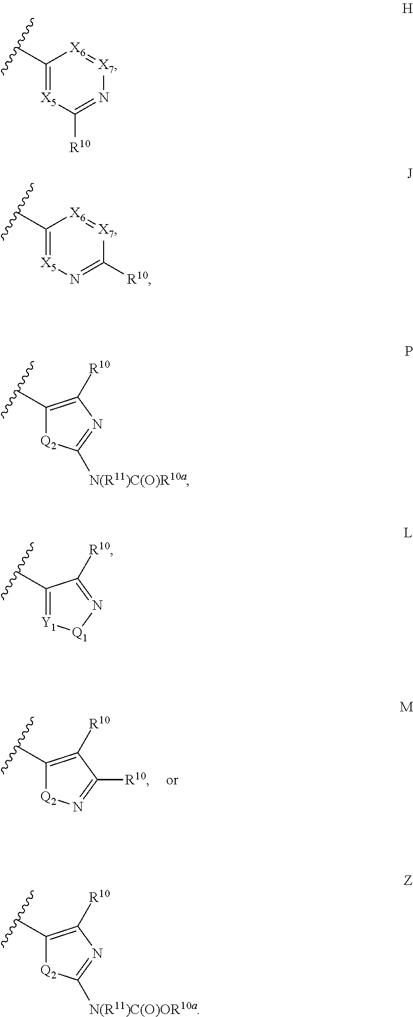

20. The compound of claim 19, wherein HY is selected from: 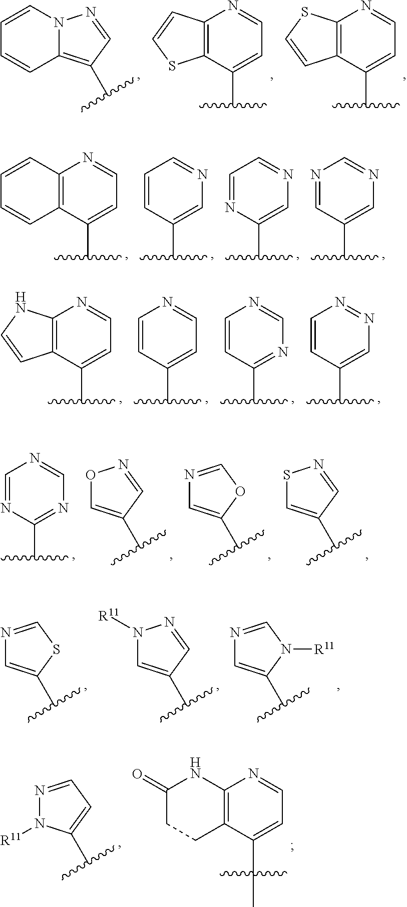
wherein each fused HY group is unsubstituted, and
each non-fused HY group is substituted with one or more occurrences of R¹⁰ or R^{10′}, and at least one occurrence of R¹⁰ or R^{10′} is —N(R¹¹)C(O)R^{10a}, —N(R¹¹)C(O)OR^{10a} or —C(O)N(R¹¹)₂, and the dashed line represents a single bond or a double bond.

21. The compound of claim 1, wherein R^{10a} is C₁₆ aliphatic substituted with a 5-10-membered heteroaryl having 1-5 heteroatoms independently selected from nitrogen, oxygen, or sulfur.

22. The compound of claim 20, wherein HY is selected from: 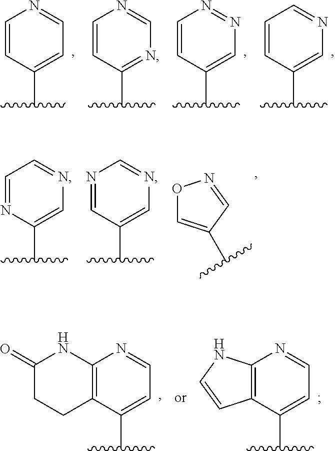
wherein each fused HY group is unsubstituted, and
each non-fused HY group is substituted with one or more occurrences of R¹⁰ or R^{10′}, and at least one occurrence of R¹⁰ or R^{10′} is —N(R¹¹)C(O)R^{10a}, —N(R¹¹)C(O)OR^{10a} or —C(O)N(R¹¹)₂, and the dashed line represents a single bond or a double bond.

23. The compound of claim 22, wherein HY is 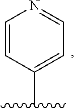 [SMILES: *c1ccncc1]
and HY is substituted with one or more occurrences of R¹⁰ or R^{10′}.

24. The compound of claim 23, wherein HY is 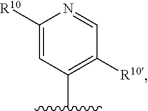
wherein R^{10′} is hydrogen, methyl, chloro, bromo, fluoro, CN, CF₃, OR^{10c}, COR^{10c}, and R¹⁰ is NHCOR^{10c} or —NHC(O)OR^{10c}.

25. The compound of claim 24, wherein R¹⁰ is hydrogen, methyl, or chloro.

26. The compound of claim 25, wherein R¹⁰ is methyl, and R¹⁰ is —NHCOR^{10c}.

27. The compound of claim 23, wherein R¹⁰ is —NHR¹¹, wherein R¹¹ is an optionally substituted 5- to 10-membered heteroaryl having 1-5 heteroatoms independently selected from nitrogen, oxygen, or sulfur.

28. The compound of claim 1, wherein R^{10a} is cyclopropyl, methyl, ethyl, or isopropyl.

29. The compound of claim 1, wherein R² is a 6-10-membered aryl or 5-10-membered heteroaryl having 1-5 heteroatoms independently selected from nitrogen, oxygen, or sulfur; optionally substituted with 1-3 occurrences of R^{2a}.

30. The compound of claim 29, wherein R² is a phenyl group; optionally substituted with one or more independent occurrences of halogen, C₁₋₃ alkyl, —CN, C₁₋₃ haloalkyl, —(CH₂)ₚN(R^{12b})₂, —OR^{12b}, —NHC(O)R^{12b}, —NHC(O)NHR^{12b}, —NHS(O)₂R^{12b}, —S(O)₂R^{12c}, —S(O)₂N(R¹²)₂, C(O)OR^{12b}, —C(O)N(R^{12b})₂, or —C(O)R^{12b}, and wherein p is 0 to 3.

31. The compound of claim 30, wherein R² is a phenyl group; optionally substituted with one or more independent occurrences of halogen, C₁₋₃ alkyl, —CN, C₁₋₃ haloalkyl, —CH₂N(CH₃)₂, —OC₁₋₃ alkyl, —OC₁₋₃ haloalkyl, —NHC(O)C₁₋₃ alkyl, —NHC(O)NHC₁₋₃ alkyl, —NHS(O)₂C₁₋₃ alkyl, or —C(O)H.

32. The compound of claim 31, wherein R² is a phenyl group substituted with 1 or 2 occurrences of halogen.

33. The compound of claim 1, wherein R² is a 3-10-membered cycloaliphatic, 4-10-membered heterocyclyl having 1-5 heteroatoms independently selected from nitrogen, oxygen, or sulfur.

34. The compound of claim 33, wherein R² is an optionally substituted N-linked 3-, 4-, 5-, 6-, or 7-membered heterocyclyl ring, optionally substituted with one or more occurrences of R^{2a}.

35. The compound of claim 34, wherein R² is optionally substituted with one or more C₁₋₃ alkyl groups, —OR^{12b}, or —NR^{12b}.

36. The compound of claim 1, wherein R² is a C₁₋₆ aliphatic and each occurrence of R^{2a} is independently —C(O)OR^{12b}, —C(O)N(R^{12b})₂, —S(O)₂N(R^{12b})₂, —N(R^{12e})C(O)R^{12b}, or —N(R^{12e})SO₂R^{12c}.

37. The compound of claim 1, wherein R¹ is CY, —CON(R⁴)₂, —NHCOR⁴, or —COOR⁴; R² is optionally substituted aryl or heteroaryl; and HY is selected from 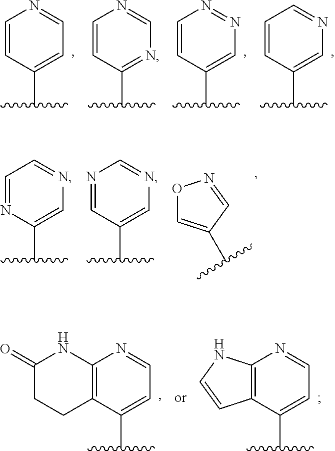
wherein each fused HY group is unsubstituted, and
each non-fused HY group is substituted with one or more occurrences of R¹⁰ or R^{10′}, and at least one occurrence of R¹⁰ or R^{10′} is —N(R¹¹)C(O)R^{10a} or —C(O)N(R¹¹)₂, and the dashed line represents a single bond or a double bond.

38. The compound of claim 1 having the structure of formula IIB: 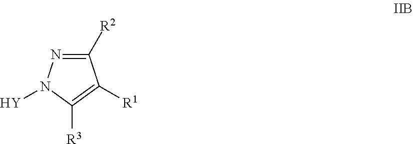
or a pharmaceutically acceptable salt thereof.

39. The compound of claim 1 having the structure of formula IIC: 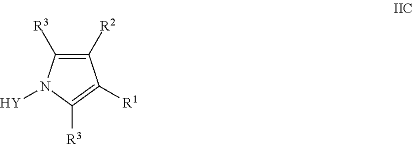
or a pharmaceutically acceptable salt thereof.

40. The compound of claim 1 having the structure of formula VB: 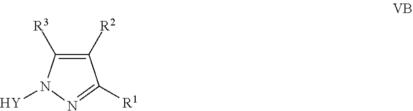
or a pharmaceutically acceptable salt thereof.

41. The compound of claim 1, wherein the compound is selected from: 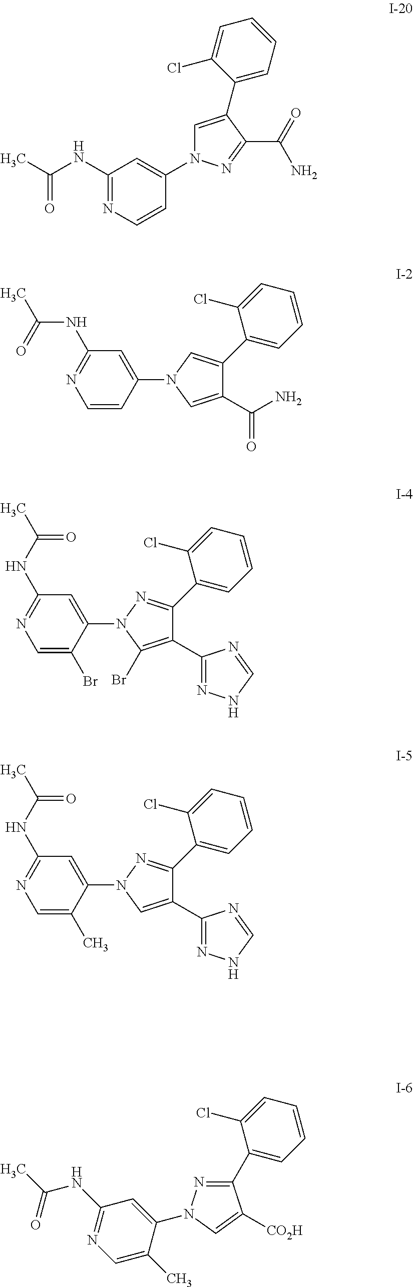 [SMILES: CC(=O)Nc1cc(-n2cc(-c3ccccc3Cl)c(C(N)=O)n2)ccn1 ; CC(=O)Nc1cc(-n2cc(C(N)=O)c(-c3ccccc3Cl)c2)ccn1 ; CC(=O)Nc1cc(-n2nc(-c3ccccc3Cl)c(-c3nc[nH]n3)c2Br)c(Br)cn1 ; CC(=O)Nc1cc(-n2cc(-c3nc[nH]n3)c(-c3ccccc3Cl)n2)c(C)cn1 ; CC(=O)Nc1cc(-n2cc(C(=O)O)c(-c3ccccc3Cl)n2)c(C)cn1] 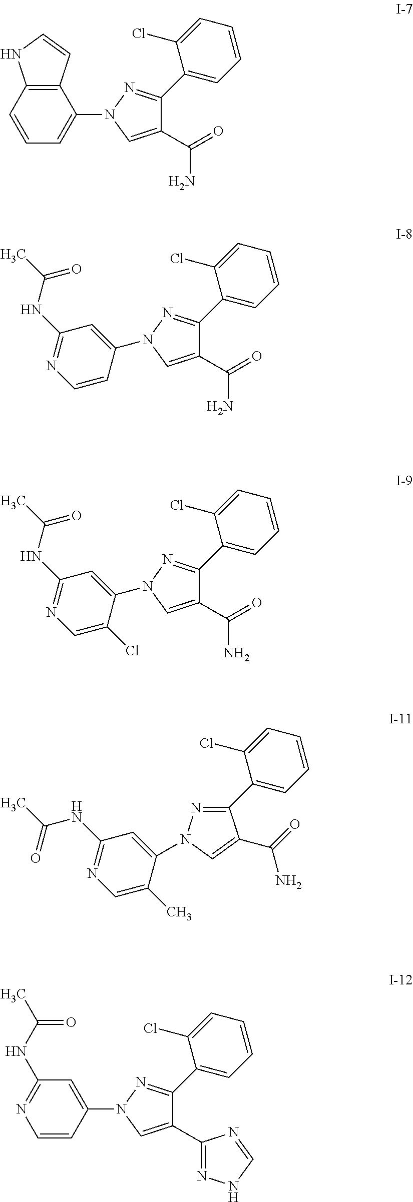 [SMILES: NC(=O)c1cn(-c2cccc3[nH]ccc23)nc1-c1ccccc1Cl ; CC(=O)Nc1cc(-n2cc(C(N)=O)c(-c3ccccc3Cl)n2)ccn1 ; CC(=O)Nc1cc(-n2cc(C(N)=O)c(-c3ccccc3Cl)n2)c(Cl)cn1 ; CC(=O)Nc1cc(-n2cc(C(N)=O)c(-c3ccccc3Cl)n2)c(C)cn1 ; CC(=O)Nc1cc(-n2cc(-c3nc[nH]n3)c(-c3ccccc3Cl)n2)ccn1] 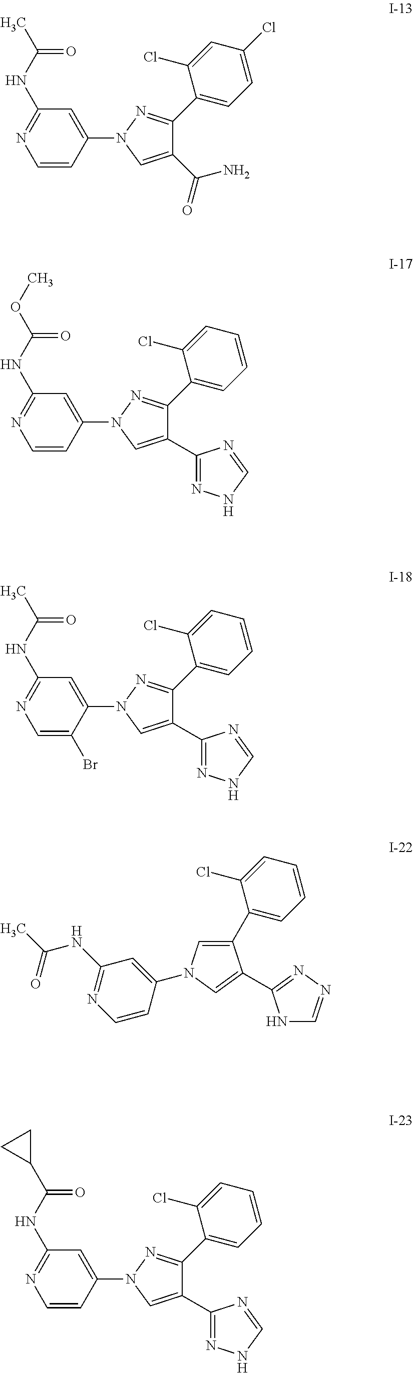 [SMILES: CC(=O)Nc1cc(-n2cc(C(N)=O)c(-c3ccc(Cl)cc3Cl)n2)ccn1 ; COC(=O)Nc1cc(-n2cc(-c3nc[nH]n3)c(-c3ccccc3Cl)n2)ccn1 ; CC(=O)Nc1cc(-n2cc(-c3nc[nH]n3)c(-c3ccccc3Cl)n2)c(Br)cn1 ; CC(=O)Nc1cc(-n2cc(-c3nnc[nH]3)c(-c3ccccc3Cl)c2)ccn1 ; O=C(Nc1cc(-n2cc(-c3nc[nH]n3)c(-c3ccccc3Cl)n2)ccn1)C1CC1] 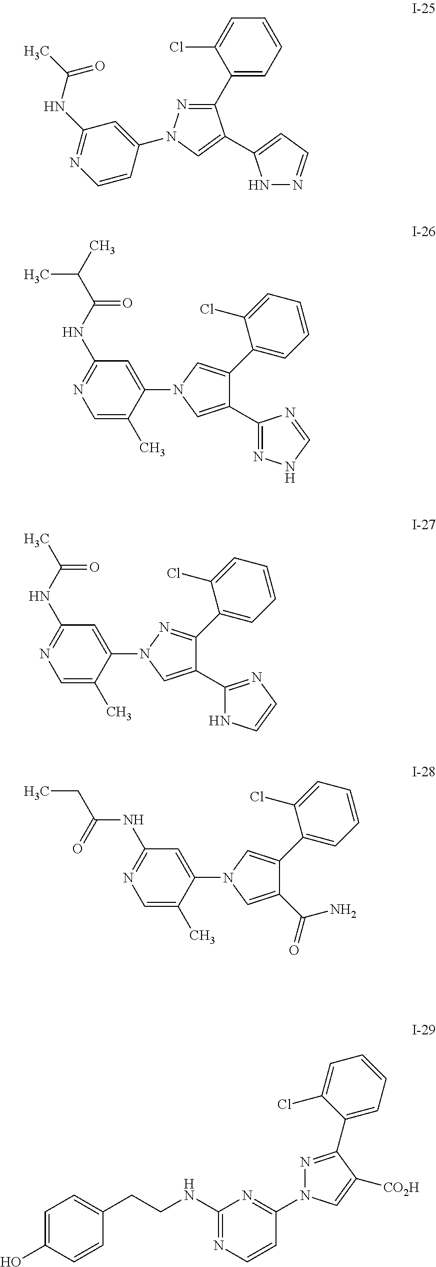 [SMILES: CC(=O)Nc1cc(-n2cc(-c3ccn[nH]3)c(-c3ccccc3Cl)n2)ccn1 ; Cc1cnc(NC(=O)C(C)C)cc1-n1cc(-c2nc[nH]n2)c(-c2ccccc2Cl)c1 ; CC(=O)Nc1cc(-n2cc(-c3ncc[nH]3)c(-c3ccccc3Cl)n2)c(C)cn1 ; CCC(=O)Nc1cc(-n2cc(C(N)=O)c(-c3ccccc3Cl)c2)c(C)cn1 ; O=C(O)c1cn(-c2ccnc(NCCc3ccc(O)cc3)n2)nc1-c1ccccc1Cl] 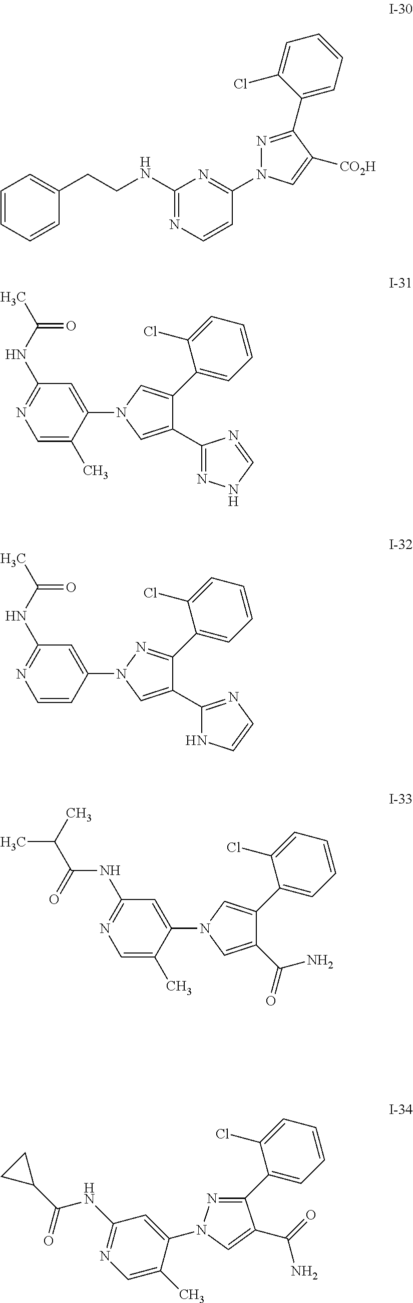 [SMILES: O=C(O)c1cn(-c2ccnc(NCCc3ccccc3)n2)nc1-c1ccccc1Cl ; CC(=O)Nc1cc(-n2cc(-c3nc[nH]n3)c(-c3ccccc3Cl)c2)c(C)cn1 ; CC(=O)Nc1cc(-n2cc(-c3ncc[nH]3)c(-c3ccccc3Cl)n2)ccn1 ; Cc1cnc(NC(=O)C(C)C)cc1-n1cc(C(N)=O)c(-c2ccccc2Cl)c1 ; Cc1cnc(NC(=O)C2CC2)cc1-n1cc(C(N)=O)c(-c2ccccc2Cl)n1] 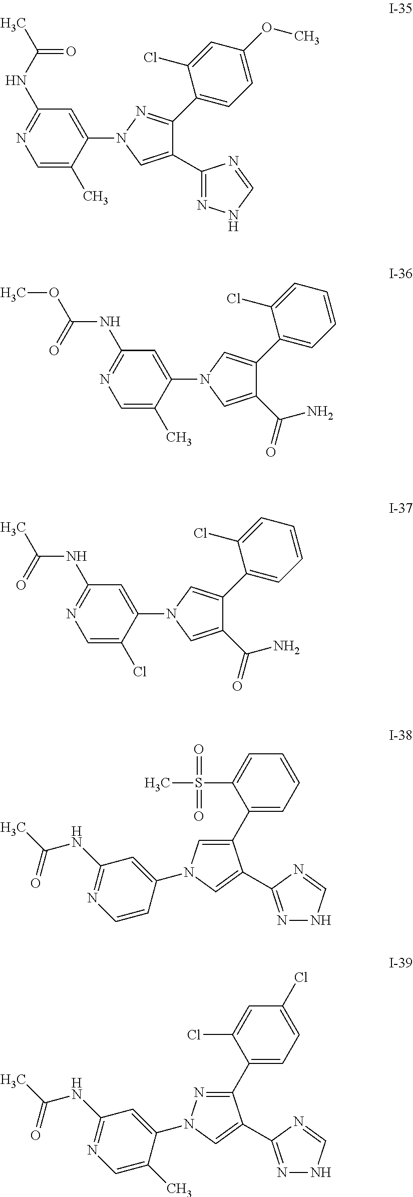 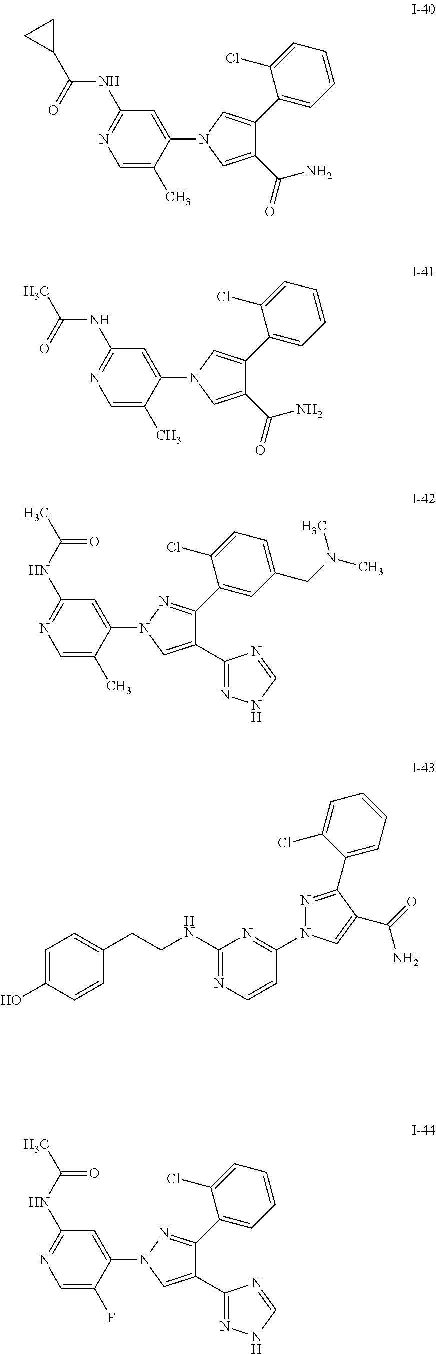 [SMILES: Cc1cnc(NC(=O)C2CC2)cc1-n1cc(C(N)=O)c(-c2ccccc2Cl)c1 ; CC(=O)Nc1cc(-n2cc(C(N)=O)c(-c3ccccc3Cl)c2)c(C)cn1 ; CC(=O)Nc1cc(-n2cc(-c3nc[nH]n3)c(-c3cc(CN(C)C)ccc3Cl)n2)c(C)cn1 ; NC(=O)c1cn(-c2ccnc(NCCc3ccc(O)cc3)n2)nc1-c1ccccc1Cl ; CC(=O)Nc1cc(-n2cc(-c3nc[nH]n3)c(-c3ccccc3Cl)n2)c(F)cn1] 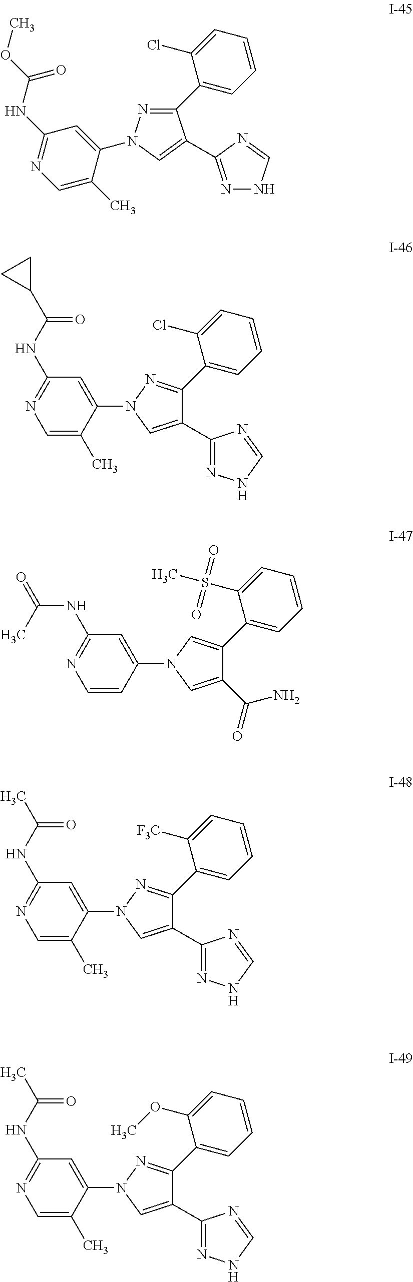 [SMILES: COC(=O)Nc1cc(-n2cc(-c3nc[nH]n3)c(-c3ccccc3Cl)n2)c(C)cn1 ; Cc1cnc(NC(=O)C2CC2)cc1-n1cc(-c2nc[nH]n2)c(-c2ccccc2Cl)n1 ; CC(=O)Nc1cc(-n2cc(C(N)=O)c(-c3ccccc3S(C)(=O)=O)c2)ccn1 ; CC(=O)Nc1cc(-n2cc(-c3nc[nH]n3)c(-c3ccccc3C(F)(F)F)n2)c(C)cn1 ; COc1ccccc1-c1nn(-c2cc(NC(C)=O)ncc2C)cc1-c1nc[nH]n1] 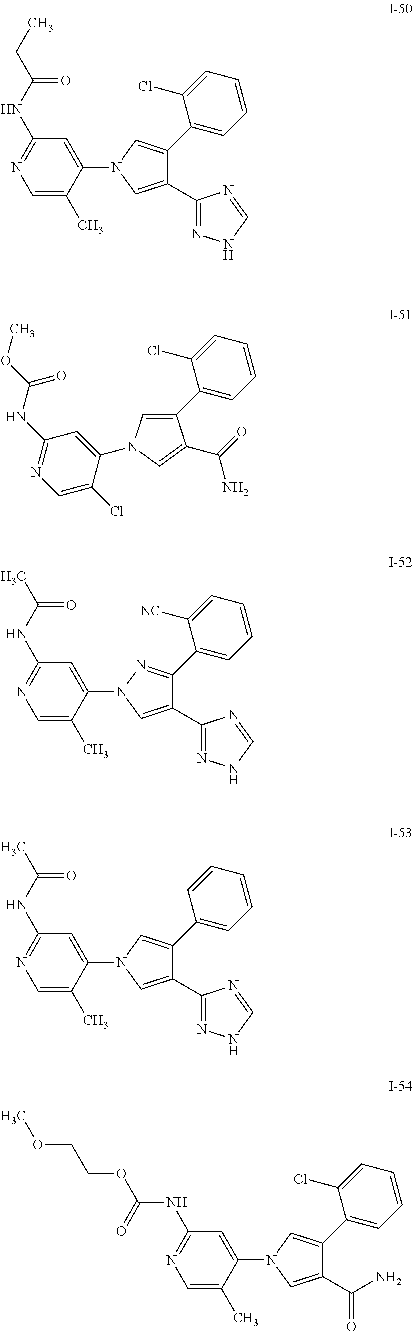 [SMILES: CCC(=O)Nc1cc(-n2cc(-c3nc[nH]n3)c(-c3ccccc3Cl)c2)c(C)cn1 ; COC(=O)Nc1cc(-n2cc(C(N)=O)c(-c3ccccc3Cl)c2)c(Cl)cn1 ; CC(=O)Nc1cc(-n2cc(-c3nc[nH]n3)c(-c3ccccc3C#N)n2)c(C)cn1 ; CC(=O)Nc1cc(-n2cc(-c3ccccc3)c(-c3nc[nH]n3)c2)c(C)cn1 ; COCCOC(=O)Nc1cc(-n2cc(C(N)=O)c(-c3ccccc3Cl)c2)c(C)cn1] 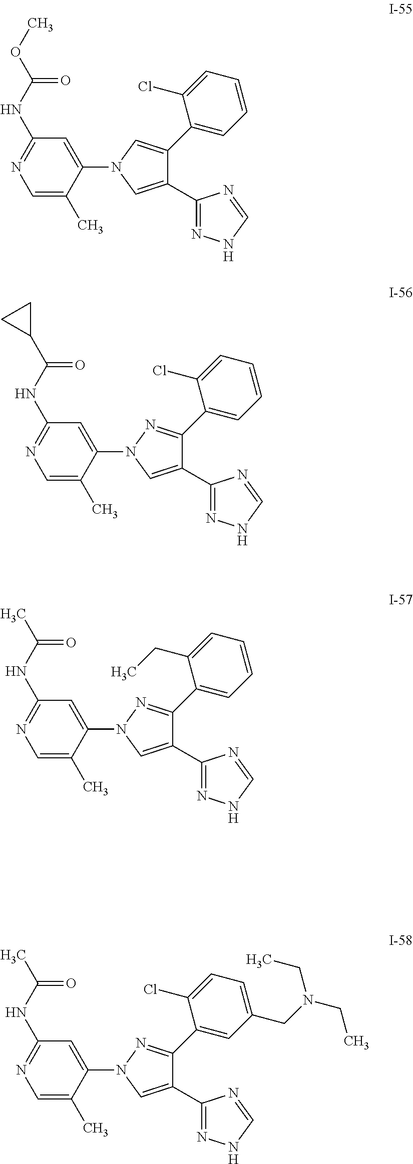 [SMILES: COC(=O)Nc1cc(-n2cc(-c3nc[nH]n3)c(-c3ccccc3Cl)c2)c(C)cn1 ; Cc1cnc(NC(=O)C2CC2)cc1-n1cc(-c2nc[nH]n2)c(-c2ccccc2Cl)n1 ; CCc1ccccc1-c1nn(-c2cc(NC(C)=O)ncc2C)cc1-c1nc[nH]n1 ; CCN(CC)Cc1ccc(Cl)c(-c2nn(-c3cc(NC(C)=O)ncc3C)cc2-c2nc[nH]n2)c1] 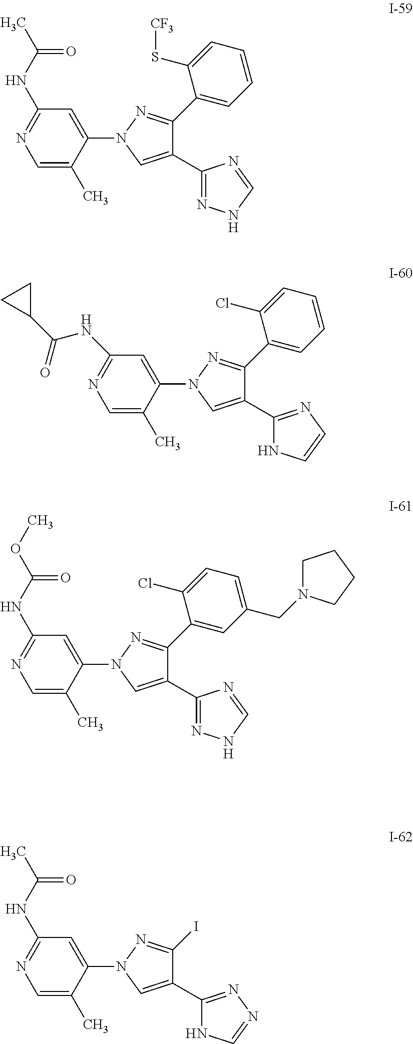 [SMILES: CC(=O)Nc1cc(-n2cc(-c3nc[nH]n3)c(-c3ccccc3SC(F)(F)F)n2)c(C)cn1 ; Cc1cnc(NC(=O)C2CC2)cc1-n1cc(-c2ncc[nH]2)c(-c2ccccc2Cl)n1 ; COC(=O)Nc1cc(-n2cc(-c3nc[nH]n3)c(-c3cc(CN4CCCC4)ccc3Cl)n2)c(C)cn1 ; CC(=O)Nc1cc(-n2cc(-c3nnc[nH]3)c(I)n2)c(C)cn1] 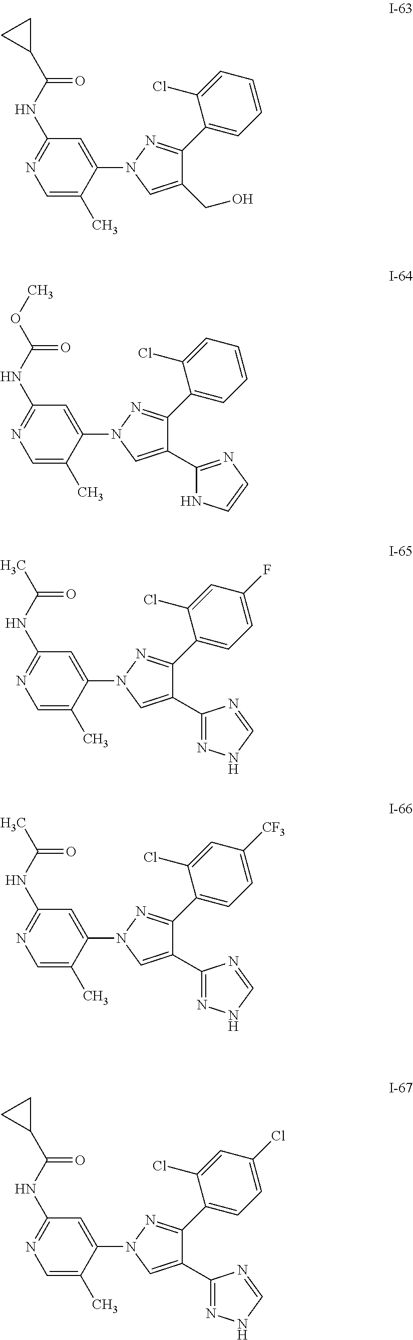 [SMILES: Cc1cnc(NC(=O)C2CC2)cc1-n1cc(CO)c(-c2ccccc2Cl)n1 ; COC(=O)Nc1cc(-n2cc(-c3ncc[nH]3)c(-c3ccccc3Cl)n2)c(C)cn1 ; CC(=O)Nc1cc(-n2cc(-c3nc[nH]n3)c(-c3ccc(F)cc3Cl)n2)c(C)cn1 ; CC(=O)Nc1cc(-n2cc(-c3nc[nH]n3)c(-c3ccc(C(F)(F)F)cc3Cl)n2)c(C)cn1 ; Cc1cnc(NC(=O)C2CC2)cc1-n1cc(-c2nc[nH]n2)c(-c2ccc(Cl)cc2Cl)n1] 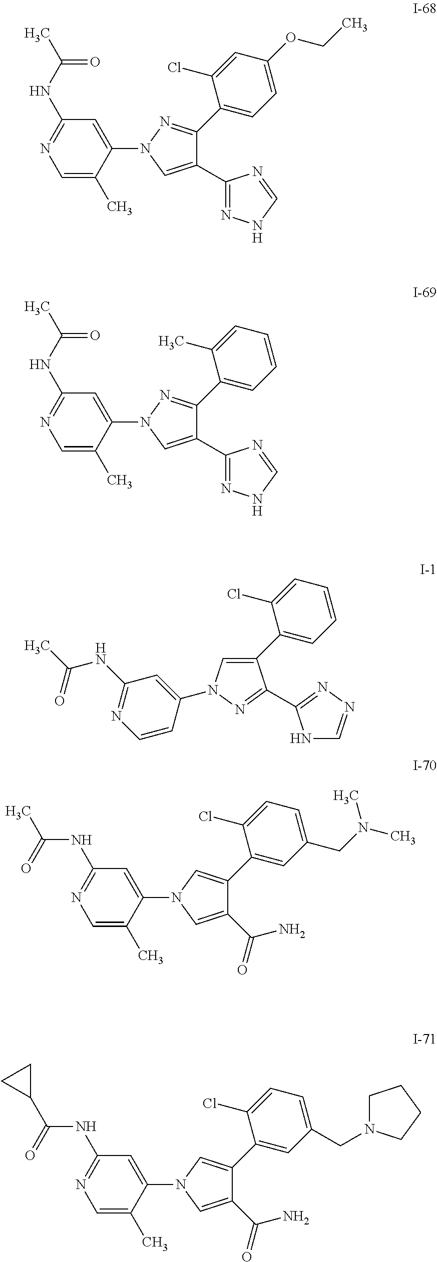 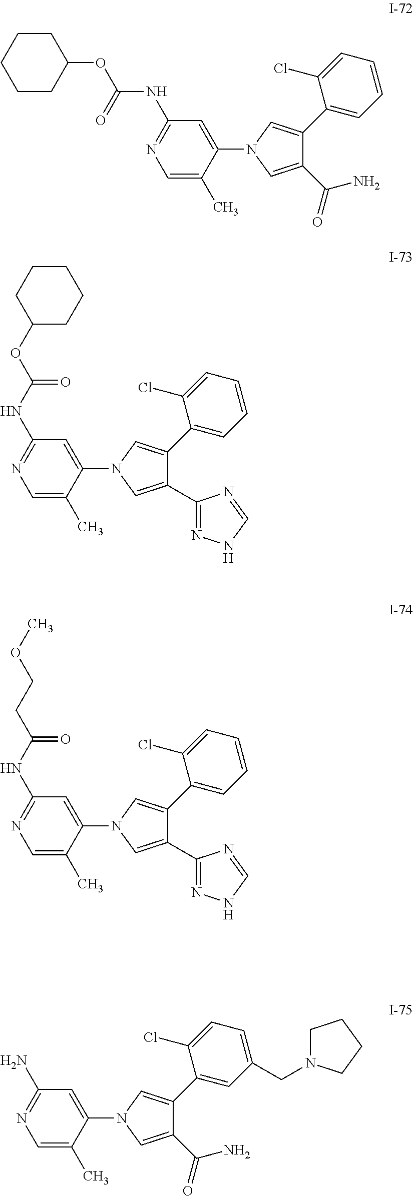 [SMILES: Cc1cnc(NC(=O)OC2CCCCC2)cc1-n1cc(C(N)=O)c(-c2ccccc2Cl)c1 ; Cc1cnc(NC(=O)OC2CCCCC2)cc1-n1cc(-c2nc[nH]n2)c(-c2ccccc2Cl)c1 ; COCCC(=O)Nc1cc(-n2cc(-c3nc[nH]n3)c(-c3ccccc3Cl)c2)c(C)cn1 ; Cc1cnc(N)cc1-n1cc(C(N)=O)c(-c2cc(CN3CCCC3)ccc2Cl)c1] 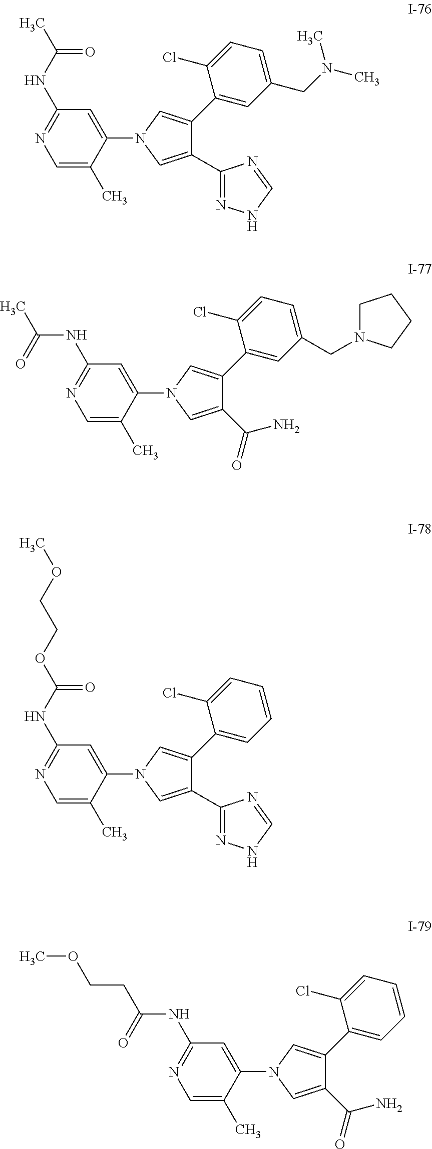 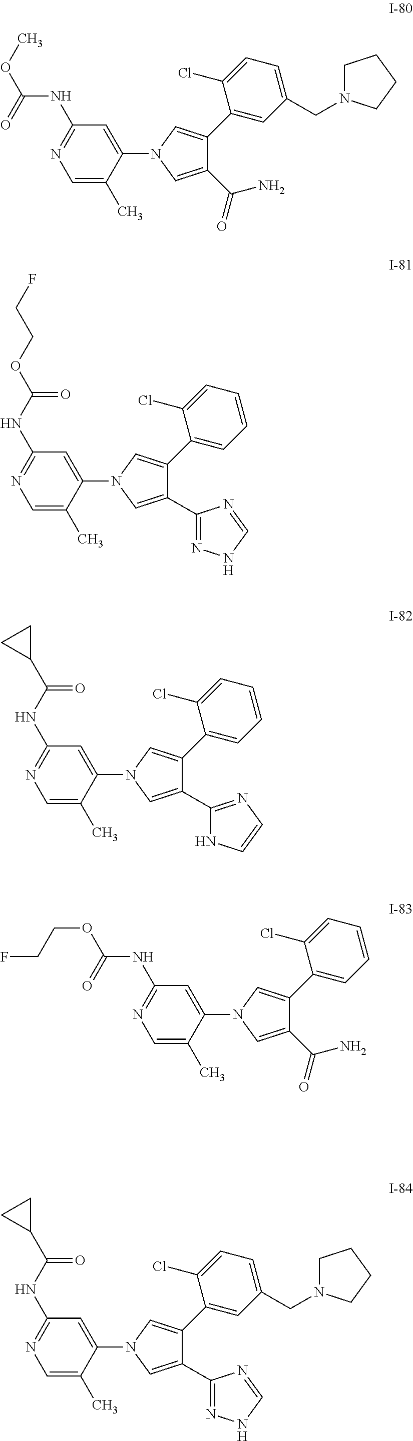 [SMILES: COC(=O)Nc1cc(-n2cc(C(N)=O)c(-c3cc(CN4CCCC4)ccc3Cl)c2)c(C)cn1 ; Cc1cnc(NC(=O)OCCF)cc1-n1cc(-c2nc[nH]n2)c(-c2ccccc2Cl)c1 ; Cc1cnc(NC(=O)C2CC2)cc1-n1cc(-c2ncc[nH]2)c(-c2ccccc2Cl)c1 ; Cc1cnc(NC(=O)OCCF)cc1-n1cc(C(N)=O)c(-c2ccccc2Cl)c1 ; Cc1cnc(NC(=O)C2CC2)cc1-n1cc(-c2nc[nH]n2)c(-c2cc(CN3CCCC3)ccc2Cl)c1] 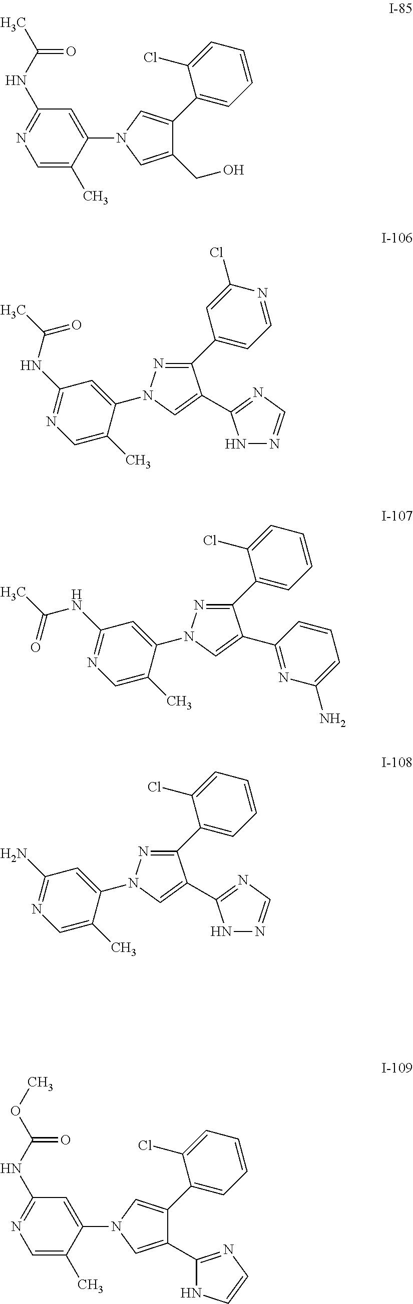 [SMILES: CC(=O)Nc1cc(-n2cc(CO)c(-c3ccccc3Cl)c2)c(C)cn1 ; CC(=O)Nc1cc(-n2cc(-c3ncn[nH]3)c(-c3ccnc(Cl)c3)n2)c(C)cn1 ; CC(=O)Nc1cc(-n2cc(-c3cccc(N)n3)c(-c3ccccc3Cl)n2)c(C)cn1 ; Cc1cnc(N)cc1-n1cc(-c2ncn[nH]2)c(-c2ccccc2Cl)n1 ; COC(=O)Nc1cc(-n2cc(-c3ncc[nH]3)c(-c3ccccc3Cl)c2)c(C)cn1]
or a pharmaceutically acceptable salt thereof.

42. A pharmaceutical composition comprising a compound of claim 1, and a pharmaceutically acceptable carrier.

43. The pharmaceutical composition of claim 42, further comprising another therapeutic agent.

44. A method of treating a proliferative disorder in a patient comprising administering to said patient a therapeutically effective amount of a compound of claim 1.

45. The method of claim 44, wherein the proliferative disorder is breast cancer, bladder cancer, colon cancer, glioma, glioblastoma, lung cancer, hepatocellular cancer, gastric cancer, melanoma, thyroid cancer, endometrial cancer, renal cancer, cervical cancer, pancreatic cancer, esophageal cancer, prostate cancer, brain cancer, or ovarian cancer.

46. A method of treating an inflammatory or cardiovascular disorder in a patient comprising administering to said patient a therapeutically effective amount of a compound of claim 1.

47. The method of claim 46, wherein the inflammatory or cardiovascular disorder is selected from allergies/anaphylaxis, acute and chronic inflammation, rheumatoid arthritis, autoimmunity disorders, thrombosis, hypertension, cardiac hypertrophy, and heart failure.

48. A method for inhibiting VPS34 or PI3K activity in a patient comprising administering a composition comprising a therapeutically effective amount of a compound of claim 1.
